(19) United States
(12) Patent Application Publication
(10) Pub. No.: US 2024/0358020 A1
(43) Pub. Date: Oct. 31, 2024

(54) FORMAMIDE DERIVATIVE AND AGRICULTURAL OR HORTICULTURAL PLANT DISEASE CONTROL AGENT

(71) Applicant: Kumiai Chemical Industry Co., Ltd., Tokyo (JP)

(72) Inventors: Kosuke TANAKA, Tokyo (JP); Kento HORINOUCHI, Tokyo (JP); Takuto OE, Tokyo (JP); Shunsuke YOKOCHI, Tokyo (JP); Isao KANEKO, Tokyo (JP)

(73) Assignee: Kumiai Chemical Industry Co., Ltd., Tokyo (JP)

(21) Appl. No.: 18/578,919

(22) PCT Filed: Jul. 15, 2022
(86) PCT No.: PCT/JP2022/027824

§ 371 Date: Jan. 12, 2024

(30) Foreign Application Priority Data
Jul. 15, 2021 (JP) 2021-117258

(51) Int. Cl. A01N 43/82; A01N 37/52; A01P 3/00; C07C 259/18; C07D 271/06

(52) U.S. Cl. CPC A01N 43/82; A01N 37/52; A01P 3/00; C07C 259/18; C07D 271/06

(57) ABSTRACT

The purpose of the present invention is to provide an excellent agricultural or horticultural plant disease control agent. Provided are: a formamide derivative represented by general formula [I] and the like (in the formula, X¹, X², X³, and X⁴ each independently represents a carbon atom and the like, Y represents an oxygen atom or a sulfur atom, R¹ represents a halogen atom, and R² represents a hydrogen atom, a C₁-C₆ alkyl group, and the like) or an agriculturally acceptable salt thereof; and an agricultural or horticultural plant disease control agent containing the same as an active ingredient.

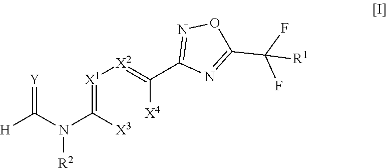

### TECHNICAL FIELD

The present invention relates to a novel formamide derivative or an agriculturally acceptable salt thereof, an agricultural and horticultural plant disease control agent containing the derivative or the salt thereof as an active ingredient, and a production intermediate of the derivative or the salt thereof.

### BACKGROUND ART

Patent Document 1 describes an amide derivative containing aryl oxadiazole.

Patent Documents 2 to 6 describe an amide derivative containing aryl oxadiazole and heteroaryl oxadiazole.

Patent Documents 7 and 8 describe an aniline derivative containing aryl oxadiazole and heteroaryl oxadiazole.

Patent Document 9 describes a di-substituted amide derivative containing aryl oxadiazole.

In the patent documents described above, a formamide derivative of the present invention is not disclosed at all.

### CITATION LIST

### Patent Document

Patent Document 1: JP 63-162680 A
Patent Document 2: WO 2017/081309 A
Patent Document 3: WO 2017/081310 A
Patent Document 4: WO 2009/019656 A
Patent Document 5: WO 2013/080120 A
Patent Document 6: WO 2017/222951 A
Patent Document 7: WO 2020/070610 A
Patent Document 8: WO 2020/254494 A
Patent Document 9: WO 2017/110862 A

### SUMMARY OF THE INVENTION

### Problems to be Solved by the Invention

In cultivation of agricultural and horticultural crops, a large number of control agents are used for diseases of crops. However, there are few control agents according to the background art which fully satisfy the requirements for use as a control agent from the viewpoint of an insufficient control effect thereof, limited use thereof due to the emergence of agent-resistant pathogens, phytotoxic or contaminating effects on plants, or toxicity to humans, domestic animals, and fish, and an adverse effect on the environment. Therefore, there is a strong demand for the development of a control agent that has few such defects and can be safely used.

### Means for Solving the Problems

As a result of conducting intensive studies, the present inventors have found that an agent containing a formamide derivative of formula [I] or an agriculturally acceptable salt thereof as an active ingredient has a high control effect on diseases of useful crops, thereby completing the present invention.

That is, the gist of the present invention is as follows.

(1) A formamide derivative of formula [I] or an agriculturally acceptable salt thereof,

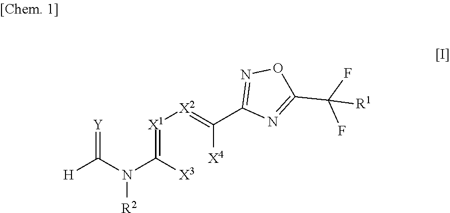

wherein in the formula [I],
X¹, X², X³, and X⁴ each independently represent a carbon atom (the carbon atom is optionally substituted with R³) or a nitrogen atom,
Y represents an oxygen atom or a sulfur atom,
R¹ represents a hydrogen atom or a halogen atom,
R² represents a hydrogen atom, a (C1-C6)alkyl (the (C1-C6)alkyl is optionally mono-substituted or poly-substituted with R⁴), a (C2-C6)alkenyl (the (C2-C6)alkenyl is optionally mono-substituted or poly-substituted with R⁴), a (C2-C6)alkynyl (the (C2-C6)alkynyl is optionally mono-substituted or poly-substituted with R⁴), a (C3-C6)cycloalkyl (the (C3-C6)cycloalkyl is optionally mono-substituted or poly-substituted with R⁴), a (C6-C12)aryl (the (C6-C12)aryl is optionally mono-substituted or poly-substituted with R⁵), a 3- to 6-membered heterocyclyl (the 3- to 6-membered heterocyclyl is optionally mono-substituted or poly-substituted with R⁵), or a 5- to 12-membered heteroaryl (the 5- to 12-membered heteroaryl is optionally mono-substituted or poly-substituted with R⁵),
R³ each independently represent a hydrogen atom, a halogen atom, or a (C1-C6)alkyl,
R⁴ each independently represent a hydrogen atom, a halogen atom, a cyano, a thiocyanato, a thiol, a hydroxy, an amino, a tri((C1-C6)alkyl)silyl, a (C3-C6)cycloalkyl, a (C3-C6)halocycloalkyl, a (C1-C6)alkylamino (the (C1-C6)alkyl moiety is optionally mono-substituted or poly-substituted with R³, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R³, and the amino moiety is optionally substituted with R⁶), a formyl, a (C1-C6)alkylcarbonyl (the (C1-C6)alkylcarbonyl is optionally mono-substituted or poly-substituted with R³), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R³), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6) cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryl (the (C6-C12)aryl is optionally mono-substituted or poly-substituted with R⁵), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵),
a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyl (the 3- to 6-membered heterocyclyl is optionally mono-substituted or poly-substituted with R⁵), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryl (the 5- to 12-membered heteroaryl is optionally mono-substituted or poly-substituted with R⁵), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a 1,3-benzodioxole (the 1,3-benzodioxole is optionally mono-substituted or poly-substituted with R⁵), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the (C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl,
R⁵ each independently represent a hydrogen atom, a halogen atom, a cyano, a nitro, an amino, a hydroxy, a thiol, a formyl, an oxo, a (C1-C6)alkyl, a (C2-C6)alkenyl, a (C2-C6)alkynyl, a (C3-C6)cycloalkyl, a (C1-C6)haloalkyl, a (C1-C6)alkoxy, a (C1-C6)haloalkoxy, a (C1-C6)alkoxy (C1-C6)alkyl, a (C1-C6)haloalkoxy (C1-C6)alkyl, a cyano (C1-C6)alkyl, a cyano (C3-C6)cycloalkyl, a (C1-C6)alkylthio, a (C1-C6)haloalkylthio, a (C1-C6)alkylsulfinyl, a (C1-C6)haloalkylsulfinyl, a (C1-C6)alkylsulfonyl, a (C1-C6)haloalkylsulfonyl, a (C1-C6)alkylcarbonylthio, a (C1-C6)alkylamino (the amino moiety is optionally substituted with R⁶), a (C1-C6)haloalkylamino (the amino moiety is optionally substituted with R⁶), a formylamino (the amino moiety is optionally substituted with R⁶), a (C1-C6)alkylcarbonylamino (the amino moiety is optionally substituted with R⁶), a carbamoyl, a (C1-C6)alkylaminocarbonyl (the amino moiety is optionally substituted with R⁶), a carboxyl, a (C1-C6)alkylcarbonyl, a (C1-C6)haloalkylcarbonyl, a (C1-C6)alkoxycarbonyl, a hydroxyimino (C1-C6)alkyl, a (C1-C6)alkoxyimino (C1-C6)alkyl, a (C6-C12)aryl (the (C6-C12)aryl is optionally mono-substituted or poly-substituted with R⁷), a (C6-C12)aryl (C1-C6)alkyl (the (C6-C12)aryl moiety is optionally mono-substituted or poly-substituted with R⁷), a (C6-C12)arylcarbonyl (the (C6-C12)arylcarbonyl is optionally mono-substituted or poly-substituted with R⁷), a (C6-C12)aryloxy (the (C6-C12)aryloxy is optionally mono-substituted or poly-substituted with R⁷), a (C6-C12)arylthio (the (C6-C12)arylthio is optionally mono-substituted or poly-substituted with R⁷), a (C6-C12)arylsulfinyl (the (C6-C12)arylsulfinyl is optionally mono-substituted or poly-substituted with R⁷), a (C6-C12)arylsulfonyl (the (C6-C12)arylsulfonyl is optionally mono-substituted or poly-substituted with R⁷), a (C6-C12)arylamino (the (C6-C12)aryl moiety is optionally mono-substituted or poly-substituted with R⁷, and the amino moiety is optionally substituted with R⁶), a 3- to 6-membered heterocyclyl (the 3- to 6-membered heterocyclyl is optionally mono-substituted or poly-substituted with R⁷), a 3- to 6-membered heterocyclyl (C1-C6)alkyl (the 3- to 6-membered heterocyclyl moiety is optionally mono-substituted or poly-substituted with R⁷), a 3- to 6-membered heterocyclylcarbonyl (the 3- to 6-membered heterocyclylcarbonyl is optionally mono-substituted or poly-substituted with R⁷), a 3- to 6-membered heterocyclyloxy (the 3- to 6-membered heterocyclyloxy is optionally mono-substituted or poly-substituted with R⁷), a 3- to 6-membered heterocyclylthio (the 3- to 6-membered heterocyclylthio is optionally mono-substituted or poly-substituted with R⁷), a 3- to 6-membered heterocyclylsulfinyl (the 3- to 6-membered heterocyclylsulfinyl is optionally mono-substituted or poly-substituted with R⁷), 3- to 6-membered heterocyclylsulfonyl (the 3- to 6-membered heterocyclylsulfonyl is optionally mono-substituted or poly-substituted with R⁷), a 3- to 6-membered heterocyclylamino (the 3- to 6-membered heterocyclyl moiety is optionally mono-substituted or poly-substituted with R⁷, and the amino moiety is optionally substituted with R⁶), a 5- to 12-membered heteroaryl (the 5- to 12-membered heteroaryl is optionally mono-substituted or poly-substituted with R⁷), a 5- to 12-membered heteroaryl (C1-C6)alkyl (the 5- to 12-membered heteroaryl moiety is optionally mono-substituted or poly-substituted with R⁷), a 5- to 12-membered heteroarylcarbonyl (the 5- to 12-membered heteroarylcarbonyl is optionally mono-substituted or poly-substituted with R⁷), a 5- to 12-membered heteroaryloxy (the 5- to 12-membered heteroaryloxy is optionally mono-substituted or poly-substituted with R⁷), a 5- to 12-membered heteroarylthio (the 5- to 12-membered heteroarylthio is optionally mono-substituted or poly-substituted with R⁷), a 5- to 12-membered heteroarylsulfinyl (the 5- to 12-membered heteroarylsulfinyl is optionally mono-substituted or poly-substituted with R⁷), a 5- to 12-membered heteroarylsulfonyl (the 5- to 12-membered heteroarylsulfonyl is optionally mono-substituted or poly-substituted with R⁷), or a 5- to 12-membered heteroarylamino (the 5- to 12-membered heteroaryl moiety is optionally mono-substituted or poly-substituted with R⁷, and the amino moiety is optionally substituted with R⁶),
R⁶ each independently represent a hydrogen atom, a (C1-C6)alkyl, a (C1-C6)haloalkyl, a (C3-C6)cycloalkyl, a (C1-C6)alkoxy (C1-C6)alkyl, a (C1-C6)haloalkoxy (C1-C6)alkyl, a cyano (C1-C6)alkyl, a (C1-C6)alkylcarbonyl, a (C1-C6)haloalkylcarbonyl, a (C1-C6)alkoxycarbonyl, or a (C6-C12)arylcarbonyl (the (C6-C12)arylcarbonyl is optionally mono-substituted or poly-substituted with R⁷),
R⁷ each independently represent a hydrogen atom, a halogen atom, a cyano, a (C1-C6)alkyl, or a (C1-C6)alkoxy, and
R⁶ each independently represent a halogen atom, a cyano, a (C1-C6)haloalkyl, a (C3-C6)cycloalkyl, a (C1-C6)alkoxy, a (C1-C6)alkylcarbonyl, a (C1-C6)alkoxycarbonyl, a (C1-C6)haloalkoxycarbonyl, a (C6-C12)aryl (the (C6-C12)aryl is optionally mono-substituted or poly-substituted with R⁷), a 3- to 6-membered heterocyclyl (the 3- to 6-membered heterocyclyl is optionally mono-substituted or poly-substituted with R⁷), or a 5- to 12-membered heteroaryl (the 5- to 12-membered heteroaryl is optionally mono-substituted or poly-substituted with R⁷).

(2) The formamide derivative of formula [I] or the agriculturally acceptable salt thereof according to (1), wherein in the formula [I], R² represents a (C1-C6)alkyl (the (C1-C6)alkyl is optionally mono-substituted or poly-substituted with R⁴), a (C3-C6)cycloalkyl (the (C3-C6)cycloalkyl is optionally mono-substituted or poly-substituted with R⁴), or a 3- to 6-membered heterocyclyl (the 3- to 6-membered heterocyclyl is optionally mono-substituted or poly-substituted with R⁵), and
R⁴ each independently represent a (C3-C6)cycloalkyl, a (C3-C6)halocycloalkyl, a (C6-C12)aryl (the (C6-C12)aryl is optionally mono-substituted or poly-substituted with R⁵), a 3- to 6-membered heterocyclyl (the 3- to 6-membered heterocyclyl is optionally mono-substituted or poly-substituted with R⁵), a 5- to 12-membered heteroaryl (the 5- to 12-membered heteroaryl is optionally mono-substituted or poly-substituted with R⁵), or a 1,3-benzodioxole (the 1,3-benzodioxole is optionally mono-substituted or poly-substituted with R⁵).

(3) The formamide derivative of formula [I] or the agriculturally acceptable salt thereof according to (2), wherein in the formula [I], R² represents a (C1-C6)alkyl (the (C1-C6)alkyl is optionally mono-substituted or poly-substituted with R⁴), and
R⁵ each independently represent a hydrogen atom, a halogen atom, a cyano, a nitro, a formyl, an oxo, a (C1-C6)alkyl, a (C1-C6)haloalkyl, a (C1-C6)alkoxy, a (C1-C6)haloalkoxy, a (C1-C6)alkoxy (C1-C6)alkyl, or a (C1-C6)alkoxycarbonyl.

(4) The formamide derivative of formula [I] or the agriculturally acceptable salt thereof according to (1), wherein in the formula [I], R⁴ each independently represent a halogen atom, a cyano, a thiocyanato, a thiol, a hydroxy, an amino, a tri((C1-C6)alkyl)silyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6) cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the (C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl group.

(5) The formamide derivative of formula [I] or the agriculturally acceptable salt thereof according to (4), wherein in the formula [I], R² represents a hydrogen atom, a (C1-C6)alkyl (the (C1-C6)alkyl is optionally mono-substituted or poly-substituted with R⁴), a (C2-C6)alkenyl (the (C2-C6)alkenyl is optionally mono-substituted or poly-substituted with R⁴), a (C2-C6)alkynyl (the (C2-C6)alkynyl is optionally mono-substituted or poly-substituted with R⁴), a (C6-C12)aryl (the (C6-C12)aryl is optionally mono-substituted or poly-substituted with R⁵), or a 3- to 6-membered heterocyclyl (the 3- to 6-membered heterocyclyl is optionally mono-substituted or poly-substituted with R⁵),
R⁴ each independently represent a halogen atom, a cyano, a thiol, a hydroxy, a tri((C1-C6)alkyl)silyl, a (C1-C6)alkylamino (the (C1-C6)alkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C1-C6)alkylcarbonyloxy (the (C1-C6)alkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C1-C6)alkoxycarbonyloxy (the (C1-C6)alkoxycarbonyloxy is optionally mono-substituted or poly-substituted with R⁸), a hydrazinocarbonyl (the hydrazino moiety is optionally substituted with R⁶), or a pentafluorosulfanyl,
R⁵ each independently represent a hydrogen atom, a halogen atom, a cyano, a nitro, a formyl, an oxo, a (C1-C6)alkyl, a (C1-C6)haloalkyl, a (C1-C6)alkoxy, a (C1-C6)haloalkoxy, a (C1-C6)alkoxy (C1-C6)alkyl, or a (C1-C6)alkoxycarbonyl, and
R⁶ each independently represent a hydrogen atom, a (C1-C6)alkyl, a (C1-C6)haloalkylcarbonyl, or a (C1-C6)alkoxycarbonyl.

(6) The formamide derivative of formula [I] or the agriculturally acceptable salt thereof according to (2) or (3), wherein in the formula [I], X¹, X², X³, and X⁴ each independently represent a carbon atom (the carbon atom is optionally substituted with R³), and R¹ is a halogen atom.

(7) The formamide derivative of formula [I} or the agriculturally acceptable salt thereof according to (2) or (3), wherein in the formula [I], R³ is a hydrogen atom.

(8) The formamide derivative of formula[I] or the agriculturally acceptable salt thereof according to (2) or (3), wherein in the formula [I], R² represents a (C1-C6)alkyl mono-substituted or poly-substituted with substituent(s) selected from a substituent group R⁴.

(9) The formamide derivative of formula [I] or the agriculturally acceptable salt thereof according to (4) or (5), wherein in the formula [I], X¹, X², X³, and X⁴ each independently represent a carbon atom (the carbon atom is optionally substituted with R³), and R¹ is a halogen atom.

(10) The formamide derivative of formula [I] or the agriculturally acceptable salt thereof according to (4) or (5), wherein in the formula [I], R³ is a hydrogen atom.

(11) The formamide derivative of formula [I] or the agriculturally acceptable salt thereof according to (4), wherein in the formula [I], R² represents a hydrogen atom, a (C1-C6)alkyl (the (C1-C6)alkyl is optionally mono-substituted or poly-substituted with R⁴), a (C2-C6)alkenyl (the (C2-C6)alkenyl is optionally mono-substituted or poly-substituted with R⁴), a (C2-C6)alkynyl (the (C2-C6)alkynyl is optionally mono-substituted or poly-substituted with R⁴), a (C3-C6)cycloalkyl (the (C3-C6)cycloalkyl is optionally mono-substituted or poly-substituted with R⁴), a (C6-C12)aryl (the (C6-C12)aryl is optionally mono-substituted or poly-substituted with R⁵), or a 5- to 12-membered heteroaryl (the 5- to 12-membered heteroaryl is optionally mono-substituted or poly-substituted with R⁵).

(12) The formamide derivative of formula [I] or the agriculturally acceptable salt thereof according to (4), wherein in the formula [I], R² represents a hydrogen atom or a (C1-C6)alkyl mono-substituted or poly-substituted with substituent(s) selected from a substituent group R⁴.

(13) An agricultural and horticultural plant disease control agent containing the formamide derivative of formula [I] or the agriculturally acceptable salt thereof according to any one of (1) to (12) as an active ingredient.

(14) The agricultural and horticultural plant disease control agent according to (13), in which phytopathogenic fungi are basidiomycetes, ascomycetes, deuteromycetes, oomycetes, and/or zygomycetes.

(15) An agricultural and horticultural plant disease control agent composition containing the formamide derivative of formula [I] or the agriculturally acceptable salt thereof according to any one of (1) to (12), and an agriculturally acceptable carrier.

(16) A method for controlling a disease of a useful plant, comprising a step of spraying an effective amount of the formamide derivative of formula [I] or the agriculturally acceptable salt thereof according to any one of (1) to (12) on the useful plant, a part of the useful plant, or a place where the useful plant grows.

(17) The method according to (16), in which phytopathogenic fungi are basidiomycetes, ascomycetes, deuteromycetes, oomycetes, and/or zygomycetes.

(18) A use of the formamide derivative of formula [I] or the agriculturally acceptable salt thereof according to any one of (1) to (12) as an agricultural and horticultural plant disease control agent for a useful plant.

(19) An aniline derivative of formula [II] or an agriculturally acceptable salt thereof,

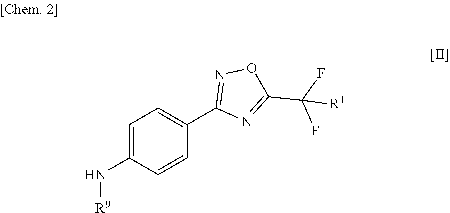

wherein in the formula [II],
R¹ represents a hydrogen atom or a halogen atom,
R⁹ represents a (C3-C6)alkyl, a (C1-C6)alkyl mono-substituted or poly-substituted with substituent(s) selected from a substituent group R¹⁰, a (C2-C6)alkenyl mono-substituted or poly-substituted with substituent(s) selected from a substituent group R¹¹, or a (C2-C6)alkynyl mono-substituted or poly-substituted with substituent(s) selected from a substituent group R¹²,
R¹⁰ and R¹² each independently represent a halogen atom, a cyano, a thiocyanato, a thiol, a hydroxy, an amino, a tri((C1-C6)alkyl)silyl, a (C3-C6)cycloalkyl, a (C3-C6)halocycloalkyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6) cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryl (the (C6-C12)aryl is optionally mono-substituted or poly-substituted with R⁵), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyl (the 3- to 6-membered heterocyclyl is optionally mono-substituted or poly-substituted with R⁵), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryl (the 5- to 12-membered heteroaryl is optionally mono-substituted or poly-substituted with R⁵), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a 1,3-benzodioxole (the 1,3-benzodioxole is optionally mono-substituted or poly-substituted with R⁵), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the (C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl,
R¹¹ each independently represent a halogen atom, a thiocyanato, a thiol, a hydroxy, an amino, a tri((C1-C6)alkyl)silyl, a (C3-C6)cycloalkyl, a (C3-C6)halocycloalkyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6)cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryl (the (C6-C12)aryl is optionally mono-substituted or poly-substituted with R⁵), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyl (the 3- to 6-membered heterocyclyl is optionally mono-substituted or poly-substituted with R⁵), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryl (the 5- to 12-membered heteroaryl is optionally mono-substituted or poly-substituted with R⁵), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a 1,3-benzodioxole (the 1,3-benzodioxole is optionally mono-substituted or poly-substituted with R⁵), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁵), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁵), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁵), a (C6-C12)aryloxycarbonyloxy (the (C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl,
R⁵ each independently represent a hydrogen atom, a halogen atom, a cyano, a nitro, an amino, a hydroxy, a thiol, a formyl, an oxo, a (C1-C6)alkyl, a (C2-C6)alkenyl, a (C2-C6)alkynyl, a (C3-C6)cycloalkyl, a (C1-C6)haloalkyl, a (C1-C6)alkoxy, a (C1-C6)haloalkoxy, a (C1-C6)alkoxy (C1-C6)alkyl, a (C1-C6)haloalkoxy (C1-C6)alkyl, a cyano (C1-C6)alkyl, a cyano (C3-C6)cycloalkyl, a (C1-C6)alkylthio, a (C1-C6)haloalkylthio, a (C1-C6)alkylsulfinyl, a (C1-C6)haloalkylsulfinyl, a (C1-C6)alkylsulfonyl, a (C1-C6)haloalkylsulfonyl, a (C1-C6)alkylcarbonylthio, a (C1-C6)alkylamino (the amino moiety is optionally substituted with R⁶), a (C1-C6)haloalkylamino (the amino moiety is optionally substituted with R⁶), a formylamino (the amino moiety is optionally substituted with R⁶), a (C1-C6)alkylcarbonylamino (the amino moiety is optionally substituted with R⁶), a carbamoyl, a (C1-C6)alkylaminocarbonyl (the amino moiety is optionally substituted with R⁶), a carboxyl, a (C1-C6)alkylcarbonyl, a (C1-C6)haloalkylcarbonyl, a (C1-C6)alkoxycarbonyl, a hydroxyimino (C1-C6)alkyl, a (C1-C6)alkoxyimino (C1-C6)alkyl, a (C6-C12)aryl (the (C6-C12)aryl is optionally mono-substituted or poly-substituted with R⁷), a (C6-C12)aryl (C1-C6)alkyl (the (C6-C12)aryl moiety is optionally mono-substituted or poly-substituted with R⁷), a (C6-C12)arylcarbonyl (the (C6-C12)arylcarbonyl is optionally mono-substituted or poly-substituted with R⁷), a (C6-C12)aryloxy (the (C6-C12)aryloxy is optionally mono-substituted or poly-substituted with R⁷), a (C6-C12)arylthio (the (C6-C12)arylthio is optionally mono-substituted or poly-substituted with R⁷), a (C6-C12)arylsulfinyl (the (C6-C12)arylsulfinyl is optionally mono-substituted or poly-substituted with R⁷), a (C6-C12)arylsulfonyl (the (C6-C12)arylsulfonyl is optionally mono-substituted or poly-substituted with R⁷), a (C6-C12)arylamino (the (C6-C12)aryl moiety is optionally mono-substituted or poly-substituted with R⁷, and the amino moiety is optionally substituted with R⁶), a 3- to 6-membered heterocyclyl (the 3- to 6-membered heterocyclyl is optionally mono-substituted or poly-substituted with R⁷), a 3- to 6-membered heterocyclyl (C1-C6)alkyl (the 3- to 6-membered heterocyclyl moiety is optionally mono-substituted or poly-substituted with R⁷), a 3- to 6-membered heterocyclylcarbonyl (the 3- to 6-membered heterocyclylcarbonyl is optionally mono-substituted or poly-substituted with R⁷), a 3- to 6-membered heterocyclyloxy (the 3- to 6-membered heterocyclyloxy is optionally mono-substituted or poly-substituted with R⁷), a 3- to 6-membered heterocyclylthio (the 3- to 6-membered heterocyclylthio is optionally mono-substituted or poly-substituted with R⁷), a 3- to 6-membered heterocyclylsulfinyl (the 3- to 6-membered heterocyclylsulfinyl is optionally mono-substituted or poly-substituted with R⁷), 3- to 6-membered heterocyclylsulfonyl (the 3- to 6-membered heterocyclylsulfonyl is optionally mono-substituted or poly-substituted with R⁷), a 3- to 6-membered heterocyclylamino (the 3- to 6-membered heterocyclyl moiety is optionally mono-substituted or poly-substituted with R⁷, and the amino moiety is optionally substituted with R⁶), a 5- to 12-membered heteroaryl (the 5- to 12-membered heteroaryl is optionally mono-substituted or poly-substituted with R⁷), a 5- to 12-membered heteroaryl (C1-C6)alkyl (the 5- to 12-membered heteroaryl moiety is optionally mono-substituted or poly-substituted with R⁷), a 5- to 12-membered heteroarylcarbonyl (the 5- to 12-membered heteroarylcarbonyl is optionally mono-substituted or poly-substituted with R⁷), a 5- to 12-membered heteroaryloxy (the 5- to 12-membered heteroaryloxy is optionally mono-substituted or poly-substituted with R⁷), a 5- to 12-membered heteroarylthio (the 5- to 12-membered heteroarylthio is optionally mono-substituted or poly-substituted with R⁷), a 5- to 12-membered heteroarylsulfinyl (the 5- to 12-membered heteroarylsulfinyl is optionally mono-substituted or poly-substituted with R⁷), a 5- to 12-membered heteroarylsulfonyl (the 5- to 12-membered heteroarylsulfonyl is optionally mono-substituted or poly-substituted with R⁷), or a 5- to 12-membered heteroarylamino (the 5- to 12-membered heteroaryl moiety is optionally mono-substituted or poly-substituted with R⁷, and the amino moiety is optionally substituted with R⁶),
R⁶ each independently represent a hydrogen atom, a (C1-C6)alkyl, a (C1-C6)haloalkyl, a (C3-C6)cycloalkyl, a (C1-C6)alkoxy (C1-C6)alkyl, a (C1-C6)haloalkoxy (C1-C6)alkyl, a cyano (C1-C6)alkyl, a (C1-C6)alkylcarbonyl, a (C1-C6)haloalkylcarbonyl, a (C1-C6)alkoxycarbonyl, or a (C6-C12)arylcarbonyl (the (C6-C12)arylcarbonyl is optionally mono-substituted or poly-substituted with R⁷),
R⁷ each independently represent a hydrogen atom, a halogen atom, a cyano, a (C1-C6)alkyl, or a (C1-C6)alkoxy, and
R⁶ each independently represent a halogen atom, a cyano, a (C1-C6)haloalkyl, a (C3-C6)cycloalkyl, a (C1-C6)alkoxy, a (C1-C6)alkylcarbonyl, a (C1-C6)alkoxycarbonyl, a (C1-C6)haloalkoxycarbonyl, a (C6-C12)aryl (the (C6-C12)aryl is optionally mono-substituted or poly-substituted with R⁷), a 3- to 6-membered heterocyclyl (the 3- to 6-membered heterocyclyl is optionally mono-substituted or poly-substituted with R⁷), or a 5- to 12-membered heteroaryl (the 5- to 12-membered heteroaryl is optionally mono-substituted or poly-substituted with R⁷).

(20) The aniline derivative of formula [II] or the agriculturally acceptable salt thereof according to (19), wherein in the formula [II], R⁹ represents a (C1-C6)alkyl mono-substituted or poly-substituted with substituent(s) selected from a substituent group R¹⁰, and
R¹⁰ each independently represent a (C3-C6)cycloalkyl, a (C3-C6)halocycloalkyl, a (C6-C12)aryl (the (C6-C12)aryl is optionally mono-substituted or poly-substituted with R⁵), a 3- to 6-membered heterocyclyl (the 3- to 6-membered heterocyclyl is optionally mono-substituted or poly-substituted with R⁵), a 5- to 12-membered heteroaryl (the 5- to 12-membered heteroaryl is optionally mono-substituted or poly-substituted with R⁵), or a 1,3-benzodioxole (the 1,3-benzodioxole is optionally mono-substituted or poly-substituted with R⁵).

(21) The aniline derivative of formula [II] or the agriculturally acceptable salt thereof according to (20), wherein in the formula [II], R⁵ each independently represent a hydrogen atom, a halogen atom, a cyano, a nitro, a formyl, an oxo, a (C1-C6)alkyl, a (C1-C6)haloalkyl, a (C1-C6)alkoxy, a (C1-C6)haloalkoxy, a (C1-C6)alkoxy (C1-C6)alkyl, or a (C1-C6)alkoxycarbonyl.

(22) The aniline derivative of formula [II] or the agriculturally acceptable salt thereof according to (19), wherein in the formula [II], R¹⁰ and R¹² each independently represent a halogen atom, a cyano, a thiocyanato, a thiol, a hydroxy, an amino, a tri((C1-C6)alkyl)silyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6) cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the (C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl,
R¹¹ each independently represent a halogen atom, a thiocyanato, a thiol, a hydroxy, an amino, a tri((C1-C6)alkyl)silyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6) cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the (C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl group.

(23) The aniline derivative of formula [II] or the agriculturally acceptable salt thereof according to (22), wherein in the formula [II], R¹⁰ and R¹² each independently represent a halogen atom, a cyano, a thiol, a hydroxy, a tri((C1-C6)alkyl)silyl, a (C1-C6)alkylamino (the (C1-C6)alkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C1-C6)alkylcarbonyloxy (the (C1-C6)alkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C1-C6)alkoxycarbonyloxy (the (C1-C6)alkoxycarbonyloxy is optionally mono-substituted or poly-substituted with R⁸), a hydrazinocarbonyl (the hydrazino moiety is optionally substituted with R⁶), or a pentafluorosulfanyl,
R¹¹ each independently represent a halogen atom, a thiol, a hydroxy, a tri((C1-C6)alkyl)silyl, a (C1-C6)alkylamino (the (C1-C6)alkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C1-C6)alkylcarbonyloxy (the (C1-C6)alkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C1-C6)alkoxycarbonyloxy (the (C1-C6)alkoxycarbonyloxy is optionally mono-substituted or poly-substituted with R⁸), a hydrazinocarbonyl (the hydrazino moiety is optionally substituted with R⁶), or a pentafluorosulfanyl,
R⁵ each independently represent a hydrogen atom, a halogen atom, a cyano, a nitro, a formyl, an oxo, a (C1-C6)alkyl, a (C1-C6)haloalkyl, a (C1-C6)alkoxy, a (C1-C6)haloalkoxy, a (C1-C6)alkoxy (C1-C6)alkyl, or a (C1-C6)alkoxycarbonyl, and
R⁶ each independently represent a hydrogen atom, a (C1-C6)alkyl, a (C1-C6)haloalkylcarbonyl, or a (C1-C6)alkoxycarbonyl.

(24) An amidoxime derivative of formula [III] or an agriculturally acceptable salt thereof,

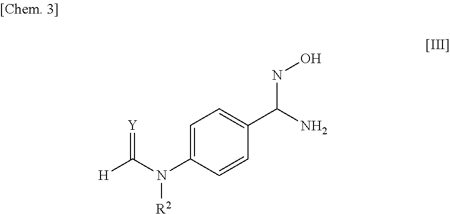

wherein in the formula [III],
Y represents an oxygen atom or a sulfur atom,
R² represents a hydrogen atom, a (C1-C6)alkyl (the (C1-C6)alkyl is optionally mono-substituted or poly-substituted with R⁴), a (C2-C6)alkenyl (the (C2-C6)alkenyl is optionally mono-substituted or poly-substituted with R⁴), a (C2-C6)alkynyl (the (C2-C6)alkynyl is optionally mono-substituted or poly-substituted with R⁴), a (C3-C6)cycloalkyl (the (C3-C6)cycloalkyl is optionally mono-substituted or poly-substituted with R⁴), a (C6-C12)aryl (the (C6-C12)aryl is optionally mono-substituted or poly-substituted with R⁵), a 3- to 6-membered heterocyclyl (the 3- to 6-membered heterocyclyl is optionally mono-substituted or poly-substituted with R⁵), or a 5- to 12-membered heteroaryl (the 5- to 12-membered heteroaryl is optionally mono-substituted or poly-substituted with R⁵),
R⁴ each independently represent a hydrogen atom, a halogen atom, a cyano, a thiocyanato, a thiol, a hydroxy, an amino, a tri((C1-C6)alkyl)silyl, a (C3-C6)cycloalkyl, a (C3-C6)halocycloalkyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6)cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryl (the (C6-C12)aryl is optionally mono-substituted or poly-substituted with R⁵), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyl (the 3- to 6-membered heterocyclyl is optionally mono-substituted or poly-substituted with R⁵), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryl (the 5- to 12-membered heteroaryl is optionally mono-substituted or poly-substituted with R⁵), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a 1,3-benzodioxole (the 1,3-benzodioxole is optionally mono-substituted or poly-substituted with R⁵), a hydroxyimino (C1-C6)alkyl, a (C1-C6)alkoxyimino (C1-C6)alkyl (the (C1-C6)alkoxy moiety is optionally mono-substituted or poly-substituted with R⁵), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁵), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁵), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁵), a (C6-C12)aryloxycarbonyloxy (the (C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl,
R⁵ each independently represent a hydrogen atom, a halogen atom, a cyano, a nitro, an amino, a hydroxy, a thiol, a formyl, an oxo, a (C1-C6)alkyl, a (C2-C6)alkenyl, a (C2-C6)alkynyl, a (C3-C6)cycloalkyl, a (C1-C6)haloalkyl, a (C1-C6)alkoxy, a (C1-C6)haloalkoxy, a (C1-C6)alkoxy (C1-C6)alkyl, a (C1-C6)haloalkoxy (C1-C6)alkyl, a cyano (C1-C6)alkyl, a cyano (C3-C6)cycloalkyl, a (C1-C6)alkylthio, a (C1-C6)haloalkylthio, a (C1-C6)alkylsulfinyl, a (C1-C6)haloalkylsulfinyl, a (C1-C6)alkylsulfonyl, a (C1-C6)haloalkylsulfonyl, a (C1-C6)alkylcarbonylthio, a (C1-C6)alkylamino (the amino moiety is optionally substituted with R⁶), a (C1-C6)haloalkylamino (the amino moiety is optionally substituted with R⁶), a formylamino (the amino moiety is optionally substituted with R⁶), a (C1-C6)alkylcarbonylamino (the amino moiety is optionally substituted with R⁶), a carbamoyl, a (C1-C6)alkylaminocarbonyl (the amino moiety is optionally substituted with R⁶), a carboxyl, a (C1-C6)alkylcarbonyl, a (C1-C6)haloalkylcarbonyl, a (C1-C6)alkoxycarbonyl, a hydroxyimino (C1-C6)alkyl, a (C1-C6)alkoxyimino (C1-C6)alkyl, a (C6-C12)aryl (the (C6-C12)aryl is optionally mono-substituted or poly-substituted with R⁷), a (C6-C12)aryl (C1-C6)alkyl (the (C6-C12)aryl moiety is optionally mono-substituted or poly-substituted with R⁷), a (C6-C12)arylcarbonyl (the (C6-C12)arylcarbonyl is optionally mono-substituted or poly-substituted with R⁷), a (C6-C12)aryloxy (the (C6-C12)aryloxy is optionally mono-substituted or poly-substituted with R⁷), a (C6-C12)arylthio (the (C6-C12)arylthio is optionally mono-substituted or poly-substituted with R⁷), a (C6-C12)arylsulfinyl (the (C6-C12)arylsulfinyl is optionally mono-substituted or poly-substituted with R⁷), a (C6-C12)arylsulfonyl (the (C6-C12)arylsulfonyl is optionally mono-substituted or poly-substituted with R⁷), a (C6-C12)arylamino (the (C6-C12)aryl moiety is optionally mono-substituted or poly-substituted with R⁷, and the amino moiety is optionally substituted with R⁶), a 3- to 6-membered heterocyclyl (the 3- to 6-membered heterocyclyl is optionally mono-substituted or poly-substituted with R⁷), a 3- to 6-membered heterocyclyl (C1-C6)alkyl (the 3- to 6-membered heterocyclyl moiety is optionally mono-substituted or poly-substituted with R⁷), a 3- to 6-membered heterocyclylcarbonyl (the 3- to 6-membered heterocyclylcarbonyl is optionally mono-substituted or poly-substituted with R⁷), a 3- to 6-membered heterocyclyloxy (the 3- to 6-membered heterocyclyloxy is optionally mono-substituted or poly-substituted with R⁷), a 3- to 6-membered heterocyclylthio (the 3- to 6-membered heterocyclylthio is optionally mono-substituted or poly-substituted with R⁷), a 3- to 6-membered heterocyclylsulfinyl (the 3- to 6-membered heterocyclylsulfinyl is optionally mono-substituted or poly-substituted with R⁷), 3- to 6-membered heterocyclylsulfonyl (the 3- to 6-membered heterocyclylsulfonyl is optionally mono-substituted or poly-substituted with R⁷), a 3- to 6-membered heterocyclylamino (the 3- to 6-membered heterocyclyl moiety is optionally mono-substituted or poly-substituted with R⁷, and the amino moiety is optionally substituted with R⁶), a 5- to 12-membered heteroaryl (the 5- to 12-membered heteroaryl is optionally mono-substituted or poly-substituted with R⁷), a 5- to 12-membered heteroaryl (C1-C6)alkyl (the 5- to 12-membered heteroaryl moiety is optionally mono-substituted or poly-substituted with R⁷), a 5- to 12-membered heteroarylcarbonyl (the 5- to 12-membered heteroarylcarbonyl is optionally mono-substituted or poly-substituted with R⁷), a 5- to 12-membered heteroaryloxy (the 5- to 12-membered heteroaryloxy is optionally mono-substituted or poly-substituted with R⁷), a 5- to 12-membered heteroarylthio (the 5- to 12-membered heteroarylthio is optionally mono-substituted or poly-substituted with R⁷), a 5- to 12-membered heteroarylsulfinyl (the 5- to 12-membered heteroarylsulfinyl is optionally mono-substituted or poly-substituted with R⁷), a 5- to 12-membered heteroarylsulfonyl (the 5- to 12-membered heteroarylsulfonyl is optionally mono-substituted or poly-substituted with R⁷), or a 5- to 12-membered heteroarylamino (the 5- to 12-membered heteroaryl moiety is optionally mono-substituted or poly-substituted with R⁷, and the amino moiety is optionally substituted with R⁶),
R⁶ each independently represent a hydrogen atom, a (C1-C6)alkyl, a (C1-C6)haloalkyl, a (C3-C6)cycloalkyl, a (C1-C6)alkoxy (C1-C6)alkyl, a (C1-C6)haloalkoxy (C1-C6)alkyl, a cyano (C1-C6)alkyl, a (C1-C6)alkylcarbonyl, a (C1-C6)haloalkylcarbonyl, a (C1-C6)alkoxycarbonyl, or a (C6-C12)arylcarbonyl (the (C6-C12)arylcarbonyl is optionally mono-substituted or poly-substituted with R⁷),
R⁷ each independently represent a hydrogen atom, a halogen atom, a cyano, a (C1-C6)alkyl, or a (C1-C6)alkoxy, and
R⁶ each independently represent a halogen atom, a cyano, a (C1-C6)haloalkyl, a (C3-C6)cycloalkyl, a (C1-C6)alkoxy, a (C1-C6)alkylcarbonyl, a (C1-C6)alkoxycarbonyl, a (C1-C6)haloalkoxycarbonyl, a (C6-C12)aryl (the (C6-C12)aryl is optionally mono-substituted or poly-substituted with R⁷), a 3- to 6-membered heterocyclyl (the 3- to 6-membered heterocyclyl is optionally mono-substituted or poly-substituted with R⁷), or a 5- to 12-membered heteroaryl (the 5- to 12-membered heteroaryl is optionally mono-substituted or poly-substituted with R⁷).

(25) The amidoxime derivative of formula [III] or the agriculturally acceptable salt thereof according to (24), wherein in the formula [III], R² represents a (C1-C6)alkyl (the (C1-C6)alkyl is optionally mono-substituted or poly-substituted with R⁴), a (C3-C6)cycloalkyl (the (C3-C6)cycloalkyl is optionally mono-substituted or poly-substituted with R⁴), or a 3- to 6-membered heterocyclyl (the 3- to 6-membered heterocyclyl is optionally mono-substituted or poly-substituted with R⁵), and
R⁴ each independently represent a (C3-C6)cycloalkyl, a (C3-C6)halocycloalkyl, a (C6-C12)aryl (the (C6-C12)aryl is optionally mono-substituted or poly-substituted with R⁵), a 3- to 6-membered heterocyclyl (the 3- to 6-membered heterocyclyl is optionally mono-substituted or poly-substituted with R⁵), a 5- to 12-membered heteroaryl (the 5- to 12-membered heteroaryl is optionally mono-substituted or poly-substituted with R⁵), or a 1,3-benzodioxole (the 1,3-benzodioxole is optionally mono-substituted or poly-substituted with R⁵).

(26) The amidoxime derivative of formula [III] or the agriculturally acceptable salt thereof according to (25), wherein in the formula [III], R² represents a (C1-C6)alkyl (the (C1-C6)alkyl is optionally mono-substituted or poly-substituted with R⁴), and R⁵ each independently represent a hydrogen atom, a halogen atom, a cyano, a nitro, a formyl, an oxo, a (C1-C6)alkyl, a (C1-C6)haloalkyl, a (C1-C6)alkoxy, a (C1-C6)haloalkoxy, a (C1-C6)alkoxy (C1-C6)alkyl, or a (C1-C6)alkoxycarbonyl.

(27) The amidoxime derivative of formula [III] or the agriculturally acceptable salt thereof according to (24), wherein in the formula [III], R⁴ each independently represent a halogen atom, a cyano, a thiocyanato, a thiol, a hydroxy, an amino, a tri((C1-C6)alkyl)silyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6) cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the (C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl group.

(28) The amidoxime derivative of formula [III] or the agriculturally acceptable salt thereof according to (27), wherein in the formula [III], R² represents a hydrogen atom, a (C1-C6)alkyl (the (C1-C6)alkyl is optionally mono-substituted or poly-substituted with R⁴), a (C2-C6)alkenyl (the (C2-C6)alkenyl is optionally mono-substituted or poly-substituted with R⁴), a (C2-C6)alkynyl (the (C2-C6)alkynyl is optionally mono-substituted or poly-substituted with R⁴), a (C6-C12)aryl (the (C6-C12)aryl is optionally mono-substituted or poly-substituted with R⁵), or a 3- to 6-membered heterocyclyl (the 3- to 6-membered heterocyclyl is optionally mono-substituted or poly-substituted with R⁵),
R⁴ each independently represent a halogen atom, a cyano, a thiol, a hydroxy, a tri((C1-C6)alkyl)silyl, a (C1-C6)alkylamino (the (C1-C6)alkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C1-C6)alkylcarbonyloxy (the (C1-C6)alkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C1-C6)alkoxycarbonyloxy (the (C1-C6)alkoxycarbonyloxy is optionally mono-substituted or poly-substituted with R⁸), a hydrazinocarbonyl (the hydrazino moiety is optionally substituted with R⁶), or a pentafluorosulfanyl,
R⁵ each independently represent a hydrogen atom, a halogen atom, a cyano, a nitro, a formyl, an oxo, a (C1-C6)alkyl, a (C1-C6)haloalkyl, a (C1-C6)alkoxy, a (C1-C6)haloalkoxy, a (C1-C6)alkoxy (C1-C6)alkyl, or a (C1-C6)alkoxycarbonyl, and
R⁶ each independently represent a hydrogen atom, a (C1-C6)alkyl, a (C1-C6)haloalkylcarbonyl, or a (C1-C6)alkoxycarbonyl.

### Effects of the Invention

The compound of the present invention is useful as an active ingredient of an agricultural chemical such as an agricultural and horticultural plant disease control agent.

An agricultural and horticultural plant disease control agent containing the compound of the present invention can exhibit an excellent control effect on a wide range of plant diseases caused by basidiomycetes, ascomycetes, deuteromycetes, oomycetes, zygomycetes, and the like, and can also control fungi that have acquired chemical resistance.

### DESCRIPTION OF EMBODIMENTS

Symbols and terms used in the present specification will be described.

In the present invention, the “halogen atom” refers to a fluorine atom, a chlorine atom, a bromine atom, or an iodine atom.

In the present invention, the expression of “(C1-C6)” or the like indicates that the number of carbon atoms of the subsequent substituent is 1 to 6 in this case.

In the present invention, unless otherwise specified, the “(C1-C6)alkyl” refers to a linear or branched alkyl group having 1 to 6 carbon atoms, and examples thereof include methyl, ethyl, n-propyl, isopropyl, n-butyl, sec-butyl, isobutyl, tert-butyl, n-pentyl, 1-methylbutyl, 2-methylbutyl, 3-methylbutyl, 1-ethylpropyl, 1,1-dimethylpropyl, 1,2-dimethylpropyl, neopentyl, n-hexyl, 1-methylpentyl, 2-methylpentyl, 3-methylpentyl, 4-methylpentyl, 1-ethylbutyl, 2-ethylbutyl, 1,1-dimethylbutyl, 1,2-dimethylbutyl, 1,3-dimethylbutyl, 2,2-dimethylbutyl, 2,3-dimethylbutyl, 3,3-dimethylbutyl, 1,1,2-trimethylpropyl, 1,2,2-trimethylpropyl, 1-ethyl-1-methylpropyl, and 1-ethyl-2-methylpropyl groups.

In the present invention, unless otherwise specified, the “(C3-C6)alkyl” refers to a linear or branched alkyl group having 3 to 6 carbon atoms, and examples thereof include groups excluding methyl and ethyl among the “(C1-C6)alkyl”.

In the present invention, unless otherwise specified, the “(C2-C6)alkenyl” refers to a linear or branched alkenyl group having 2 to 6 carbon atoms, and examples thereof include vinyl, 1-propenyl, isopropenyl, 2-propenyl, 1-butenyl, 1-methyl-1-propenyl, 2-butenyl, 1-methyl-2-propenyl, 3-butenyl, 2-methyl-1-propenyl, 2-methyl-2-propenyl, 1,3-butadienyl, 1-pentenyl, 1-ethyl-2-propenyl, 2-pentenyl, 1-methyl-1-butenyl, 3-pentenyl, 1-methyl-2-butenyl, 4-pentenyl, 1-methyl-3-butenyl, 3-methyl-1-butenyl, 1,2-dimethyl-2-propenyl, 1,1-dimethyl-2-propenyl, 2-methyl-2-butenyl, 3-methyl-2-butenyl, 1,2-dimethyl-1-propenyl, 2-methyl-3-butenyl, 3-methyl-3-butenyl, 1,3-pentadienyl, 1-vinyl-2-propenyl, 1-hexenyl, 1-propyl-2-propenyl, 2-hexenyl, 1-methyl-1-pentenyl, 1-ethyl-2-butenyl, 3-hexenyl, 4-hexenyl, 5-hexenyl, 1-methyl-4-pentenyl, 1-ethyl-3-butenyl, 1-(isobutyl)vinyl, 1-ethyl-1-methyl-2-propenyl, 1-ethyl-2-methyl-2-propenyl, 1-(isopropyl)-2-propenyl, 2-methyl-2-pentenyl, 3-methyl-3-pentenyl, 4-methyl-3-pentenyl, 1,3-dimethyl-2-butenyl, 1,1-dimethyl-3-butenyl, 3-methyl-4-pentenyl, 4-methyl-4-pentenyl, 1,2-dimethyl-3-butenyl, 1,3-dimethyl-3-butenyl, 1,1,2-trimethyl-2-propenyl, 1,5-hexadienyl, 1-vinyl-3-butenyl, and 2,4-hexadienyl groups.

In the present invention, unless otherwise specified, the “(C2-C6)alkynyl” refers to a linear or branched alkynyl group having 2 to 6 carbon atoms, and examples thereof include ethynyl, 1-propynyl, 2-propynyl, 1-butynyl, 1-methyl-2-propynyl, 2-butynyl, 3-butynyl, 1-pentynyl, 1-ethyl-2-propynyl, 2-pentynyl, 3-pentynyl, 1-methyl-2-butynyl, 4-pentynyl, 1-methyl-3-butynyl, 2-methyl-3-butynyl, 1-hexynyl, 1-(n-propyl)-2-propynyl, 2-hexynyl, 1-ethyl-2-butynyl, 3-hexynyl, 1-methyl-2-pentynyl, 1-methyl-3-pentynyl, 4-methyl-1-pentynyl, 3-methyl-1-pentynyl, 5-hexynyl, 1-ethyl-3-butynyl, 1-ethyl-1-methyl-2-propynyl, 1-(isopropyl)-2-propynyl, 1,1-dimethyl-2-butynyl, and 2,2-dimethyl-3-butynyl groups.

In the present invention, unless otherwise specified, the “(C3-C6)cycloalkyl” refers to a cycloalkyl group having 3 to 6 carbon atoms, and the ring may be optionally substituted with an alkyl group in a range of a specific number of carbon atoms. Examples thereof include cyclopropyl, 1-methylcyclopropyl, 2-methylcyclopropyl, 2,2-dimethylcyclopropyl, cyclobutyl, 1-methylcyclobutyl, cyclopentyl, 1-methylcyclopentyl, and cyclohexyl groups.

In the present invention, unless otherwise specified, the “(C6-C12)aryl” refers to an aromatic hydrocarbon group having 6 to 12 carbon atoms, and examples thereof include phenyl, biphenyl, naphthalen-1-yl, and naphthalen-2-yl groups.

In the present invention, unless otherwise specified, the “3- to 6-membered heterocyclyl” refers to a 3- to 6-membered non-aromatic hetero ring containing one or more atoms selected from an oxygen atom, a nitrogen atom, a sulfur atom, and a boron atom in addition to a carbon atom, and examples thereof include aziridinyl, oxylanyl, oxetanyl, thietanyl, tetrahydrofuranyl, tetrahydropyranyl, azetidinyl, pyrrolidinyl, piperidinyl, piperazinyl, dihydroisoxazolyl, dioxaborolanyl, dioxolanyl, dioxanyl, and morpholinyl groups.

In the present invention, unless otherwise specified, the “5- to 12-membered heteroaryl” refers to a 5- to 12-membered monocyclic or condensed bicyclic aromatic hetero ring containing one or more heteroatoms selected from a nitrogen atom, an oxygen atom, and a sulfur atom in addition to a carbon atom. Examples of the monocyclic hetero ring include pyridyl, pyrimidinyl, pyrazinyl, pyridazinyl, triazolyl, thienyl, furanyl, pyrrolyl, pyrazolyl, thiazolyl, isothiazolyl, oxazolyl, isooxazolyl, imidazolyl, triazolyl, thiadiazolyl, oxadiazolyl, and tetrazolyl groups. The condensed bicyclic aromatic hetero ring has a condensed bicyclic ring moiety formed by condensing one of the monocyclic groups and the phenyl ring or any one of the heteroaromatic monocyclic groups to form a C5-C10 bicyclic group, and examples thereof include indolyl, benzoimidazoly, indazolyl, benzotriazolyl, isoquinolinyl, quinolinyl, benzothiazolyl, benzofuranyl, benzothienyl, benzoisoxazolyl, pyrazolopyridyl, pyrazolopyrimidinyl, pyrrolopyridyl, triazoropyridyl, triazolopyrimidinyl, quinazolinyl, quinoxalinyl, and cinnolinyl groups.

In the present invention, unless otherwise specified, the “tri((C1-C6)alkyl)silyl” refers to a ((C1-C6)alkyl)₃-Si— whose alkyl moiety is as defined above, in which three alkyl groups may be the same as or different from each other, and examples thereof include trimethylsilyl, triethylsilyl, triisopropylsilyl, dimethylisopropylsilyl, and tert-butyldimethylsilyl groups.

In the present invention, unless otherwise specified, the “(C1-C6)haloalkyl” refers to a linear or branched alkyl group having 1 to 6 carbon atoms that is substituted with 1 to 13 same or different halogen atoms, and examples thereof include fluoromethyl, difluoromethyl, trifluoromethyl, chloromethyl, dichloromethyl, trichloromethyl, bromomethyl, dibromomethyl, tribromomethyl, iodomethyl, chlorodifluoromethyl, dichlorofluoromethyl, 1-fluoroethyl, 2-fluoroethyl, 1,1-difluoroethyl, 2,2-difluoroethyl, 2,2,2-trifluoroethyl, 1,1,2,2-tetrafluoroethyl, pentafluoroethyl, 1-chloroethyl, 2-chloroethyl, 1,1-dichloroethyl, 2,2-dichloroethyl, 2,2,2-trichloroethyl, 1,1,2,2-tetrachloroethyl, pentachloroethyl, 1-bromoethyl, 2-bromoethyl, 2,2,2-tribromoethyl, 1-iodoethyl, 2-iodoethyl, 2-chloro-2,2-difluoroethyl, 2,2-dichloro-2-fluoroethyl, 1-fluoropropyl, 2-fluoropropyl, 3-fluoropropyl, 1,1-difluoropropyl, 2,2-difluoropropyl, 3,3-difluoropropyl, 3,3,3-trifluoropropyl, 2,2,3,3,3-pentafluoropropyl, 1,1,2,3,3,3-hexafluoropropyl, heptafluoropropyl, 1-fluoro-2-propyl, 2-fluoro-2-propyl, 1,1-difluoro-2-propyl, 1,2-difluoro-2-propyl, 1,3-difluoro-2-propyl, 1,2,3-trifluoro-2-propyl, 1,1,3,3-tetrafluoro-2-propyl, 1,1,1,3,3,3-hexafluoro-2-propyl, heptafluoro-2-propyl, 1-chloropropyl, 2-chloropropyl, 3-chloropropyl, 1,1-dichloropropyl, 2,2-dichloropropyl, 3,3-dichloropropyl, 3,3,3-trichloropropyl, 2,2,3,3,3-pentachloropropyl, heptachloropropyl, 1-chloro-2-propyl, 2-chloro-2-propyl, 1,1-dichloro-2-propyl, 1,2-dichloro-2-propyl, 1,3-dichloro-2-propyl, 1,2,3-trichloro-2-propyl, 1,1,3,3-tetrachloro-2-propyl, 1,1,1,3,3,3-hexachloro-2-propyl, heptachloro-2-propyl, 1-bromopropyl, 2-bromopropyl, 3-bromopropyl, 1-bromo-2-propyl, 2-bromo-2-propyl, 1-iodopropyl, 2-iodopropyl, 3-iodopropyl, 1-iodo-2-propyl, 2-iodo-2-propyl, 1-fluorobutyl, 2-fluorobutyl, 3-fluorobutyl, 4-fluorobutyl, 4,4-difluorobutyl, 4,4,4-trifluorobutyl, 4,4,4-trifluoro-3-methylbutyl, 3,3,4,4,4-pentafluorobutyl, 2,2,3,4,4,4-hexafluorobutyl, 2,2,3,3,4,4,4-heptafluorobutyl, nonafluorobutyl, 1,1,1-trifluoro-2-butyl, 4,4,4-trifluoro-2-butyl, 3,3,4,4,4-pentafluoro-2-butyl, nonafluoro-2-butyl, 1,1,1,3,3,3-hexafluoro-2-(trifluoromethyl)-2-propyl, 1-chlorobutyl, 2-chlorobutyl, 3-chlorobutyl, 4-chlorobutyl, 4,4-dichlorobutyl, 4,4,4-trichlorobutyl, nonachlorobutyl, 1,1,1-trichloro-2-butyl, 4,4,4-trichloro-2-butyl, nonachloro-2-butyl, 1-bromobutyl, 2-bromobutyl, 3-bromobutyl, 4-bromobutyl, 1-iodobutyl, 2-iodobutyl, 3-iodobutyl, 4-iodobutyl, 4-chloro-1,1,2,2,3,3,4,4-octafluorobutyl, 4-bromo-1,1,2,2,3,3,4,4-octafluorobutyl, 1-fluoropentyl, 2-fluoropentyl, 3-fluoropentyl, 4-fluoropentyl, 5-fluoropentyl, 5,5,5-trifluoropentyl, 4,4,5,5,5-pentafluoropentyl, 3,3,4,4,5,5,5-heptafluoropentyl, 2,2,3,3,4,4,5,5-octafluoropentyl, 2,2,3,3,4,4,5,5,5-nonafluoropentyl, undecafluoropentyl, 1-chloropentyl, 2-chloropentyl, 3-chloropentyl, 4-chloropentyl, 5-chloropentyl, 5,5,5-trichloropentyl, 4,4,5,5,5-pentachloropentyl, 3,3,4,4,5,5,5-heptachloropentyl, 2,2,3,3,4,4,5,5,5-nonachloropentyl, undecachloropentyl, 1-bromopentyl, 2-bromopentyl, 3-bromopentyl, 4-bromopentyl, 5-bromopentyl, 5-iodopentyl, 1-fluorohexyl, 2-fluorohexyl, 3-fluorohexyl, 4-fluorohexyl, 5-fluorohexyl, 6-fluorohexyl, 6,6,6-trifluorohexyl, 5,5,6,6,6-pentafluorohexyl, 4,4,5,5,6,6,6-heptafluorohexyl, 3,3,4,4,5,5,6,6,6-nonafluorohexyl, 2,2,3,3,4,4,5,5,6,6,6-undecafluorohexyl, 2,2,3,3,4,4,5,5,6,6-decafluorohexyl, tridecafluorohexyl, 1-chlorohexyl, 2-chlorohexyl, 3-chlorohexyl, 4-chlorohexyl, 5-chlorohexyl, 6-chlorohexyl, 5-bromohexyl, 6-bromohexyl, 5-iodohexyl, and 6-iodohexyl groups.

In the present invention, unless otherwise specified, the “(C3-C6)halocycloalkyl” refers to a cycloalkyl group having 3 to 6 carbon atoms that is substituted with the same or different 1 to 11 halogen atoms, and examples thereof include 1-fluorocyclopropyl, 2-fluorocyclopropyl, 2,2-difluorocyclopropyl, 2,2,3,3-tetrafluorocyclopropyl, 1-chlorocyclopropyl, 2-chlorocyclopropyl, 2,2-dichlorocyclopropyl, 2,2,3,3-tetrachlorocyclopropyl, 2,2-dibromocyclopropyl, 2,2-diiodocyclopropyl, 1-fluorocyclobutyl, 2-fluorocyclobutyl, 3-fluorocyclobutyl, 3,3-difluorocyclobutyl, heptafluorocyclobutyl, 2-chlorocyclobutyl, 3-chlorocyclobutyl, 3,3-dichlorocyclobutyl, 3,3-dibromocyclobutyl, 3,3-diiodocyclobutyl, 1-fluorocyclopentyl, 2-fluorocyclopentyl, 3-fluorocyclopentyl, 2,2-difluorocyclopentyl, 3,3-difluorocyclopentyl, nonafluorocyclopentyl, 2,2-dichlorocyclopentyl, 3,3-dichlorocyclopentyl, 2,2-dibromocyclopentyl, 3,3-dibromocyclopentyl, 2,2-diiodocyclopentyl, 3,3-diiodocyclopentyl, 1-fluorocyclohexyl, 2-fluorocyclohexyl, 3-fluorocyclohexyl, 4-fluorocyclohexyl, 2,2-difluorocyclohexyl, 3,3-difluorocyclohexyl, 4,4-difluorocyclohexyl, 1-chlorocyclohexyl, 2-chlorocyclohexyl, 3-chlorocyclohexyl, 4-chlorocyclohexyl, 2,2-dichlorocyclohexyl, 3,3-dichlorocyclohexyl, 4,4-dichlorocyclohexyl, 3,3-dibromocyclohexyl, 4,4-dibromocyclohexyl, 3,3-diiodocyclohexyl, and 4,4-diiodocyclohexyl groups.

In the present invention, unless otherwise specified, the “(C1-C6)alkylamino” refers to a ((C1-C6)alkyl)-NH— group whose alkyl moiety is as defined above, and examples thereof include methylamino, ethylamino, n-propylamino, and isopropyl amino groups.

In the present invention, unless otherwise specified, the “(C2-C6)alkenylamino” refers to a ((C2-C6)alkenyl)-NH— group whose alkenyl moiety is as defined above, and examples thereof include vinylamino, 1-propenylamino, isopropenylamino, 2-propenylamino, 1-butenylamino, 1-methyl-1-propenylamino, 2-butenylamino, 1-methyl-2-propenylamino, 3-butenylamino, 2-methyl-1-propenylamino, 2-methyl-2-propenylamino, 1,3-butadienylamino, 1-pentenylamino, 1-ethyl-2-propenylamino, 2-pentenylamino, 1-methyl-1-butenylamino, 3-pentenylamino, 1-methyl-2-butenylamino, 4-pentenylamino, 1-methyl-3-butenylamino, 3-methyl-1-butenylamino, 1,2-dimethyl-2-propenylamino, 1,1-dimethyl-2-propenylamino, 2-methyl-2-butenylamino, 3-methyl-2-butenylamino, 1,2-dimethyl-1-propenylamino, 2-methyl-3-butenylamino, 3-methyl-3-butenylamino, 1,3-pentadienylamino, 1-vinyl-2-propenylamino, 1-hexenylamino, 1-propyl-2-propenylamino, 2-hexenylamino, 1-methyl-1-pentenylamino, 1-ethyl-2-butenylamino, 3-hexenylamino, 4-hexenylamino, 5-hexenylamino, 1-methyl-4-pentenylamino, 1-ethyl-3-butenylamino, 1-(isobutyl)vinylamino, 1-ethyl-1-methyl-2-propenylamino, 1-ethyl-2-methyl-2-propenylamino, 1-(isopropyl)-2-propenylamino, 2-methyl-2-pentenylamino, 3-methyl-3-pentenylamino, 4-methyl-3-pentenylamino, 1,3-dimethyl-2-butenylamino, 1,1-dimethyl-3-butenylamino, 3-methyl-4-pentenylamino, 4-methyl-4-pentenylamino, 1,2-dimethyl-3-butenylamino, 1,3-dimethyl-3-butenylamino, 1,1,2-trimethyl-2-propenylamino, 1,5-hexadienylamino, 1-vinyl-3-butenylamino, and 2,4-hexadienylamino groups.

In the present invention, unless otherwise specified, the “(C2-C6)alkynylamino” refers to a ((C2-C6)alkynyl)-NH— group whose alkynyl moiety is as defined above, and examples thereof include ethynylamino, 1-propynylamino, 2-propynylamino, 1-methyl-2-propynylamino, 1-ethyl-2-propynylamino, 1-butynylamino, 2-butynylamino, 3-butynylamino, 1-methyl-2-butynylamino, 1-pentynylamino, 2-pentynylamino, 3-pentynylamino, 4-pentynylamino, and 4,4-dimethyl-2-pentynylamino groups.

In the present invention, unless otherwise specified, the “(C3-C6)cycloalkylamino” refers to a ((C3-C6)cycloalkyl)-NH— group whose cycloalkyl moiety is as defined above, and examples thereof include cyclopropylamino, cyclobutylamino, cyclopentylamino, and cyclohexylamino groups.

In the present invention, unless otherwise specified, the “(C1-C6)alkylcarbonyl” refers to a ((C1-C6)alkyl)-C(═O)— group whose alkyl moiety is as defined above, and examples thereof include acetyl, propionyl, 2-methylpropionyl, 2,2-dimethylpropionyl, butanoyl, pivaloyl, 2-methylbutanoyl, 3-methylbutanoyl, 2-ethylbutanoyl, 2,2-dimethylbutanoyl, 2,3-dimethylbutanoyl, 3,3-dimethylbutanoyl, pentanoyl, 2-methylpentanoyl, 3-methylpentanoyl, 4-methylpentanoyl, and hexanoyl groups.

In the present invention, unless otherwise specified, the “(C2-C6)alkenylcarbonyl” refers to a ((C2-C6)alkenyl)-C(═O)— group whose alkenyl moiety is as defined above, and examples thereof include vinylcarbonyl, 1-propenylcarbonyl, isopropenylcarbonyl, 2-propenylcarbonyl, 1-methyl-2-propenylcarbonyl, 2-methyl-2-propenylcarbonyl, 2-butenylcarbonyl, 3-butenylcarbonyl, 3-methyl-2-butenylcarbonyl, 2-methyl-2-butenylcarbonyl, 2-pentenylcarbonyl, 3-pentenylcarbonyl, 4-pentenylcarbonyl, 2-hexenylcarbonyl, 3-hexenylcarbonyl, 4-hexenylcarbonyl, and 5-hexenylcarbonyl groups.

In the present invention, unless otherwise specified, the “(C2-C6)alkynylcarbonyl” refers to a ((C2-C6)alkynyl)-C(═O)— group whose alkynyl moiety is as defined above, and examples thereof include ethynylcarbonyl, 1-propynylcarbonyl, 2-propynylcarbonyl, 1-methyl-2-propynylcarbonyl, 1-ethyl-2-propynylcarbonyl, 1-butynylcarbonyl, 2-butynylcarbonyl, 3-butynylcarbonyl, 1-methyl-2-butynylcarbonyl, 1-pentynylcarbonyl, 2-pentynylcarbonyl, 3-pentynylcarbonyl, 4-pentynylcarbonyl, and 4,4-dimethyl-2-pentynylcarbonyl groups.

In the present invention, unless otherwise specified, the “(C3-C6)cycloalkylcarbonyl” refers to a ((C3-C6)cycloalkyl)-C(═O)— group whose cycloalkyl moiety is as defined above, and examples thereof include cyclopropylcarbonyl, cyclobutylcarbonyl, cyclopentylcarbonyl, and cyclohexylcarbonyl groups.

In the present invention, unless otherwise specified, the “(C6-C12)arylcarbonyl” refers to a ((C6-C12)aryl)-C(═O)— group whose aryl moiety is as defined above, and examples thereof include benzoyl, biphenylcarbonyl, naphthalen-1-ylcarbonyl, and naphthalen-2-ylcarbonyl groups.

In the present invention, unless otherwise specified, the “3- to 6-membered heterocyclylcarbonyl” refers to a (3- to 6-membered heterocyclyl)-C(═O)— group whose heterocyclyl moiety is as defined above, and examples thereof include aziridinylcarbonyl, oxylanylcarbonyl, oxetanylcarbonyl, thietanylcarbonyl, tetrahydrofuranylcarbonyl, tetrahydropyranylcarbonyl, azetidinylcarbonyl, pyrrolidinylcarbonyl, piperidinylcarbonyl, piperazinylcarbonyl, dihydroisoxazolylcarbonyl, dioxaborolanylcarbonyl, dioxolanylcarbonyl, dioxanylcarbonyl, and morpholinylcarbonyl groups.

In the present invention, unless otherwise specified, the “5- to 12-membered heteroarylcarbonyl” refers to a (5- to 12-membered heteroaryl)-C(═O)— group whose heteroaryl moiety is as defined above, and examples thereof include pyridylcarbonyl, pyrimidinylcarbonyl, pyrazinylcarbonyl, pyridadinylcarbonyl, triazinylcarbonyl, thienylcarbonyl, furanylcarbonyl, pyrrolylcarbonyl, pyrazolylcarbonyl, thiazolylcarbonyl, isothiazolylcarbonyl, oxazolylcarbonyl, isoxazolylcarbonyl, imidazolylcarbonyl, triazolylcarbonyl, thiadiazolylcarbonyl, oxadiazolylcarbonyl, tetrazolylcarbonyl, indolylcarbonyl, benzoimidazolylcarbonyl, indazolylcarbonyl, benzotriazolylcarbonyl, isoquinolinylcarbonyl, quinolinylcarbonyl, benzothiazolylcarbonyl, benzofuranylcarbonyl, benzothienylcarbonyl, benzoisoxazolylcarbonyl, pyrazolopyridylcarbonyl, pyrazolopyrimidinylcarbonyl, pyrrolopyridylcarbonyl, triazolopyridylcarbonyl, triazolopyrimidinylcarbonyl, quinazolinylcarbonyl, quinoxalinylcarbonyl, and cinnolinylcarbonyl groups.

In the present invention, unless otherwise specified, the “(C1-C6)alkoxycarbonyl” refers to a ((C1-C6)alkyl)-O—C(═O)— group whose alkyl moiety is as defined above, and examples thereof include methoxycarbonyl, ethoxycarbonyl, n-propoxycarbonyl, isopropoxycarbonyl, n-butoxycarbonyl, isobutoxycarbonyl, sec-butoxycarbonyl, tert-butoxycarbonyl, n-pentoxycarbonyl, 1-methyl butoxycarbonyl, 2-methyl butoxycarbonyl, 3-methyl butoxycarbonyl, 1-ethylpropoxycarbonyl, 1,1-dimethylpropoxycarbonyl, 1,2-dimethylpropoxycarbonyl, and 2,2-dimethylpropoxycarbonyl groups.

In the present invention, unless otherwise specified, the “(C2-C6)alkenyloxycarbonyl” refers to a ((C2-C6)alkenyl)-O—C(═O)— group whose alkenyl moiety is as defined above, and examples thereof include vinyloxycarbonyl, 1-propenyloxycarbonyl, isopropenyloxycarbonyl, 2-propenyloxycarbonyl, 1-methyl-2-propenyloxycarbonyl, 2-methyl-2-propenyloxycarbonyl, 2-butenyloxycarbonyl, 3-butenyloxycarbonyl, 3-methyl-2-butenyloxycarbonyl, 2-methyl-2-butenyloxycarbonyl, 2-pentenyloxycarbonyl, 3-pentenyloxycarbonyl, 4-pentenyloxycarbonyl, 2-hexenyloxycarbonyl, 3-hexenyloxycarbonyl, 4-hexenyloxycarbonyl, and 5-hexenyloxycarbonyl groups.

In the present invention, unless otherwise specified, the “(C2-C6)alkynyloxycarbonyl” refers to a ((C2-C6)alkynyl)-O—C(═O)— group whose alkynyl moiety is as defined above, and examples thereof include ethynyloxycarbonyl, 1-propynyloxycarbonyl, 2-propynyloxycarbonyl, 1-methyl-2-propynyloxycarbonyl, 1-ethyl-2-propynyloxycarbonyl, 1-butynyloxycarbonyl, 2-butynyloxycarbonyl, 3-butynyloxycarbonyl, 1-methyl-2-butynyloxycarbonyl, 1-pentynyloxycarbonyl, 2-pentynyloxycarbonyl, 3-pentynyloxycarbonyl, 4-pentynyloxycarbonyl, and 4,4-dimethyl-2-pentynyloxycarbonyl groups.

In the present invention, unless otherwise specified, the “(C3-C6)cycloalkyloxycarbonyl” refers to a ((C3-C6)cycloalkyl)-O—C(═O)— group whose cycloalkyl moiety is as defined above, and examples thereof include cyclopropyloxycarbonyl, cyclobutyloxycarbonyl, cyclopentyloxycarbonyl, and cyclohexyloxycarbonyl groups.

In the present invention, unless otherwise specified, the “(C6-C12)aryloxycarbonyl” refers to a ((C6-C12)aryl)-O—C(═O)— group whose aryl moiety is as defined above, and examples thereof include phenyloxycarbonyl, biphenyloxycarbonyl, naphthalen-1-yloxycarbonyl, and naphthalen-2-yloxycarbonyl groups.

In the present invention, unless otherwise specified, the “3- to 6-membered heterocyclyloxycarbonyl” refers to a (3- to 6-membered heterocyclyl)-O—C(═O)— group whose heterocyclyl moiety is as defined above, and examples thereof include aziridinyloxycarbonyl, oxylanyloxycarbonyl, oxetanyloxycarbonyl, thietanyloxycarbonyl, tetrahydrofuranyloxycarbonyl, tetrahydropyranyloxycarbonyl, azetidinyloxycarbonyl, pyrrolidinyloxycarbonyl, piperidinyloxycarbonyl, piperazinyloxycarbonyl, dihydroisoxazolyloxycarbonyl, dioxaborolanyloxycarbonyl, dioxolanyloxycarbonyl, dioxanyloxycarbonyl, and morpholinyloxycarbonyl groups.

In the present invention, unless otherwise specified, the “5- to 12-membered heteroaryloxycarbonyl” refers to a (5- to 12-membered heteroaryl)-O—C(═O)— group whose heteroaryl moiety is as defined above, and examples thereof include pyridyloxycarbonyl, pyrimidinyloxycarbonyl, pyrazinyloxycarbonyl, pyridadinyloxycarbonyl, triazinyloxycarbonyl, thienyloxycarbonyl, furanyloxycarbonyl, pyrrolyloxycarbonyl, pyrazolyloxycarbonyl, thiazolyloxycarbonyl, isothiazolyloxycarbonyl, oxazolyloxycarbonyl, isoxazolyloxycarbonyl, imidazolyloxycarbonyl, triazolyloxycarbonyl, thiadiazolyloxycarbonyl, oxadiazolyloxycarbonyl, tetrazolyloxycarbonyl, indolyloxycarbonyl, benzoimidazolyloxycarbonyl, indazolyloxycarbonyl, benzotriazolyloxycarbonyl, isoquinolinyloxycarbonyl, quinolinyloxycarbonyl, benzothiazolyloxycarbonyl, benzofuranyloxycarbonyl, benzothienyloxycarbonyl, benzoisooxazolyloxycarbonyl, pyrazolopyridyloxycarbonyl, pyrazolopyrimidinyloxycarbonyl, pyrrolopyridyloxycarbonyl, triazolopyridyloxycarbonyl, triazolopyrimidinyloxycarbonyl, quinazolinyloxycarbonyl, quinoxalinyloxycarbonyl, and cinnolinyloxycarbonyl groups.

In the present invention, unless otherwise specified, the “(C1-C6)alkylaminocarbonyl” refers to a ((C1-C6)alkyl)-NH—C(═O)— group whose alkyl moiety is as defined above, and examples thereof include methylaminocarbonyl, ethylaminocarbonyl, n-propylaminocarbonyl, isopropylaminocarbonyl, n-butylaminocarbonyl, isobutylaminocarbonyl, sec-butylaminocarbonyl, tert-butylaminocarbonyl, n-pentylaminocarbonyl, 1-methylbutylaminocarbonyl, 2-methylbutylaminocarbonyl, 3-methylbutylaminocarbonyl, 1-ethylpropylaminocarbonyl, 1,1-dimethylpropylaminocarbonyl, 1,2-dimethylpropylaminocarbonyl, 2,2-dimethylpropylaminocarbonyl, and n-hexylaminocarbonyl groups.

In the present invention, unless otherwise specified, the “(C2-C6)alkenylaminocarbonyl” refers to a ((C2-C6)alkenyl)-NH—C(═O)— group whose alkenyl moiety is as defined above, and examples thereof include vinylaminocarbonyl, 1-propenylaminocarbonyl, isopropenylaminocarbonyl, 2-propenylaminocarbonyl, 1-methyl-2-propenylaminocarbonyl, 2-methyl-2-propenylaminocarbonyl, 2-butenylaminocarbonyl, 3-butenylaminocarbonyl, 3-methyl-2-butenylaminocarbonyl, 2-methyl-2-butenylaminocarbonyl, 2-pentenylaminocarbonyl, 3-pentenylaminocarbonyl, 4-pentenylaminocarbonyl, 2-hexenylaminocarbonyl, 3-hexenylaminocarbonyl, 4-hexenylaminocarbonyl, and 5-hexenylaminocarbonyl groups.

In the present invention, unless otherwise specified, the “(C2-C6)alkynylaminocarbonyl” refers to a ((C2-C6)alkynyl)-NH—C(═O)— group whose alkynyl moiety is as defined above, and examples thereof include ethynylaminocarbonyl, 1-propynylaminocarbonyl, 2-propynylaminocarbonyl, 1-methyl-2-propynylaminocarbonyl, 1-ethyl-2-propynylaminocarbonyl, 1-butynylaminocarbonyl, 2-butynylaminocarbonyl, 3-butynylaminocarbonyl, 1-methyl-2-butynylaminocarbonyl, 1-pentynylaminocarbonyl, 2-pentynylaminocarbonyl, 3-pentynylaminocarbonyl, 4-pentynylaminocarbonyl, and 4,4-dimethyl-2-pentynylaminocarbonyl groups.

In the present invention, unless otherwise specified, the “(C3-C6)cycloalkylaminocarbonyl” refers to a ((C3-C6)cycloalkyl)-NH—C(═O)— group whose cycloalkyl moiety is as defined above, and examples thereof include cyclopropylaminocarbonyl, cyclobutylaminocarbonyl, cyclopentylaminocarbonyl, and cyclohexylaminocarbonyl groups.

In the present invention, unless otherwise specified, the “(C6-C12)arylaminocarbonyl” refers to a ((C6-C12)aryl)-NH—C(═O)— group whose aryl moiety is as defined above, and examples thereof include phenylaminocarbonyl, biphenylaminocarbonyl, naphthalen-1-ylaminocarbonyl, and naphthalen-2-ylaminocarbonyl groups.

In the present invention, unless otherwise specified, the “3- to 6-membered heterocyclylaminocarbonyl” refers to a (3- to 6-membered heterocyclyl)-NH—C(═O)— group whose heterocyclyl moiety is as defined above, and examples thereof include aziridinylaminocarbonyl, oxylanylaminocarbonyl, oxetanylaminocarbonyl, thietanylaminocarbonyl, tetrahydrofuranylaminocarbonyl, tetrahydropyranylaminocarbonyl, azetidinylaminocarbonyl, pyrrolidinylaminocarbonyl, piperidinylaminocarbonyl, piperazinylaminocarbonyl, dihydroisoxazolylaminocarbonyl, dioxaborolanylaminocarbonyl, dioxolanylaminocarbonyl, dioxanylaminocarbonyl, and morpholinylaminocarbonyl groups.

In the present invention, unless otherwise specified, the “5- to 12-membered heteroarylaminocarbonyl” refers to a (5- to 12-membered heteroaryl)-NH—C(═O)— group whose heteroaryl moiety is as defined above, and examples thereof include pyridylaminocarbonyl, pyrimidinylaminocarbonyl, pyrazinylaminocarbonyl, pyridadinylaminocarbonyl, triazinylaminocarbonyl, thienylaminocarbonyl, furanylaminocarbonyl, pyrrolylaminocarbonyl, pyrazolylaminocarbonyl, thiazolylaminocarbonyl, isothiazolylaminocarbonyl, oxazolylaminocarbonyl, isoxazolylaminocarbonyl, imidazolylaminocarbonyl, triazolylaminocarbonyl, thiadiazolylaminocarbonyl, oxadiazolylaminocarbonyl, tetrazolylaminocarbonyl, indolylaminocarbonyl, benzoimidazolylaminocarbonyl, indazolylaminocarbonyl, benzotriazolylaminocarbonyl, isoquinolinylaminocarbonyl, quinolinylaminocarbonyl, benzothiazolylaminocarbonyl, benzofuranylaminocarbonyl, benzothienylaminocarbonyl, benzoisooxazolylaminocarbonyl, pyrazolopyridylaminocarbonyl, pyrazolopyrimidinylaminocarbonyl, pyrrolopyridylaminocarbonyl, triazolopyridylaminocarbonyl, triazolopyrimidinylaminocarbonyl, quinazolinylaminocarbonyl, quinoxalinylaminocarbonyl, and cinnolinylaminocarbonyl groups.

In the present invention, unless otherwise specified, the “(C1-C6)alkylcarbonylamino” refers to a ((C1-C6)alkyl)-C(═O)—NH— group whose alkyl moiety is as defined above, and examples thereof include acetylamino, propionylamino, butyrylamino, and isobutyrylamino groups.

In the present invention, unless otherwise specified, the “(C1-C6)alkoxycarbonylamino” refers to a ((C1-C6)alkyl)-O—C(═O)—NH— group whose alkyl moiety is as defined above, and examples thereof include methoxycarbonylamino, ethoxycarbonylamino, isopropoxycarbonylamino, tert-butoxycarbonylamino, and isobutoxycarbonylamino groups.

In the present invention, unless otherwise specified, the “(C2-C6)alkenylcarbonylamino” refers to a ((C2-C6)alkenyl)-C(═O)—NH— group whose alkenyl moiety is as defined above, and examples thereof include vinylcarbonylamino, 1-propenylcarbonylamino, isopropenylcarbonylamino, 2-propenylcarbonylamino, 1-methyl-2-propenylcarbonylamino, 2-methyl-2-propenylcarbonylamino, 2-butenylcarbonylamino, 3-butenylcarbonylamino, 3-methyl-2-butenylcarbonylamino, 2-methyl-2-butenylcarbonylamino, 2-pentenylcarbonylamino, 3-pentenylcarbonylamino, 4-pentenylcarbonylamino, 2-hexenylcarbonylamino, 3-hexenylcarbonylamino, 4-hexenylcarbonylamino, and 5-hexenylcarbonylamino groups.

In the present invention, unless otherwise specified, the “(C2-C6)alkynylcarbonylamino” refers to a ((C2-C6)alkynyl)-C(═O)—NH— group whose alkynyl moiety is as defined above, and examples thereof include ethynylcarbonylamino, 1-propynylcarbonylamino, 2-propynylcarbonylamino, 1-methyl-2-propynylcarbonylamino, 1-ethyl-2-propynylcarbonylamino, 1-butynylcarbonylamino, 2-butynylcarbonylamino, 3-butynylcarbonylamino, 1-methyl-2-butynylcarbonylamino, 1-pentynylcarbonylamino, 2-pentynylcarbonylamino, 3-pentynylcarbonylamino, 4-pentynylcarbonylamino, and 4,4-dimethyl-2-pentynylcarbonylamino groups.

In the present invention, unless otherwise specified, the “(C3-C6)cycloalkylcarbonylamino” refers to a (C3-C6 cycloalkyl)-C(═O)—NH— group whose cycloalkyl moiety is as defined above, and examples thereof include cyclopropylcarbonylamino, cyclobutylcarbonylamino, cyclopentylcarbonylamino, and cyclohexylcarbonylamino groups.

In the present invention, unless otherwise specified, the “(C6-C12)arylcarbonylamino” refers to a ((C6-C12)aryl)-C(═O)—NH— group whose aryl moiety is as defined above, and examples thereof include benzoylamino, biphenylcarbonylamino, naphthalen-1-ylcarbonylamino, and naphthalen-2-ylcarbonylamino groups.

In the present invention, unless otherwise specified, the “3- to 6-membered heterocyclylcarbonylamino” refers to a (3- to 6-membered heterocyclyl)-C(═O)—NH— group whose heterocyclyl moiety is as defined above, and examples thereof include aziridinylcarbonylamino, oxylanylcarbonylamino, oxetanylcarbonylamino, thietanylcarbonylamino, tetrahydrofuranylcarbonylamino, tetrahydropyranylcarbonylamino, azetidinylcarbonylamino, pyrrolidinylcarbonylamino, piperidinylcarbonylamino, piperazinylcarbonylamino, dihydroisoxazolylcarbonylamino, dioxaborolanylcarbonylamino, dioxolanylcarbonylamino, dioxanylcarbonylamino, and morpholinylcarbonylamino groups.

In the present invention, unless otherwise specified, the “5- to 12-membered heteroarylcarbonylamino” refers to a (5- to 12-membered heteroaryl)-C(═O)—NH— group whose heteroaryl moiety is as defined above, and examples thereof include pyridylcarbonylamino, pyrimidinylcarbonylamino, pyrazinylcarbonylamino, pyridadinylcarbonylamino, triazinylcarbonylamino, thienylcarbonylamino, furanylcarbonylamino, pyrrolylcarbonylamino, pyrazolylcarbonylamino, thiazolylcarbonylamino, isothiazolylcarbonylamino, oxazolylcarbonylamino, isoxazolylcarbonylamino, imidazolylcarbonylamino, triazolylcarbonylamino, thiadiazolylcarbonylamino, oxadiazolylcarbonylamino, tetrazolylcarbonylamino, indolylcarbonylamino, benzoimidazolylcarbonylamino, indazolylcarbonylamino, benzotriazolylcarbonylamino, isoquinolinylcarbonylamino, quinolinylcarbonylamino, benzothiazolylcarbonylamino, benzofuranylcarbonylamino, benzothienylcarbonylamino, benzoisooxazolylcarbonylamino, pyrazolopyridylcarbonylamino, pyrazolopyrimidinylcarbonylamino, pyrrolopyridylcarbonylamino, triazolopyridylcarbonylamino, triazolopyrimidinylcarbonylamino, quinazolinylcarbonylamino, quinoxalinylcarbonylamino, and cinnolinylcarbonylamino groups.

In the present invention, unless otherwise specified, “(C1-C6)alkylcarbonyloxy” refers to a ((C1-C6)alkyl)-C(═O)— O— group whose alkyl moiety is as defined above, and examples thereof include methylcarbonyloxy, ethylcarbonyloxy, n-propylcarbonyloxy, isopropylcarbonyloxy, n-butylcarbonyloxy, isobutylcarbonyloxy, sec-butylcarbonyloxy, and tert-butylcarbonyloxy groups.

In the present invention, unless otherwise specified, the “(C2-C6)alkenylcarbonyloxy” refers to a ((C2-C6)alkenyl)-C(═O)—O— group whose alkenyl moiety is as defined above, and examples thereof include vinylcarbonyloxy, 1-propenylcarbonyloxy, isopropenylcarbonyloxy, 2-propenylcarbonyloxy, 1-methyl-2-propenylcarbonyloxy, 2-methyl-2-propenylcarbonyloxy, 2-butenylcarbonyloxy, 3-butenylcarbonyloxy, 3-methyl-2-butenylcarbonyloxy, 2-methyl-2-butenylcarbonyloxy, 2-pentenylcarbonyloxy, 3-pentenylcarbonyloxy, 4-pentenylcarbonyloxy, 2-hexenylcarbonyloxy, 3-hexenylcarbonyloxy, 4-hexenylcarbonyloxy, and 5-hexenylcarbonyloxy groups.

In the present invention, unless otherwise specified, the “(C2-C6)alkynylcarbonyloxy” refers to a ((C2-C6)alkynyl)-C(═O)—O— group whose alkynyl moiety is as defined above, and examples thereof include ethynylcarbonyloxy, 1-propynylcarbonyloxy, 2-propynylcarbonyloxy, 1-methyl-2-propynylcarbonyloxyl, 1-ethyl-2-propynylcarbonyloxy, 1-butynylcarbonyloxy, 2-butynylcarbonyloxy, 3-butynylcarbonyloxy, 1-methyl-2-butynylcarbonyloxy, 1-pentynylcarbonyloxy, 2-pentynylcarbonyloxy, 3-pentynylcarbonyloxy, 4-pentynylcarbonyloxy, and 4,4-dimethyl-2-pentynylcarbonyloxy groups.

In the present invention, unless otherwise specified, the “(C3-C6)cycloalkylcarbonyloxy” refers to a ((C3-C6)cycloalkyl)-C(═O)—O— group whose cycloalkyl moiety is as defined above, and examples thereof include cyclopropylcarbonyloxy, cyclobutylcarbonyloxy, cyclopentylcarbonyloxy, and cyclohexylcarbonyloxy groups.

In the present invention, unless otherwise specified, the “(C6-C12)arylcarbonyloxy” refers to a ((C6-C12)aryl)-C(═O)—O— group whose aryl moiety is as defined above, and examples thereof include benzoyloxy, biphenylcarbonyloxy, naphthalen-1-ylcarbonyloxy, and naphthalen-2-ylcarbonyloxy groups.

In the present invention, unless otherwise specified, the “3- to 6-membered heterocyclylcarbonyloxy” refers to a (3- to 6-membered heterocyclyl)-C(═O)—O— group whose heterocyclyl moiety is as defined above, and examples thereof include aziridinylcarbonyloxy, oxylanylcarbonyloxy, oxetanylcarbonyloxy, thietanylcarbonyloxy, tetrahydrofuranylcarbonyloxy, tetrahydropyranylcarbonyloxy, azetidinylcarbonyloxy, pyrrolidinylcarbonyloxyy, piperidinylcarbonyloxy, piperazinylcarbonyloxy, dihydroisoxazolylcarbonyloxy, dioxaborolanylcarbonyloxy, dioxolanylcarbonyloxy, dioxanylcarbonyloxyl, and morpholinylcarbonyloxy groups.

In the present invention, unless otherwise specified, the “5- to 12-membered heteroarylcarbonyloxy” refers to a (5- to 12-membered heteroaryl)-C(═O)—O— group whose heteroaryl moiety is as defined above, and examples thereof include pyridylcarbonyloxy, pyrimidinylcarbonyloxy, pyrazinylcarbonyloxy, pyridadinylcarbonyloxy, triazinylcarbonyloxy, thienylcarbonyloxy, furanylcarbonyloxy, pyrrolylcarbonyloxy, pyrazolylcarbonyloxy, thiazolylcarbonyloxy, isothiazolylcarbonyloxy, oxazolylcarbonyloxy, isoxazolylcarbonyloxy, imidazolylcarbonyloxy, triazolylcarbonyloxy, thiadiazolylcarbonyloxy, oxadiazolylcarbonyloxy, tetrazolylcarbonyloxy, indolylcarbonyloxy, benzoimidazolylcarbonyloxy, indazolylcarbonyloxy, benzotriazolylcarbonyloxy, isoquinolinylcarbonyloxy, quinolinylcarbonyloxy, benzothiazolylcarbonyloxy, benzofuranylcarbonyloxy, benzothienylcarbonyloxy, benzoisooxazolylcarbonyloxy, pyrazolopyridylcarbonyloxy, pyrazolopyrimidinylcarbonyloxy, pyrrolopyridylcarbonyloxy, triazolopyridylcarbonyloxy, triazolopyrimidinylcarbonyloxy, quinazolinylcarbonyloxy, quinoxalinylcarbonyloxy, and cinnolinylcarbonyloxy groups.

In the present invention, unless otherwise specified, the “(C1-C6)alkoxy” refers to a ((C1-C6)alkyl)-O— group whose alkyl moiety is as defined above, and examples thereof include methoxy, ethoxy, n-propoxy, isopropoxy, n-butoxy, isobutoxy, sec-butoxy, tert-butoxy, n-pentoxy, 1-methylbutoxy, 2-methylbutoxy, 3-methylbutoxy, 1-ethylpropoxy, 1,1-dimethylpropoxy, 1,2-dimethylpropoxy, and n-hexyloxy groups.

In the present invention, unless otherwise specified, the “(C1-C6)haloalkoxy” refers to a ((C1-C6)haloalkyl)-O— group whose haloalkyl moiety is as defined above, and examples thereof include difluoromethoxy, dichloromethoxy, trifluoromethoxy, trichloromethoxy, tribromomethoxy, chlorodifluoromethoxy, bromodifluoromethoxy, 2-fluoroethoxy, 1-chloroethoxy, 2-chloroethoxy, 1-bromoethoxy, 2-bromoethoxy, 2,2-difluoroethoxy, 1,2-dichloroethoxy, 2,2-dichloroethoxy, 2,2,2-trifluoroethoxy, 2,2,2-trichloroethoxy, 1,1,2,2-tetrafluoroethoxy, pentafluoroethoxy, 2-bromo-2-chloroethoxy, 2-chloro-1,1,2,2-tetrafluoroethoxy, 1-chloro-1,2,2,2-tetrafluoroethoxy, 1-chloropropoxy, 2-chloropropoxy, 3-chloropropoxy, 2-bromopropoxy, 3-bromopropoxy, 2-bromo-1-methylethoxy, 3-iodopropoxy, 2,3-dichloropropoxy, 2,3-dibromopropoxy, 3,3,3-trifluoropropoxy, 3,3,3-trifluoro-2-propoxy, 3,3,3-trichloropropoxy, 3-bromo-3,3-difluoropropoxy, 2,2-difluoropropoxy, 3,3-dichloro-3-fluoropropoxy, 2,2,3,3-tetrafluoropropoxy, 1-bromo-3,3,3-trifluoropropoxy, 2,2,3,3,3-pentafluoropropoxy, 2,2,2-trifluoro-1-trifluoromethylethoxy, heptafluoropropoxy, heptafluoro-2-propoxy, 1,2,2,2-tetrafluoro-1-trifluoromethylethoxy, 1,1,2,3,3,3-hexafluoropropoxy, 2-chlorobutoxy, 3-chlorobutoxy, 4-chlorobutoxy, 2-chloro-1,1-dimethylethoxy, 4-bromobutoxy, 3-bromo-2-methylpropoxy, 2-bromo-1,1-dimethylethoxy, 2,2-dichloro-1,1-dimethylethoxy, 2-chloro-1-chloromethyl-2-methylethoxy, 4,4,4-trifluorobutoxy, 3,3,3-trifluoro-1-methylpropoxy, 3,3,3-trifluoro-2-methylpropoxy, 2,3,4-trichlorobutoxy, 2,2,2-trichloro-1,1-dimethylethoxy, 4-chloro-4,4-difluorobutoxy, 4,4-dichloro-4-fluorobutoxy, 4-bromo-4,4-difluorobutoxy, 2,4-dibromo-4,4-difluorobutoxy, 3,4-dichloro-3,4,4-trifluorobutoxy, 3,3-dichloro-4,4,4-trifluorobutoxy, 4-bromo-3,3,4,4-tetrafluorobutoxy, 4-bromo-3-chloro-3,4,4-trifluorobutoxy, 2,2,3,3,4,4-hexafluorobutoxy, 2,2,3,4,4,4-hexafluorobutoxy, 2,2,2-trifluoro-1-methyl-1-trifluoromethylethoxy, 3,3,3-trifluoro-2-trifluoromethylpropoxy, 2,2,3,3,4,4,4-heptafluorobutoxy, 3,3,4,4,4-pentafluoro-2-butoxy, 2,3,3,3-tetrafluoro-2-trifluoromethylpropoxy, 1,1,2,2,3,3,4,4-octafluorobutoxy, nonafluorobutoxy, perfluoro-tert-butoxy, 4-chloro-1,1,2,2,3,3,4,4-octafluorobutoxy, 5,5,5-trifluoropentoxy, 4,4,5,5,5-pentafluoropentoxy, 3,3,4,4,5,5,5-heptafluoropentoxy, 3,3,4,4,5,5,5-heptafluoro-2-pentoxy, 2,2,3,3,4,4,5,5,5-nonafluoropentoxy, 2,2,3,3,4,4,5,5-octafluoropentoxy, perfluoropentoxy, 4,4,5,5,5-pentafluoro-2-butoxy, 2,2-bis(trifluoromethyl)propoxy, 2,2,3,3,4,4,5,5,6, 6,6-undecafluorohexyloxy, 3,3,4,4,5,5,6,6,6-nonafluorohexyloxy, 4,4,5,5,6,6,6-heptafluorohexyloxy, 2,2,3,3,4,4,5,5,6,6-decafluorohexyloxy, 4,4,4-trifluoro-3,3-bis(trifluoromethyl)butyloxy, and perfluorohexyloxy groups.

In the present invention, unless otherwise specified, the “(C2-C6)alkenyloxy” refers to a ((C2-C6)alkenyl)-O— group whose alkenyl moiety is as defined above, and examples thereof include vinyloxy, 1-propenyloxy, isopropenyloxy, 2-propenyloxy, 1-methyl-2-propenyloxy, 2-methyl-2-propenyloxy, 2-butenyloxy, 3-butenyloxy, 3-methyl-2-butenyloxy, 2-methyl-2-butenyloxy, 2-pentenyloxy, 3-pentenyloxy, 4-pentenyloxy, 2-hexenyloxy, 3-hexenyloxy, 4-hexenyloxy, and 5-hexenyloxy groups.

In the present invention, unless otherwise specified, the “(C2-C6)alkynyloxy” refers to a ((C2-C6)alkynyl)-O— group whose alkynyl moiety is as defined above, and examples thereof include ethynyloxy, 1-propynyloxy, 2-propynyloxy, 1-methyl-2-propynyloxy, 1-ethyl-2-propynyloxy, 1-butynyloxy, 2-butynyloxy, 3-butynyloxy, 1-methyl-2-butynyloxy, 1-pentynyloxy, 2-pentynyloxy, 3-pentynyloxy, 4-pentynyloxy, and 4,4-dimethyl-2-pentynyloxy groups.

In the present invention, unless otherwise specified, the “(C3-C6)cycloalkyloxy” refers to a ((C3-C6)cycloalkyl)-O— group whose cycloalkyl moiety is as defined above, and examples thereof include cyclopropyloxy, cyclobutyloxy, cyclopentyloxy, and cyclohexyloxy groups.

In the present invention, unless otherwise specified, the “(C1-C6)alkylsulfonylamino” refers to a ((C1-C6)alkyl)-S(═O)₂—NH— group whose alkyl moiety is as defined above, and examples thereof include methylsulfonylamino, ethylsulfonylamino, n-propylsulfonylamino, isopropylsulfonylamino, and tert-butylsulfonylamino groups.

In the present invention, unless otherwise specified, the “(C2-C6)alkenylsulfonylamino” refers to a ((C2-C6)alkenyl)-S(═O)₂—NH— group whose alkenyl moiety is as defined above, and examples thereof include vinylsulfonylamino, 1-propenylsulfonylamino, isopropenylsulfonylamino, 2-propenylsulfonylamino, 1-methyl-2-propenylsulfonylamino, 2-methyl-2-propenylsulfonylamino, 2-butenylsulfonylamino, 3-butenylsulfonylamino, 3-methyl-2-butenylsulfonylamino, 2-methyl-2-butenylsulfonylamino, 2-pentenylsulfonylamino, 3-pentenylsulfonylamino, 4-pentenylsulfonylamino, 2-hexenylsulfonylamino, 3-hexenylsulfonylamino, 4-hexenylsulfonylamino, and 5-hexenylsulfonylamino groups.

In the present invention, unless otherwise specified, the “(C2-C6)alkynylsulfonylamino” refers to a ((C2-C6)alkynyl)-S(═O)₂—NH— group whose alkynyl moiety is as defined above, and examples thereof include ethynylsulfonylamino, 1-propynylsulfonylamino, 2-propynylsulfonylamino, 1-methyl-2-propynylsulfonylamino, 1-ethyl-2-propynylsulfonylamino, 1-butynylsulfonylamino, 2-butynylsulfonylamino, 3-butynylsulfonylamino, 1-methyl-2-butynylsulfonylamino, 1-pentynylsulfonylamino, 2-pentynylsulfonylamino, 3-pentynylsulfonylamino, 4-pentynylsulfonylamino, and 4,4-dimethyl-2-pentynylsulfonylamino groups.

In the present invention, unless otherwise specified, the “(C3-C6)cycloalkylsulfonylamino” refers to a ((C3-C6)cycloalkyl)-S(═O)₂—NH— group whose cycloalkyl moiety is as defined above, and examples thereof include cyclopropylsulfonylamino, cyclobutylsulfonylamino, cyclopentylsulfonylamino, and cyclohexylsulfonylamino groups.

In the present invention, unless otherwise specified, the “(C6-C12)arylsulfonylamino” refers to a ((C6-C12)aryl)-S(═O)₂—NH— group whose aryl moiety is as defined above, and examples thereof include phenylsulfonylamino, biphenylsulfonylamino, naphthalen-1-ylsulfonylamino, and naphthalen-2-ylsulfonylamino groups.

In the present invention, unless otherwise specified, the “3- to 6-membered heterocyclylsulfonylamino” refers to a (3- to 6-membered heterocyclyl)-S(═O)₂—NH— group whose heterocyclyl moiety is as defined above, and examples thereof include aziridinylsulfonylamino, oxiranylsulfonylamino, oxetanylsulfonylamino, thietanylsulfonylamino, tetrahydrofuranylsulfonylamino, tetrahydropyranylsulfonylamino, azetidinylsulfonylamino, pyrrolidinylsulfonylamino, piperidinylsulfonylamino, piperazinylsulfonylamino, dihydroisoxazolylsulfonylamino, dioxaborolanylsulfonylamino, dioxolanylsulfonylamino, dioxanylsulfonylamino, and morpholinylsulfonylamino groups.

In the present invention, unless otherwise specified, the “5- to 12-membered heteroarylsulfonylamino” refers to a (5- to 12-membered heteroaryl)-S(═O)₂—NH— group whose heteroaryl moiety is as defined above, and examples thereof include pyridylsulfonylamino, pyrimidinylsulfonylamino, pyrazinylsulfonylamino, pyridadinylsulfonylamino, triazinylsulfonylamino, thienylsulfonylamino, furanylsulfonylamino, pyrrolylsulfonylamino, pyrazolylsulfonylamino, thiazolylsulfonylamino, isothiazolylsulfonylamino, oxazolylsulfonylamino, isoxazolylsulfonylamino, imidazolylsulfonylamino, triazolylsulfonylamino, thiadiazolylsulfonylamino, oxadiazolylsulfonylamino, tetrazolylsulfonylamino, indolylsulfonylamino, benzoimidazolylsulfonylamino, indazolylsulfonylamino, benzotriazolylsulfonylamino, isoquinolinylsulfonylamino, quinolinylsulfonylamino, benzothiazolylsulfonylamino, benzofuranylsulfonylamino, benzothienylsulfonylamino, benzoisooxazolylsulfonylamino, pyrazolopyridylsulfonylamino, pyrazolopyrimidinylsulfonylamino, pyrrolopyridylsulfonylamino, triazolopyridylsulfonylamino, triazolopyrimidinylsulfonylamino, quinazolinylsulfonylamino, quinoxalinylsulfonylamino, and cinnolinylsulfonylamino groups.

In the present invention, unless otherwise specified, the “(C1-C6)alkylaminosulfonyl” refers to a ((C1-C6)alkyl)-NH—S(═O)₂— group whose alkyl moiety is as defined above, and examples thereof include methylaminosulfonyl, ethylaminosulfonyl, n-propylaminosulfonyl, isopropylaminosulfonyl, and tert-butylaminosulfonyl groups.

In the present invention, unless otherwise specified, the “(C2-C6)alkenylaminosulfonyl” refers to a ((C2-C6)alkenyl)-NH—S(═O)₂— group whose alkenyl moiety is as defined above, and examples thereof include vinylaminosulfonyl, 1-propenylaminosulfonyl, isopropenylaminosulfonyl, 2-propenylaminosulfonyl, 1-methyl-2-propenylaminosulfonyl, 2-methyl-2-propenylaminosulfonyl, 2-butenylaminosulfonyl, 3-butenylaminosulfonyl, 3-methyl-2-butenylaminosulfonyl, 2-methyl-2-butenylaminosulfonyl, 2-pentenylaminosulfonyl, 3-pentenylaminosulfonyl, 4-pentenylaminosulfonyl, 2-hexenylaminosulfonyl, 3-hexenylaminosulfonyl, 4-hexenylaminosulfonyl, and 5-hexenylaminosulfonyl groups.

In the present invention, unless otherwise specified, the “(C2-C6)alkynylaminosulfonyl” refers to a ((C2-C6)alkynyl)-NH—S(═O)₂— group whose alkynyl moiety is as defined above, and examples thereof include ethynylaminosulfonyl, 1-propynylaminosulfonyl, 2-propynylaminosulfonyl, 1-methyl-2-propynylaminosulfonyl, 1-ethyl-2-propynylaminosulfonyl, 1-butynylaminosulfonyl, 2-butynylaminosulfonyl, 3-butynylaminosulfonyl, 1-methyl-2-butynylaminosulfonyl, 1-pentynylaminosulfonyl, 2-pentynylaminosulfonyl, 3-pentynylaminosulfonyl, 4-pentynylaminosulfonyl, and 4,4-dimethyl-2-pentynylaminosulfonyl groups.

In the present invention, unless otherwise specified, the “(C3-C6)cycloalkylaminosulfonyl” refers to a ((C3-C6)cycloalkyl)-NH—S(═O)₂— group whose cycloalkyl moiety is as defined above, and examples thereof include cyclopropylaminosulfonyl, cyclobutylaminosulfonyl, cyclopentylaminosulfonyl, and cyclohexylaminosulfonyl groups.

In the present invention, unless otherwise specified, the “(C6-C12)arylaminosulfonyl” refers to a ((C6-C12)aryl)-NH—S(═O)₂— group whose aryl moiety is as defined above, and examples thereof include phenylaminosulfonyl, biphenylaminosulfonyl, naphthalen-1-ylaminosulfonyl, and naphthalen-2-ylaminosulfonyl groups.

In the present invention, unless otherwise specified, the “3- to 6-membered heterocyclylaminosulfonyl” refers to a (3- to 6-membered heterocyclyl)-NH—S(═O)₂— group whose heterocyclyl moiety is as defined above, and examples thereof include aziridinylaminosulfonyl, oxiranylaminosulfonyl, oxetanylaminosulfonyl, thietanylaminosulfonyl, tetrahydrofuranylaminosulfonyl, tetrahydropyranylaminosulfonyl, azetidinylaminosulfonyl, pyrrolidinylaminosulfonyl, piperidinylaminosulfonyl, piperazinylaminosulfonyl, dihydroisoxazolylaminosulfonyl, dioxaborolanylaminosulfonyl, dioxolanylaminosulfonyll, dioxanylaminosulfonyl, and morpholinylaminosulfonyl groups.

In the present invention, unless otherwise specified, the “5- to 12-membered heteroarylaminosulfonyl” refers to a (5- to 12-membered heteroaryl)-NH—S(═O)₂— group whose heteroaryl moiety is as defined above, and examples thereof include pyridylaminosulfonyl, pyrimidinylaminosulfonyl, pyrazinylaminosulfonyl, pyridadinylaminosulfonyl, triazinylaminosulfonyl, thienylaminosulfonyl, furanylaminosulfonyl, pyrrolylaminosulfonyl, pyrazolylaminosulfonyl, thiazolylaminosulfonyl, isothiazolylaminosulfonyl, oxazolylaminosulfonyl, isoxazolylaminosulfonyl, imidazolylaminosulfonyl, triazolylaminosulfonyl, thiadiazolylaminosulfonyl, oxadiazolylaminosulfonyl, tetrazolylaminosulfonyl, indolylaminosulfonyl, benzoimidazolylaminosulfonyl, indazolylaminosulfonyl, benzotriazolylaminosulfonyl, isoquinolinylaminosulfonyl, quinolinylaminosulfonyl, benzothiazolylaminosulfonyl, benzofuranylaminosulfonyl, benzothienylaminosulfonyl, benzoisooxazolylaminosulfonyl, pyrazolopyridylaminosulfonyl, pyrazolopyrimidinylaminosulfonyl, pyrrolopyridylaminosulfonyl, triazolopyridylaminosulfonyl, triazolopyrimidinylaminosulfonyl, quinazolinylaminosulfonyl, quinoxalinylaminosulfonyl, and cinnolinylaminosulfonyl groups.

In the present invention, unless otherwise specified, “(C1-C6)alkylcarbonylthio” refers to a ((C1-C6)alkyl)-C(═O)—S— group whose alkyl moiety is as defined above, and examples thereof include methylcarbonylthio, ethylcarbonylthio, n-propylcarbonylthio, isopropylcarbonylthio, n-butylcarbonylthio, isobutylcarbonylthio, sec-butylcarbonylthio, and tert-butylcarbonylthio groups.

In the present invention, unless otherwise specified, the “(C1-C6)alkylthio group” refers to a ((C1-C6)alkyl)-S— group whose alkyl moiety is as defined above, and examples thereof include methylthio, ethylthio, n-propylthio, isopropylthio, n-butylthio, isobutylthio, sec-butylthio, and tert-butylthio groups.

In the present invention, unless otherwise specified, the “(C1-C6)alkylsulfinyl” refers to a (C1-C6 alkyl) —S(═O)— group whose alkyl moiety is as defined above, and examples thereof include methylsulfinyl, ethylsulfinyl, n-propylsulfinyl, isopropylsulfinyl, n-butylsulfinyl, isobutylsulfinyl, sec-butylsulfinyl, and tert-butylsulfinyl groups.

In the present invention, unless otherwise specified, the “(C1-C6)alkylsulfonyl” refers to a ((C1-C6)alkyl)-S(═O)₂— group whose alkyl moiety is as defined above, and examples thereof include methylsulfonyl, ethylsulfonyl, n-propylsulfonyl, isopropylsulfonyl, n-butylsulfonyl, isobutylsulfonyl, sec-butylsulfonyl, and tert-butylsulfonyl groups.

In the present invention, unless otherwise specified, the “(C1-C6)haloalkylthio” refers to a ((C1-C6)haloalkyl)-S— group whose haloalkyl moiety is as defined above, and examples thereof include fluoromethylthio, difluoromethylthio, trifluoromethylthio, trichloromethylthio, 2,2,2-trifluoroethylthio, pentafluoroethylthio, 2,2,2-trichloroethylthio, 3,3,3-trifluoropropylthio, 1,1,2,3,3,3-hexafluoropropyl, heptafluoropropylthio, 1,1,1,3,3,3-hexafluoropropan-2-ylthio, heptafluoropropan-2-ylthio, and 4,4,4-trifluorobutylthio groups.

In the present invention, unless otherwise specified, the “(C1-C6)haloalkylsulfinyl” refers to a ((C1-C6)haloalkyl)-S(═O)— group whose haloalkyl moiety is as defined above, and examples thereof include difluoromethylsulfinyl, trifluoromethylsulfinyl, trichloromethylsulfinyl, 2,2,2-trifluoroethylsulfinyl, 2,2,2-trichloroethylsulfinyl, pentafluoroethylsulfinyl, 3,3,3-trifluoropropylsulfinyl, heptafluoropropylsulfinyl, and heptafluoro-2-propylsulfinyl groups.

In the present invention, unless otherwise specified, the “(C1-C6)haloalkylsulfonyl” refers to a (C1-C6 haloalkyl)-S(═O)₂— group whose haloalkyl moiety is as defined above, and examples thereof include difluoromethylsulfonyl, trifluoromethylsulfonyl, trichloromethylsulfonyl, 2,2,2-trifluoroethylsulfonyl, pentafluoroethylsulfonyl, 3,3,3-trifluoropropylsulfonyl, heptafluoropropylsulfonyl, and heptafluoro-2-propylsulfonyl groups.

In the present invention, unless otherwise specified, the “(C2-C6)alkenylthio” refers to a ((C2-C6)alkenyl)-S— group whose alkenyl moiety is as defined above, and examples thereof include vinylthio, 1-propenylthio, isopropenylthio, 2-propenylthio, 1-methyl-2-propenylthio, 2-methyl-2-propenylthio, 2-butenylthio, 3-butenylthio, 3-methyl-2-butenylthio, 2-methyl-2-butenylthio, 2-pentenylthio, 3-pentenylthio, 4-pentenylthio, 2-hexenylthio, 3-hexenylthio, 4-hexenylthio, and 5-hexenylthio groups.

In the present invention, unless otherwise specified, the “(C2-C6)alkenylsulfinyl” refers to a ((C2-C6)alkenyl)-S(═O)— group whose alkenyl moiety is as defined above, and examples thereof include vinylsulfinyl, 1-propenylsulfinyl, isopropenylsulfinyl, 2-propenylsulfinyl, 1-methyl-2-propenylsulfinyl, 2-methyl-2-propenylsulfinyl, 2-butenylsulfinyl, 3-butenylsulfinyl, 3-methyl-2-butenylsulfinyl, 2-methyl-2-butenylsulfinyl, 2-pentenylsulfinyl, 3-pentenylsulfinyl, 4-pentenylsulfinyl, 2-hexenylsulfinyl, 3-hexenylsulfinyl, 4-hexenylsulfinyl, and 5-hexenylsulfinyl groups.

In the present invention, unless otherwise specified, the “(C2-C6)alkenylsulfonyl” refers to a ((C2-C6)alkenyl)-S(═O)₂— group whose alkenyl moiety is as defined above, and examples thereof include vinylsulfonyl, 1-propenylsulfonyl, isopropenylsulfonyl, 2-propenylsulfonyl, 1-methyl-2-propenylsulfonyl, 2-methyl-2-propenylsulfonyl, 2-butenylsulfonyl, 3-butenylsulfonyl, 3-methyl-2-butenylsulfonyl, 2-methyl-2-butenylsulfonyl, 2-pentenylsulfonyl, 3-pentenylsulfonyl, 4-pentenylsulfonyl, 2-hexenylsulfonyl, 3-hexenylsulfonyl, 4-hexenylsulfonyl, and 5-hexenylsulfonyl groups.

In the present invention, unless otherwise specified, the “(C2-C6)alkynylthio” refers to a ((C2-C6)alkynyl)-S— group whose alkynyl moiety is as defined above, and examples thereof include ethynylthio, 1-propynylthio, 2-propynylthio, 1-methyl-2-propynylthio, 1-ethyl-2-propynylthio, 1-butynylthio, 2-butynylthio, 3-butynylthio, 1-methyl-2-butynylthio, 1-pentynylthio, 2-pentynylthio, 3-pentynylthio, 4-pentynylthio, and 4,4-dimethyl-2-pentynylthio groups.

In the present invention, unless otherwise specified, the “(C2-C6)alkynylsulfinyl” refers to a ((C2-C6)alkynyl)-S(═O)— group whose alkynyl moiety is as defined above, and examples thereof include ethynylsulfinyl, 1-propynylsulfinyl, 2-propynylsulfinyl, 1-methyl-2-propynylsulfinyl, 1-ethyl-2-propynylsulfinyl, 1-butynylsulfinyl, 2-butynylsulfinyl, 3-butynylsulfinyl, 1-methyl-2-butynylsulfinyl, 1-pentynylsulfinyl, 2-pentynylsulfinyl, 3-pentynylsulfinyl, 4-pentynylsulfinyl, and 4,4-dimethyl-2-pentynylsulfinyl groups.

In the present invention, unless otherwise specified, the “(C2-C6)alkynylsulfonyl” refers to a (C2-C6 alkynyl)-S(═O)₂— group whose alkynyl moiety is as defined above, and examples thereof include ethynylsulfonyl, 1-propynylsulfonyl, 2-propynylsulfonyl, 1-methyl-2-propynylsulfonyl, 1-ethyl-2-propynylsulfonyl, 1-butynylsulfonyl, 2-butynylsulfonyl, 3-butynylsulfonyl, 1-methyl-2-butynylsulfonyl, 1-pentynylsulfonyl, 2-pentynylsulfonyl, 3-pentynylsulfonyl, 4-pentynylsulfonyl, and 4,4-dimethyl-2-pentynylsulfonyl groups.

In the present invention, unless otherwise specified, the “(C3-C6)cycloalkylthio” refers to a ((C3-C6)cycloalkyl)-S— group whose cycloalkyl moiety is as defined above, and examples thereof include cyclopropylthio, cyclobutylthio, cyclopentylthio, and cyclohexylthio groups.

In the present invention, unless otherwise specified, the “(C3-C6)cycloalkylsulfinyl” refers to a ((C3-C6)cycloalkyl)-S(═O)— group whose cycloalkyl moiety is as defined above, and examples thereof include cyclopropylsulfinyl, cyclobutylsulfinyl, cyclopentylsulfinyl, and cyclohexylsulfinyl groups.

In the present invention, unless otherwise specified, the “(C3-C6)cycloalkylsulfonyl” refers to a ((C3-C6)cycloalkyl)-S(═O)₂— group whose cycloalkyl moiety is as defined above, and examples thereof include cyclopropylsulfonyl, cyclobutylsulfonyl, cyclopentylsulfonyl, and cyclohexylsulfonyl groups.

In the present invention, unless otherwise specified, the “(C6-C12)aryloxy” refers to a ((C6-C12)aryl)-O— group whose aryl moiety is as defined above, and examples thereof include phenyloxy, biphenyloxy, naphthalen-1-yloxy, and naphthalen-2-yloxy groups.

In the present invention, unless otherwise specified, the “(C6-C12)arylthio” refers to a ((C6-C12)aryl)-S— group whose aryl moiety is as defined above, and examples thereof include phenylthio, biphenylthio, naphthalen-1-ylthio, and naphthalen-2-ylthio groups.

In the present invention, unless otherwise specified, the “(C6-C12)arylsulfinyl” refers to a ((C6-C12)aryl)-S(═O)— group whose aryl moiety is as defined above, and examples thereof include phenylsulfinyl, biphenylsulfinyl, naphthalen-1-ylsulfinyl, and naphthalen-2-ylsulfinyl groups.

In the present invention, unless otherwise specified, the “(C6-C12)arylsulfonyl” refers to a ((C6-C12)aryl)-S(═O)₂— group whose aryl moiety is as defined above, and examples thereof include phenylsulfonyl, biphenylsulfonyl, naphthalen-1-ylsulfonyl, and naphthalen-2-ylsulfonyl groups.

In the present invention, unless otherwise specified, the “(C6-C12)arylamino” refers to a ((C6-C12)aryl)-NH— group whose aryl moiety is as defined above, and examples thereof include phenylamino, biphenylamino, naphthalen-1-ylamino, and naphthalen-2-ylamino groups.

In the present invention, unless otherwise specified, the “3- to 6-membered heterocyclyloxy” refers to a (3- to 6-membered heterocyclyl)-O— group whose heterocyclyl moiety is as defined above, and examples thereof include aziridinyloxy, oxylanyloxy, oxetanyloxy, thietanyloxy, tetrahydrofuranyloxy, tetrahydropyranyloxy, azetidinyloxy, pyrrolidinyloxy, piperidinyloxy, piperazinyloxy, dihydroisoxazolyloxy, dioxaborolanyloxy, dioxolanyloxy, dioxanyloxy, and morpholinyloxy groups.

In the present invention, unless otherwise specified, the “3- to 6-membered heterocyclylthio” refers to a (3- to 6-membered heterocyclyl)-S— group whose heterocyclyl moiety is as defined above, and examples thereof include aziridinylthio, oxylanylthio, oxetanylthio, thietanylthio, tetrahydrofuranylthio, tetrahydropyranylthio, azetidinylthio, pyrrolidinylthio, piperidinylthio, piperazinylthio, dihydroisoxazolylthio, dioxaborolanylthio, dioxolanylthio, dioxanylthio, and morpholinylthio groups.

In the present invention, unless otherwise specified, the “3- to 6-membered heterocyclylsulfinyl” refers to a (3- to 6-membered heterocyclyl)-S(═O)— group whose heterocyclyl moiety is as defined above, and examples thereof include aziridinylsulfinyl, oxylanylsulfinyl, oxetanylsulfinyl, thietanylsulfinyl, tetrahydrofuranylsulfinyl, tetrahydropyranylsulfinyl, azetidinylsulfinyl, pyrrolidinylsulfinyl, piperidinylsulfinyl, piperazinylsulfinyl, dihydroisoxazolylsulfinyl, dioxaborolanylsulfinyl, dioxolanylsulfinyl, dioxanylsulfinyl, and morpholinylsulfinyl groups.

In the present invention, unless otherwise specified, the “3- to 6-membered heterocyclylsulfonyl” refers to a (3- to 6-membered heterocyclyl)-S(═O)₂— group whose heterocyclyl moiety is as defined above, and examples thereof include aziridinylsulfonyl, oxylanylsulfonyl, oxetanylsulfonyl, thietanylsulfonyl, tetrahydrofuranylsulfonyl, tetrahydropyranylsulfonyl, azetidinylsulfonyl, pyrrolidinylsulfonyl, piperidinylsulfonyl, piperazinylsulfonyl, dihydroisoxazolylsulfonyl, dioxaborolanylsulfonyl, dioxolanylsulfonyl, dioxanylsulfonyl, and morpholinylsulfonyl groups.

In the present invention, unless otherwise specified, the “3- to 6-membered heterocyclylamino” refers to a (3- to 6-membered heterocyclyl)-NH— group whose heterocyclyl moiety is as defined above, and examples thereof include aziridinylamino, oxylanylamino, oxetanylamino, thietanylamino, tetrahydrofuranylamino, tetrahydropyranylamino, azetidinylamino, pyrrolidinylamino, piperidinylamino, piperazinylamino, dihydroisoxazolylamino, dioxaborolanylamino, dioxolanylamino, dioxanylamino, and morpholinylamino groups.

In the present invention, unless otherwise specified, the “5- to 12-membered heteroaryloxy” refers to a (5- to 12-membered heteroaryl)-O— group whose heteroaryl moiety is as defined above, and examples thereof include pyridyloxy, pyrimidinyloxy, pyrazinyloxy, pyridadinyloxy, triazinyloxy, thienyloxy, furanyloxy, pyrrolyloxy, pyrazolyloxy, thiazolyloxy, isothiazolyloxy, oxazolyloxy, isoxazolyloxy, imidazolyloxy, triazolyloxy, thiadiazolyloxy, oxadiazolyloxy, tetrazolyloxy, indolyloxy, benzoimidazolyloxy, indazolyloxy, benzotriazolyloxy, isoquinolinyloxy, quinolinyloxy, benzothiazolyloxy, benzofuranyloxy, benzothienyloxy, benzoisooxazolyloxy, pyrazolopyridyloxy, pyrazolopyrimidinyloxy, pyrrolopyridyloxy, triazolopyridyloxy, triazolopyrimidinyloxy, quinazolinyloxy, quinoxalinyloxy, and cinnolinyloxy groups.

In the present invention, unless otherwise specified, the “5- to 12-membered heteroarylthio” refers to a (5- to 12-membered heteroaryl)-S— group whose heteroaryl moiety is as defined above, and examples thereof include pyridylthio, pyrimidinylthio, pyrazinylthio, pyridadinylthio, triazinylthio, thienylthio, furanylthio, pyrrolylthio, pyrazolylthio, thiazolylthio, isothiazolylthio, oxazolylthio, isoxazolylthio, imidazolylthio, triazolylthio, thiadiazolylthio, oxadiazolylthio, tetrazolylthio, indolylthio, benzoimidazolylthio, indazolylthio, benzotriazolylthio, isoquinolinylthio, quinolinylthio, benzothiazolylthio, benzofuranylthio, benzothienylthio, benzoisooxazolylthio, pyrazolopyridylthio, pyrazolopyrimidinylthio, pyrrolopyridylthio, triazolopyridylthio, triazolopyrimidinylthio, quinazolinylthio, quinoxalinylthio, and cinnolinylthio groups.

In the present invention, unless otherwise specified, the “5- to 12-membered heteroarylsulfinyl” refers to a (5- to 12-membered heteroaryl)-S(═O)— group whose heteroaryl moiety is as defined above, and examples thereof include pyridylsulfinyl, pyrimidinylsulfinyl, pyrazinylsulfinyl, pyridadinylsulfinyl, triazinylsulfinyl, thienylsulfinyl, furanylsulfinyl, pyrrolylsulfinyl, pyrazolylsulfinyl, thiazolylsulfinyl, isothiazolylsulfinyl, oxazolylsulfinyl, isoxazolylsulfinyl, imidazolylsulfinyl, triazolylsulfinyl, thiadiazolylsulfinyl, oxadiazolylsulfinyl, tetrazolylsulfinyl, indolylsulfinyl, benzoimidazolylsulfinyl, indazolylsulfinyl, benzotriazolylsulfinyl, isoquinolinylsulfinyl, quinolinylsulfinyl, benzothiazolylsulfinyl, benzofuranylsulfinyl, benzothienylsulfinyl, benzoisoxazolylsulfinyl, pyrazolopyridylsulfinyl, pyrazolopyrimidinylsulfinyl, pyrrolopyridylsulfinyl, triazolopyridylsulfinyl, triazolopyrimidinylsulfinyl, quinazolinylsulfinyl, quinoxalinylsulfinyl, and cinnolinylsulfinyl groups.

In the present invention, unless otherwise specified, the “5- to 12-membered heteroarylsulfonyl” refers to a (5- to 12-membered heteroaryl)-S(═O)₂— group whose heteroaryl moiety is as defined above, and examples thereof include pyridylsulfonyl, pyrimidinylsulfonyl, pyrazinylsulfonyl, pyridadinylsulfonyl, triazinylsulfonyl, thienylsulfonyl, furanylsulfonyl, pyrrolylsulfonyl, pyrazolylsulfonyl, thiazolylsulfonyl, isothiazolylsulfonyl, oxazolylsulfonyl, isoxazolylsulfonyl, imidazolylsulfonyl, triazolylsulfonyl, thiadiazolylsulfonyl, oxadiazolylsulfonyl, tetrazolylsulfonyl, indolylsulfonyl, benzoimidazolylsulfonyl, indazolylsulfonyl, benzotriazolylsulfonyl, isoquinolinylsulfonyl, quinolinylsulfonyl, benzothiazolylsulfonyl, benzofuranylsulfonyl, benzothienylsulfonyl, benzoisooxazolylsulfonyl, pyrazolopyridylsulfonyl, pyrazolopyrimidinylsulfonyl, pyrrolopyridylsulfonyl, triazolopyridylsulfonyl, triazolopyrimidinylsulfonyl, quinazolinylsulfonyl, quinoxalinylsulfonyl, and cinnolinylsulfonyl groups.

In the present invention, unless otherwise specified, the “5- to 12-membered heteroarylamino” refers to a (5- to 12-membered heteroaryl)-NH— group whose heteroaryl moiety is as defined above, and examples thereof include pyridylamino, pyrimidinylamino, pyrazinylamino, pyridadinylamino, triazinylamino, thienylamino, furanylamino, pyrrolylamino, pyrazolylamino, thiazolylamino, isothiazolylamino, oxazolylamino, isoxazolylamino, imidazolylamino, triazolylamino, thiadiazolylamino, oxadiazolylamino, tetrazolylamino, indolylamino, benzoimidazolylamino, indazolylamino, benzotriazolylamino, isoquinolinylamino, quinolinylamino, benzothiazolylamino, benzofuranylamino, benzothienylamino, benzoisooxazolylamino, pyrazolopyridylamino, pyrazolopyrimidinylamino, pyrrolopyridylamino, triazolopyridylamino, triazolopyrimidinylamino, quinazolinylamino, quinoxalinylamino, and cinnolinylamino groups.

In the present invention, unless otherwise specified, the “(C2-C6)alkenyloxyimino (C1-C6)alkyl” refers to a ((C2-C6)alkenyl)-O—N═((C1-C6)alkyl) group whose alkenyl moiety and alkyl moiety are as defined above, and examples thereof include 1-(vinyloxyimino)methyl, 1-(vinyloxyimino)ethyl, 1-(vinyloxyimino)propyl, 1-(vinyloxyimino)butyl, 1-(2-propenyloxyimino)methyl, 1-(2-propenyloxyimino)ethyl, 1-(2-propenyloxyimino)propyl, and 1-(2-propenyloxyimino)butyl groups.

In the present invention, unless otherwise specified, the “(C2-C6)alkynyloxyimino (C1-C6)alkyl” refers to a ((C2-C6)alkynyl)-O—N═((C1-C6)alkyl) group whose alkynyl moiety and alkyl moiety are as defined above, and examples thereof include 1-(ethynyloxyimino)methyl, 1-(2-propynyloxyimino)methyl, 1-(ethynyloxyimino)ethyl, 1-(2-propynyloxyimino)ethyl, 1-(2-propynyloxyimino)propyl, and 1-(ethynyloxyimino)propyl groups.

In the present invention, unless otherwise specified, the “(C3-C6)cycloalkyloxyimino (C1-C6)alkyl group” refers to a ((C3-C6)cycloalkyl)-O—N═((C1-C6)alkyl) group whose cycloalkyl moiety and alkyl moiety are as defined above, and examples thereof include 1-(cyclopropyloxyimino)methyl, 1-(cyclopropyloxyimino)ethyl, 2-(cyclopropyloxyimino)ethyl, 1-(cyclopropyloxyimino)propyl, and 2-(cyclopropyloxyimino)propyl groups.

In the present invention, unless otherwise specified, the “(C6-C12)aryloxyimino (C1-C6)alkyl” refers to a ((C6-C12)aryl)-O—N═((C1-C6)alkyl) group whose aryl moiety and alkyl moiety are as defined above, and examples thereof include 1-(phenoxyimino)methyl, 1-(phenoxyimino)ethyl, and 2-(phenoxyimino)ethyl groups.

In the present invention, unless otherwise specified, the “3- to 6-membered heterocyclyloxyimino (C1-C6)alkyl” refers to a (3- to 6-membered heterocyclyl)-O—N═((C1-C6)alkyl) group whose heterocyclyl moiety and alkyl moiety are as defined above, and examples thereof include 1-(aziridinyloxyimino)ethyl, 1-(oxylanyloxyimino)ethyl, 1-(oxetanyloxyimino)ethyl, 1-(thietanyloxyimino)ethyl, 1-(tetrahydrofuranyloxyimino)ethyl, 1-(tetrahydropyranyloxyimino)ethyl, 1-(azetidinyloxyimino)ethyl, 1-(pyrrolidinyloxyimino)ethyl, 1-(piperidinyloxyimino)ethyl, and 1-(piperazinyloxyimino)ethyl groups.

In the present invention, unless otherwise specified, the “5- to 12-membered heteroaryloxyimino (C1-C6)alkyl” refers to a (5- to 12-membered heteroaryl)-O—N═((C1-C6)alkyl) group whose heteroaryl moiety and alkyl moiety are as defined above, and examples thereof include 1-(pyridyloxyimino)ethyl, 1-(pyrimidinyloxyimino)ethyl, 1-(pyrazinyloxyimino)ethyl, 1-(pyridadinyloxyimino)ethyl, 1-(triazinyloxyimino)ethyl, 1-(thienyloxyimino)ethyl, 1-(furanyloxyimino)ethyl, 1-(pyrrolyloxyimino)ethyl, 1-(pyrazolyloxyimino)ethyl, 1-(thiazolyloxyimino)ethyl, 1-(isothiazolyloxyimino)ethyl, 1-(oxazolyloxyimino)ethyl, 1-(isoxazolyloxyimino)ethyl, 1-(imidazolyloxyimino)ethyl, 1-(triazolyloxyimino)ethyl, 1-(thiadiazolyloxyimino)ethyl, 1-(oxadiazolyloxyimino)ethyl, and 1-(tetrazolyloxyimino)ethyl groups.

In the present invention, unless otherwise specified, the “(C2-C6)alkenyloxycarbonyloxy” refers to a ((C2-C6)alkenyl)-O—C(═O)—O— group whose alkenyl moiety is as defined above, and examples thereof include vinyloxycarbonyloxy, 1-propenyloxycarbonyloxy, isopropenyloxycarbonyloxy, 2-propenyloxycarbonyloxy, 1-methyl-2-propenyloxycarbonyloxy, 2-methyl-2-propenyloxycarbonyloxy, 2-butenyloxycarbonyloxy, 3-butenyloxycarbonyloxy, 3-methyl-2-butenyloxycarbonyloxy, 2-methyl-2-butenyloxycarbonyloxy, 2-pentenyloxycarbonyloxy, 3-pentenyloxycarbonyloxy, 4-pentenyloxycarbonyloxy, 2-hexenyloxycarbonyloxy, 3-hexenyloxycarbonyloxy, 4-hexenyloxycarbonyloxy, and 5-hexenyloxycarbonyloxy groups.

In the present invention, unless otherwise specified, the “(C2-C6)alkynyloxycarbonyloxy” refers to a ((C2-C6)alkynyl)-O—C(═O)—O— group whose alkynyl moiety is as defined above, and examples thereof include ethynyloxycarbonyloxy, 1-propynyloxycarbonyloxy, 2-propynyloxycarbonyloxy, 1-methyl-2-propynyloxycarbonyloxy, 1-ethyl-2-propynyloxycarbonyloxy, 1-butynyloxycarbonyloxy, 2-butynyloxycarbonyloxy, 3-butynyloxycarbonyloxy, 1-methyl-2-butynyloxycarbonyloxy, 1-pentynyloxycarbonyloxy, 2-pentynyloxycarbonyloxy, 3-pentynyloxycarbonyloxy, 4-pentynyloxycarbonyloxy, and 4,4-dimethyl-2-pentynyloxycarbonyloxy groups.

In the present invention, unless otherwise specified, the “(C3-C6)cycloalkyloxycarbonyloxy” refers to a ((C3-C6)cycloalkyl)-O—C(═O)—O— group whose cycloalkyl moiety is as defined above, and examples thereof include cyclopropyloxycarbonyloxy, cyclobutyloxycarbonyloxy, cyclopentyloxycarbonyloxy, and cyclohexyloxycarbonyloxy groups.

In the present invention, unless otherwise specified, the “(C6-C12)aryloxycarbonyloxy” refers to a ((C6-C12)aryl)-O—C(═O)—O— group whose aryl moiety is as defined above, and examples thereof include phenyloxycarbonyl, biphenyloxycarbonyloxy, naphthalen-1-yloxycarbonyloxy, and naphthalen-2-yloxycarbonyloxy groups.

In the present invention, unless otherwise specified, the “3- to 6-membered heterocyclyloxycarbonyloxy” refers to a (3- to 6-membered heterocyclyl)-O—C(═O)—O— group whose heterocyclyl moiety is as defined above, and examples thereof include aziridinyloxycarbonyloxy, oxiranyloxycarbonyloxy, oxetanyloxycarbonyloxy, thietanyloxycarbonyloxy, tetrahydrofuranyloxycarbonyloxy, tetrahydropyranyloxycarbonyloxy, azetidinyloxycarbonyloxy, pyrrolidinyloxycarbonyloxy, piperidinyloxycarbonyloxy, piperazinyloxycarbonyloxy, dihydroisoxazolyloxycarbonyloxy, dioxaborolanyloxycarbonyloxy, dioxolanyloxycarbonyloxy, dioxanyloxycarbonyloxy, and morpholinyloxycarbonyloxy groups.

In the present invention, unless otherwise specified, the “5- to 12-membered heteroaryloxycarbonyloxy” refers to a (5- to 12-membered heteroaryl)-O—C(═O)—O— group whose heteroaryl moiety is as defined above, and examples thereof include pyridyloxycarbonyloxy, pyrimidinyloxycarbonyloxy, pyrazinyloxycarbonyloxy, pyridadinyloxycarbonyloxy, triazinyloxycarbonyloxy, thienyloxycarbonyloxy, furanyloxycarbonyloxy, pyrrolyloxycarbonyloxy, pyrazolyloxycarbonyloxy, thiazolyloxycarbonyloxy, isothiazolyloxycarbonyloxy, oxazolyloxycarbonyloxy, isoxazolyloxycarbonyloxy, imidazolyloxycarbonyloxy, triazolyloxycarbonyloxy, thiadiazolyloxycarbonyloxy, oxadiazolyloxycarbonyloxy, tetrazolyloxycarbonyloxy, indolyloxycarbonyloxy, benzoimidazolyloxycarbonyloxy, indazolyloxycarbonyloxy, benzotriazolyloxycarbonyloxy, isoquinolinyloxycarbonyloxy, quinolinyloxycarbonyloxy, benzothiazolyloxycarbonyloxy, benzofuranyloxycarbonyloxy, benzothienyloxycarbonyloxy, benzoisooxazolyloxycarbonyloxy, pyrazolopyridyloxycarbonyloxy, pyrazolopyrimidinyloxycarbonyloxy, pyrrolopyridyloxycarbonyloxy, triazolopyridyloxycarbonyloxy, triazolopyrimidinyloxycarbonyloxy, quinazolinyloxycarbonyloxy, quinoxalinyloxycarbonyloxy, and cinnolinyloxycarbonyloxy groups.

In the present invention, unless otherwise specified, the “(C1-C6)alkylaminocarbonylamino” refers to a (C1-C6)alkyl)-NH—C(═O)—NH— group whose alkyl moiety is as defined above, and examples thereof include methylaminocarbonylamino, ethylaminocarbonylamino, n-propylaminocarbonylamino, isopropylaminocarbonylamino, and tert-butylaminocarbonylamino groups.

In the present invention, unless otherwise specified, the “(C2-C6)alkenylaminocarbonylamino” refers to a ((C2-C6)alkenyl)-NH—C(═O)—NH— group whose alkenyl moiety is as defined above, and examples thereof include vinylaminocarbonylamino, 1-propenylaminocarbonylamino, isopropenylaminocarbonylamino, 2-propenylaminocarbonylamino, 1-methyl-2-propenylaminocarbonylamino, 2-methyl-2-propenylaminocarbonylamino, 2-butenylaminocarbonylamino, 3-butenylaminocarbonylamino, 3-methyl-2-butenylaminocarbonylamino, 2-methyl-2-butenylaminocarbonylamino, 2-pentenylaminocarbonylamino, 3-pentenylaminocarbonylamino, 4-pentenylaminocarbonylamino, 2-hexenylaminocarbonylamino, 3-hexenylaminocarbonylamino, 4-hexenylaminocarbonylamino, and 5-hexenylaminocarbonylamino groups.

In the present invention, unless otherwise specified, the “(C2-C6)alkynylaminocarbonylamino” refers to a ((C2-C6)alkynyl)-NH—C(═O)—NH— group whose alkynyl moiety is as defined above, and examples thereof include ethynylaminocarbonylamino, 1-propynylaminocarbonylamino, 2-propynylaminocarbonylamino, 1-methyl-2-propynylaminocarbonylamino, 1-ethyl-2-propynylaminocarbonylamino, 1-butynylaminocarbonylamino, 2-butynylaminocarbonylamino, 3-butynylaminocarbonylamino, 1-methyl-2-butynylaminocarbonylamino, 1-pentynylaminocarbonylamino, 2-pentynylaminocarbonylamino, 3-pentynylaminocarbonylamino, 4-pentynylaminocarbonylamino, and 4,4-dimethyl-2-pentynylaminocarbonylamino groups.

In the present invention, unless otherwise specified, the “(C3-C6)cycloalkylaminocarbonylamino” refers to a ((C3-C6)cycloalkyl)-NH—C(═O)—NH— group whose cycloalkyl moiety is as defined above, and examples thereof include cyclopropylaminocarbonylamino, cyclobutylaminocarbonylamino, cyclopentylaminocarbonylamino, and cyclohexylaminocarbonylamino groups.

In the present invention, unless otherwise specified, the “(C6-C12)arylaminocarbonylamino” refers to a ((C6-C12)aryl)-NH—C(═O)—NH— group whose aryl moiety is as defined above, and examples thereof include phenylaminocarbonylamino, biphenylaminocarbonylamino, naphthalen-1-ylaminocarbonylamino, and naphthalen-2-ylaminocarbonylamino groups.

In the present invention, unless otherwise specified, the “3- to 6-membered heterocyclylaminocarbonylamino” refers to a (3- to 6-membered heterocyclyl)-NH—C(═O)—NH— group whose heterocyclyl moiety is as defined above, and examples thereof include aziridinylaminocarbonylamino, oxiranylaminocarbonylamino, oxetanylaminocarbonylamino, thietanylaminocarbonylamino, tetrahydrofuranylaminocarbonylamino, tetrahydropyranylaminocarbonylamino, azetidinylaminocarbonylamino, pyrrolidinylaminocarbonylamino, piperidinylaminocarbonylamino, piperazinylaminocarbonylamino, dihydroisoxazolylaminocarbonylamino, dioxaborolanylaminocarbonylamino, dioxolanylaminocarbonylamino, dioxanylaminocarbonylamino, and morpholinylaminocarbonylamino groups.

In the present invention, unless otherwise specified, the “5- to 12-membered heteroarylaminocarbonylamino” refers to a (5- to 12-membered heteroaryl)-NH—C(═O)—NH— group whose heteroaryl moiety is as defined above, and examples thereof include pyridylaminocarbonylamino, pyrimidinylaminocarbonylamino, pyrazinylaminocarbonylamino, pyridadinylaminocarbonylamino, triazinylaminocarbonylamino, thienylaminocarbonylamino, furanylaminocarbonylamino, pyrrolylaminocarbonylamino, pyrazolylaminocarbonylamino, thiazolylaminocarbonylamino, isothiazolylaminocarbonylamino, oxazolylaminocarbonylamino, isoxazolylaminocarbonylamino, imidazolylaminocarbonylamino, triazolylaminocarbonylamino, thiadiazolylaminocarbonylamino, oxadiazolylaminocarbonylamino, tetrazolylaminocarbonylamino, indolylaminocarbonylamino, benzoimidazolylaminocarbonylamino, indazolylaminocarbonylamino, benzotriazolylaminocarbonylamino, isoquinolinylaminocarbonylamino, quinolinylaminocarbonylamino, benzothiazolylaminocarbonylamino, benzofuranylaminocarbonylamino, benzothienylaminocarbonylamino, benzoisooxazolylaminocarbonylamino, pyrazolopyridylaminocarbonylamino, pyrazolopyrimidinylaminocarbonylamino, pyrrolopyridylaminocarbonylamino, triazolopyridylaminocarbonylamino, triazolopyrimidinylaminocarbonylamino, quinazolinylaminocarbonylamino, quinoxalinylaminocarbonylamino, and cinnolinylaminocarbonylamino groups.

In the present invention, unless otherwise specified, the “(C1-C6)alkoxy (C1-C6)alkyl” refers to a ((C1-C6)alkoxy)-((C1-C6)alkyl)- group whose alkoxy moiety and alkyl moiety are as defined above, and examples thereof include methoxymethyl, ethoxymethyl, n-propoxymethyl, isopropoxymethyl, tert-butoxymethyl, 1-methoxyethyl, 1-methoxy-1-methylethyl, 2-methoxyethyl, 1-ethoxyethyl, 2-ethoxyethyl, 1-isopropoxyethyl, 1-methoxypropyl, 2-methoxypropyl, 1-ethoxypropyl, 1-methoxybutyl, and 1-ethoxybutyl groups.

In the present invention, unless otherwise specified, the “(C1-C6)haloalkoxy (C1-C6)alkyl” refers to a ((C1-C6)haloalkyl)-O—((C1-C6)alkyl)- group whose haloalkyl moiety and alkyl moiety are as defined above, and examples thereof include 2-(difluoromethoxy)ethyl, 2-(trifluoromethoxy)ethyl, 2-(2,2-difluoroethoxy)ethyl, 2-(2,2,2-trifluoroethoxy)ethyl, 2-(3,3-difluoropropioxy)ethyl, 2-(3,3,3-trifluoropropioxy)ethyl, 3-(difluoromethoxy)propyl, 3-(trifluoromethoxy)propyl, 3-(2,2-difluoroethoxy)propyl, 3-(2,2,2-trifluoroethoxy)propyl, 3-(3,3-difluoropropioxy)propyl, 3-(3,3,3-trifluoropropioxy)propyl, 4-(trifluoromethoxy)butyl, and 5-(trifluoromethoxy)pentyl groups.

In the present invention, unless otherwise specified, the “cyano (C1-C6)alkyl” refers to a (cyano)-((C1-C6)alkyl)- group whose alkyl moiety is as defined above, and examples thereof include cyanomethyl, 1-cyanoethyl, 2-cyanoethyl, 1-cyanopropyl, 3-cyanopropyl, 2-cyano-2-propyl, 1-cyanobutyl, 4-cyanobutyl, 5-cyanopentyl, and 6-cyanohexyl groups.

In the present invention, unless otherwise specified, the “cyano (C3-C6)cycloalkyl” refers to a (cyano)-((C3-C6)cycloalkyl)- group whose cycloalkyl moiety is as defined above, and examples thereof include 1-cyanocyclopropyl, 2-cyanocyclopropyl, 1-cyanomethylcyclopropyl, 1-cyanocyclobutyl, 3-cyanocyclobutyl, 1-cyanocyclopentyl, and 1-cyanocyclohexyl groups.

In the present invention, unless otherwise specified, the “(C1-C6)haloalkylamino” refers to a ((C1-C6)haloalkyl)-NH— group whose haloalkyl moiety is as defined above, and examples thereof include trifluoromethylamino, 2-fluoroethylamino, 2,2-difluoroethylamino, 2,2,2-trifluoroethylamino, 2,2,2-trichloroethylamino, pentafluoroethylamino, 3,3,3-trifluoropropylamino, and 1,1,1,3,3,3-hexafluoro-2-propylamino groups.

In the present invention, unless otherwise specified, the “(C1-C6)haloalkylcarbonyl” refers to a ((C1-C6)haloalkyl)-C(═O)— group whose haloalkyl moiety is as defined above, and examples thereof include fluoroacetyl, difluoroacetyl, trifluoroacetyl, chloroacetyl, trichloroacetyl, tribromoacetyl, 3,3,3-trifluoropropionyl, 3,3-difluoropropionyl, and 4,4,4-trifluorobutyryl groups.

In the present invention, unless otherwise specified, the “hydroxyimino (C1-C6)alkyl” refers to a HO—N═((C1-C6)alkyl) group whose alkyl moiety is as defined above, and examples thereof include 1-(hydroxyimino)methyl, 1-(hydroxyimino)ethyl, 2-(hydroxyimino)ethyl, 1-(hydroxyimino)propyl, 2-(hydroxyimino)propyl, and 3-(hydroxyimino)propyl groups.

In the present invention, unless otherwise specified, the “(C1-C6)alkoxyimino (C1-C6)alkyl” refers to a ((C1-C6)alkyl)-O—N═((C1-C6)alkyl) group whose alkyl moiety is as defined above, and examples thereof include 1-(methoxyimino)methyl, 1-(methoxyimino)ethyl, 2-(methoxyimino)ethyl, 1-(methoxyimino)propyl, 2-(methoxyimino)propyl, 3-(methoxyimino)propyl, 1-(ethoxyimino)methyl, 1-(ethoxyimino)ethyl, 2-(ethoxyimino)ethyl, 1-(ethoxyimino)propyl, 2-(ethoxyimino)propyl, 3-(ethoxyimino)propyl, 1-(isopropoxyimino)ethyl, and 2-(isopropoxymino)ethyl groups.

In the present invention, unless otherwise specified, the “(C6-C12)aryl (C1-C6)alkyl” refers to a ((C6-C12)aryl)-((C1-C6)alkyl) group whose aryl moiety and alkyl moiety are as defined above, and examples thereof include benzyl, 1-phenylethyl, 2-phenylethyl, 1-phenylpropyl, 2-phenylpropyl, and 3-phenylpropyl groups.

In the present invention, unless otherwise specified, the “3- to 6-membered heterocyclyl (C1-C6)alkyl” refers to a (3- to 6-membered heterocyclyl)-((C1-C6)alkyl) group whose heterocyclyl moiety and alkyl moiety are as defined above, and examples thereof include aziridinylmethyl, oxylanylmethyl, oxetanylmethyl, thietanylmethyl, tetrahydrofuranylmethyl, tetrahydropyranylmethyl, azetidinylmethyl, pyrrolidinylmethyl, piperidinylmethyl, piperazinylmethyl, dihydroisoxazolylmethyl, dioxaborolanylmethyl, dioxolanylmethyl, dioxanylmethyl, and morpholinylmethyl groups.

In the present invention, unless otherwise specified, the “5- to 12-membered heteroaryl (C1-C6)alkyl” refers to a (5- to 12-membered heteroaryl)-((C1-C6)alkyl) group whose heteroaryl moiety and alkyl moiety are as defined above, and examples thereof include pyridylmethyl, pyrimidinylmethyl, pyrazinylmethyl, pyridadinylmethyl, triazinylmethyl, thienylmethyl, furanylmethyl, pyrrolylmethyl, pyrazolylmethyl, thiazolylmethyl, isothiazolylmethyl, oxazolylmethyl, isoxazolylmethyl, imidazolylmethyl, triazolylmethyl, thiadiazolylmethyl, oxadiazolylmethyl, tetrazolylmethyl, indolylmethyl, benzoimidazolylmethyl, indazolylmethyl, benzotriazolylmethyl, isoquinolinylmethyl, quinolinylmethyl, benzothiazolylmethyl, benzofuranylmethyl, benzothienylmethyl, benzoisooxazolylmethyl, pyrazolopyridylmethyl, pyrazolopyrimidinylmethyl, pyrrolopyridylmethyl, triazolopyridylmethyl, triazolopyrimidinylmethyl, quinazolinylmethyl, quinoxalinylmethyl, and cinnolinylmethyl groups.

In the present invention, unless otherwise specified, “(C1-C6)alkylsulfonyloxy” refers to a ((C1-C6)alkyl)-S(═O)₂—O— group whose alkyl moiety is as defined above, and examples thereof include methylsulfonyloxy, ethylsulfonyloxy, n-propylsulfonyloxy, isopropylsulfonyloxy, n-butylsulfonyloxy, isobutylsulfonyloxy, sec-butylsulfonyloxy, and tert-butylsulfonyloxy groups.

In the present invention, unless otherwise specified, the “(C1-C6)haloalkylsulfonyloxy” refers to a ((C1-C6)haloalkyl)-S(═O)₂-O— group whose haloalkyl moiety is as defined above, and examples thereof include difluoromethylsulfonyloxy, trifluoromethylsulfonyloxy, trichloromethylsulfonyloxy, chlorodifluoromethylsulfonyloxy, 2,2,2-trifluoroethylsulfonyloxy, 2,2,2-trichloroethylsulfonyloxy, pentafluoroethylsulfonyloxy, 3,3,3-trifluoropropylsulfonyloxy, heptafluoropropylsulfonyloxy, and heptafluoro-2-propylsulfonyloxy groups.

In the present invention, unless otherwise specified, “(C1-C6)alkoxycarbonyloxy” refers to a ((C1-C6)alkyl)-O—C(═O)—O— group whose alkyl moiety is as defined above, and examples thereof include methoxycarbonyloxy, ethoxycarbonyloxy, n-propoxycarbonyloxy, isopropoxycarbonyloxy, n-butoxycarbonyloxy, isobutoxycarbonyloxy, sec-butoxycarbonyloxy, tert-butoxycarbonyloxy, n-pentoxycarbonyloxy, 1-methylbutoxycarbonyloxy, 2-methylbutoxycarbonyloxy, 3-methylbutoxycarbonyloxy, 1-ethylpropoxycarbonyloxy, 1,1-dimethylpropoxycarbonyloxy, 1,2-dimethylpropoxycarbonyloxy, 2,2-dimethylpropoxycarbonyloxy, and n-hexoxycarbonyloxy groups.

In the present invention, unless otherwise specified, the “(C1-C6)haloalkylcarbonyloxy” refers to a ((C1-C6)haloalkyl)-C(═O)—O— group whose alkyl moiety is as defined above, and examples thereof include difluoromethylcarbonyloxy, trifluoromethylcarbonyloxy, trichloromethylcarbonyloxy, chlorodifluoromethylcarbonyloxy, 2,2,2-trifluoroethylcarbonyloxy, 2,2,2-trichloroethylcarbonyloxy, pentafluoroethylcarbonyloxy, 3,3,3-trifluoropropylcarbonyloxy, heptafluoropropylcarbonyloxy, and heptafluoro-2-propylcarbonyloxy groups.

In the present invention, unless otherwise specified, the “(C1-C6)haloalkoxycarbonyloxy” refers to a ((C1-C6)haloalkyl)-O—C(═O)—O— group whose haloalkyl moiety is as defined above, and examples thereof include difluoromethoxycarbonyloxy, dichloromethoxycarbonyloxy, trifluoromethoxycarbonyloxy, trichloromethoxycarbonyloxy, tribromomethoxycarbonyloxy, chlorodifluoromethoxycarbonyloxy, bromodifluoromethoxycarbonyloxy, 2-fluoroethoxycarbonyloxy, 1-chloroethoxycarbonyloxy, 2-chloroethoxycarbonyloxy, 1-bromoethoxycarbonyloxy, 2-bromoethoxycarbonyloxy, 2,2-difluoroethoxycarbonyloxy, 1,2-dichloroethoxycarbonyloxy, 2,2-dichloroethoxycarbonyloxy, 2,2,2-trifluoroethoxycarbonyloxy, 2,2,2-trichloroethoxycarbonyloxy, 1,1,2,2-tetrafluoroethoxycarbonyloxy, pentafluoroethoxycarbonyloxy, 2-bromo-2-chloroethoxycarbonyloxy, 2-chloro-1,1,2,2-tetrafluoroethoxycarbonyloxy, 1-chloro-1,2,2,2-tetrafluoroethoxycarbonyloxy, 1-chloropropoxycarbonyloxy, 2-chloropropoxycarbonyloxy, 3-chloropropoxycarbonyloxy, 2-bromopropoxycarbonyloxy, 3-bromopropoxycarbonyloxy, 2-bromo-1-methylethoxycarbonyloxy, 3-iodopropoxycarbonyloxy, 2,3-dichloropropoxycarbonyloxy, 2,3-dibromopropoxycarbonyloxy, 3,3,3-trifluoropropoxycarbonyloxy, 3,3,3-trifluoro-2-propoxycarbonyloxy, 3,3,3-trichloropropoxycarbonyloxy, 3-bromo-3,3-difluoropropoxycarbonyloxy, 2,2-difluoropropoxycarbonyloxy, 3,3-dichloro-3-fluoropropoxycarbonyloxy, 2,2,3,3-tetrafluoropropoxycarbonyloxy, 1-bromo-3,3,3-trifluoropropoxycarbonyloxy, 2,2,3,3,3-pentafluoropropoxycarbonyloxy, 2,2,2-trifluoro-1-trifluoromethylethoxycarbonyloxy, heptafluoropropoxycarbonyloxy, heptafluoro-2-propoxycarbonyloxy, 1,2,2,2-tetrafluoro-1-trifluoromethylethoxycarbonyloxy, 1,1,2,3,3,3-hexafluoropropoxycarbonyloxy, 2-chlorobutoxycarbonyloxy, 3-chlorobutoxycarbonyloxy, 4-chlorobutoxycarbonyloxy, 2-chloro-1,1-dimethylethoxycarbonyloxy, 4-bromobutoxycarbonyloxy, 3-bromo-2-methylpropoxycarbonyloxy, 2-bromo-1,1-dimethylethoxycarbonyloxy, 2,2-dichloro-1,1-dimethylethoxycarbonyloxy, 2-chloro-1-chloromethyl-2-methylethoxycarbonyloxy, 4,4,4-trifluorobutoxycarbonyloxy, 3,3,3-trifluoro-1-methylpropoxycarbonyloxy, 3,3,3-trifluoro-2-methylpropoxycarbonyloxy, 2,3,4-trichlorobutoxycarbonyloxy, 2,2,2-trichloro-1,1-dimethylethoxycarbonyloxy, 4-chloro-4,4-difluorobutoxycarbonyloxy, 4,4-dichloro-4-fluorobutoxycarbonyloxy, 4-bromo-4,4-difluorobutoxycarbonyloxy, 2,4-dibromo-4,4-difluorobutoxycarbonyloxy, 3,4-dichloro-3,4,4-trifluorobutoxycarbonyloxy, 3,3-dichloro-4,4,4-trifluorobutoxycarbonyloxy, 4-bromo-3,3,4,4-tetrafluorobutoxycarbonyloxy, 4-bromo-3-chloro-3,4,4-trifluorobutoxycarbonyloxy, 2,2,3,3,4,4-hexafluorobutoxycarbonyloxy, 2,2,3,4,4,4-hexafluorobutoxycarbonyloxy, 2,2,2-trifluoro-1-methyl-1-trifluoromethylethoxycarbonyloxy, 3,3,3-trifluoro-2-trifluoromethylpropoxycarbonyloxy, 2,2,3,3,4,4,4-heptafluorobutoxycarbonyloxy, 3,3,4,4,4-pentafluoro-2-butoxycarbonyloxy, 2,3,3,3-tetrafluoro-2-trifluoromethylpropoxycarbonyloxy, 1,1,2,2,3,3,4,4-octafluorobutoxycarbonyloxy, nonafluorobutoxycarbonyloxy, perfluoro-tert-butoxycarbonyloxy, 4-chloro-1,1,2,2,3,3,4,4-octafluorobutoxycarbonyloxy, 5,5,5-trifluoropentoxycarbonyloxy, 4,4,5,5,5-pentafluoropentoxycarbonyloxy, 3,3,4,4,5,5,5-heptafluoropentoxycarbonyloxy, 3,3,4,4,5,5,5-heptafluoro-2-pentoxycarbonyloxy, 2,2,3,3,4,4,5,5,5-nonafluoropentoxycarbonyloxy, 2,2,3,3,4,4,5,5-octafluoropentoxycarbonyloxy, perfluoropentoxycarbonyloxy, 4,4,5,5,5-pentafluoro-2-butoxycarbonyloxy, 2,2-bis(trifluoromethyl)propoxycarbonyloxy, 2,2,3,3,4,4,5,5,6,6,6-undecafluorohexyloxycarbonyloxy, 3,3,4,4,5,5,6,6,6-nonafluorohexyloxycarbonyloxy, 4,4,5,5,6,6,6-heptafluorohexyloxycarbonyloxy, 2,2,3,3,4,4,5,5,6,6-decafluorohexyloxycarbonyloxy, 4,4,4-trifluoro-3,3-bis(trifluoromethyl)butyloxycarbonyloxy, and perfluorohexyloxycarbonyloxy groups.

In the present invention, the expression of “is optionally mono-substituted or poly-substituted with R⁴” refers to “is optionally substituted with one or two or more R⁴”. In a case of being substituted with two or more R⁴, the two or more R⁴ may be the same as or different from each other, and R⁴ each independently represent a hydrogen atom, a halogen atom, a cyano, a thiocyanato, a thiol, a hydroxy, an amino, a tri((C1-C6)alkyl)silyl, a (C3-C6)cycloalkyl, a (C3-C6)halocycloalkyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁵), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁵), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁵), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁵, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6) cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁵), a (C1-C6)alkylsulfonyl (the (C1-C6)alkylsulfonyl is optionally mono-substituted or poly-substituted with R⁵), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁵), a (C3-C6)cycloalkylsulfinyl (the (C3-C6)cycloalkylsulfinyl is optionally mono-substituted or poly-substituted with R⁵), a (C3-C6)cycloalkylsulfonyl (the (C3-C6)cycloalkylsulfonyl is optionally mono-substituted or poly-substituted with R⁵), a (C6-C12)aryl (the (C6-C12)aryl is optionally mono-substituted or poly-substituted with R⁵), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyl (the 3- to 6-membered heterocyclyl is optionally mono-substituted or poly-substituted with R⁵), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryl (the 5- to 12-membered heteroaryl is optionally mono-substituted or poly-substituted with R⁵), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a 1,3-benzodioxole (the 1,3-benzodioxole is optionally mono-substituted or poly-substituted with R⁵), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the (C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl group.

In the present invention, the expression of “is optionally mono-substituted or poly-substituted with R⁵” refers to “is optionally substituted with one or two or more R⁵”. In a case of being substituted with two or more R⁵, the two or more R⁵ may be the same as or different from each other, and R⁵ each independently represent a hydrogen atom, a halogen atom, a cyano, a nitro, an amino, a hydroxy, a thiol, a formyl, an oxo, a (C1-C6)alkyl, a (C2-C6)alkenyl, a (C2-C6)alkynyl, a (C3-C6)cycloalkyl, a (C1-C6)haloalkyl, a (C1-C6)alkoxy, a (C1-C6)haloalkoxy, a (C1-C6)alkoxy (C1-C6)alkyl, a (C1-C6)haloalkoxy (C1-C6)alkyl, a cyano (C1-C6)alkyl, a cyano (C3-C6)cycloalkyl, a (C1-C6)alkylthio, a (C1-C6)haloalkylthio, a (C1-C6)alkylsulfinyl, a (C1-C6)haloalkylsulfinyl, a (C1-C6)alkylsulfonyl, a (C1-C6)haloalkylsulfonyl, a (C1-C6)alkylcarbonylthio, a (C1-C6)alkylamino (the amino moiety is optionally substituted with R⁶), a (C1-C6)haloalkylamino (the amino moiety is optionally substituted with R⁶), a formylamino (the amino moiety is optionally substituted with R⁶), a (C1-C6)alkylcarbonylamino (the amino moiety is optionally substituted with R⁶), a carbamoyl, a (C1-C6)alkylaminocarbonyl (the amino moiety is optionally substituted with R⁶), a carboxyl, a (C1-C6)alkylcarbonyl, a (C1-C6)haloalkylcarbonyl, a (C1-C6)alkoxycarbonyl, a hydroxyimino (C1-C6)alkyl, a (C1-C6)alkoxyimino (C1-C6)alkyl, a (C6-C12)aryl (the (C6-C12)aryl is optionally mono-substituted or poly-substituted with R⁷), a (C6-C12)aryl (C1-C6)alkyl (the (C6-C12)aryl moiety is optionally mono-substituted or poly-substituted with R⁷), a (C6-C12)arylcarbonyl group (the (C6-C12)aryl moiety may be mono-substituted or poly-substituted with R⁷), a (C6-C12)aryloxy (the (C6-C12)aryl moiety is optionally mono-substituted or poly-substituted with R⁷), a (C6-C12)arylthio group (the (C6-C12)aryl moiety is optionally mono-substituted or poly-substituted with R⁷), a (C6-C12)arylsulfinyl (the (C6-C12)aryl moiety is optionally mono-substituted or poly-substituted with R⁷), a (C6-C12)arylsulfonyl (the (C6-C12)aryl moiety is optionally mono-substituted or poly-substituted with R⁷), a (C6-C12)arylamino (the (C6-C12)aryl moiety is optionally mono-substituted or poly-substituted with R⁷, and the amino moiety is optionally substituted with R⁶), a 3- to 6-membered heterocyclyl (the 3- to 6-membered heterocyclyl is optionally mono-substituted or poly-substituted with R⁷), a 3- to 6-membered heterocyclyl (C1-C6)alkyl (the 3- to 6-membered heterocyclyl moiety is optionally mono-substituted or poly-substituted with R⁷), a 3- to 6-membered heterocyclylcarbonyl (the 3- to 6-membered heterocyclyl moiety is optionally mono-substituted or poly-substituted with R⁷), a 3- to 6-membered heterocyclyloxy (the 3- to 6-membered heterocyclyl moiety is optionally mono-substituted or poly-substituted with R⁷), a 3- to 6-membered heterocyclylthio (the 3- to 6-membered heterocyclyl moiety is optionally mono-substituted or poly-substituted with R⁷), a 3- to 6-membered heterocyclylsulfinyl (the 3- to 6-membered heterocyclyl moiety is optionally mono-substituted or poly-substituted with R⁷), 3- to 6-membered heterocyclylsulfonyl (the 3- to 6-membered heterocyclyl moiety is optionally mono-substituted or poly-substituted with R⁷), a 3- to 6-membered heterocyclylamino (the 3- to 6-membered heterocyclyl moiety is optionally mono-substituted or poly-substituted with R⁷, and the amino moiety is optionally substituted with R⁶), a 5- to 12-membered heteroaryl (the 5- to 12-membered heteroaryl is optionally mono-substituted or poly-substituted with R⁷), a 5- to 12-membered heteroaryl (C1-C6)alkyl (the 5- to 12-membered heteroaryl moiety is optionally mono-substituted or poly-substituted with R⁷), a 5- to 12-membered heteroarylcarbonyl (the 5- to 12-membered heteroaryl moiety is optionally mono-substituted or poly-substituted with R⁷), a 5- to 12-membered heteroaryloxy (the 5- to 12-membered heteroaryl moiety is optionally mono-substituted or poly-substituted with R⁷), a 5- to 12-membered heteroarylthio (the 5- to 12-membered heteroaryl moiety is optionally mono-substituted or poly-substituted with R⁷), a 5- to 12-membered heteroarylsulfinyl (the 5- to 12-membered heteroaryl moiety is optionally mono-substituted or poly-substituted with R⁷), a 5- to 12-membered heteroarylsulfonyl (the 5- to 12-membered heteroaryl moiety is optionally mono-substituted or poly-substituted with R⁷), or a 5- to 12-membered heteroarylamino (the 5- to 12-membered heteroaryl moiety is optionally mono-substituted or poly-substituted with R⁷, and the amino moiety is optionally substituted with R⁶).

In the present invention, the expression of “is optionally mono-substituted or poly-substituted with R⁷” refers to “is optionally substituted with one or two or more R⁷”. In a case of being substituted with two or more R⁷, the two or more R⁷ may be the same as or different from each other, and R⁷ each independently represent a hydrogen atom, a halogen atom, a cyano, a (C1-C6)alkyl, or a (C1-C6)alkoxy.

In the present invention, the expression of “is optionally mono-substituted or poly-substituted with R⁸” refers to “is optionally substituted with one or two or more R⁸”. In a case of being substituted with two or more R⁸, the two or more R⁸ may be the same as or different from each other, and R⁸ each independently represent a halogen atom, a cyano, a (C1-C6)haloalkyl, a (C3-C6)cycloalkyl, a (C1-C6)alkoxy, a (C1-C6)alkylcarbonyl, a (C1-C6)alkoxycarbonyl, a (C1-C6)haloalkoxycarbonyl, a (C6-C12)aryl (the (C6-C12)aryl is optionally mono-substituted or poly-substituted with R⁷), a 3- to 6-membered heterocyclyl (the 3- to 6-membered heterocyclyl is optionally mono-substituted or poly-substituted with R⁷), or a 5- to 12-membered heteroaryl (the 5- to 12-membered heteroaryl is optionally mono-substituted or poly-substituted with R⁷).

In the present invention, the expression of “mono-substituted or poly-substituted with substituent(s) selected from a substituent group R⁴” refers to “substituted with one or two or more substituents selected from a substituent group R⁴”. In a case of being substituted with two or more substituents selected from the substituent group R⁴, the two or more substituents selected from the substituent group R⁴ may be the same as or different from each other, and R⁴ each independently represent a halogen atom, a cyano, a thiocyanato, a thiol, a hydroxy, an amino, a tri((C1-C6)alkyl)silyl, a (C3-C6)cycloalkyl, a (C3-C6)halocycloalkyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁵), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁵), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁵), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6) cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁵), a (C1-C6)alkylsulfinyl (the (C1-C6)alkylsulfinyl is optionally mono-substituted or poly-substituted with R⁵), a (C1-C6)alkylsulfonyl (the (C1-C6)alkylsulfonyl is optionally mono-substituted or poly-substituted with R⁵), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁵), a (C3-C6)cycloalkylsulfinyl (the (C3-C6)cycloalkylsulfinyl is optionally mono-substituted or poly-substituted with R⁵), a (C3-C6)cycloalkylsulfonyl (the (C3-C6)cycloalkylsulfonyl is optionally mono-substituted or poly-substituted with R⁵), a (C6-C12)aryl (the (C6-C12)aryl is optionally mono-substituted or poly-substituted with R⁵), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyl (the 3- to 6-membered heterocyclyl is optionally mono-substituted or poly-substituted with R⁵), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryl (the 5- to 12-membered heteroaryl is optionally mono-substituted or poly-substituted with R⁵), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a 1,3-benzodioxole (the 1,3-benzodioxole is optionally mono-substituted or poly-substituted with R⁵), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the (C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl group.

In the present invention, the expression of “mono-substituted or poly-substituted with substituent(s) selected from a substituent group R¹⁰” refers to “substituted with one or two or more substituents selected from a substituent group R¹⁰”. In a case of being substituted with two or more substituents selected from the substituent group R¹⁰, the two or more substituents selected from the substituent group R¹⁰ may be the same as or different from each other, and R¹⁰ each independently represent a halogen atom, a cyano, a thiocyanato, a thiol, a hydroxy, an amino, a tri((C1-C6)alkyl)silyl, a (C3-C6)cycloalkyl, a (C3-C6)halocycloalkyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6)cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryl (the (C6-C12)aryl is optionally mono-substituted or poly-substituted with R⁵), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyl (the 3- to 6-membered heterocyclyl is optionally mono-substituted or poly-substituted with R⁵), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryl (the 5- to 12-membered heteroaryl is optionally mono-substituted or poly-substituted with R⁵), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a 1,3-benzodioxole (the 1,3-benzodioxole is optionally mono-substituted or poly-substituted with R⁵), a hydroxyimino (C1-C6)alkyl, a (C1-C6)alkoxyimino (C1-C6)alkyl (the (C1-C6)alkoxy moiety is optionally mono-substituted or poly-substituted with R⁵), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁵), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁵), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁵), a (C6-C12)aryloxycarbonyloxy (the (C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl group.

In the present invention, the expression of “mono-substituted or poly-substituted with substituent(s) selected from a substituent group R¹¹” refers to “substituted with one or two or more substituents selected from a substituent group R¹¹”. In a case of being substituted with two or more substituents selected from the substituent group R¹¹, the two or more substituents selected from the substituent group R¹¹ may be the same as or different from each other, and R¹¹ each independently represent a halogen atom, a thiocyanato, a thiol, a hydroxy, an amino, a tri((C1-C6)alkyl)silyl, a (C3-C6)cycloalkyl, a (C3-C6)halocycloalkyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁵), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁵), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁵), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁵), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6) cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryl (the (C6-C12)aryl is optionally mono-substituted or poly-substituted with R⁵), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyl (the 3- to 6-membered heterocyclyl is optionally mono-substituted or poly-substituted with R⁵), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryl (the 5- to 12-membered heteroaryl is optionally mono-substituted or poly-substituted with R⁵), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a 1,3-benzodioxole (the 1,3-benzodioxole is optionally mono-substituted or poly-substituted with R⁵), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the (C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁶, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl group.

In the present invention, the expression of “mono-substituted or poly-substituted with substituent(s) selected from a substituent group R¹²” refers to “substituted with one or two or more substituents selected from a substituent group R¹²”. In a case of being substituted with two or more substituents selected from the substituent group R¹², the two or more substituents selected from the substituent group R¹² may be the same as or different from each other, and R¹² each independently represent a halogen atom, a cyano, a thiocyanato, a thiol, a hydroxy, an amino, a tri((C1-C6)alkyl)silyl, a (C3-C6)cycloalkyl, a (C3-C6)halocycloalkyl, a (C1-C6)alkylamino (the (C1-C6)alkyl moiety is optionally mono-substituted or poly-substituted with R⁶, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁶, and the amino moiety is optionally substituted with R⁶), a formyl, a (C1-C6)alkylcarbonyl (the (C1-C6)alkylcarbonyl is optionally mono-substituted or poly-substituted with R⁶), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁶), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6)cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryl (the (C6-C12)aryl is optionally mono-substituted or poly-substituted with R⁵), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyl (the 3- to 6-membered heterocyclyl is optionally mono-substituted or poly-substituted with R⁵), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryl (the 5- to 12-membered heteroaryl is optionally mono-substituted or poly-substituted with R⁵), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a 1,3-benzodioxole (the 1,3-benzodioxole is optionally mono-substituted or poly-substituted with R⁵), a hydroxyimino (C1-C6)alkyl, a (C1-C6)alkoxyimino (C1-C6)alkyl (the (C1-C6)alkoxy moiety is optionally mono-substituted or poly-substituted with R⁵), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁵), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁵), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁵), a (C6-C12)aryloxycarbonyloxy (the (C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl group.

In the present invention, the agriculturally acceptable salt or salt means a salt of any compound of the present invention of formula [I], [II], or [III], which contains a nitrogen-containing heterocyclic group, a hydroxyl group, a carboxyl group, an amino group, or the like in a structure thereof, with metals or organic bases or mineral acids or organic acids. Examples of the metal include alkali metals such as sodium and potassium and alkaline earth metals such as magnesium and calcium, examples of the organic base include triethylamine and diisopropylamine, examples of the mineral acid include hydrochloric acid, hydrobromic acid, and sulfuric acid, and examples of the organic acid include formic acid, acetic acid, methanesulfonic acid, 4-toluenesulfonic acid, and trifluoromethanesulfonic acid.

Next, representative examples of the compound included in the formamide derivative of the present invention of formula [I] are shown in Tables 1 to 119, examples of the representative compounds included in the aniline derivative of the present invention of formula [II] are shown in Tables 120 to 160, and examples of the representative compounds included in the amidoxime derivative of the present invention of formula [III] are shown in Tables 161 to 202. However, the compounds included in the present invention are not limited thereto. In addition, the compound numbers in the tables are referred to in the following description.

The compounds included in the formamide derivative, the aniline derivative, or the amidoxime derivative of the present invention can have geometrical isomers of E-body and Z-body depending on the kind of the substituent, and the present invention encompasses such E-body, Z-body, or a mixture containing E-body and Z-body in any ratio. In addition, the compounds included in the present invention can have optical isomers caused by the existence of one or two or more asymmetric carbon atoms and asymmetric sulfur atoms, and the present invention encompasses any optically active substances, racemic bodies, or diastereomers.

In the present specification, the following notations in the tables represent the corresponding groups, respectively, as follows.
Me: methyl;
Et: ethyl;
n-Pr: n-propyl;
i-Pr: isopropyl;
n-Bu: n-butyl
i-Bu: isobutyl
s-Bu: sec-butyl;
t-Bu: tert-butyl;
n-Pen: n-pentyl;
n-Hex: n-hexyl;
c-Pr: cyclopropyl;
c-Bu: cyclobutyl;
c-Pen: cyclopentyl;
c-Hex: cyclohexyl;
c-Pr(2,2-Cl₂): 2,2-dichlorocyclopropyl;
CF₃: trifluoromethyl;
CHF₂: difluoromethyl;
COMe: acetyl;
CO₂Me: methyl ester;
TMS: trimethylsilyl;
Ph(2-F): 2-fluorophenyl;
Ph(2,3-F₂): 2,3-difluorophenyl;
pyridin-2-yl(3-F): 3-fluoro-pyridin-2-yl;

Lengthy table referenced here
US20240358020A1-20241031-T00001
Please refer to the end of the specification for access instructions.

Lengthy table referenced here
US20240358020A1-20241031-T00002
Please refer to the end of the specification for access instructions.

Lengthy table referenced here
US20240358020A1-20241031-T00003
Please refer to the end of the specification for access instructions.

Lengthy table referenced here
US20240358020A1-20241031-T00004
Please refer to the end of the specification for access instructions.

Lengthy table referenced here
US20240358020A1-20241031-T00005
Please refer to the end of the specification for access instructions.

Lengthy table referenced here
US20240358020A1-20241031-T00006
Please refer to the end of the specification for access instructions.

Lengthy table referenced here
US20240358020A1-20241031-T00007
Please refer to the end of the specification for access instructions.

Lengthy table referenced here
US20240358020A1-20241031-T00008
Please refer to the end of the specification for access instructions.

Lengthy table referenced here
US20240358020A1-20241031-T00009
Please refer to the end of the specification for access instructions.

Lengthy table referenced here
US20240358020A1-20241031-T00010
Please refer to the end of the specification for access instructions.

Lengthy table referenced here
US20240358020A1-20241031-T00011
Please refer to the end of the specification for access instructions.

Lengthy table referenced here
US20240358020A1-20241031-T00012
Please refer to the end of the specification for access instructions.

Lengthy table referenced here
US20240358020A1-20241031-T00013
Please refer to the end of the specification for access instructions.

Lengthy table referenced here
US20240358020A1-20241031-T00014
Please refer to the end of the specification for access instructions.

Lengthy table referenced here
US20240358020A1-20241031-T00015
Please refer to the end of the specification for access instructions.

Lengthy table referenced here
US20240358020A1-20241031-T00016
Please refer to the end of the specification for access instructions.

Lengthy table referenced here
US20240358020A1-20241031-T00017
Please refer to the end of the specification for access instructions.

Lengthy table referenced here
US20240358020A1-20241031-T00018
Please refer to the end of the specification for access instructions.

Lengthy table referenced here
US20240358020A1-20241031-T00019
Please refer to the end of the specification for access instructions.

Lengthy table referenced here
US20240358020A1-20241031-T00020
Please refer to the end of the specification for access instructions.

Lengthy table referenced here
US20240358020A1-20241031-T00021
Please refer to the end of the specification for access instructions.

Lengthy table referenced here
US20240358020A1-20241031-T00022
Please refer to the end of the specification for access instructions.

Lengthy table referenced here
US20240358020A1-20241031-T00023
Please refer to the end of the specification for access instructions.

Lengthy table referenced here
US20240358020A1-20241031-T00024
Please refer to the end of the specification for access instructions.

Lengthy table referenced here
US20240358020A1-20241031-T00025
Please refer to the end of the specification for access instructions.

Lengthy table referenced here
US20240358020A1-20241031-T00026
Please refer to the end of the specification for access instructions.

Lengthy table referenced here
US20240358020A1-20241031-T00027
Please refer to the end of the specification for access instructions.

Lengthy table referenced here
US20240358020A1-20241031-T00028
Please refer to the end of the specification for access instructions.

Lengthy table referenced here
US20240358020A1-20241031-T00029
Please refer to the end of the specification for access instructions.

Lengthy table referenced here
US20240358020A1-20241031-T00030
Please refer to the end of the specification for access instructions.

Lengthy table referenced here
US20240358020A1-20241031-T00031
Please refer to the end of the specification for access instructions.

Lengthy table referenced here
US20240358020A1-20241031-T00032
Please refer to the end of the specification for access instructions.

Lengthy table referenced here
US20240358020A1-20241031-T00033
Please refer to the end of the specification for access instructions.

Lengthy table referenced here
US20240358020A1-20241031-T00034
Please refer to the end of the specification for access instructions.

Lengthy table referenced here
US20240358020A1-20241031-T00035
Please refer to the end of the specification for access instructions.

Lengthy table referenced here
US20240358020A1-20241031-T00036
Please refer to the end of the specification for access instructions.

Lengthy table referenced here
US20240358020A1-20241031-T00037
Please refer to the end of the specification for access instructions.

Lengthy table referenced here
US20240358020A1-20241031-T00038
Please refer to the end of the specification for access instructions.

Lengthy table referenced here
US20240358020A1-20241031-T00039
Please refer to the end of the specification for access instructions.

Lengthy table referenced here
US20240358020A1-20241031-T00040
Please refer to the end of the specification for access instructions.

Lengthy table referenced here
US20240358020A1-20241031-T00041
Please refer to the end of the specification for access instructions.

Lengthy table referenced here
US20240358020A1-20241031-T00042
Please refer to the end of the specification for access instructions.

Lengthy table referenced here
US20240358020A1-20241031-T00043
Please refer to the end of the specification for access instructions.

Lengthy table referenced here
US20240358020A1-20241031-T00044
Please refer to the end of the specification for access instructions.

Lengthy table referenced here
US20240358020A1-20241031-T00045
Please refer to the end of the specification for access instructions.

Lengthy table referenced here
US20240358020A1-20241031-T00046
Please refer to the end of the specification for access instructions.

Lengthy table referenced here
US20240358020A1-20241031-T00047
Please refer to the end of the specification for access instructions.

Lengthy table referenced here
US20240358020A1-20241031-T00048
Please refer to the end of the specification for access instructions.

Lengthy table referenced here
US20240358020A1-20241031-T00049
Please refer to the end of the specification for access instructions.

Lengthy table referenced here
US20240358020A1-20241031-T00050
Please refer to the end of the specification for access instructions.

Lengthy table referenced here
US20240358020A1-20241031-T00051
Please refer to the end of the specification for access instructions.

Lengthy table referenced here
US20240358020A1-20241031-T00052
Please refer to the end of the specification for access instructions.

Lengthy table referenced here
US20240358020A1-20241031-T00053
Please refer to the end of the specification for access instructions.

Lengthy table referenced here
US20240358020A1-20241031-T00054
Please refer to the end of the specification for access instructions.

Lengthy table referenced here
US20240358020A1-20241031-T00055
Please refer to the end of the specification for access instructions.

Lengthy table referenced here
US20240358020A1-20241031-T00056
Please refer to the end of the specification for access instructions.

Lengthy table referenced here
US20240358020A1-20241031-T00057
Please refer to the end of the specification for access instructions.

Lengthy table referenced here
US20240358020A1-20241031-T00058
Please refer to the end of the specification for access instructions.

Lengthy table referenced here
US20240358020A1-20241031-T00059
Please refer to the end of the specification for access instructions.

Lengthy table referenced here
US20240358020A1-20241031-T00060
Please refer to the end of the specification for access instructions.

Lengthy table referenced here
US20240358020A1-20241031-T00061
Please refer to the end of the specification for access instructions.

Lengthy table referenced here
US20240358020A1-20241031-T00062
Please refer to the end of the specification for access instructions.

Lengthy table referenced here
US20240358020A1-20241031-T00063
Please refer to the end of the specification for access instructions.

Lengthy table referenced here
US20240358020A1-20241031-T00064
Please refer to the end of the specification for access instructions.

Lengthy table referenced here
US20240358020A1-20241031-T00065
Please refer to the end of the specification for access instructions.

Lengthy table referenced here
US20240358020A1-20241031-T00066
Please refer to the end of the specification for access instructions.

Lengthy table referenced here
US20240358020A1-20241031-T00067
Please refer to the end of the specification for access instructions.

Lengthy table referenced here
US20240358020A1-20241031-T00068
Please refer to the end of the specification for access instructions.

Lengthy table referenced here
US20240358020A1-20241031-T00069
Please refer to the end of the specification for access instructions.

Lengthy table referenced here
US20240358020A1-20241031-T00070
Please refer to the end of the specification for access instructions.

Lengthy table referenced here
US20240358020A1-20241031-T00071
Please refer to the end of the specification for access instructions.

Lengthy table referenced here
US20240358020A1-20241031-T00072
Please refer to the end of the specification for access instructions.

Lengthy table referenced here
US20240358020A1-20241031-T00073
Please refer to the end of the specification for access instructions.

Lengthy table referenced here
US20240358020A1-20241031-T00074
Please refer to the end of the specification for access instructions.

Lengthy table referenced here
US20240358020A1-20241031-T00075
Please refer to the end of the specification for access instructions.

Lengthy table referenced here
US20240358020A1-20241031-T00076
Please refer to the end of the specification for access instructions.

Lengthy table referenced here
US20240358020A1-20241031-T00077
Please refer to the end of the specification for access instructions.

Lengthy table referenced here
US20240358020A1-20241031-T00078
Please refer to the end of the specification for access instructions.

Lengthy table referenced here
US20240358020A1-20241031-T00079
Please refer to the end of the specification for access instructions.

Lengthy table referenced here
US20240358020A1-20241031-T00080
Please refer to the end of the specification for access instructions.

Lengthy table referenced here
US20240358020A1-20241031-T00081
Please refer to the end of the specification for access instructions.

Lengthy table referenced here
US20240358020A1-20241031-T00082
Please refer to the end of the specification for access instructions.

Lengthy table referenced here
US20240358020A1-20241031-T00083
Please refer to the end of the specification for access instructions.

Lengthy table referenced here
US20240358020A1-20241031-T00084
Please refer to the end of the specification for access instructions.

Lengthy table referenced here
US20240358020A1-20241031-T00085
Please refer to the end of the specification for access instructions.

Lengthy table referenced here
US20240358020A1-20241031-T00086
Please refer to the end of the specification for access instructions.

Lengthy table referenced here
US20240358020A1-20241031-T00087
Please refer to the end of the specification for access instructions.

Lengthy table referenced here
US20240358020A1-20241031-T00088
Please refer to the end of the specification for access instructions.

Lengthy table referenced here
US20240358020A1-20241031-T00089
Please refer to the end of the specification for access instructions.

Lengthy table referenced here
US20240358020A1-20241031-T00090
Please refer to the end of the specification for access instructions.

Lengthy table referenced here
US20240358020A1-20241031-T00091
Please refer to the end of the specification for access instructions.

Lengthy table referenced here
US20240358020A1-20241031-T00092
Please refer to the end of the specification for access instructions.

Lengthy table referenced here
US20240358020A1-20241031-T00093
Please refer to the end of the specification for access instructions.

Lengthy table referenced here
US20240358020A1-20241031-T00094
Please refer to the end of the specification for access instructions.

Lengthy table referenced here
US20240358020A1-20241031-T00095
Please refer to the end of the specification for access instructions.

Lengthy table referenced here
US20240358020A1-20241031-T00096
Please refer to the end of the specification for access instructions.

Lengthy table referenced here
US20240358020A1-20241031-T00097
Please refer to the end of the specification for access instructions.

Lengthy table referenced here
US20240358020A1-20241031-T00098
Please refer to the end of the specification for access instructions.

Lengthy table referenced here
US20240358020A1-20241031-T00099
Please refer to the end of the specification for access instructions.

Lengthy table referenced here
US20240358020A1-20241031-T00100
Please refer to the end of the specification for access instructions.

Lengthy table referenced here
US20240358020A1-20241031-T00101
Please refer to the end of the specification for access instructions.

Lengthy table referenced here
US20240358020A1-20241031-T00102
Please refer to the end of the specification for access instructions.

Lengthy table referenced here
US20240358020A1-20241031-T00103
Please refer to the end of the specification for access instructions.

Lengthy table referenced here
US20240358020A1-20241031-T00104
Please refer to the end of the specification for access instructions.

Lengthy table referenced here
US20240358020A1-20241031-T00105
Please refer to the end of the specification for access instructions.

Lengthy table referenced here
US20240358020A1-20241031-T00106
Please refer to the end of the specification for access instructions.

Lengthy table referenced here
US20240358020A1-20241031-T00107
Please refer to the end of the specification for access instructions.

Lengthy table referenced here
US20240358020A1-20241031-T00108
Please refer to the end of the specification for access instructions.

Lengthy table referenced here
US20240358020A1-20241031-T00109
Please refer to the end of the specification for access instructions.

Lengthy table referenced here
US20240358020A1-20241031-T00110
Please refer to the end of the specification for access instructions.

Lengthy table referenced here
US20240358020A1-20241031-T00111
Please refer to the end of the specification for access instructions.

Lengthy table referenced here
US20240358020A1-20241031-T00112
Please refer to the end of the specification for access instructions.

Lengthy table referenced here
US20240358020A1-20241031-T00113
Please refer to the end of the specification for access instructions.

Lengthy table referenced here
US20240358020A1-20241031-T00114
Please refer to the end of the specification for access instructions.

Lengthy table referenced here
US20240358020A1-20241031-T00115
Please refer to the end of the specification for access instructions.

Lengthy table referenced here
US20240358020A1-20241031-T00116
Please refer to the end of the specification for access instructions.

Lengthy table referenced here
US20240358020A1-20241031-T00117
Please refer to the end of the specification for access instructions.

Lengthy table referenced here
US20240358020A1-20241031-T00118
Please refer to the end of the specification for access instructions.

Lengthy table referenced here
US20240358020A1-20241031-T00119
Please refer to the end of the specification for access instructions.

Lengthy table referenced here
US20240358020A1-20241031-T00120
Please refer to the end of the specification for access instructions.

Lengthy table referenced here
US20240358020A1-20241031-T00121
Please refer to the end of the specification for access instructions.

Lengthy table referenced here
US20240358020A1-20241031-T00122
Please refer to the end of the specification for access instructions.

Lengthy table referenced here
US20240358020A1-20241031-T00123
Please refer to the end of the specification for access instructions.

Lengthy table referenced here
US20240358020A1-20241031-T00124
Please refer to the end of the specification for access instructions.

Lengthy table referenced here
US20240358020A1-20241031-T00125
Please refer to the end of the specification for access instructions.

Lengthy table referenced here
US20240358020A1-20241031-T00126
Please refer to the end of the specification for access instructions.

Lengthy table referenced here
US20240358020A1-20241031-T00127
Please refer to the end of the specification for access instructions.

Lengthy table referenced here
US20240358020A1-20241031-T00128
Please refer to the end of the specification for access instructions.

Lengthy table referenced here
US20240358020A1-20241031-T00129
Please refer to the end of the specification for access instructions.

Lengthy table referenced here
US20240358020A1-20241031-T00130
Please refer to the end of the specification for access instructions.

Lengthy table referenced here
US20240358020A1-20241031-T00131
Please refer to the end of the specification for access instructions.

Lengthy table referenced here
US20240358020A1-20241031-T00132
Please refer to the end of the specification for access instructions.

Lengthy table referenced here
US20240358020A1-20241031-T00133
Please refer to the end of the specification for access instructions.

Lengthy table referenced here
US20240358020A1-20241031-T00134
Please refer to the end of the specification for access instructions.

Lengthy table referenced here
US20240358020A1-20241031-T00135
Please refer to the end of the specification for access instructions.

Lengthy table referenced here
US20240358020A1-20241031-T00136
Please refer to the end of the specification for access instructions.

Lengthy table referenced here
US20240358020A1-20241031-T00137
Please refer to the end of the specification for access instructions.

Lengthy table referenced here
US20240358020A1-20241031-T00138
Please refer to the end of the specification for access instructions.

Lengthy table referenced here
US20240358020A1-20241031-T00139
Please refer to the end of the specification for access instructions.

Lengthy table referenced here
US20240358020A1-20241031-T00140
Please refer to the end of the specification for access instructions.

Lengthy table referenced here
US20240358020A1-20241031-T00141
Please refer to the end of the specification for access instructions.

Lengthy table referenced here
US20240358020A1-20241031-T00142
Please refer to the end of the specification for access instructions.

Lengthy table referenced here
US20240358020A1-20241031-T00143
Please refer to the end of the specification for access instructions.

Lengthy table referenced here
US20240358020A1-20241031-T00144
Please refer to the end of the specification for access instructions.

Lengthy table referenced here
US20240358020A1-20241031-T00145
Please refer to the end of the specification for access instructions.

Lengthy table referenced here
US20240358020A1-20241031-T00146
Please refer to the end of the specification for access instructions.

Lengthy table referenced here
US20240358020A1-20241031-T00147
Please refer to the end of the specification for access instructions.

Lengthy table referenced here
US20240358020A1-20241031-T00148
Please refer to the end of the specification for access instructions.

Lengthy table referenced here
US20240358020A1-20241031-T00149
Please refer to the end of the specification for access instructions.

Lengthy table referenced here
US20240358020A1-20241031-T00150
Please refer to the end of the specification for access instructions.

Lengthy table referenced here
US20240358020A1-20241031-T00151
Please refer to the end of the specification for access instructions.

Lengthy table referenced here
US20240358020A1-20241031-T00152
Please refer to the end of the specification for access instructions.

Lengthy table referenced here
US20240358020A1-20241031-T00153
Please refer to the end of the specification for access instructions.

Lengthy table referenced here
US20240358020A1-20241031-T00154
Please refer to the end of the specification for access instructions.

Lengthy table referenced here
US20240358020A1-20241031-T00155
Please refer to the end of the specification for access instructions.

Lengthy table referenced here
US20240358020A1-20241031-T00156
Please refer to the end of the specification for access instructions.

Lengthy table referenced here
US20240358020A1-20241031-T00157
Please refer to the end of the specification for access instructions.

Lengthy table referenced here
US20240358020A1-20241031-T00158
Please refer to the end of the specification for access instructions.

Lengthy table referenced here
US20240358020A1-20241031-T00159
Please refer to the end of the specification for access instructions.

Lengthy table referenced here
US20240358020A1-20241031-T00160
Please refer to the end of the specification for access instructions.

Lengthy table referenced here
US20240358020A1-20241031-T00161
Please refer to the end of the specification for access instructions.

Lengthy table referenced here
US20240358020A1-20241031-T00162
Please refer to the end of the specification for access instructions.

Lengthy table referenced here
US20240358020A1-20241031-T00163
Please refer to the end of the specification for access instructions.

Lengthy table referenced here
US20240358020A1-20241031-T00164
Please refer to the end of the specification for access instructions.

Lengthy table referenced here
US20240358020A1-20241031-T00165
Please refer to the end of the specification for access instructions.

Lengthy table referenced here
US20240358020A1-20241031-T00166
Please refer to the end of the specification for access instructions.

Lengthy table referenced here
US20240358020A1-20241031-T00167
Please refer to the end of the specification for access instructions.

Lengthy table referenced here
US20240358020A1-20241031-T00168
Please refer to the end of the specification for access instructions.

Lengthy table referenced here
US20240358020A1-20241031-T00169
Please refer to the end of the specification for access instructions.

Lengthy table referenced here
US20240358020A1-20241031-T00170
Please refer to the end of the specification for access instructions.

Lengthy table referenced here
US20240358020A1-20241031-T00171
Please refer to the end of the specification for access instructions.

Lengthy table referenced here
US20240358020A1-20241031-T00172
Please refer to the end of the specification for access instructions.

Lengthy table referenced here
US20240358020A1-20241031-T00173
Please refer to the end of the specification for access instructions.

Lengthy table referenced here
US20240358020A1-20241031-T00174
Please refer to the end of the specification for access instructions.

Lengthy table referenced here
US20240358020A1-20241031-T00175
Please refer to the end of the specification for access instructions.

Lengthy table referenced here
US20240358020A1-20241031-T00176
Please refer to the end of the specification for access instructions.

Lengthy table referenced here
US20240358020A1-20241031-T00177
Please refer to the end of the specification for access instructions.

Lengthy table referenced here
US20240358020A1-20241031-T00178
Please refer to the end of the specification for access instructions.

Lengthy table referenced here
US20240358020A1-20241031-T00179
Please refer to the end of the specification for access instructions.

Lengthy table referenced here
US20240358020A1-20241031-T00180
Please refer to the end of the specification for access instructions.

Lengthy table referenced here
US20240358020A1-20241031-T00181
Please refer to the end of the specification for access instructions.

Lengthy table referenced here
US20240358020A1-20241031-T00182
Please refer to the end of the specification for access instructions.

Lengthy table referenced here
US20240358020A1-20241031-T00183
Please refer to the end of the specification for access instructions.

Lengthy table referenced here
US20240358020A1-20241031-T00184
Please refer to the end of the specification for access instructions.

Lengthy table referenced here
US20240358020A1-20241031-T00185
Please refer to the end of the specification for access instructions.

Lengthy table referenced here
US20240358020A1-20241031-T00186
Please refer to the end of the specification for access instructions.

Lengthy table referenced here
US20240358020A1-20241031-T00187
Please refer to the end of the specification for access instructions.

Lengthy table referenced here
US20240358020A1-20241031-T00188
Please refer to the end of the specification for access instructions.

Lengthy table referenced here
US20240358020A1-20241031-T00189
Please refer to the end of the specification for access instructions.

Lengthy table referenced here
US20240358020A1-20241031-T00190
Please refer to the end of the specification for access instructions.

Lengthy table referenced here
US20240358020A1-20241031-T00191
Please refer to the end of the specification for access instructions.

Lengthy table referenced here
US20240358020A1-20241031-T00192
Please refer to the end of the specification for access instructions.

Lengthy table referenced here
US20240358020A1-20241031-T00193
Please refer to the end of the specification for access instructions.

Lengthy table referenced here
US20240358020A1-20241031-T00194
Please refer to the end of the specification for access instructions.

Lengthy table referenced here
US20240358020A1-20241031-T00195
Please refer to the end of the specification for access instructions.

Lengthy table referenced here
US20240358020A1-20241031-T00196
Please refer to the end of the specification for access instructions.

Meanwhile, the compound of the present invention of formula [I], [II], or [III] can be produced according to the following production method, but the production method is not limited to these methods. Hereinafter, for example, a “compound of formula [I-1]” is synonymous with a “compound [I-1]”.

### <Production Method 1>

A compound of formula [I-1] among the compounds of the present invention can be produced, for example, according to a method based on the reaction scheme exemplified below.

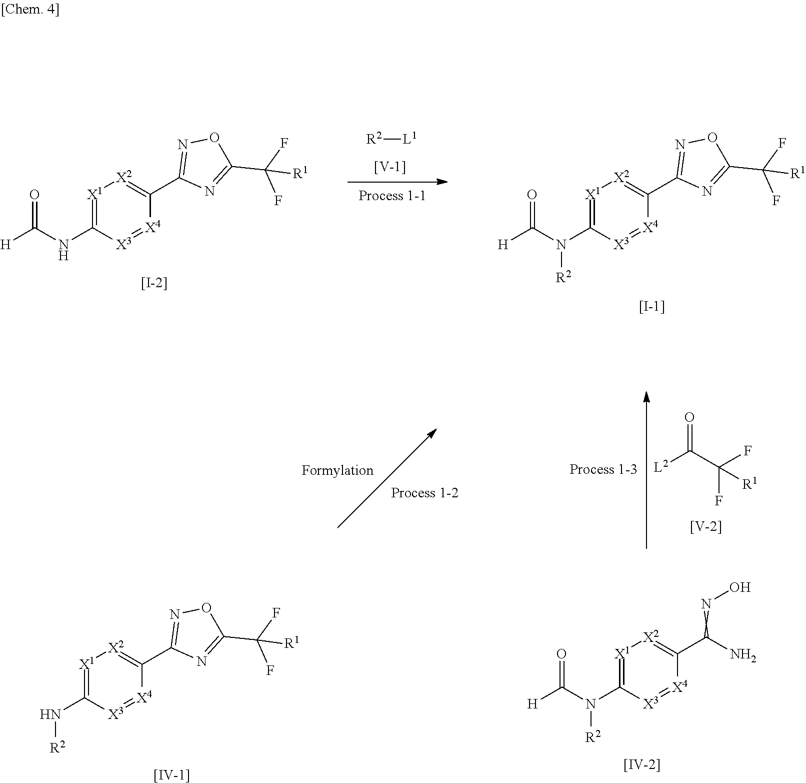

(in the formula, L¹ represents a halogen atom, a (C1-C6)alkylsulfonyloxy, a (C1-C6)haloalkylsulfonyloxy, a phenylsulfonyloxy, a 4-toluenesulfonyloxy, or a 1-imidazolyl, L² represents a halogen atom, a (C1-C6)alkoxy, a (C1-C6)haloalkoxy, a phenoxy, a (C1-C6)alkylsulfonyloxy, a (C1-C6)haloalkylsulfonyloxy, a phenylsulfonyloxy, a 4-toluenesulfonyloxy, a 1-imidazolyl, a (C1-C6)alkoxycarbonyloxy, a (C1-C6)alkylcarbonyloxy, a (C1-C6)haloalkylcarbonyloxy, or a (C1-C6)haloalkoxycarbonyloxy, and X¹, X², X³, X⁴, R¹, and R² are as defined above.)

### (Process 1-1)

That is, the compound of formula [I-1] can be produced by reacting a compound [I-2] and a compound [V-1] in the presence or absence of an appropriate solvent, in the presence or absence of an appropriate base, and in the presence or absence of an appropriate catalyst.

The amount of the compound [V-1] used in the present reaction may be appropriately selected from a range of usually 1.0 to 20 mol, and is preferably 1.0 to 3.0 mol, based on 1 mol of the compound [I-2].

Examples of the base that can be used in the present reaction include hydroxides of alkali metal, such as lithium hydroxide, sodium hydroxide, and potassium hydroxide; hydroxides of alkali earth metal, such as calcium hydroxide and magnesium hydroxide; carbonates of alkali metal, such as lithium carbonate, sodium carbonate, potassium carbonate, and cesium carbonate; bicarbonates of alkali metal, such as sodium bicarbonate and potassium bicarbonate; metal hydrides of alkali metal, such as lithium hydride, sodium hydride, and potassium hydride; metal salts of alcohol, such as sodium methoxide, sodium ethoxide, and potassium tert-butoxide; and organic bases such as triethylamine, tributylamine, diisopropylethylamine, N,N-dimethylaniline, pyridine, 2,6-lutidine, 4-N,N-dimethylaminopyridine, and 1,8-diazacyclo[5.4.0]-7-undecene. The amount of the base used may be appropriately selected from a range of 0.5 to 10 mol, and is preferably 1.0 to 5.0 mol, based on 1 mol of the compound [I-2].

Examples of the catalyst that can be used in the present reaction include iodides of alkali metal, such as sodium iodide and potassium iodide. The amount of the catalyst used may be appropriately selected from a range of 0.01 to 0.5 mol, and is preferably 0.01 to 0.1 mol, based on 1 mol of the compound [I-2].

Examples of the solvent that can be used in the present reaction include ethers such as diethyl ether, diisopropyl ether, methyl tert-butyl ether, cyclopentyl methyl ether, tetrahydrofuran, 2-methyl tetrahydrofuran, 4-methyltetrahydropyran, 1,4-dioxane, and monoglyme; aromatic hydrocarbons such as benzene, toluene, xylene, mesitylene, and chlorobenzene; halogenated hydrocarbons such as dichloromethane, chloroform, and 1,2-dichloroethane; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; nitriles such as acetonitrile and propionitrile; aliphatic hydrocarbons such as pentane, hexane, cyclohexane, and heptane; pyridines such as pyridine, picoline, and lutidine; tertiary amines such as triethylamine and tributylamine; water; and a mixed solvent thereof. The amount of the solvent used is 0.1 to 100 liters, and preferably 0.1 to 20 liters, based on 1 mol of the compound [1-2].

The reaction temperature of the present reaction may be selected from a range of any temperature usually from −78° C. to a reflux temperature in a reaction system, and may be preferably in a range of 0° C. to 150° C. The reaction time of the present reaction is usually 10 minutes to 72 hours although it varies depending on the reaction temperature, the reactant, the reaction amount, and the like.

After the completion of the reaction, the compound [I-1] can be isolated by subjecting the reaction mixture to operations such as pouring into water, extraction with an organic solvent, and concentration. The isolated compound [I-1] can be further purified by column chromatography, recrystallization, or the like, as necessary.

### (Process 1-2)

That is, the compound of formula [I-1] can be produced by reacting a compound [IV-1] and an appropriate formylating agent in the presence or absence of an appropriate solvent and in the presence or absence of an appropriate acid catalyst.

The compound [IV-1] can be produced in accordance with the method described in of the description of EP 3,187,497 A, page 68, or the method thereof.

Examples of the formylating agent that can be used in the present reaction include formic acid, acetic anhydride, sodium formate, methyl formate, ethyl formate, cyanomethyl formate, N-formyl saccharin, and 1-formyl-1H-benzotriazole. The amount of the formylating agent used may be appropriately selected from a range of usually 1.0 to 20 mol, and is preferably 1.0 to 10 mol, based on 1 mol of the compound [IV-1].

Examples of the solvent that can be used in the present reaction include ethers such as diethyl ether, diisopropyl ether, methyl tert-butyl ether, cyclopentyl methyl ether, tetrahydrofuran, 2-methyl tetrahydrofuran, 4-methyltetrahydropyran, 1,4-dioxane, and monoglyme; aromatic hydrocarbons such as benzene, toluene, xylene, mesitylene, and chlorobenzene; halogenated hydrocarbons such as dichloromethane, chloroform, and 1,2-dichloroethane; aliphatic hydrocarbons such as pentane, hexane, cyclohexane, and heptane; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; alcohols such as methanol, ethanol, 2-propanol, tert-butyl alcohol, and methyl cellosolve; carboxylic acids such as formic acid, acetic acid, propionic acid, and trifluoroacetic acid; water; and a mixed solvent thereof. The amount of the solvent used is 0.1 to 100 liters, and preferably 0.1 to 20 liters, based on 1 mol of the compound [IV-1].

Examples of the acid catalyst that can be used in the present reaction include mineral acids such as hydrochloric acid, hydrobromic acid, and sulfuric acid, carboxylic acids such as acetic acid, propionic acid, dichloroacetic acid, and trifluoroacetic acid; and sulfonic acids such as methanesulfonic acid, benzenesulfonic acid, 4-toluenesulfonic acid, and trifluoromethanesulfonic acid. The amount of the acid catalyst used may be appropriately selected from a range of usually 0.01 to 10 mol, and is preferably 0.01 to 5.0 mol, based on 1 mol of the compound [IV-1].

The reaction temperature of the present reaction may be selected from a range of any temperature usually from 0° C. to a reflux temperature in a reaction system, and may be preferably in a range of a reflux temperature in a reaction system from room temperature. The reaction time of the present reaction is usually 10 minutes to 72 hours although it varies depending on the reaction temperature, the reactant, the reaction amount, and the like.

After the completion of the reaction, the compound [I-1] can be isolated by subjecting the reaction mixture to operations such as pouring into water, extraction with an organic solvent, and concentration. The isolated compound [I-1] can be further purified by column chromatography, recrystallization, or the like, as necessary.

### (Process 1-3)

That is, the compound of formula [I-1] can be produced by reacting a compound [IV-2] and a compound [V-2] in the presence or absence of an appropriate solvent and in the presence or absence of an appropriate base.

The amount of the compound [V-2] used in the present reaction may be appropriately selected from a range of usually 1.0 to 20 mol, and is preferably 1.0 to 3.0 mol, based on 1 mol of the compound [IV-2].

Examples of the base that can be used in the present reaction include hydroxides of alkali metal, such as lithium hydroxide, sodium hydroxide, and potassium hydroxide; hydroxides of alkali earth metal, such as calcium hydroxide and magnesium hydroxide; carbonates of alkali metal, such as lithium carbonate, sodium carbonate, potassium carbonate, and cesium carbonate; bicarbonates of alkali metal, such as sodium bicarbonate and potassium bicarbonate; metal hydrides of alkali metal, such as lithium hydride, sodium hydride, and potassium hydride; metal salts of alcohol, such as sodium methoxide, sodium ethoxide, and potassium tert-butoxide; and organic bases such as triethylamine, tributylamine, diisopropylethylamine, N,N-dimethylaniline, pyridine, 2,6-lutidine, 4-N,N-dimethylaminopyridine, and 1,8-diazacyclo[5.4.0]-7-undecene. The amount of the base used may be appropriately selected from a range of 0.5 to 10 mol, and is preferably 1.0 to 5.0 mol, based on 1 mol of the compound [IV-2].

Examples of the solvent that can be used in the present reaction include ethers such as diethyl ether, diisopropyl ether, methyl tert-butyl ether, cyclopentyl methyl ether, tetrahydrofuran, 2-methyl tetrahydrofuran, 4-methyltetrahydropyran, 1,4-dioxane, and monoglyme; aromatic hydrocarbons such as benzene, toluene, xylene, mesitylene, and chlorobenzene; halogenated hydrocarbons such as dichloromethane, chloroform, and 1,2-dichloroethane; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; alcohols such as methanol, ethanol, 2-propanol, tert-butyl alcohol, and methyl cellosolve; nitriles such as acetonitrile and propionitrile; aliphatic hydrocarbons such as pentane, hexane, cyclohexane, and heptane; pyridines such as pyridine, picoline, and lutidine; tertiary amines such as triethylamine and tributylamine; water; and a mixed solvent thereof. The amount of the solvent used is 0.1 to 100 liters, and preferably 0.1 to 20 liters, based on 1 mol of the compound [IV-2].

The reaction temperature of the present reaction may be selected from a range of any temperature usually from −78° C. to a reflux temperature in a reaction system, and may be preferably in a range of a reflux temperature in a reaction system from 0° C. The reaction time of the present reaction is usually 10 minutes to 72 hours although it varies depending on the reaction temperature, the reactant, the reaction amount, and the like.

After the completion of the reaction, the compound [I-1] can be isolated by subjecting the reaction mixture to operations such as pouring into water, extraction with an organic solvent, and concentration. The isolated compound [I-1] can be further purified by column chromatography, recrystallization, or the like, as necessary.

### <Production Method 2>

A compound of formula [I-2] among the compounds of the present invention can be produced, for example, according to a method based on the reaction scheme exemplified below.

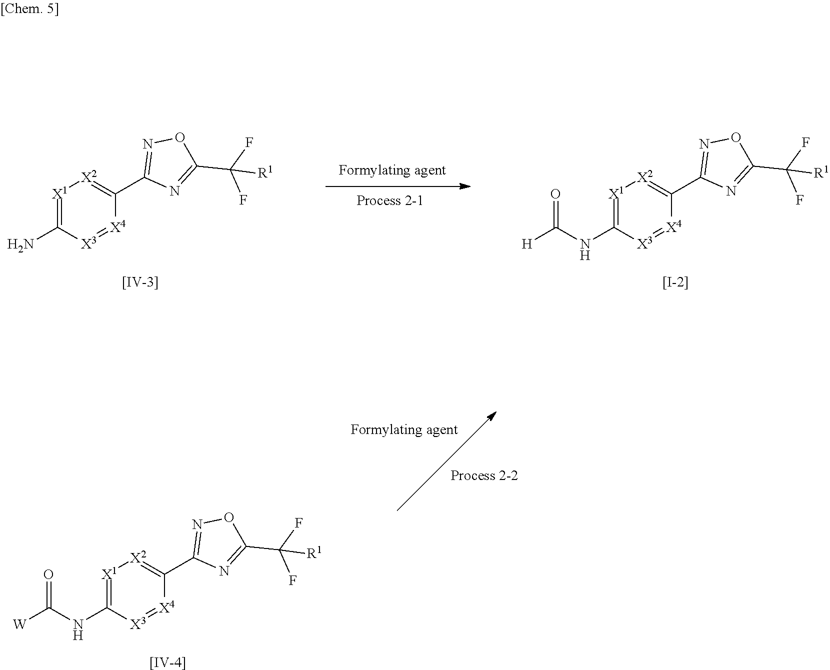

(in the formula, W represents (C1-C6) alkoxy or (C1-C6)haloalkyl, and X¹, X², X³, X⁴, and R¹ are as defined above.)

### (Process 2-1)

That is, the compound of formula [I-2] can be produced by reacting a compound [IV-3] and an appropriate formylating agent in the presence or absence of an appropriate solvent and in the presence or absence of an appropriate acid catalyst. The compound [IV-3] can be produced in accordance with the method described in the description of WO 2007/111212 A, page 323, or the method thereof.

The formylating agent, the solvent, the acid catalyst, the reaction temperature, and the reaction time that can be used in the present reaction are the same as those in Process 1-2 of Production Method 1.

After the completion of the reaction, the compound [I-2] can be isolated by subjecting the reaction mixture to operations such as pouring into water, extraction with an organic solvent, and concentration. The isolated compound [I-2] can be further purified by column chromatography, recrystallization, or the like, as necessary.

### (Process 2-2)

That is, the compound of formula [I-2] can be produced by reacting a compound [IV-4] and an appropriate formylating agent in the presence or absence of an appropriate solvent and in the presence or absence of an appropriate acid catalyst. The compound [IV-4] can be produced in accordance with the method described in the description of WO 2007/111212 A, page 323, or the method thereof.

The formylating agent, the solvent, the acid catalyst, the reaction temperature, and the reaction time that can be used in the present reaction are the same as those in Process 1-2 of Production Method 1.

After the completion of the reaction, the compound [I-2] can be isolated by subjecting the reaction mixture to operations such as pouring into water, extraction with an organic solvent, and concentration. The isolated compound [I-2] can be further purified by column chromatography, recrystallization, or the like, as necessary.

### <Production Method 3>

A compound of formula [IV-2] can be produced, for example, according to a method based on the reaction scheme exemplified below.

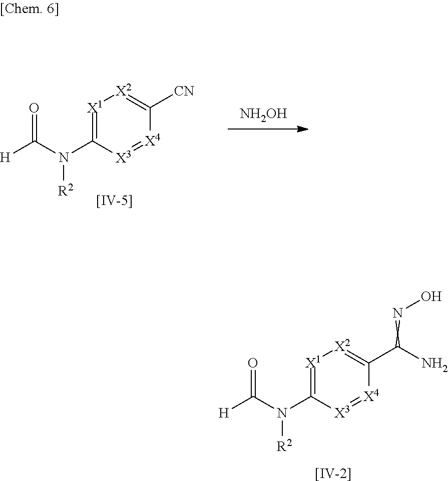

(in the formula, X¹, X², X³, X⁴, and R² are as defined above.)

That is, the compound of formula [IV-2] can be produced by reacting the compound [IV-5] and appropriate hydroxylamine in the presence of an appropriate solvent and in the presence or absence of an appropriate base.

Examples of the hydroxylamine that can be used in the present reaction include a hydroxylamine aqueous solution, hydroxylamine hydrochloride, and hydroxylamine sulfate. The amount of the hydroxylamine used may be appropriately selected from a range of 1.0 to 20 mol, and is preferably 1.0 to 10 mol, based on 1 mol of the compound [IV-5].

Examples of the base that can be used in the present reaction include hydroxides of alkali metal, such as lithium hydroxide, sodium hydroxide, and potassium hydroxide; hydroxides of alkali earth metal, such as calcium hydroxide and magnesium hydroxide; carbonates of alkali metal, such as lithium carbonate, sodium carbonate, potassium carbonate, and cesium carbonate; bicarbonates of alkali metal, such as sodium bicarbonate and potassium bicarbonate; metal hydrides of alkali metal, such as lithium hydride, sodium hydride, and potassium hydride; metal salts of alcohol, such as sodium methoxide, sodium ethoxide, and potassium tert-butoxide; and organic bases such as triethylamine, tributylamine, diisopropylethylamine, N,N-dimethylaniline, pyridine, 2,6-lutidine, 4-N,N-dimethylaminopyridine, and 1,8-diazacyclo[5.4.0]-7-undecene. The amount of the base used may be appropriately selected from a range of 0.5 to 10 mol, and is preferably 1.0 to 10 mol, based on 1 mol of the compound [IV-5].

Examples of the solvent that can be used in the present reaction include ethers such as diethyl ether, diisopropyl ether, methyl tert-butyl ether, cyclopentyl methyl ether, tetrahydrofuran, 2-methyl tetrahydrofuran, 4-methyltetrahydropyran, 1,4-dioxane, and monoglyme; aromatic hydrocarbons such as benzene, toluene, xylene, mesitylene, and chlorobenzene; halogenated hydrocarbons such as dichloromethane, chloroform, and 1,2-dichloroethane; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; alcohols such as methanol, ethanol, 2-propanol, tert-butyl alcohol, and methyl cellosolve; nitriles such as acetonitrile and propionitrile; aliphatic hydrocarbons such as pentane, hexane, cyclohexane, and heptane; pyridines such as pyridine, picoline, and lutidine; tertiary amines such as triethylamine and tributylamine; water; and a mixed solvent thereof. The amount of the solvent used is 0.1 to 100 liters, and preferably 0.1 to 20 liters, based on 1 mol of the compound [IV-5].

The reaction temperature of the present reaction may be selected from a range of any temperature usually from 0° C. to a reflux temperature in a reaction system, and may be preferably in a range of a reflux temperature in a reaction system from room temperature. The reaction time of the present reaction is usually 10 minutes to 72 hours although it varies depending on the reaction temperature, the reactant, the reaction amount, and the like.

After the completion of the reaction, the compound [IV-2] can be isolated by subjecting the reaction mixture to operations such as pouring into water, extraction with an organic solvent, and concentration. The isolated compound [IV-2] can be further purified by column chromatography, recrystallization, or the like, as necessary.

### <Production Method 4>

A compound of formula [IV-5] can be produced, for example, according to a method based on the reaction scheme exemplified below.

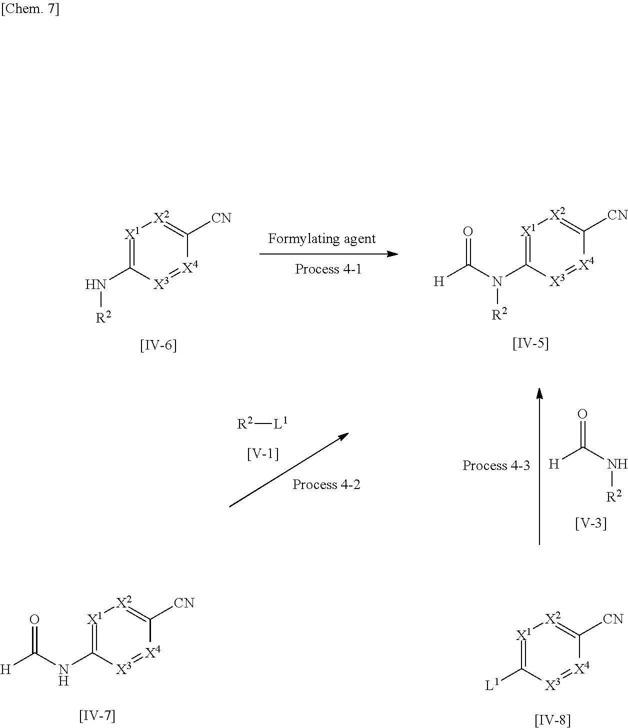

(in the formula, L¹, X¹, X², X³, X⁴, and R² are as defined above.)

### (Process 4-1)

That is, the compound of formula [IV-5] can be produced by reacting a compound [IV-6] and an appropriate formylating agent in the presence or absence of an appropriate solvent and in the presence or absence of an appropriate acid catalyst.

The formylating agent, the solvent, the acid catalyst, the reaction temperature, and the reaction time that can be used in the present reaction are the same as those in Process 1-2 of Production Method 1.

After the completion of the reaction, the compound [IV-5] can be isolated by subjecting the reaction mixture to operations such as pouring into water, extraction with an organic solvent, and concentration. The isolated compound [IV-5] can be further purified by column chromatography, recrystallization, or the like, as necessary.

### (Process 4-2)

That is, the compound of formula [IV-5] can be produced by reacting a compound [IV-7] and a compound [V-1] in the presence or absence of an appropriate solvent, in the presence or absence of an appropriate base, and in the presence or absence of an appropriate catalyst. The compound [IV-7] can be produced in accordance with the method described in European Journal of Organic Chemistry, vol. 28, page 4,539 page, 2019, or the method thereof.

The amount of the compound [V-1] used in the present reaction may be appropriately selected from a range of usually 1.0 to 20 mol, and is preferably 1.0 to 3.0 mol, based on 1 mol of the compound [IV-7].

The base, the catalyst, and the solvent that can be used in the present reaction are the same as those in Process 1-1 of Production Method 1.

The reaction temperature of the present reaction may be selected from a range of any temperature usually from 0° C. to a reflux temperature in a reaction system, and may be preferably in a range of 0° C. to 100° C. The reaction time of the present reaction is usually 10 minutes to 72 hours although it varies depending on the reaction temperature, the reactant, the reaction amount, and the like.

After the completion of the reaction, the compound [IV-5] can be isolated by subjecting the reaction mixture to operations such as pouring into water, extraction with an organic solvent, and concentration. The isolated compound [IV-5] can be further purified by column chromatography, recrystallization, or the like, as necessary.

### (Process 4-3)

That is, the compound of formula [IV-5] can be produced by reacting a compound [IV-8] and a compound [V-3] in the presence or absence of an appropriate solvent, in the presence or absence of an appropriate base, in the presence or absence of an appropriate catalyst, and in the presence or absence of a copper salt.

The amount of the compound [V-3] used in the present reaction may be appropriately selected from a range of usually 1.0 to 20 mol, and is preferably 1.0 to 3.0 mol, based on 1 mol of the compound [IV-8].

Examples of the base that can be used in the present reaction include hydroxides of alkali metal, such as lithium hydroxide, sodium hydroxide, and potassium hydroxide; hydroxides of alkali earth metal, such as calcium hydroxide and magnesium hydroxide; carbonates of alkali metal, such as lithium carbonate, sodium carbonate, potassium carbonate, and cesium carbonate; bicarbonates of alkali metal, such as sodium bicarbonate and potassium bicarbonate; metal hydrides of alkali metal, such as lithium hydride, sodium hydride, and potassium hydride; inorganic bases such as fluoride of alkali metal, such as sodium fluoride and potassium fluoride, and tripotassium phosphate; metal salts of alcohol, such as sodium methoxide, sodium ethoxide, and potassium tert-butoxide; and organic bases such as triethylamine, tributylamine, diisopropylethylamine, N,N-dimethylaniline, pyridine, 2,6-lutidine, 4-N,N-dimethylaminopyridine, and 1,8-diazacyclo[5.4.0]-7-undecene. The amount of the base used may be appropriately selected from a range of 0.5 to 10 mol, and is preferably 1.0 to 5.0 mol, based on 1 mol of the compound [IV-8].

As the catalyst that can be used in the present reaction, a transition metal complex or a combination of a transition metal and a ligand may be used. Examples of the transition metal complex include tetrakis(triphenylphosphine)palladium and a dichloro[1,1′-bis(diphenylphosphino)ferrocene]palladium-dichloromethane adduct. Examples of the transition metal include palladium chloride, palladium bromide, and palladium acetate. Examples of the ligand include trimethylphosphine, triethylphosphine, tributylphosphine, triphenylphosphine, 1,3-bisdimethylphosphinopropane, 1,2-bisdiphenylphosphinoetane, and 1,3-bisdiphenylphosphinopropane. The amount of the catalyst used is 0.001 to 0.5 mol, and preferably 0.01 to 0.2 mol, based on 1 mol of the compound [IV-8].

Examples of the copper salt that can be used in the present reaction include copper(I) chloride, copper(I) bromide, and copper(I) iodide. The amount of the copper salt used is 0.1 to 10 mol, and preferably 0.1 to 4.0 mol, based on 1 mol of the compound [IV-8].

Examples of the solvent that can be used in the present reaction include ethers such as diethyl ether, diisopropyl ether, methyl tert-butyl ether, cyclopentyl methyl ether, tetrahydrofuran, 2-methyl tetrahydrofuran, 4-methyltetrahydropyran, 1,4-dioxane, and monoglyme; aromatic hydrocarbons such as benzene, toluene, xylene, mesitylene, and chlorobenzene; halogenated hydrocarbons such as dichloromethane, chloroform, and 1,2-dichloroethane; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; nitriles such as acetonitrile and propionitrile; aliphatic hydrocarbons such as pentane, hexane, cyclohexane, and heptane; pyridines such as pyridine, picoline, and lutidine; tertiary amines such as triethylamine and tributylamine; water; and a mixed solvent thereof. The amount of the solvent used is 0.1 to 100 liters, and preferably 0.1 to 20 liters, based on 1 mol of the compound [IV-8].

The reaction temperature of the present reaction may be selected from a range of any temperature usually from 0° C. to a reflux temperature in a reaction system, and may be preferably in a range of 0° C. to 150° C. The reaction time of the present reaction is usually 10 minutes to 72 hours although it varies depending on the reaction temperature, the reactant, the reaction amount, and the like.

After the completion of the reaction, the compound [IV-5] can be isolated by subjecting the reaction mixture to operations such as pouring into water, extraction with an organic solvent, and concentration. The isolated compound [IV-5] can be further purified by column chromatography, recrystallization, or the like, as necessary.

### <Production Method 5>

A compound of formula [IV-6] can be produced, for example, according to the method shown by the following reaction formula using a compound of formula [IV-8].

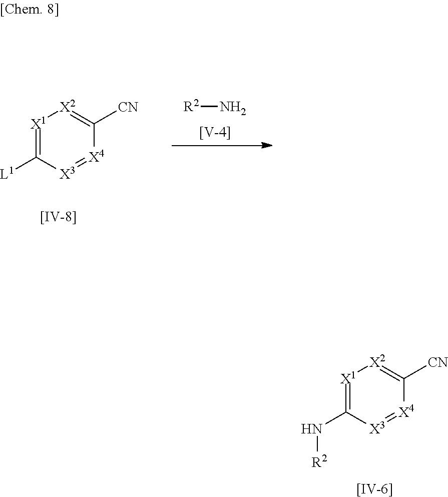

(in the formula, L¹, X¹, X², X³, X⁴, and R² are as defined above.)

That is, the compound of formula [IV-6] can be produced by reacting a compound [IV-8] and a compound [V-4] in the presence or absence of an appropriate solvent, in the presence or absence of an appropriate base, in the presence or absence of an appropriate catalyst, and in the presence or absence of a copper salt.

The amount of the compound [V-4] used in the present reaction may be appropriately selected from a range of usually 1.0 to 20 mol, and is preferably 1.0 to 3.0 mol, based on 1 mol of the compound [IV-8].

The base, the catalyst, the copper salt, the solvent, the reaction temperature, and the reaction time that can be used in the present reaction are the same as those in Process 4-3 of Production Method 4.

After the completion of the reaction, the compound [IV-6] can be isolated by subjecting the reaction mixture to operations such as pouring into water, extraction with an organic solvent, and concentration. The isolated compound [IV-6] can be further purified by column chromatography, recrystallization, or the like, as necessary.

### <Production Method 6>

A compound of formula [IV-9] can be produced, for example, according to a method based on the reaction scheme exemplified below.

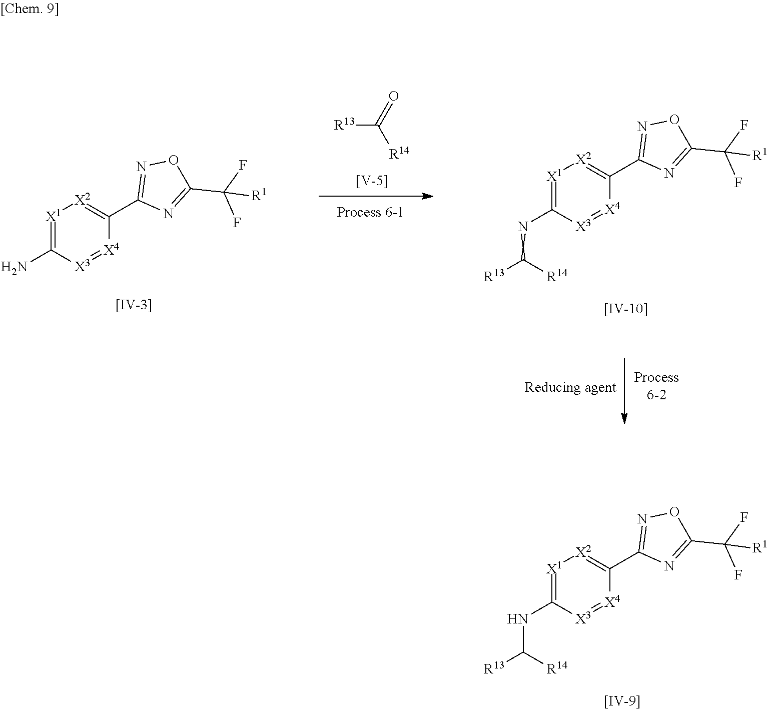

(in the formula, R¹³ and R¹⁴ are each independently selected and each represent a hydrogen atom, a (C1-C5)alkyl (the (C1-C5)alkyl is optionally mono-substituted or poly-substituted with R⁴), a (C2-C5)alkenyl (the (C2-C5)alkenyl is optionally mono-substituted or poly-substituted with R⁴), a (C2-C5) alkynyl group (the (C2-C5) alkynyl is optionally mono-substituted or poly-substituted with R⁴), a (C3-C6)cycloalkyl, a (C6-C12)aryl (the (C6-C12)aryl is optionally mono-substituted or poly-substituted with R⁵), a 3- to 6-membered heterocyclyl (the 3- to 6-membered heterocyclyl is optionally mono-substituted or poly-substituted with R⁵), or a 5- to 12-membered heteroaryl (the 5- to 12-membered heteroaryl is optionally mono-substituted or poly-substituted with R⁵), and X¹, X², X³, X⁴, and R¹ are as defined above.)

### (Process 6-1)

That is, the compound of formula [IV-10] can be produced by reacting a compound of formula [IV-3] and a compound [V-5] in the presence or absence of an appropriate solvent and in the presence or absence of an appropriate acid catalyst.

The amount of the compound [V-5] used in the present reaction may be appropriately selected from a range of usually 1.0 to 20 mol, and is preferably 1.0 to 5.0 mol, based on 1 mol of the compound [IV-3].

Examples of the acid catalyst that can be used in the present reaction include sulfonic acids such as methanesulfonic acid, trifluoromethanesulfonic acid, benzenesulfonic acid, and 4-toluenesulfonic acid; and Lewis acids such as titanium tetrachloride. The amount of the acid catalyst used may be appropriately selected from a range of 0.01 to 5.0 mol, and is preferably 0.01 to 2.0 mol, based on 1 mol of the compound [IV-3].

Examples of the solvent that can be used in the present reaction include ethers such as diethyl ether, diisopropyl ether, methyl tert-butyl ether, cyclopentyl methyl ether, tetrahydrofuran, 2-methyl tetrahydrofuran, 4-methyltetrahydropyran, 1,4-dioxane, and monoglyme; aromatic hydrocarbons such as benzene, toluene, xylene, mesitylene, and chlorobenzene; nitriles such as acetonitrile and propionitrile; aprotic polar solvents such as N,N-dimethylformamide, N,N-dimethylacetamide, N-methyl-2-pyrrolidone, dimethyl sulfoxide, sulfolane, and 1,3-dimethyl-2-imidazolidinone; alcohols such as methanol, ethanol, 2-propanol, tert-butyl alcohol, and methyl cellosolve; aliphatic hydrocarbons such as pentane, hexane, cyclohexane, and heptane; pyridines such as pyridine and picoline; and a mixed solvent thereof. The amount of the solvent used is 0.1 to 100 liters, and preferably 0.1 to 20 liters, based on 1 mol of the compound [IV-3].

The reaction temperature of the present reaction may be selected from a range of any temperature usually from 0° C. to a reflux temperature in a reaction system, and may be preferably in a range of a reflux temperature in a reaction system from room temperature. The reaction time of the present reaction is usually 10 minutes to 72 hours although it varies depending on the reaction temperature, the reactant, the reaction amount, and the like.

After the completion of the reaction, the compound [IV-10] can be isolated by subjecting the reaction mixture to operations such as pouring into water, extraction with an organic solvent, and concentration. The isolated compound [IV-10] can be further purified by column chromatography, recrystallization, or the like, as necessary.

### (Process 6-2)

That is, the compound of formula [IV-9] can be produced by reacting a compound of formula [IV-10] and an appropriate reducing agent in the presence or absence of an appropriate solvent and in the presence or absence of an appropriate acid catalyst.

Examples of the reducing agent that can be used in the present reaction include alkali metal salts of borohydride compounds, such as sodium borohydride, sodium triacetoxyborohydride, lithium borohydride, lithium triethylborohydride, and sodium borohydride; borane complexes of organic Lewis bases, such as a borane-tert-butylamine complex, a borane-dimethylsulfide complex, and a borane-2-picoline complex; alkali metal salts of aluminum hydride compounds, such as lithium aluminum hydride and bis(2-methoxyethoxy)aluminum sodium hydride; and organic aluminum hydride compounds such as diisobutylaluminum hydride. The amount of the reducing agent used may be appropriately selected from a range of usually 0.25 to 5.0 mol, and is preferably 0.25 to 2.0 mol, based on 1 mol of the compound [IV-10].

Examples of the acid catalyst that can be used in the present reaction include sulfonic acids such as methanesulfonic acid, trifluoromethanesulfonic acid, benzenesulfonic acid, and 4-toluenesulfonic acid; and carboxylic acids such as formic acid, acetic acid, propionic acid, dichloroacetic acid, and trifluoroacetic acid. The amount of the acid catalyst used may be appropriately selected from a range of 0.01 to 10 mol, and is preferably 0.01 to 3.0 mol, based on 1 mol of the compound [IV-10].

Examples of the solvent that can be used in the present reaction include ethers such as diethyl ether, diisopropyl ether, methyl tert-butyl ether, cyclopentyl methyl ether, tetrahydrofuran, 2-methyl tetrahydrofuran, 4-methyltetrahydropyran, 1,4-dioxane, and monoglyme; aromatic hydrocarbons such as benzene, toluene, xylene, mesitylene, and chlorobenzene; halogenated hydrocarbons such as dichloromethane, chloroform, and 1,2-dichloroethane; alcohols such as methanol, ethanol, 2-propanol, tert-butyl alcohol, and methyl cellosolve; aliphatic hydrocarbons such as pentane, hexane, cyclohexane, and heptane; water; and a mixed solvent thereof. The amount of the solvent used is 0.1 to 100 liters, and preferably 0.1 to 20 liters, based on 1 mol of the compound [IV-10].

The reaction temperature of the present reaction may be selected from a range of any temperature usually from −78° C. to a reflux temperature in a reaction system, and may be preferably in a range of a reflux temperature in a reaction system from 0° C. The reaction time of the present reaction is usually 10 minutes to 72 hours although it varies depending on the reaction temperature, the reactant, the reaction amount, and the like.

After the completion of the reaction, the compound [IV-9] can be isolated by subjecting the reaction mixture to operations such as pouring into water, extraction with an organic solvent, and concentration. The isolated compound [IV-9] can be further purified by column chromatography, recrystallization, or the like, as necessary.

### <Production Method 7>

A compound of formula [IV-3] can be produced, for example, according to a method based on the reaction scheme exemplified below.

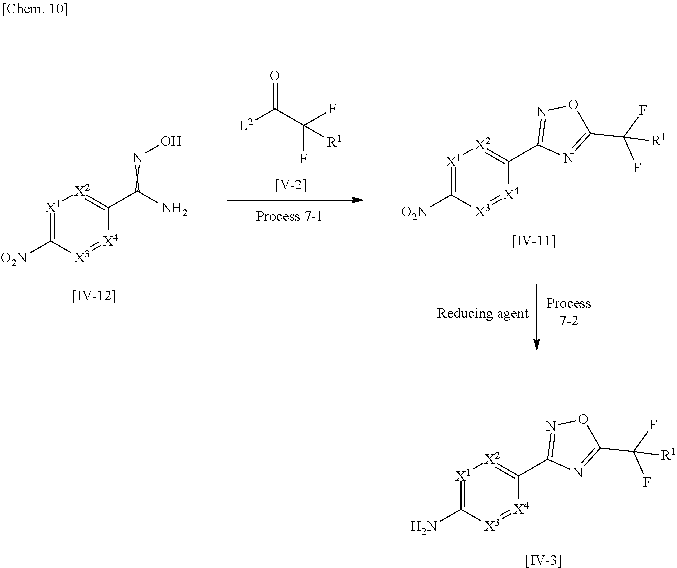

(in the formula, L², X¹, X², X³, X⁴, and R¹ are as defined above.)

### (Process 7-1)

That is, the compound of formula [IV-11] can be produced by reacting a compound [IV-12] and a compound [V-2] in the presence or absence of an appropriate solvent and in the presence or absence of an appropriate base. The compound [IV-12] can be produced in accordance with the method described in European Journal of Organic Chemistry, vol. 34, page 7,472 page, 2015, or the method thereof.

The amount of the compound [V-2] used in the present reaction may be appropriately selected from a range of usually 1.0 to 20 mol, and is preferably 1.0 to 5.0 mol, based on 1 mol of the compound [IV-12].

The base, the solvent, the reaction temperature, and the reaction time that can be used in the present reaction are the same as those in Process 1-3 of Production Method 1.

After the completion of the reaction, the compound [IV-11] can be isolated by subjecting the reaction mixture to operations such as pouring into water, extraction with an organic solvent, and concentration. The isolated compound [IV-11] can be further purified by column chromatography, recrystallization, or the like, as necessary.

### (Process 7-2)

That is, the compound of formula [IV-3] can be produced by reacting a compound of formula [IV-11] and an appropriate reducing agent in the presence or absence of an appropriate acid catalyst and in the presence or absence of an appropriate phase transfer catalyst.

Examples of the reducing agent that can be used in the present reaction include transition metals such as iron and zinc; typical metals such as tin; alkali metal salts of borohydride compounds, such as sodium borohydride, lithium borohydride, and lithium triethylborohydride; alkali metal salts of dithionous acid such as sodium dithionite; borane complexes of organic Lewis bases, such as a borane-tert-butylamine complex and a borane-dimethylsulfide complex; alkali metal salts of aluminum hydride compounds, such as lithium aluminum hydride and bis(2-methoxyethoxy)aluminum sodium hydride; and organic aluminum hydride compounds such as diisobutylaluminum hydride. The amount of the reducing agent used may be appropriately selected from a range of usually 0.25 to 50 mol, and is preferably 0.25 to 50 mol, based on 1 mol of the compound [IV-11].

Examples of the acid catalyst that can be used in the present reaction include sulfonic acids such as methanesulfonic acid, trifluoromethanesulfonic acid, benzenesulfonic acid, and 4-toluenesulfonic acid; and carboxylic acids such as formic acid, acetic acid, propionic acid, dichloroacetic acid, and trifluoroacetic acid. The amount of the acid catalyst used may be appropriately selected from a range of 0.01 to 10 mol, and is preferably 0.01 to 2.0 mol, based on 1 mol of the compound [IV-11].

Examples the phase transfer catalyst that can be used in the present reaction include tetrabutylammonium salts such as tetrabutylammonium bromide and tetrabutylammonium chloride. The amount of the phase transfer catalyst used may be appropriately selected from a range of 0.01 to 10 mol, and is preferably 0.01 to 2.0 mol, based on 1 mol of the compound [IV-11].

Examples of the solvent that can be used in the present reaction include ethers such as diethyl ether, diisopropyl ether, methyl tert-butyl ether, cyclopentyl methyl ether, tetrahydrofuran, 2-methyl tetrahydrofuran, 4-methyltetrahydropyran, 1,4-dioxane, and monoglyme; aromatic hydrocarbons such as benzene, toluene, xylene, mesitylene, and chlorobenzene; halogenated hydrocarbons such as dichloromethane, chloroform, and 1,2-dichloroethane; alcohols such as methanol, ethanol, 2-propanol, tert-butyl alcohol, and methyl cellosolve; aliphatic hydrocarbons such as pentane, hexane, cyclohexane, and heptane; water; and a mixed solvent thereof. The amount of the solvent used is 0.1 to 100 liters, and preferably 0.1 to 20 liters, based on 1 mol of the compound [IV-11].

The reaction temperature of the present reaction may be selected from a range of any temperature usually from −78° C. to a reflux temperature in a reaction system, and may be preferably in a range of a reflux temperature in a reaction system from 0° C. The reaction time of the present reaction is usually 10 minutes to 72 hours although it varies depending on the reaction temperature, the reactant, the reaction amount, and the like.

After the completion of the reaction, the compound [IV-3] can be isolated by subjecting the reaction mixture to operations such as pouring into water, extraction with an organic solvent, and concentration. The isolated compound [IV-3] can be further purified by column chromatography, recrystallization, or the like, as necessary.

### <Production Method 8>

A compound of formula [IV-12] can be produced, for example, according to the following methods.

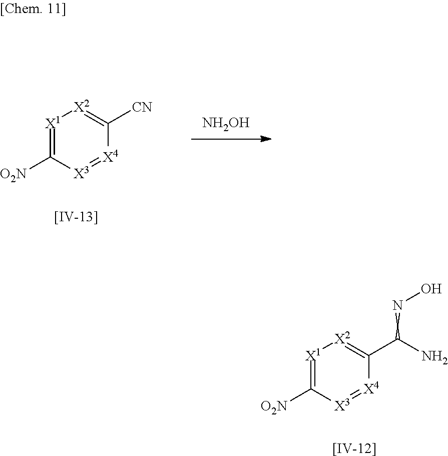

(in the formula, X¹, X², X³, and X⁴ are as defined above.)

That is, the compound of formula [IV-12] can be produced by reacting the compound [IV-13] and appropriate hydroxylamine in the presence of an appropriate solvent and in the presence or absence of an appropriate base.

The hydroxylamine, the base, the solvent, the reaction temperature, and the reaction time that can be used in the present reaction are the same as those in Production Method 3.

After the completion of the reaction, the compound [IV-12] can be isolated by subjecting the reaction mixture to operations such as pouring into water, extraction with an organic solvent, and concentration. The isolated compound [IV-12] can be further purified by column chromatography, recrystallization, or the like, as necessary.

### <Production Method 9>

A compound of formula [I-3] among the compounds of the present invention can be produced, for example, according to the following methods.

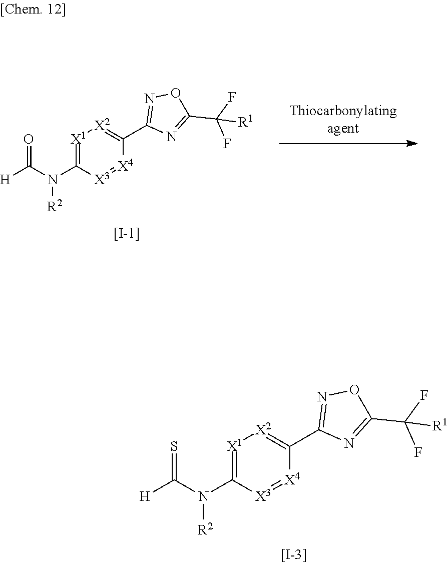

(in the formula, X¹, X², X³, X⁴, R¹, and R² are as defined above.)

That is, the compound of formula [I-3] can be produced by reacting a compound of formula [I-1] and an appropriate thiocarbonylating agent in the presence or absence of an appropriate base and in the presence or absence of an appropriate solvent.

Examples of the thiocarbonylating agent used in the present reaction include phosphorus pentasulfide and Lawesson's Reagent (2,4-bis(4-methoxyphenyl)-1,3,2,4-dithiadiphosphetan-2,4-disulfide). The amount of the thiocarbonylating agent used may be appropriately selected from a range of 0.25 to 100 mol, and is preferably 0.5 to 3.0 mol, based on 1 mol of the compound [I-1].

Examples of the base that can be used in the present reaction include inorganic bases such as carbonates of alkali metals such as sodium carbonate and potassium carbonate; and organic bases such as triethylamine, N,N-dimethylaniline, pyridine, 4-N,N-dimethylaminopyridine, and 1,8-diazabicyclo[5.4.0]-7-undecene. The amount of the base used may be appropriately selected from a range of 0.01 to 10 mol, and is preferably 0.1 to 2.0 mol, based on 1 mol of the compound [I-1].

Examples of the solvent that can be used in the present reaction include ethers such as diethyl ether, diisopropyl ether, methyl tert-butyl ether, cyclopentyl methyl ether, tetrahydrofuran, 2-methyl tetrahydrofuran, 4-methyltetrahydropyran, 1,4-dioxane, and monoglyme; aromatic hydrocarbons such as benzene, toluene, xylene, mesitylene, and chlorobenzene; aprotic polar solvents such as dimethyl sulfoxide and sulfolane; halogenated hydrocarbons such as dichloromethane, chloroform, and 1,2-dichloroethane; aliphatic hydrocarbons such as pentane, hexane, cyclohexane, and heptane; and a mixed solvent thereof. The amount of the solvent used is 0.1 to 100 liters, and preferably 0.1 to 50 liters, based on 1 mol of the compound [I-1].

The reaction temperature of the present reaction may be selected from a range of any temperature from 0° C. to a reflux temperature in a reaction system, and may be preferably in a range of 0° C. to 150° C.

The reaction time of the present reaction is usually 10 minutes to 72 hours although it varies depending on the reaction temperature, the reactant, the reaction amount, and the like.

After the completion of the reaction, the compound [I-3] can be isolated by concentrating the reaction mixture, or subjecting the reaction mixture to operations such as concentration, pouring into water, extraction with an organic solvent, and concentration. The isolated compound [I-3] can be further purified by column chromatography, recrystallization, or the like, as necessary.

### <Production Method 10>

A compound of formula [IV-1] can be produced, for example, according to a method based on the reaction scheme exemplified below.

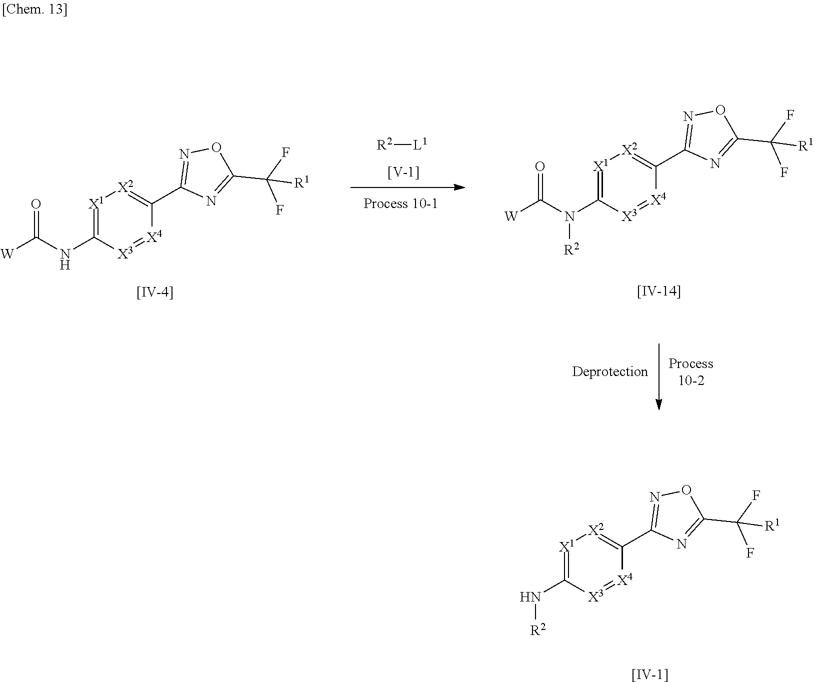

(in the formula, L¹, W, X¹, X², X³, X⁴, R¹, and R² are as defined above.)

### (Process 10-1)

That is, the compound of formula [IV-14] can be produced by reacting a compound [IV-4] and a compound [V-1] in the presence or absence of an appropriate solvent, in the presence or absence of an appropriate base, and in the presence or absence of an appropriate catalyst.

The amount of the compound [V-1] used in the present reaction may be appropriately selected from a range of usually 1.0 to 20 mol, and is preferably 1.0 to 3.0 mol, based on 1 mol of the compound [IV-4].

The base, the catalyst, the solvent, the reaction temperature, and the reaction time that can be used in the present reaction are the same as those in Process 1-1 of Production Method 1.

After the completion of the reaction, the compound [IV-14] can be isolated by subjecting the reaction mixture to operations such as pouring into water, extraction with an organic solvent, and concentration. The isolated compound [IV-14] can be further purified by column chromatography, recrystallization, or the like, as necessary.

### (Process 10-2)

That is, the compound of formula [IV-1] can be produced from the compound [IV-14] in accordance with the method described in GREEN'S PROTECTIVE GROUPS in Organic Synthesis; 5^{th} Edition (John Wiley and Sons, Peter G. M. Wuts, 2014).

The compound [IV-1] can be further purified by column chromatography, recrystallization, or the like, as necessary.

### <Production Method 1 of Intermediate>

A compound of formula [II-1] among the compounds of the present invention can be produced, for example, according to the following methods.

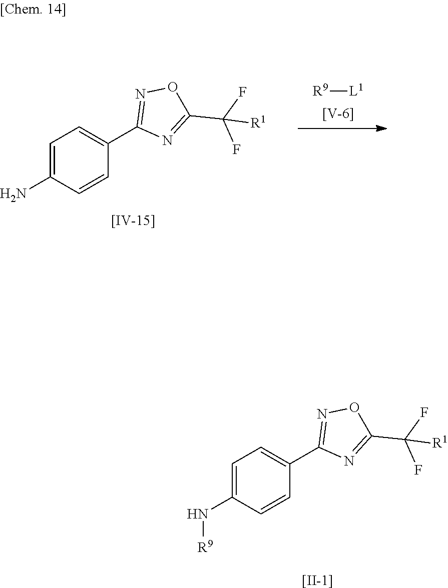

(in the formula, L¹, R¹, and R⁹ are as defined above.)

That is, the compound of formula [II-1] can be produced by reacting a compound [IV-15] and a compound [V-6] in the presence or absence of an appropriate solvent, in the presence or absence of an appropriate base, and in the presence or absence of an appropriate catalyst.

The amount of the compound [V-6] used in the present reaction may be appropriately selected from a range of usually 1.0 to 20 mol, and is preferably 1.0 to 3.0 mol, based on 1 mol of the compound [IV-15].

The base, the catalyst, and the solvent that can be used in the present reaction are the same as those in Process 1-1 of Production Method 1.

The reaction temperature of the present reaction may be selected from a range of any temperature usually from −78° C. to a reflux temperature in a reaction system, and may be preferably in a range of 0° C. to 150° C.

The reaction time of the present reaction is usually 10 minutes to 72 hours although it varies depending on the reaction temperature, the reactant, the reaction amount, and the like.

After the completion of the reaction, the compound [II-1] can be isolated by subjecting the reaction mixture to operations such as pouring into water, extraction with an organic solvent, and concentration. The isolated compound [II-1] can be further purified by column chromatography, recrystallization, or the like, as necessary.

### <Production Method 2 of Intermediate>

A compound of formula [II-2] among the compounds of the present invention can be produced, for example, according to a method based on the reaction scheme exemplified below.

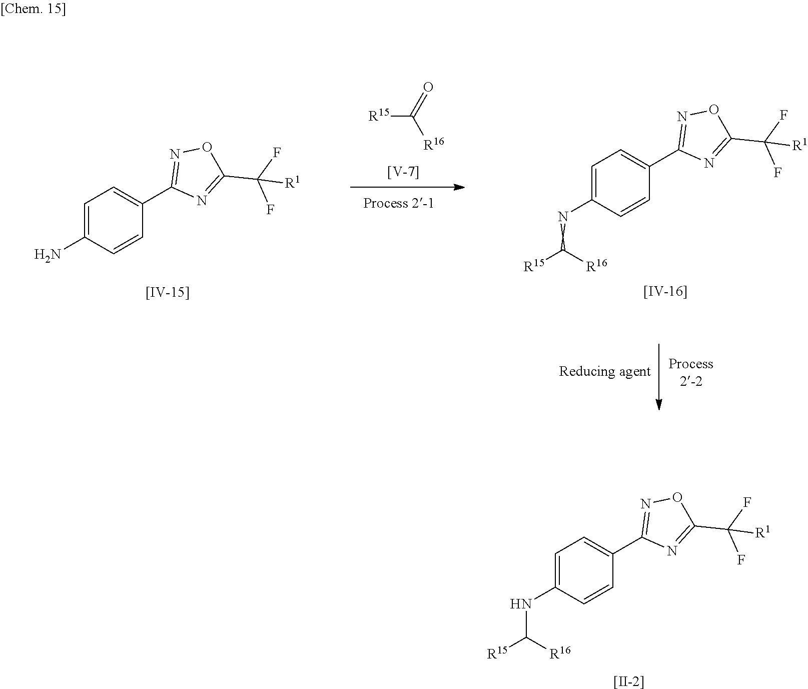

(in the formula, R¹⁵ represents a hydrogen atom, R¹⁶ represents a (C1-C5)alkyl mono-substituted or poly-substituted with substituent(s) selected from a substituent group R¹⁰, a (C2-C5)alkenyl mono-substituted or poly-substituted with substituent(s) selected from a substituent group R¹¹, a (C2-C5)alkynyl mono-substituted or poly-substituted with substituent(s) selected from a substituent group R¹², a (C3-C6)cycloalkyl, a (C6-C12)aryl (the (C6-C12)aryl is optionally mono-substituted or poly-substituted with R⁵), a 3- to 6-membered heterocyclyl (the 3- to 6-membered heterocyclyl is optionally mono-substituted or poly-substituted with R⁵), or a 5- to 12-membered heteroaryl (the 5- to 12-membered heteroaryl is optionally mono-substituted or poly-substituted with R⁵), and R¹ is as defined above.)

### (Process 2′-1)

That is, the compound of formula [IV-16] can be produced by reacting a compound of formula [IV-15] and a compound [V-7] in the presence or absence of an appropriate solvent and in the presence or absence of an appropriate acid catalyst.

The amount of the compound [V-7] used in the present reaction may be appropriately selected from a range of usually 1.0 to 20 mol, and is preferably 1.0 to 5.0 mol, based on 1 mol of the compound [IV-15].

The acid catalyst, the solvent, the reaction temperature, and the reaction time that can be used in the present reaction are the same as those in Process 6-1 of Production Method 6.

After the completion of the reaction, the compound [IV-16] can be isolated by subjecting the reaction mixture to operations such as pouring into water, extraction with an organic solvent, and concentration. The isolated compound [IV-16] can be further purified by column chromatography, recrystallization, or the like, as necessary.

### (Process 2′-2)

That is, the compound of formula [II-2] can be produced by reacting a compound of formula [IV-16] and an appropriate reducing agent in the presence or absence of an appropriate solvent and in the presence or absence of an appropriate acid catalyst.

The reducing agent, the acid catalyst, the solvent, the reaction temperature, and the reaction time that can be used in the present reaction are the same as those in Process 6-2 of Production Method 6.

After the completion of the reaction, the compound [II-2] can be isolated by subjecting the reaction mixture to operations such as pouring into water, extraction with an organic solvent, and concentration. The isolated compound [II-2] can be further purified by column chromatography, recrystallization, or the like, as necessary.

### <Production Method 3 of Intermediate>

A compound of formula [II-1] among the compounds of the present invention can be produced, for example, according to a method based on the reaction scheme exemplified below.

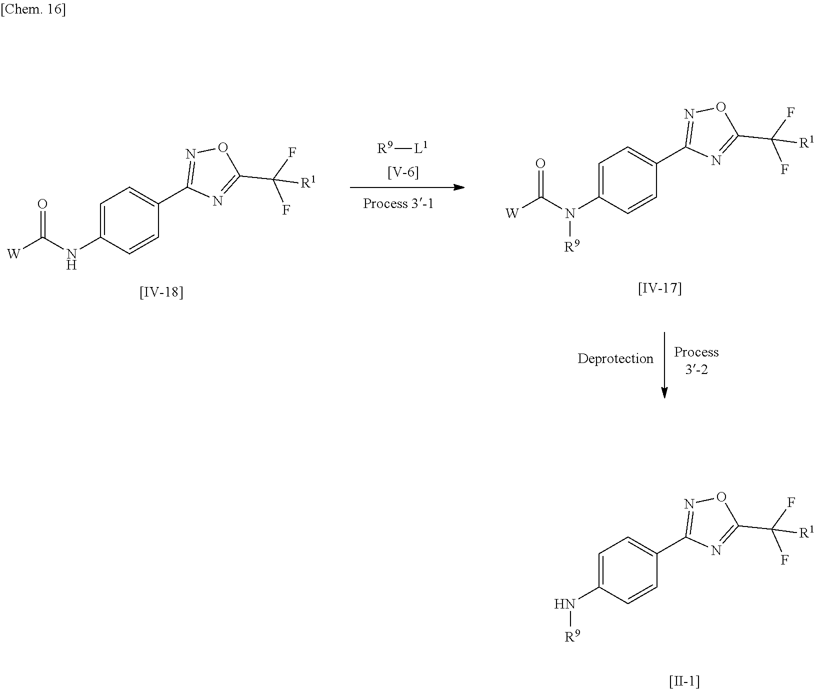

(in the formula, L¹, W, R¹, and R⁹ are as defined above.)

### (Process 3′-1)

That is, the compound of formula [IV-17] can be produced by reacting a compound [IV-18] and a compound [V-6] in the presence or absence of an appropriate solvent, in the presence or absence of an appropriate base, and in the presence or absence of an appropriate catalyst.

The amount of the compound [V-6] used in the present reaction may be appropriately selected from a range of usually 1.0 to 20 mol, and is preferably 1.0 to 3.0 mol, based on 1 mol of the compound [IV-18].

The base, the catalyst, the solvent, the reaction temperature, and the reaction time that can be used in the present reaction are the same as those in Process 1-1 of Production Method 1.

After the completion of the reaction, the compound [IV-17] can be isolated by subjecting the reaction mixture to operations such as pouring into water, extraction with an organic solvent, and concentration. The isolated compound [IV-17] can be further purified by column chromatography, recrystallization, or the like, as necessary.

### (Process 3′-2)

That is, the compound of formula [II-1] can be produced from the compound [IV-17] in accordance with the method described in GREEN'S PROTECTIVE GROUPS in Organic Synthesis; 5^{th} Edition (John Wiley and Sons, Peter G. M. Wuts, 2014).

The compound [II-1] can be further purified by column chromatography, recrystallization, or the like, as necessary.

### <Production Method 4 of Intermediate>

A compound of formula [III-1] among the compounds of the present invention can be produced, for example, according to the following methods.

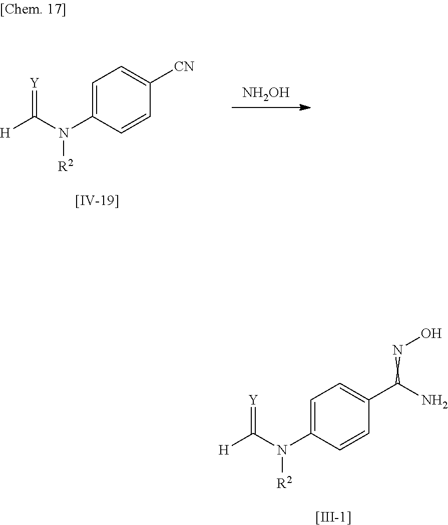

(in the formula, Y and R² are as defined above.)

That is, the compound of formula [III-1] can be produced by reacting [IV-19] and appropriate hydroxylamine in the presence of an appropriate solvent and in the presence or absence of an appropriate base.

The hydroxylamine, the base, the solvent, the reaction temperature, and the reaction time that can be used in the present reaction are the same as those in Production Method 3.

After the completion of the reaction, the compound [III-1] can be isolated by subjecting the reaction mixture to operations such as pouring into water, extraction with an organic solvent, and concentration. The isolated compound [III-1] can be further purified by column chromatography, recrystallization, or the like, as necessary.

The agricultural and horticultural plant disease control agent of the present invention contains the formamide derivative of formula [I] of the present invention or the agriculturally acceptable salt thereof as an active ingredient.

The agricultural and horticultural plant disease control agent of the present invention can contain an additive component (carrier) that is usually used in an agrochemical formulation, as necessary.

Examples of the additive component include a carrier such as a solid carrier or a liquid carrier, a surfactant, a binder or a tackifier, a thickener, a coloring agent, a spreader, a sticker, an antifreezing agent, an anticaking agent, a disintegrating agent, and a decomposition inhibitor, and in addition, an antiseptic, a plant piece, or the like may be added to the additive component, as necessary. These additive components may be used alone or in combination of two or more thereof.

Hereinafter, the additive components described above are described.

Examples of the solid carrier include mineral carriers such as pyrophyllite clay, kaolin clay, silica clay, talc, diatomite, zeolite, bentonite, Japanese acid clay, activated clay, attapulgus clay, vermiculite, pearlite, pumice, white carbon (synthetic silicic acid, synthetic silicate, or the like), and titanium dioxide; vegetal carriers such as woody powder, corn haulm, walnut shell, fruit stone, chaff, sawdust, bran, soybean meal, powdered cellulose, starch, dextrin, and saccgarudes; inorganic salt carriers such as calcium carbonate, ammonium sulfate, sodium sulfate, and potassium chloride; and polymeric carriers such as polyethylene, polypropylene, polyvinyl chloride, polyvinyl acetate, an ethylene-vinyl acetate copolymer, and a urea-aldehyde resin.

Examples of the liquid carrier include monohydric alcohols such as methanol, ethanol, propanol, 2-propanol, butanol, and cyclohexanol; polyhydric alcohols such as ethylene glycol, diethylene glycol, propylene glycol, hexylene glycol, polyethylene glycol, polypropylene glycol, and glycerin; polyhydric alcohol derivatives such as polypropylene glycol ether; ketones such as acetone, methyl ethyl ketone, methyl isobutyl ketone, diisobutyl ketone, cyclohexanone, and isophoron; ethers such as ethyl ether, 1,4-dioxane, cellosolve, dipropyl ether, and tetrahydrofuran; aliphatic hydrocarbons such as normal paraffin, naphthene, isoparaffin, kerosene, and mineral oil; aromatic hydrocarbons such as toluene, C₉-C₁₀ alkylbenzene, xylene, solvent naphtha, alkyl naphthalene, and high boiling point aromatic hydrocarbons; halogenated hydrocarbons such as 1,2-dichloroethane, chloroform, and carbon tetrachloride; esters such as ethyl acetate, diisopropyl phthalate, dibutyl phthalate, dioctyl phthalate, and dimethyl adipate; lactones such as γ-butyrolactone; amides such as N,N-dimethylformamide, N,N-diethylformamide, N,N-dimethylacetamide, and N-methyl-2-pyrrolidone; nitriles such as acetonitrile; sulfur compounds such as dimethyl sulfoxide; vegetable oils such as soybean oil, rapeseed oil, cottonseed oil, coconut oil, and castor oil; lower alkyl ester of fatty acid derived from the vegetable oils; and water.

The surfactant is not particularly limited, and is preferably a gel in water or exhibits swellability. Examples of the surfactant include noionic surfactants such as sorbitan fatty acid ester, polyoxyethylene sorbitan fatty acid ester, sucrose fatty acid ester, polyoxyethylene fatty acid ester, polyoxyethylene resin acid ester, polyoxyethylene fatty acid diester, polyoxyethylene alkyl ether, polyoxyethylene alkylphenyl ether, polyoxyethylene dialkylphenyl ether, polyoxyethylene alkylphenyl ether formalin condensate, polyoxyethylene polyoxypropylene block polymer, alkylpolyoxyethylene polypropylene block polymer ether, polyoxyethylene alkyl amine, polyoxyethylene fatty acid amide, polyoxyethylene fatty acid bisphenyl ether, polyalkylene benzyl phenyl ether, polyoxyalkylene styryl phenyl ether, acetylene diol, polyoxyalkylene-added acetylene diol, polyoxyethylene ether silicone, ester silicone, fluorine surfactant, polyoxyethylene castor oil, and polyoxyethylene hardened castor oil; anionic surfactants such as alkyl sulfates, polyoxyethylene alkyl ether sulfates, polyoxyethylene alkyl phenyl ether sulfates, polyoxyethylene styryl phenyl ether sulfate, alkyl benzenesulfonates, lignin sulfonate, alkyl sulfosuccinates, naphthalene sulfonate, alkylnaphthalene sulfonates, salts of formalin condensate of naphthalene sulfonate, salts of formalin condensate of alkylnaphthalene sulfonate, fatty acid salts, polycarboxylates, N-methyl-fatty acid sarcosinates, resinates, polyoxyethylene alkyl ether phosphates, and polyoxyethylene alkyl phenyl ether phosphates; cationic surfactants such as alkyl amine salts such as lauryl amine hydrochloride, stearyl amine hydrochloride, oleyl amine hydrochloride, stearyl amine acetate, stearyl aminopropylamine acetate, alkyl trimethyl ammonium chlorides, and alkyl dimethylbenzarconium chloride; and amphoteric surfactants including a betaine type such as dialkyl diaminoethyl betaine and alkyl dimethylbenzyl betaine, and an amino acid type such as dialkylaminoethyl glycine and alkyl dimethyl benzyl glycine.

Examples of the binder or the tackifier include carboxymethyl cellulose or a salt thereof, dextrin, soluble starch, xanthan gum, guar gum, sucrose, polyvinylpyrrolidone, gum arabic, polyvinyl alcohol, polyvinyl acetate, sodium polyacrylate, polyethylene glycol having an average molecular weight of 6,000 to 20,000, polyethylene oxide having an average molecular weight of 100,000 to 5,000,000, and naturally-occurring phospholipid (for example, cephalin acid, lecithin, or the like).

Examples of the thickener include water-soluble polymers such as xanthan gum, guar gum, carboxymethylcellulose, polyvinylpyrrolidone, carboxyvinyl polymers, acrylic polymers, starch derivatives, and polysaccharides; and inorganic fine powders such as high purity bentonite and white carbon.

Examples of the coloring agent include inorganic pigments such as iron oxide, titanium oxide, and Prussian blue; and organic dyes such as an alizarin dye, an azo dye, and a metal phthalocyanine dye.

Examples of the spreader include a silicone-based surfactant, cellulose powder, dextrin, processed starch, a polyaminocarboxylic acid chelate compound, crosslinked polyvinylpyrrolidone, a maleic acid/styrene copolymer, a methacrylic acid copolymer, a half ester of a polyhydric alcohol polymer and a dicarboxylic acid anhydride, a water-soluble salt of polystyrenesulfonic acid, polyoxyethylene alkanediols, polyoxyethylene alkynediols, and alkynediols.

Examples of the sticker include various surfactants such as sodium dialkyl sulfosuccinate, polyoxyethylene alkyl ether, polyoxyethylene alkylphenyl ether, and polyoxyethylene fatty acid ester; paraffin, terpene, a polyamide resin, polyacrylate, polyoxyethylene, wax, polyvinylalkyl ether, an alkylphenol formalin condensate, and a synthetic resin emulsion.

Examples of the antifreezing agent include polyhydric alcohols such as ethylene glycol, diethylene glycol, propylene glycol, and glycerin.

Examples of the anticaking agent include polysaccharides such as starch, alginic acid, mannose, and galactose; polyvinylpyrrolidone, white carbon, ester gum, and a petroleum resin.

Examples of the disintegrating agent include sodium tripolyphosphate, sodium hexametaphosphate, a stearic metal salt, cellulose powder, dextrin, a copolymer of methacrylic ester, polyvinylpyrrolidone, a polyaminocarboxylic chelate compound, a styrene sulfonate-isobutylene-maleic anhydride copolymer, and a starch-polyacrylonitrile graft copolymer.

Examples of the decomposition inhibitor include a desiccant such as zeolite, calcined lime, or magnesium oxide; a phenolic, amine-based, sulfur-based, or phosphoric acid-based antioxidant; and a salicylic acid-based or benzophenone-based ultraviolet absorber.

Examples of the antiseptic include potassium sorbate and 1,2-benzothiazol-3(2H)-one.

Examples of the plant piece include sawdust, coconut shellflower, corncob, and tobacco stem.

Meanwhile, in the agricultural and horticultural plant disease control agent of the present invention, when the additive component is contained, a content thereof is selected usually in a range of 5 to 95%, and preferably in a range of 20 to 90% in a case of a carrier such as a solid carrier or a liquid carrier, is selected usually in a range of 0.1 to 30%, and preferably in a range of 0.5 to 10% in a case of a surfactant, and is selected usually in a range of 0.1 to 30%, and preferably in a range of 0.5 to 10% in a case of other additives, on a mass basis.

The agricultural and horticultural plant disease control agent of the present invention is used in any formulation such as dusts, dusts and granules, granules, wettable powders, water-soluble powders, wettable granules, tablets, Jumbo formulations, emulsifiable concentrates, oils, solutions, flowable concentrates, emulsions, microemulsions, suspoemulsions, ultra-low volume formulations, microcapsules, smoking formulations, aerosols, baiting formulations, and pastes.

In actual use of the formulation, the formulation can be used as it is or after dilution with a diluent such as water in a predetermined concentration. Application of various formulations containing the compound of the present invention or dilution products thereof can be conducted by a method ordinarily used, that is, dispersion (for example, spraying, misting, atomizing, powder dispersion, granule dispersion, on-water-surface dispersion, or inbox dispersion), in-soil application (for example, mixing or drenching), on-surface application (for example, coating, dust coating, or covering), seed treatment (for example, smearing or dressing treatment), immersion, poison bait, or smoking. It is also possible to mix the active ingredient with a livestock feed in order to control harmful insects in excreta of livestock, particularly outbreak and growth of harmful pests.

A method for controlling an agricultural and horticultural plant disease of the present invention can be performed by using the amount of the active ingredient of an alkoxyalkoxybenzene derivative having a sulfur-containing substituent of the present invention or an agriculturally acceptable salt thereof by the application method described above.

The mixing proportion (% by mass) of the active ingredient in the agricultural and horticultural plant disease control agent of the present invention is appropriately selected, as necessary. For example, the mixing proportion may be appropriately selected in the following ranges: 0.01 to 20% and preferably 0.05 to 10% for dusts, dusts and granules, microgranules, or the like; 0.1 to 30% and preferably 0.5 to 20% for granules or the like; 1 to 70% and preferably 5 to 50% for wettable powders, wettable granules, or the like; 1 to 95% and preferably 10 to 80% for water-soluble powders, solutions, or the like; 5 to 90% and preferably 10 to 80% for emulsifiable concentrates or the like; 1 to 50% and preferably 5 to 30% for oils or the like; 5 to 60% and preferably 10 to 50% for flowable concentrates or the like; 5 to 70% and preferably 10 to 60% for emulsions, microemulsions, suspoemulsions, or the like; 1 to 80% and preferably 5 to 50% for tablets, baiting formulations, pastes, or the like; 0.1 to 50% and preferably 1 to 30% for smoking formulations or the like; and 0.05 to 20% and preferably 0.1 to 10% for aerosols or the like.

The formulation is sprayed after dilution in an appropriate concentration, or is applied directly.

When the agricultural and horticultural plant disease control agent of the present invention is used after dilution with a diluent, the concentration of the active ingredient is generally 0.1 to 5,000 ppm. When the formulation is used as it is, the application amount thereof per unit area is 0.1 to 5,000 g per 1 ha in terms of the active ingredient compound; however, the application amount is not limited thereto.

It goes without saying that the agricultural and horticultural plant disease control agent of the present invention is sufficiently effective even when the compound of the present invention is used alone as an active ingredient, and the agricultural and horticultural plant disease control agent of the present invention may be mixed or used in combination, as necessary, with other fertilizers and pesticides such as a bactericide, an antiviral agent, an insecticide, a miticide, a nematicide, a synergist, an attractant, a herbicide, and a plant growth regulator. In this case, a more excellent effect can be exhibited.

Next, examples of known bactericides (bactericidal active ingredients) or disease control agent compounds that can be mixed or used in combination are shown below.

Bactericidal active ingredients or disease control agent compounds:

Agrobacterium radiobacter, azaconazole, acibenzolar-S-methyl, azoxystrobin, anilazine, amisulbrom, aminopyrifen, ametoctradin, aldimorph, isotianil, isopyrazam, isofetamid, isoflucypram, isoprothiolane, ipconazole, ipflufenoquin, ipfentrifluconazole, iprodione, iprovalicarb, iprobenfos, imazalil, iminoctadine-albesilate, iminoctadine-triacetate, imibenconazole, inpyrfluxam, imprimatin A, imprimatin B, edifenphos, etaconazole, ethaboxam, ethirimol, ethoxyquin, etridiazole, enestroburin, enoxastrobin, epoxiconazole, organic oils, oxadixyl, oxazinylazole, oxathiapiprolin, oxycarboxin, oxine-copper, oxytetracycline, oxpoconazole-fumarate, oxolinic acid, copper dioctanoate, octhilinone, ofurace, orysastrobin, o-phenylphenol, kasugamycin, captafol, carpropamid, carbendazim, carboxin, carvone, Candida oleophila, Candida saitoana, quinoxyfen, quinofumelin, chinomethionat, captan, quinconazole, quintozene, guazatine, cufraneb, coumethoxystrobin, coumoxystrobin, Gliocradium catenulatum, Cryptococcus albidus, kresoxim-methyl, clozylacon, Clonostachys rosea, chlozolinate, chloroinconazide, chlorothalonil, chloroneb, Chaetomium cupreum, Coniothyrium minitans, cyazofamid, diethofencarb, diclocymet, dichlofluanid, dichlobentiazox, diclomezine, dicloran, dichlorophen, dithianon, diniconazole, diniconazole-M, zineb, dinocap, dipymetitrone, diphenylamine, difenoconazole, cyflufenamid, diflumetorim, cyproconazole, cyprodinil, simeconazole, dimethirimol, dimethyl disulfide, dimethomorph, cymoxanil, dimoxystrobin, Pseudozyma flocculosa, Pseudomonas aureofaciens, Pseudomonas chlororaphis, Pseudomonas syringae, Pseudomonas flurorescens, Pseudomonas rhodesiae, ziram, silthiofam, Zucchini yellow mosaic virus, streptomycin, Streptomyces griseoviridis, Streptomyces lygicus, spiroxamine, sedaxane, seboctylamine, zoxamide, solatenol, dazomet, Talaromyces flavus, tiadinil, thiabendazole, thiram, thiophanate, thiophanate-methyl, thifluzamide, thiram, tecnazene, tecloftalam, tetraconazole, debacarb, tebuconazole, tebufloquin, terbinafine, dodine, dodemorph, triadimenol, triadimefon, triazoxide, trichlamide, triclopyricarb, Trichoderma asperellum, Trichoderma atroviride, Trichoderma gamsii, Trichoderma stromaticum, Trichoderma paecilomyces, Trichoderma harzianum, Trichoderma viride, Trichoderma virens, Trichoderma polysporum, Trichoderma harzianum rifai, Trichoderma lignorum, tricyclazole, triticonazole, tridemorph, triflumizole, trifloxystrobin, triforine, tolylfluanid, tolclofos-methyl, tolnifanide, tolprocarb, nabam, natamycin, naftifine, nitrapyrin, nitrothal-isopropyl, nuarimol, copper nonyl phenol sulphonate, Paenibacillus polymyxa, Barkholderia cepacia, Bacillus amyloliquefaciens, Bacillus simplex, Bacillus subtilis, Bacillus pumilus, Bacillus mycoides, Bacillus licheniformis, harpin protein, Variovorax paradoxus, validamycin, valifenalate, Pantoea agglomerans, picarbutrazox, bixafen, picoxystrobin, Pythium oligandrum, pydiflumetofen, bitertanol, binapacryl, hinokitiol, Erwinia carotovora, Rhizobium vitis, biphenyl, piperalin, hymexazol, pyraoxystrobin, pyraclostrobin, pyraziflumid, pyrazophos, pyrapropoyne, pyrametostrobin, pyriofenone, pyrisoxazole, pyridachlometyl, pyrifenox, pyributicarb, pyribencarb, pyrimethanil, pyroquilon, vinclozolin, ferbam, famoxadone, phenazine oxide, fenamidone, fenaminstrobin, fenarimol, fenoxanil, ferimzone, fenpiclonil, fenpicoxamid, fenpyrazamine, fenbuconazole, fenfuram, fenpropidin, fenpropimorph, fenhexamid, folpet, phthalide, Fusarium oxysporum, bupirimate, fuberidazole, blasticidin-S, furametpyr, furalaxyl, furancarboxylic acid, fluazinam, fluindapyr, fluoxastrobin, fluoxapiprolin, fluoxytioconazole, fluopicolide, fluopimomide, fluopyram, fluoroimide, fluxapyroxad, fluquinconazole, furconazole, furconazole -cis, fludioxonil, flusilazole, flusulfamide, flutianil, flutolanil, flutriafol, flufenoxadiazam, flufenoxystrobin, flubeneteram, flumetylsulforim, flumetover, flumorph, Phlebiopsis gigantea, proquinazid, prochloraz, procymidone, prothiocarb, prothioconazole, bronopol, propamocarb-hydrochloride, propiconazole, propineb, probenazole, bromuconazole, flometoquin, florylpicoxamid, hexaconazole, benalaxyl, benalaxyl-M, benodanil, benomyl, pefurazoate, penconazole, pencycuron, benzovindiflupyr, benthiazole, benthiavalicarb-isopropyl, penthiopyrad, penflufen, boscalid, fosetyl (including a salt of aluminum, calcium, sodium, or the like), polyoxin, polycarbamate, Bordeaux mixture, mancopper, mancozeb, mandipropamid, mandestrobin, maneb, myclobutanil, mineral oils, mildiomycin, methasulfocarb, metam, metalaxyl, metalaxyl-M, metarylpicoxamid, metiram, metyltetraprole, metconazole, metominostrobin, metrafenone, mepanipyrim, mefentrifluconazole, meptyldinocap, mepronil, iodocarb, laminarin, phosphorous acid and salts, copper oxychloride, silver, copper(II) sulfate, cuprous oxide, copper hydroxide, potassium bicarbonate, sodium bicarbonate, sulfur, oxyquinoline sulfate, copper sulfate, UK-2A (code number), dodecylbenzenesulfonic acid bisethylenediamine copper[II] salt (DBEDC), triphenyltin acetate (TPTA), triphenyltin chloride (TPTC), triphenyltin hydroxide (TPTH), 6-chloro-N-[2-(2,4-dimethylphenyl)-2,2-difluoro-ethyl]-3-[3-(trifluoromethyl)phenoxy]pyridazine-4-carboxamide (Chemical name, CAS registry number: 2435594-93-5) (WO 2020/109391 A), rac-3-[6-chloro-3-[3-(trifluoromethyl)phenoxy]pyridazin-4-yl]-5-[(2,4-dimethylphenyl)methyl]-5,6-dihydro-4H-1,2,4-oxadiazine) (Chemical name, CAS registry number: 2446124-68-9) (WO 2020/127780 A), 6-chloro-N-[2-(2,4-dimethylphenyl)-2,2-difluoroethyl]-5-methyl-3-[3-(trifluoromethyl)phenoxy]pyridazine-4-carboxamide) (Chemical name, CAS registry number: 2435603-06-6) (WO 2020/109391 A), 6-chloro-N-[2-(2,4-dimethylphenyl)ethyl]-3-[3-(trifluoromethyl)phenoxy]pyridazine-4-carboxamide) (Chemical name, CAS registry number: 2435600-09-0) (WO 2020/109391 A), 6-chloro-N-[2-(2,4-dimethylphenyl)ethyl]-5-methyl-3-[3-(trifluoromethyl)phenoxy]pyridazine-4-carboxamide) (Chemical name, CAS registry number: 2435603-15-7) (WO 2020/109391 A), and rac-3-[6-chloro-5-methyl-3-[3-(trifluoromethyl)phenoxy]pyridazin-4-yl]-5-[(2,4-dimethylphenyl)methyl]-5,6-dihydro-4H-1,2,4-oxadiazine) (Chemical name, CAS registry number: 2446123-98-2) (WO 2020/127780 A).

Next, examples of known insecticides (insecticidal active ingredients), miticides (miticidal active ingredients), nematicides (nematicidal active ingredients), and synergistic compounds (synergistic active ingredients) that may be mixed or used in combination are shown below.

Insecticidal active ingredients, miticidal active ingredients, nematicidal active ingredient, and synergistic active ingredients:

acrinathrin, azadirachtin, azamethiphos, acynonapyr, azinphos-ethyl, azinphos-methyl, acequinocyl, acetamiprid, acetoprole, acephate, azocyclotin, abamectin, afidopyropen, afoxolaner, amidoflumet, amitraz, alanycarb, aldicarb, aldoxycarb, allethrin) [including d-cis-trans-body and d-trans-body], isazophos, isamidofos, isocarbophos, isoxathion, isocycloseram, isofenphos-methyl, isoprocarb, epsilon-metofluthrin, epsilon-momfluorothrin, ivermectin, imicyafos, imidacloprid, imiprothrin, indazapyroxamet, indoxacarb, esfenvalerate, ethiofencarb, ethion, ethiprole, ethylene dibromide, etoxazole, etofenprox, ethoprophos, etrimfos, emamectin, emamectin benzoate, endosulfan, empenthrin, oxazosulfyl, oxamyl, oxydemeton-methyl, oxydeprofos, omethoate, Nuclear polyhedrosis virus, cadusafos, kappa-tefluthrin, kappa-bifenthrin, karanjin, cartap, Granulosis virus, carbaryl, carbosulfan, carbofuran, gamma-BHC, xylylcarb, quinalphos, kinoprene, chinomethionat, Entero virus, coumaphos, cryolite, clothianidin, clofentezine, chromafenozide, chlorantraniliprole, chlorethoxyfos, chlordane, chloropicrin, chlorpyrifos, chlorpyrifos-methyl, chlorfenapyr, chlorfenvinphos, chlorfluazuron, chlormephos, chloroprallethrin, Entomopoxi virus, Irido virus, cyazypyr, cyanophos, diafenthiuron, diamidafos, cyantraniliprole, cyetpyrafen, dienochlor, cyenopyrafen, dioxabenzofos, diofenolan, Sigma virus, cyclaniliprole, cycloxaprid, dicrotophos, dichlofenthion, cyclobutrifluram, cycloprothrin, dichlorvos, dicloromezotiaz, dicofol, dicyclanil, disulfoton, dinotefuran, dinobuton, cyhalodiamide, cyhalothrin [including gamma-body and lambda-body], cyphenothrin [including (1R)-trans-body], cyfluthrin [including beta-body], diflubenzuron, cyflumetofen, diflovidazin, cyproflanilide, cyhexatin, cypermethrin [including alpha-body, beta-body, theta-body, and zeta-body], dimpropyridaz, dimethyl-2,2,2-trichloro-1-hydroxyethyl phosphate (DEP), dimethylvinphos, dimethoate, dimefluthrin, jasmone, cis-jasmone, jasmonic acid, methyl jasmonate, silafluofen, cyromazine, Steinernema carpocapsae, Steinernema kushidai, Steinernema glaseri, spidoxamat, spinetoram, spinosad, spirodiclofen, spirotetramat, spiropidion, spiromesifen, sulcofuron-sodium, sulfluramid, sulfoxaflor, sulfotep, diazinon, thiacloprid, thiamethoxam, tioxazafen, thiodicarb, thiocyclam, thiosultap, thionazin, thiofanox, thiometon, tyclopyrazoflor, tetrachlorantraniliprole, tetrachlorvinphos, tetradifon, tetraniliprole, tetramethylfluthrin, tetramethrin, tebupirimfos, tebufenozide, tebufenpyrad, tefluthrin, teflubenzuron, demeton-S-methyl, temephos, deltamethrin, terbufos, tralomethrin, transfluthrin, triazamate, triazophos, trichlorfon, Trichoderma asperellum, Trichoderma paecilomyces, Trichoderma harzianum, triflumuron, trifluenfuronate, triflumezopyrim, trimethacarb, tolfenpyrad, naled, nicotine, nicofluprole, nitenpyram, nemadectin, Denso virus, novaluron, noviflumuron, Paecilomyces lilacinus, Barkholderia cepacia, Barkholderia rinojensis, Verticillium lecanii, hydroprene, Pasteuria nishizawae, Pasteuria penetrans, Bacillus amyloliquefaciens, Bacillus firmus, Bacillus sphaericus, Bacillus subtillis, Bacillus thuringiensis, insect toxin produced by Bacillus thuringiensis, Bacillus thuringiensis subsp. Aizawai, Bacillus thuringiensis subsp. Israelensis, Bacillus thuringiensis subsp. Kurstaki, Bacillus thuringiensis subsp. Tenebrionis, Bacillus popilliae, Bacillus licheniformis, vamidothion, parathion, parathion-methyl, halfenprox, halofenozide, bioallethrin, bioallethrin S-cyclopentenyl, bioresmethrin, bis-(2-chloro-1-methylethyl)ether (DCIP), bistrifluron, hydramethylnon, bifenazate, bifenthrin, pyflubumide, piperonyl butoxide, pymetrozine, pyraclofos, pyrafluprole, pyridaphenthion, pyridaben, pyridalyl, pyrifluquinazon, pyriprole, pyriproxyfen, pirimicarb, pyrimidifen, pyriminostrobin, pirimiphos-methyl, pyrethrine, famphur, fipronil, fenazaquin, fenamiphos, fenitrothion, fenoxycarb, fenothiocarb, phenothrin [including (1R)-trans-body], fenobucarb, fenthion, phenthoate, fenvalerate, fenpyroximate, fenbutatin oxide, fenpropathrin, fenmezoditiaz, fonofos, sulfuryl fluoride, butocarboxim, butoxycarboxim, buprofezin, furathiocarb, prallethrin, fluacrypyrim, fluazaindolizine, fluazuron, fluensulfone, fluopyram, sodium fluoroacetate, fluxametamide, fluchlordiniliprole, flucycloxuron, flucythrinate, flusulfamide, fluthrin, fluvalinate [including tau-body], flupyradifurone, flupyrazofos, flupyrimin, flufiprole, flufenerim, flufenoxystrobin, flufenoxuron, fluhexafon, flubendiamide, flupentiofenox, flumethrin, fluralaner, flurimfen, prothiofos, protrifenbute, flonicamid, propaphos, propargite, prohydrojasmon, profenofos, broflanilide, profluthrin, propetamphos, propoxur, flometoquin, bromopropylate, hexythiazox, hexaflumuron, Pacilimyces tenuipes, Paecilomyces fumosoroceus, Paecilomyces lilacinus, heptafluthrin, heptenophos, permethrin, benclothiaz, benzpyrimoxan, bensultap, benzoximate, bendiocarb, benfuracarb, Pochonia chlamydosporia, Beauveria tenella, Beauveria bassiana, Beauveria brongniartii, phoxim, phosalone, fosthiazate, fosthietan, phosphamidon, phosmet, polynactins, formetanate, phorate, machine oil, malathion, milbemectin, mecarbam, mesulfenfos, methomyl, metaldehyde, metaflumizone, methamidophos, metham, methiocarb, methidathion, methyl isothiocyanate, methyl bromide, methoxychlor, methoxyfenozide, methothrin, metofluthrin, methoprene, metolcarb, mevinphos, meperfluthrin, Monacrosporium phymatophagum, Monacrosporium phymatophagum, monocrotophos, momfluorothrin, Trichoderma harzianum, litlure-A, litlure-B, aluminium phosphide, zinc phosphide, phosphine, lufenuron, rescalure, resmethrin, lepimectin, rotenone, Cytoplasmic polyhedrosis virus, fenbutatin oxide, calcium cyanide, organotins, nicotine-sulfate, (Z)-11-tetradecenyl=acetate, (Z)-11-hexadecenal, (Z)-11-hexadecenyl=acetate, (Z)-9,12-tetradecadienyl=acetate, (Z)-9-tetradecen-1-ol, (Z,E)-9,11-tetradecadienyl=acetate, (Z,E)-9,12-tetradecadienyl=acetate, 1,1,1-trichloro-2,2-bis(4-chlorophenyl)ethane (DDT), 1,3-dichloropropene, 4,6-dinitro-O-cresol (DNOC), Bt proteins (Cry1Ab, Cry1Ac, CrylFa, Cry2Ab, mCry3A, Cry3Ab, Cry3Bb, and Cry34/35Abl), methyl eugenol, 4-(p-acetoxyphenyl)-2-butanone, (Z)-10-tetradecenyl=acetate, (E,Z)-4,10-tetradecadienyl=acetate, (Z)-8-dodecenyl=acetate, (Z)-11-tetradecenyl=acetate, (Z)-13-icosen-10-one, 14-methyl-1-octadecene, AKD-1193 (code number), BCS-AA10147 (code number), CL900167 (code number), 0,0-diethyl-O-[4-(dimethylsulfamoyl)phenyl]-phosphorothioate (DSP), O-ethyl-O-4-(nitrophenyl)phenylphosphonothioate (EPN), RU15525 (code number), XMC, Z-13-icosen-10-one, ZXI8901 (code number), and F4260 (code number).

Next, examples of known herbicide compounds, herbicidal active ingredients, and plant growth regulator compounds that may be mixed or used in combination are shown below, but the present invention is not limited thereto.

Herbicide compounds and herbicidal active ingredients:

ioxynil (including a salt with a lithium salt, a sodium salt, octanoic acid, or the like), aclonifen, acrolein, azafenidin, acifluorfen (including a salt with sodium or the like), azimsulfuron, asulam, acetochlor, atrazine, anilofos, amicarbazone, amidosulfuron, amitrole, aminocyclopyrachlor, aminopyralid, amiprofos-methyl, ametryn, Araujia Mosaic Virus, alachlor, Alternaria destruens, alloxydim (including a salt with sodium or the like), ancymidol, isouron, isoxachlortole, isoxaflutole, isoxaben, isodecylalkoholethoxylate, isoproturon, ipfencarbazone, imazaquin, imazapic (including a salt with amine or the like), imazapyr (including a salt of isopropylamine or the like), imazamethabenz, imazamethabenz-methyl, imazamox, imazethapyr, imazosulfuron, indaziflam, indanofan, eglinazine-ethyl, esprocarb, ethametsulfuron-methyl, ethalfluralin, ethidimuron, ethoxysulfuron, ethoxyfen, ethoxyfen-ethyl, ethofumesate, etobenzanid, epyrifenacil, endothal-disodium, oxadiazon, oxadiargyl, oxaziclomefone, oxasulfuron, oxyfluorfen, oryzalin, Obuda Pepper Virus, orthosulfamuron, orbencarb, oleic acid, cafenstrole, caprylic acid, capric acid, carfentrazone-ethyl, karbutilate, carbetamide, quizalofop, quizalofop-ethyl, quizalofop-P-ethyl, quizalofop-P-tefuryl, Xanthomonas campestris, quinoclamine, quinclorac, quinmerac, citric acid, cumyluron, clacyfos, glyphosate (including a salt of sodium, potassium, amine, propylamine, isopropylamine, ammonium, isopropylammonium, guanidine, monoethanolamine, choline, N,N-bis(aminopropyl)methylamine (BAPMA), dimethylamine, trimesium, or the like), glufosinate (including a salt of amine, sodium, or the like), glufosinate-P, glufosinate-P-sodium, clethodim, clodinafop, clodinafop-propargyl, clopyralid (including a monoethanolamine salt), clomazone, chlomethoxyfen, clomeprop, cloransulam-methyl, chloramben, chloridazon, chlorimuron, chlorimuron-ethyl, chlorsulfuron, chlorthal-dimethyl, chlorthiamid, chlorphthalim, chlorflurenol-methyl, chlorpropham, chlorbromuron, chloroxuron, chlorotoluron, ketospiradox (including a salt of sodium, calcium, ammonia, or the like), Colletotrichum orbiculare, Colletotrichum gloeosporioides, Colletotrichum truncatum, Chondrostercum purpureum, saflufenacil, sarmentine, cyanazine, cyanamide, diuron, diethatyl-ethyl, dioxopyritrione, dicamba (including a salt of amine, diethylamine, isopropylamine, diglycolamine, dimethylammonium, diolamine, isopropylammonium, auramine, potassium, trolamine, N,N-bis (aminopropyl)methylamine (BAPMA), choline, sodium, lithium, or the like, or ester of methyl ester or the like), cycloate, cycloxydim, diclosulam, cyclosulfamuron, cyclopyranil, cyclopyrimorate, dichlobenil, diclofop, diclofop-P-methyl, diclofop-methyl, dichlorprop, dichlorprop-P (including a salt of dimethylammonium, potassium, sodium, choline, or the like, or ester of butotyl ester, 2-ethylhexyl ester, isoctyl ester, methyl ester, or the like), diquat, diquat dibromide, dithiopyr, siduron, dinitramine, cinidon-ethyl, cinosulfuron, dinoseb (including acetate), dinoterb, cyhalofop, cyhalofop-butyl, cypyrafluone, diphenamid, difenzoquat, diflufenican, diflufenzopyr, simazine, dimesulfazet, dimethachlor, dimethametryn, dimethenamid, dimethenamid-P, simetryn, dimepiperate, dimefuron, Pseudomonas fluorescens, cinmethylin, swep, sulcotrione, sulfentrazone, sulfosate, sulfosulfuron, sulfometuron-methyl, sethoxydim, Sclerotinia minor, terbacil, daimuron, thaxtomin A, Tobacco Mild Green Mosaic Tobamovirus, Tobacco Rattle Virus, dalapon, thiazopyr, tiafenacil, thiencarbazone (including a sodium salt, methyl ester, or the like), tiocarbazil, thiobencarb, thidiazimin, thidiazuron, thifensulfuron, thifensulfuron-methyl, desmedipham, desmetryne, tetflupyrolimet, thenylchlor, tebutam, tebuthiuron, epraloxydim, tefuryltrione, terbuthylazine, terbutryn, terbumeton, tembotrione, topramezone, tralkoxydim, triaziflam, triasulfuron, triafamone, tri-allate, trietazine, triclopyr, triclopyr-butotyl, triclopyr-triethylammonium, tritosulfuron, tripyrasulfone, trifludimoxazin, triflusulfuron-methyl, trifluralin, trifloxysulfuron (including a salt of sodium), tribenuron-methyl, tolpyralate, naptalam (including a salt of sodium), naproanilide, napropamide, napropamide-M, nicosulfuron, lactic acid, neburon, norflurazon, Barkholderia rinojensis, vernolate, paraquat, paraquat dichloride, halauxifen, halauxifen-benzyl, halauxifen-methyl, Burkholderia rinojensis, haloxyfop, haloxyfop-P, haloxyfop-etotyl, haloxyfop-P-methyl, halosafen, halosulfuron-methyl, bixlozone, picloram (including a salt of dichloroammonium, trolamine, or the like), picolinafen, bicyclopyrone, bispyribac-sodium, pinoxaden, bipyrazone, bifenox, piperophos, pyraclonil, pyrasulfotole, pyrazoxyfen, pyrazosulfuron-ethyl, pyrazolynate, bilanafos, pyraflufen, pyraflufen-ethyl, pyridafol, pyrithiobac-sodium, pyridate, pyriftalid, pyributicarb, pyribenzoxim, pyrimisulfan, pyriminobac-methyl, pyroxasulfone, pyroxsulam, Phytophthora palmivora, phenisopham, fenuron, fenoxasulfone, fenoxaprop (including methyl, ethyl, or isopropyl ester), fenoxaprop-P (including methyl, ethyl, or isopropyl ester), fenquinotrione, fenthiaprop-ethyl, fentrazamide, fenpyrazone, phenmedipham, Phoma chenopodicola, Phoma herbarum, Phoma macrostoma, butachlor, butafenacil, butamifos, butylate, Puccinia canaliculata, Puccinia thlaspeos, butenachlor, butralin, butroxydim, flazasulfuron, flamprop (including methyl, ethyl, or isopropyl ester), flamprop-M (including methyl, ethyl, or isopropyl ester), primisulfuron, primisulfuron-methyl, fluazifop-butyl, fluazifop-P, fluazifop-P-butyl, fluazolate, fluometuron, fluoroglycofen-ethyl, flucarbazone-sodium, fluchloralin, flucetosulfuron, fluthiacet-methyl, flupyrsulfuron-methyl (including a salt of sodium, calcium, ammonia, or the like), flufenacet, flufenpyr-ethyl, flupropanate (including a sodium salt), flupoxame, flumioxazin, flumiclorac-pentyl, flumetsulam, fluridone, flurtamone, fluroxypyr (including ester of butomethyl, meptyl, or the like, or a salt of sodium, calcium, ammonia, or the like), flurochloridone, pretilachlor, procarbazone (including a salt of sodium or the like), prodiamine, prosulfuron, prosulfocarb, propaquizafop, propachlor, propazine, propanil, propyzamide, propisochlor, propyrisulfuron, propham, profluazol, prohexadione-calcium, propoxycarbazone, propoxycarbazone-sodium, profoxydim, bromacil, brompyrazon, prometryn, prometon, bromoxynil (including ester of butyric acid, octanoic acid, enanthic acid, or the like), bromofenoxim, bromobutide, florasulam, florpyrauxifen, florpyrauxifen-benzyl, hexazinone, pethoxamid, benazolin, benazolin-ethyl, penoxsulam, Pepino Mosaic Virus, heptamaloxyloglucan, beflubutamid, beflubutamid-M, pebulate, pelargonic acid, bencarbazone, benquitrione, benzfendizone, bensulide, bensulfuron, bensulfuron-methyl, benzobicyclon, benzofenap, bentazone, pentanochlor, pendimethalin, pentoxazone, benfluralin, benfuresate, fosamine, fomesafen, foramsulfuron, forchlorfenuron, mecoprop (including a salt of sodium, potassium, isopropylamine, triethanolamine, dimethylamine, diolamine, trolamine, choline, or the like, or ester of ethadyl ester, 2-ethylhexyl ester, isoctyl ester, methyl ester, or the like), mecoprop-P-potassium, mesosulfuron (including ester of methyl or the like), mesotrione, metazachlor, metazosulfuron, methabenzthiazuron, metamitron, metamifop, metam (including a salt of sodium or the like), disodium methanearsonate (DSMA), methiozolin, methyldymuron, metoxuron, metosulam, metsulfuron-methyl, metobromuron, metobenzuron, metolachlor, metribuzin, mepiquat chloride, mefenacet, monosulfuron (including methyl, ethyl, or isopropyl ester), monolinuron, molinate, iodosulfuron, iodosulfulon-methyl-sodium, iofensulfuron, iofensulfuron-sodium, lactofen, lancotrione, linuron, rimisoxafen, rimsulfuron, lenacil, 2,2,2-trichloroacetic acid (TCA) (including a salt of sodium, calcium, ammonia, or the like), 2,3,6-trichlorobenzoic acid (2,3,6-TBA), 2,4,5-trichlorophenoxyacetic acid (2,4,5-T), 2,4-dichlorophenoxyacetic acid (2,4-D) (including a salt of amine, diethylamine, triethanolamine, isopropylamine, dimethylammonium, diolamine, dodecylammonium, heptylammonium, tetradecylammonium, triethylammonium, tris(2-hydroxypropyl)ammonium, trolamine, choline, sodium, lithium, or the like, or ester of butotyl ester, 2-butoxypropyl ester, 2-ethylhexyl ester, methyl ester, ethyl ester, butyl ester, isobutyl ester, octyl ester, pentyl ester, propyl ester, isooctyl ester, isopropyl ester, meptyl ester, tefuryl ester, or the like), 2,4-dichlorophenoxybutyric acid (2,4-DB) (including a salt of amine, diethylamine, triethanolamine, isopropylamine, dimethylammonium, choline, sodium, lithium, or the like, or ester of isoctyl ester or the like), 2-amino-3-chloro-1,4-naphthoquinone (ACN), 2-methyl-4-chlorophenoxyacetic acid (MCPA) (including a salt of sodium, dimethylammonium, choline, or the like, or ester of 2-ethylhexyl ester, isoctyl ester, ethyl ester, or the like), 2-methyl-4-chlorophenoxyacetic acid (MCPB) (including a sodium salt, ethyl ester, or the like), 4-(2,4-dichlorophenoxy)butyric acid (2,4-DB), 4,6-dinitro-O-creso (DNOC) (including a salt of amine, sodium, or the like), (5S)-3-(3,5-difluorophenyl)-N-[rel-(3R,5R)-5-(trifluoromethylsulfonylcarbamoyl)tetrahydrofuran-3-yl]-5-vinyl-4H-isoxazole-5-carboxamide (Chemical name, CAS registry number: 2266183-40-6) (WO 2018/228986 A and WO 2020/114934 A), N4-(2,6-difluorophenyl)-6-(1-fluoro-1-methylethyl)-1,3,5-triazine-2,4-diamine (Chemical name, CAS registry number: 1606999-43-2) (WO 2014/064094 A and WO 2015/162164 A), (5S)-3-(3,5-difluorophenyl)-N-[(3R)-5-(methylsulfonylcarbamoyl)-2,3-dihydrofuran-3-yl]-5-vinyl-4H-isoxazole-5-carboxamide (Chemical name, CAS registry number: 2266190-06-9) (WO 2018/228986 A and WO 2020/114934 A), (5R)-3-(3,5-difluorophenyl)-5-methyl-N-[rel-(3R,5R)-5-(methylsulfonylcarbamoyl)tetrahydrofuran-3-yl]-4H-isoxazole-5-carboxamide (Chemical name, CAS registry number: 2266164-36-5) (WO 2018/228986 A and WO 2020/114934 A), (5R)-3-(3,5-difluorophenyl)-N-[(3R)-5-(methoxycarbamoyl)-2,3-dihydrofuran-3-yl]-5-methyl-4H-isoxazole-5-carboxamide (Chemical name, CAS registry number: 2266170-31-2) (WO 2018/228986 A and WO 2020/114934 A), 4-hydroxy-1-methyl-3-[4-(trifluoromethyl)-2-pyridyl]imidazolidin-2-one (Chemical name, CAS registry number: 1708087-22-2) (WO 2015/059262 A and WO 2018/015476 A), 6-(1-fluorocyclopentyl)-N4-(2,3,5,6-tetrafluorophenyl)-1,3,5-triazine-2,4-diamine (Chemical name, CAS registry number: 1820807-75-7) (WO 2015/162164 A), 6-(1-fluoro-1-methylethyl)-N4-(2,3,5,6-tetrafluorophenyl)-1,3,5-triazine-2,4-diamine (Chemical name, CAS registry number: 1606999-21-6) (WO 2014/064094 A, WO 2015/162164 A), (5S)-3-(3-fluoro-5-methylphenyl)-N-[rel-(3R,5R)-5-(methoxycarbamoyl)tetrahydrofuran-3-yl]-5-vinyl-4H-isoxazole-5-carboxamide (Chemical name, CAS registry number: 2266292-43-5) (WO 2018/228986 A and WO 2020/114934 A), 6-(1-methylcyclobutyl)-N4-(2,3,5,6-tetrafluorophenyl)-1,3,5-triazine-4,4-diamine (Chemical name, CAS registry number: 1607001-97-7) (WO 2014/064094 A and WO 2015/162164 A), AE-F-150944 (code number), IR-6396 (code number), MCPA-thioethyl, SYP-298 (code number), SYP-300 (code number), S-ethyldipropylthiocarbamate (EPTC), S-metolachlor, 5-9750 (code number), MSMA, and HW-02 (code number).

### Plant Growth Regulators:

1-naphthylacetamide, 1-methylcyclopropene, 1,3-diphenylurea, 2,3,5-triiodobenzoic acid, 2-methyl-4-chlorophenoxyacetic acid (MCPB) [including a sodium salt, ethyl ester, or the like], 2-(naphthalene-1-yl)acetamide, 2,6-diisopropylnaphthalene, 3-[(6-chloro-4-phenylquinazoline-2-yl)amino]propane-1-ol, 4-oxo-4-(2-phenylethyl)aminobutyric acid (Chemical name, CAS registry number: 1083-55-2), 4-chlorophenoxyacetic acid (4-CPA), 5-aminolevulinic acid hydrochloride, methyl 5-(trifluoromethyl)benzo[b]thiofen-2-carboxylate, aminoethoxyvinylglycine (AVG), n-decanol, anisiflupurin, aviglycine, ancymidol, abscisic acid, isoprothiolane, inabenfide, indole acetic acid, indole butyric acid, uniconazole, uniconazole-P, Ecolyst, ethychlozate, ethephon, epocholeone, calcium chloride, choline chloride, oxine-sulfate, kinetin, calcium peroxide, carvone, calcium formate, cloxyfonac, cloxyfonac-potassium, cloprop, chlormequat, chlormequat-chloride, chlorpropham, choline, cytokinins, oxydized glutathione, cyanamide, sodium cyanate, cyclanilide, dichlorprop (including a salt of dimethylammonium, potassium, sodium, choline, or the like, or ester of butotyl ester, 2-ethylhexyl ester, isoctyl ester, methyl ester, or the like), dichlorprop-P (including a salt of sodium, potassium, dimethylammonium, or the like, or 2-ethylhexyl ester), diquat, diquat dibromide, dikegulac, gibberellinic acid, gibberellin A4, gibberellin A7, dimethipin, sintofen, jasmone, cis-jasmone, jasmonic acid, methyl jasmonate, streptomycin, calcium polysulfide, daminozide, calcium carbonate, thidiazuron, decan-1-ol, triacontanol, triapenthenol, trinexapac-ethyl, tribufos, paclobutrazol, paraffin, bispyribac-sodium, hymexazol, butralin, fluthiacet-methyl, pyraflufen-ethyl, flumetralin, flurprimidol, flurenol, pronitridine, prohydrojasmon, prohexadione-calcium, heptamaloxyloglucan, 6-benzylaminopurine, pendimethalin, forchlorfenuron, formononetin, maleic hydrazide, mepiquat chloride, mefluidide, lipochitooligosaccharides (for example, lipochitooligosaccharides SP104), and calcium sulfate.

Next, examples of known phytotoxicity alleviating compounds that can be mixed or used in combination are shown below:

### Phytotoxicity Alleviating Compounds:

isoxadifen, isoxadifen-ethyl, oxabetrinil, octane-1,8-diamine, cloquintocet, cloquintcet-mexyl, dietholate, cyometrinil, dichlormid, dicyclonone, cyprosulfamide, daimuron, 1,8-naphthalic anhydride, fenchlorazole, fenchlorazole-O-ethyl, fenclorim, furilazole, fluxofenim, flurazole, benoxacor, metcamifen, mephenate, mefenpyr, mefenpyr-ethyl, mefenpyr-diethyl, lower alkyl-substituted benzoic acid, 2,2-dichloro-N-(1,3-dioxan-2-ylmethyl)-N-(2-propenyl)acetamide (PPG-1292), 2-dichloromethyl-2-methyl-1,3-dioxane (MG-191), 3-dichloroacetyl-2,2,5-trimethyl-1,3-oxazolidine (R-29148), 4-dichloroacetyl-1-oxa-4-azaspiro[4.5]decane (AD-67), 4-carboxy-3,4-dihydro-2H-1-benzopyran-4-acetic acid (CL-304415, code number), MON4660 (code number), metcamifen, N1,N2-diallyl-N2-dichloroacetyl glycineamide (DKA-24, code number), 1-bromo-4-[(chloromethyl) sulfonyl]benzene (CSB), 2-propenyl-1-oxa-4-azaspiro[4,5]decan-4-carbodithioate (MG-838, code number), 3-(dichloroacetyl)-2,2-dimethyl-1,3-oxazolidine (R-28725, code number), R-29148 (code number), and 1-(dichloroacetyl)azepane (TI-35, code number).

Next, examples of known biological pesticides that can be mixed or used in combination are shown.

### Biological Pesticides:

Haplothrips brevitubus, Franklinothrips vespiformis, Diglyphus isaea, Encarsia formosa, Amblyseius cucumeris, Pseudaphycus malinus, Amblyseius womersleyi, Aphidius colemani, Eretmocerus eremicus, Aphidoletes aphidimyza, Amblyseius swirskii, Orius strigicollis, Phytoseiulus persimilis, Amblyseius degenerans, Phytoseiulus persimilis, Orius sauteri, Dacnusa sibirica, Amblyseius californicus, Chrysoperla nipponensis, and Anicetus beneficus.

Next, examples of known agricultural materials that can be mixed or used in combination are shown.

### Agricultural Materials:

ethylene, hypochlorous acid water (limited to those obtained by electrolyzing hydrochloric acid or an aqueous potassium chloride solution), sodium bicarbonate, vinegar, humic substances, humic acid, fulvic acid, seaweed extract, polysaccharide, amino acid, a microbial material, an animal and plant-derived functional component, microbial metabolite, a microbial activator, a soil spreading agent, a soil permeability control material, a soil water retention material, and a biostimulant.

Next, examples of known agricultural fertilizer components that can be mixed or used in combination are shown below. Examples of the fertilizer include an inorganic fertilizer and an organic fertilizer.

### Agricultural Fertilizer Components:

ammonium chloride, ammonium sulfate, ammonium nitrate, ammonium dihydrogen phosphate, urea ammonium nitrate, urea, lime nitrogen, potassium nitrate, superphosphate of lime, double superphosphate of lime, potassium dihydrogen phosphate, potassium phosphite, potassium chloride, potassium sulfate, potassium carbonate, potassium silicate, oil cake, fish powder, rice bran, bat guano, and fermented chicken manure.

Specific examples of diseases that can be controlled by the agricultural and horticultural plant disease control agent and the method for controlling agricultural and horticultural plant diseases are described below, but the diseases are not limited thereto: blast disease of rice (Pyricularia oryzae), leaf spot (Cochliobolus miyabeanus), sheath blight disease (Rhizoctonia solani), bakanae disease (Gibberella fujikuroi), damping-off (Fusarium spp., Pythium spp., Rhizopus spp., Trichoderma sp.), damping-off disease (Burkholderia plantarii), bacterial leaf spot (Xanthomonas oryzae), bacterial grain rot of rice (Burkholderia glumae), bacterial brown stripe of rice (Acidovorax avenae), bacterial palea browning (Erwinia ananas), powdery mildew of wheat (Erysiphe graminis f.sp. hordei, f.sp. tritici), mottle leaf (Pyrenophora graminea), net blotch (Pyrenophora teres), scab (Gibberella zeae), rust (Puccinia striiformis, Puccinia graminis, Puccinia recondita, Puccinia hordei), snow mold (Typhula incarnata, Typhula ishikariensis, Micronectriella nivalis, Microdochium nivale), loose smut (Ustilago tritici, U. nuda), eye spot (Pseudocercosporella herpotrichoides), leaf blotch (Rhynchosporium secalis), leaf spot (Septoria tritici), glume blotch (Septoria nodorum, Leptosphaeria nodorum), snow mold caused by the genus Pythium (Pythium iwayamai or the like), downy mildew of grape (Plasmopara viticola), powdery mildew (Uncinula necator), sphaceloma scab (Elsinoe ampelina), ripe rot (Glomerella cingulata), rust (Phakopsora ampelopsidis), black rot (Guignardia bidwellii), leaf spot (Phomopsis viticola), powdery mildew of apple (Podosphaera leucotricha), black spot (Venturia inaequalis), leaf spot (Alternaria alternata), rust (Gymnosporangium yamadae), monilinia mali (Sclerotinia mali), brown canker (Valsa ceratospema), ring spot (Physalospora piricola), black spot of pear (Alternaria kikuchiana), black spot (Venturia nashicola), rust (Gymnosporangium haraeanum), blight (Phomopsis fukushii), blight of western pear (Phytophthora cactorum), brown rot of peach (Monilinia fructicola), black spot (Cladosporium carpophilum), phomopsis black rot (Phomopsis sp.), blight (Phytophthora sp.), anthracnose of persimmon (Colletotrichum gloeosporioides), leaf spot (Pseudocercospora kaki, Mycosphaerella nawae), black rot of mandarin (Diaporthe citri), scab (Elsinoe fawcetti), downy mildew of gourds (Pseudoperonospora cubensis), blight (Phytophthora parasitica, Phytophthora melonis, Phytophthora nicotianae, Phytophthora drechsleri, Phytophthora capsici, or the like), powdery mildew (Sphaerotheca fuliginea, Sphaerotheca cucurbitae), anthracnose (Colletotrichum lagenarium), gummy stem blight (Mycosphaerella melonis), fusarium wilt (Fusarium oxysporum), damping-off (Pythium debaryanum), brown spot (Corynespora cassiicola), gray mold (Botrytis cinerea), bacterial blight (Pseudomonas syringae), blight of tomato (Phytophthora infestans), damping-off (Pythium vexans, Rhizoctonia solani), ring spot (Alternaria solani), damping-off (Pythium myriotylum, Pythium dissotocum), anthracnose (Colletotrichum phomoides), leaf mold (Cladosporium fulvum), canker (Corynebacterium michiganense), powdery mildew of eggplant (Sphaerotheca fuliginea), blight (Phytophthora infestans), brown rot (Phytophthora capsici), bacterial wilt (Ralstonia solanacearum), stem rot of rapeseed (Sclerotinia scerotiorum), downy mildew (Peronospora brassicae), black spot of brassicaceae vegetable (Alternaria japonica), silvertop (Pseudocercosporella capsellae), clubroot (Plasmodiophora brassicae), downy mildew (Peronospora brassicae), white blight of green onion (Phytophthora porri), rust (Puccinia allii), soft rot of Chinese cabbage (Erwinia carotovora), root blight of soybean (Phytophthora megasperma), rust (Phakopsora pachyrhizi), stem rot (Sclerotinia sclerotiorum), downy mildew (Peronospora manshurica), purpura (Cercospora kikuchii), sphaceloma scab (Elsinoe glycines), black rot (Diaporthe phaseololum), anthracnose of wax bean (Colletotrichum lindemuthianum), black rust of peanut (Mycosphaerella berkeleyii), brown spot (Mycosphaerella arachidis), powdery mildew of pea (Erysiphe pisi), downy mildew (Peronospora pisi), blight of potato (Phytophthora infestans), early blight (Alternaria solani), net blister blight (Exobasidium reticulatum), white scab (Elsinoe leucospila), rust of tabacco (Alternaria alternata (tabacco pathotype)), powdery mildew (Erysiphe cichoracearum), anthracnose (Colletotrichum tabacum), brown spot of beet (Cercospora beticola), downy mildew (Peronospora schachtii), black spot of rose (Diplocarpon rosae), powdery mildew (Sphaerotheca pannosa), brown spot of chrysanthemum (Septoria chrysanthemi-indici), white rust (Puccinia horiana), powdery mildew of strawberry (Sphaerotheca macularis), blight (Phytophthora nicotianae), fruit soft rot (Pythium ultimum), anthracnose (Colletotrichum fragariae), black Sigatoka disease of banana (Mycosphaerella fijiensis), yellow Sigatoka disease (Mycosphaerella musicola), fusarium wilt (Fusarium oxysporum), gray mold of cucumber, tomato, green bean, strawberry, grape, or the like (Botrytis cinerea), stem rot (Sclerotinia sclerotiorum), grass brownpatch (Rhizoctonia solani), dollar spot (Sclerotinia homoeocarpa), curvularia leaf spot (Curvularia geniculata), rust (Puccinia zoysiae), helminthosporium leaf spot (Cochliobolus sp.), leaf blotch (Rhynchosporium secalis), root rot (Gaeumannomyces graminis), anthracnose (Colletotrichum graminicola), typhula brown stem rot (Typhula incarnata), typhula black stem rot (Typhula ishikariensis), sclerotinia stem rot (Sclerotinia borealis), fairy ring (Marasmius oreades or the like), and pythium blight (Pythium aphanidermatum or the like).

The agricultural and horticultural plant disease control agent of the present invention also exhibits a control effect on the plant diseases and the like exemplified above, which have acquired resistance to the existing agricultural and horticultural plant disease control agent. Furthermore, the agricultural and horticultural plant disease control agent of the present invention can be used for plants that have acquired characteristics such as pest tolerance, disease tolerance, herbicide tolerance, or the like by gene recombination, new breeding techniques such as genome editing, artificial mating, or the like.

Next, the production methods, the formulation methods, and the applications of the compound of the present invention will be described in detail with reference to the following Examples. However, the present invention is not limited to these Examples. The melting point which is a physical property value of the compound of the present invention was determined by a MP-500V micro melting point measuring apparatus manufactured by Yanaco Corporation. The refractive index was determined by using an Abbe refractometer manufactured by ATAGO CO., LTD. 1H NMR spectrum was determined by using JNM-LA400 (400 MHz) or JNM-ECS300 (300 MHz) manufactured by JEOL Ltd. and tetramethylsilane (TMS) as the internal standard.

A method for producing a production intermediate of the compound of the present invention is also described.

### Example 1

### Production of N-benzyl-N-{4-[5-(trifluoromethyl)-1,2,4-oxadiazol-3-yl]phenyl}formamide (Compound of Present Invention: A-0096)

### (1) Production of N-{4-[5-(trifluoromethyl)-1,2,4-oxadiazol-3-yl]phenyl}formamide (Compound of Present Invention: A-0001)

8.70 g (37.96 mmol) of 4-[5-(trifluoromethyl)-1,2,4-oxadiazol-3-yl]phenylamine produced by the method described in Example 188 of WO 2007/111212 A and 2.80 g (41.17 mmol) of sodium formate were dissolved in 100 ml of formic acid, and the mixture was heated and refluxed for 3 hours. The reaction mixture was cooled to room temperature, the solvent was distilled in vacuo, ethyl acetate was added, the organic layer was washed with a saturated sodium bicarbonate aqueous solution and brine, drying with anhydrous magnesium sulfate was performed, and the solvent was distilled in vacuo. The residue was purified by silica gel column chromatography (developing solvent, n-hexane ethyl acetate=3:1) to obtain 8.30 g of a desired product (yield: 85%).

¹H-NMR data (300 MHz, DMSO-d₆/TMS δ (ppm)) 10.50-10.60 (1H, m), 8.32-9.01 (1H, m), 7.99-8.06 (2H, m), 7.20-7.84 (2H, m)

### (2) Production of N-benzyl-N-{4-[5-(trifluoromethyl)-1,2,4-oxadiazol-3-yl]phenyl}formamide (Compound of Present Invention: A-0096)

0.40 g (1.56 mmol) of N-{4-[5-(trifluoromethyl)-1,2,4-oxadiazol-3-yl]phenyl}formamide was dissolved in 20 ml of acetonitrile, and 0.43 g (3.11 mmol) of potassium carbonate, 0.53 g (3.10 mmol) of benzyl bromide, and 0.026 g (0.16 mmol) of potassium iodide were sequentially added at room temperature, and the mixture was stirred for 40 hours. Water was added at room temperature, and extraction was performed with ethyl acetate. Washing was performed with brine, drying with anhydrous sodium sulfate was performed, and the solvent was distilled in vacuo. The residue was purified by silica gel column chromatography (developing solvent, n-hexane:ethyl acetate=3:1) to obtain 0.46 g of a desired product (yield: 85%).

¹H-NMR data (300 MHz, CDCl₃/TMS δ (ppm)): 8.61-8.73 (1H, m), 8.05-8.12 (2H, m), 7.22-7.38 (7H, m), 4.94-5.08 (2H, m)

### Example 2

### Production of N-{4-[5-(trifluoromethyl)-1,2,4-oxadiazol-3-yl]phenyl}formamide (Compound of Present Invention: A-0001)

40.00 g (0.12 mol) of t-butyl {4-[5-(trifluoromethyl)-1,2,4-oxadiazol-3-yl]phenyl}carbamate produced by the method described in Example 188 of WO 2007/111212 A and 8.14 g (0.12 mol) of sodium formate were dissolved in 80 ml of formic acid, and the mixture was heated and refluxed for 3 hours. Cooling was performed at room temperature, 200 ml of water was added dropwise, and stirring was performed for 1 hour. The precipitated crystals were collected by filtration and washed with water. The crystals were dissolved in 250 ml of ethyl acetate, drying with anhydrous magnesium sulfate was performed, and the solvent was distilled in vacuo, thereby obtaining 30.00 g of a desired product (yield: 96%).

¹H-NMR data (300 MHz, DMSO-d₆/TMS δ (ppm)): 10.50-10.60 (1H, m), 8.32-9.01 (1H, m), 7.99-8.06 (2H, m), 7.20-7.84 (2H, m)

### Example 3

### Production of N-(quinolin-3-ylmethyl)-N-{4-[5-(trifluoromethyl)-1,2,4-oxadiazol-3-yl]phenyl}formamide (Compound of Present Invention: B-0004)

### (1) Production of 1-(quinolin-3-yl)-N-{4-[5-(trifluoromethyl)-1,2,4-oxadiazol-3-yl]phenyl}methenamine

0.40 g (1.75 mmol) of 4-[5-(trifluoromethyl)-1,2,4-oxadiazol-3-yl]phenylamine and 0.27 g (1.72 mmol) of quinolin-3-carbaldehyde were dissolved in 10 ml of toluene, and the mixture was heated and refluxed for 5 hours. The reaction mixture was cooled, and the solvent was distilled in vacuo, thereby obtaining 0.62 g of a desired product (yield: 96%).

¹H-NMR data (300 MHz, CDCl₃/TMS δ (ppm)) 9.52 (1H, d), 8.70 (1H, s), 8.62 (1H, d), 8.20 (3H, m), 7.96 (1H, dd), 7.84 (1H, ddd), 7.64 (1H, ddd), 7.39-7.42 (2H, m)

### (2) Production of N-(quinolin-3-ylmethyl)-N-{4-[5-(trifluoromethyl)-1,2,4-oxadiazol-3-yl]phenyl}amine (Compound of Present Invention: G-0004)

0.50 g (1.36 mmol) of 1-(quinolin-3-yl)-N-{4-[5-(trifluoromethyl)-1,2,4-oxadiazol-3-yl]phenyl}methenamine was suspended in 10 ml of ethanol, 0.08 g (1.27 mmol) of sodium cyanoborohydride was added, and the mixture was stirred at room temperature for 1 hour. Thereafter, 0.1 ml of acetic acid was added, and the mixture was stirred at room temperature for 3 hours. 30 ml of a saturated sodium bicarbonate aqueous solution was added, the mixture was stirred for 0.5 hours, and extraction with ethyl acetate was performed. The organic layer was washed with brine and dried with anhydrous magnesium sulfate, the solvent was distilled in vacuo, and the residue was purified by silica gel column chromatography (developing solvent, n-hexane ethyl acetate=1:1), thereby obtaining 0.24 g of a desired product (yield: 48%).

¹H-NMR data (300 MHz, DMSO-d₆/TMS δ (ppm)): 8.94 (1H, d), 8.27 (1H, d), 7.03-7.94 (2H, m), 7.70-7.81 (3H, m), 7.59 (1H, ddd), 7.27 (1H, t), 6.81 (2H, d), 4.60 (2H, d)

### (3) Production of N-(quinolin-3-ylmethyl)-N-{4-[5-(trifluoromethyl)-1,2,4-oxadiazol-3-yl]phenyl}formamide (Compound of Present Invention: B-0004)

0.17 g (0.46 mmol) of N-(quinolin-3-ylmethyl)-N-{4-[5-(trifluoromethyl)-1,2,4-oxadiazol-3-yl]phenylamine and 0.03 g (0.46 mmol) of sodium formate were dissolved in 1.0 ml of formic acid, and the mixture was heated and refluxed for 0.5 hours. Cooling was performed at room temperature, 30 ml of water was added, extraction with ethyl acetate was performed, and washing with a saturated sodium bicarbonate aqueous solution was performed. The organic layer was dried with anhydrous magnesium sulfate, and the solvent was distilled in vacuo, thereby obtaining 0.17 g of a desired product (yield: 93%).

¹H-NMR data (300 MHz, DMSO-d₆/TMS δ (ppm)) 8.80-8.95 (2H, m), 8.19-8.26 (1H, m), 7.90-8.05 (4H, m), 7.68-7.75 (3H, m), 7.58 (1H, ddd), 5.35 (2H, s)

### Example 4

### Production of N-[(tetrahydrofuran-2-yl)methyl]-N-{4-[5-(trifluoromethyl)-1,2,4-oxadiazol-3-yl]phenyl}formamide (Compound of Present Invention: B-0810)

### (1) Production of 4-{[(tetrahydrofuran-2-yl)methyl]amino}benzonitrile

3.00 g (24.81 mmol) of 4-fluorobenzonitrile and 2.51 g (24.81 mmol) of (tetrahydrofuran-2-yl)methanamine were dissolved in 30 ml of DMF, 3.43 g (24.81 mmol) of potassium carbonate was added, and the mixture was stirred at 120° C. for 5 hours. Cooling was performed at room temperature, water was added, and extraction with ethyl acetate was performed. Drying with anhydrous magnesium sulfate was performed, the solvent was distilled in vacuo, and the residue was purified by silica gel column chromatography (developing solvent, n-hexane:ethyl acetate=1:1), thereby obtaining 2.40 g of a desired product (yield: 48%).

¹H-NMR data (300 MHz, CDCl₃/TMS δ (ppm)): 7.39-7.44 (2H, m), 6.55-6.60 (2H, m), 4.55 (1H, s), 4.07-4.15 (1H, m), 3.76-3.93 (2H, m), 3.27-3.34 (1H, m), 3.06-3.14 (1H, m), 1.90-2.09 (3H, m), 1.60-1.67 (1H, m)

### (2) Production of N-(4-cyanophenyl)-N-[(tetrahydrofuran-2-yl)methyl]formamide

2.40 g (11.87 mmol) of 4-{[(tetrahydrofuran-2-yl)methyl]amino}benzonitrile and 0.81 g (11.87 mmol) of sodium formate were dissolved in 10 ml of formic aid, and the mixture was heated and refluxed for 0.5 hours. Cooling was performed at room temperature, water was added, and extraction with ethyl acetate was performed. Washing with a saturated sodium bicarbonate aqueous solution was performed, drying with anhydrous magnesium sulfate was performed, and the solvent was distilled in vacuo, thereby obtaining 2.20 g of a desired product (yield: 81%).

¹H-NMR data (300 MHz, CDCl₃/TMS δ (ppm)): 8.38-8.55 (1H, m), 7.68-7.72 (2H, m), 7.40-7.61 (2H, m), 3.97-4.27 (2H, m), 3.67-3.89 (3H, m), 1.80-2.10 (3H, m), 1.48-1.62 (1H, m)

### (3) Production of N′-hydroxy-4-{N-[(tetrahydrofuran-2-yl)methyl]formamid}benzimidamide

2.20 g (9.55 mmol) of N-(4-cyanophenyl)-N-[(tetrahydrofuran-2-yl)methyl]formamide and 0.80 g (11.46 mmol) of hydroxylamine hydrochloride were added to 30 ml of ethanol, 1.16 g (11.46 mmol) of triethylamine was further added, and the mixture was heated and refluxed for 4 hours. Cooling was performed at room temperature, water was added, and extraction with ethyl acetate was performed. Washing with brine was performed, drying with anhydrous magnesium sulfate was performed, and the solvent was distilled in vacuo, thereby obtaining 1.30 g of a desired product (yield: 52%).

¹H-NMR data (300 MHz, DMSO-d₆/TMS δ (ppm)): 9.64-9.66 (1H, m), 8.30-8.46 (1H, m), 7.66-7.72 (2H, m), 7.36-7.40 (2H, m), 5.83 (2H, s), 3.52-3.96 (5H, m), 1.42-1.91 (4H, m)

### (4) Production of N-[(tetrahydrofuran-2-yl)methyl]-N-{4-[5-(trifluoromethyl)-1,2,4-oxadiazol-3-yl]phenyl}formamide (Compound of Present Invention: B-0810)

1.15 g (4.37 mmol) of N′-hydroxy-4-{N-[(tetrahydrofuran-2-yl)methyl]formamid}benzimidamide was dissolved in 30 ml of tetrahydrofuran, 1.01 g (4.80 mmol) of trifluoroacetic anhydride was added under ice cooling, and the mixture was stirred at 0° C. for 2 hours. The solvent was distilled in vacuo, water was added, and extraction with ethyl acetate was performed.

Washing with a saturated sodium bicarbonate aqueous solution was performed, drying with anhydrous magnesium sulfate was performed, the solvent was distilled in vacuo, and the residue was purified by silica gel column chromatography (developing solvent, n-hexane:ethyl acetate=1:3), thereby obtaining 0.75 g of a desired product (yield: 50%).

¹H-NMR data (300 MHz, DMSO-d₆/TMS δ (ppm)): 8.38-8.64 (1H, m), 7.98-8.16 (2H, m), 7.64-7.73 (2H, m), 3.52-4.06 (5H, m), 1.68-1.94 (3H, m), 1.49-1.53 (1H, m)

### Example 5

### Production of 4-[5-(trifluoromethyl)-1,2,4-oxadiazol-3-yl]phenylamine

(1) Production of N-hydroxy-4-nitrobenzimidamide 15.00 g (0.10 mol) of 4-nitrobenzonitrile and 21.10 g (0.30 mol) of hydroxylamine hydrochloride were added to 200 ml of ethanol, 50.60 g (0.50 mol) of triethylamine was further added, and the mixture was heated and refluxed for 2 hours. Cooling was performed at room temperature, water was added, and extraction with ethyl acetate was performed. Washing with brine was performed, drying with anhydrous magnesium sulfate was performed, and the solvent was distilled in vacuo, thereby obtaining 18.34 g of a desired crude product.

¹H-NMR data (300 MHz, DMSO-d₆/TMS δ (ppm)) 10.13 (1H, s), 8.21-8.26 (2H, m), 7.92-7.97 (2H, s), 6.07 (1H, brs)

(2) Production of 3-(4-nitrophenyl)-5-(trifluoromethyl)-1,2,4-oxadiazole 18.00 g (0.10 mol) of crude N-hydroxy-4-nitrobenzimidamide was dissolved in 300 ml of toluene, 42.00 g (0.20 mol) of trifluoroacetic anhydride was added at room temperature, and the mixture was heated and refluxed for 2 hours. Cooling was performed, and the solvent was distilled in vacuo. The residue was purified by silica gel column chromatography (developing solvent, n-hexane: ethyl acetate=10:1) to obtain 26.28 g of a desired product (yield: 100%).

¹H-NMR data (300 MHz, CDCl₃/TMS δ (ppm)): 8.32-8.43 (4H, m)

### (3) Production of 4-[5-(trifluoromethyl)-1,2,4-oxadiazol-3-yl]aniline

4.96 g (19.14 mmol) of 3-(4-nitrophenyl)-5-(trifluoromethyl)-1,2,4-oxadiazole and 0.62 g (1.92 mmol) of tetrabutylammonium bromide were dissolved in a mixed solution of 40 mL of tetrahydrofuran and 40 mL of water, 8.88 g (51.01 mmol) of sodium dithionite was added under ice cooling, and the mixture was stirred under ice cooling for 2 hours. 2.00 g (11.49 mmol) of sodium dithionite was further added under ice cooling, and the mixture was stirred under ice cooling for 1 hour. At room temperature, water was added, and extraction with ethyl acetate was performed.

The organic layer was dried with anhydrous magnesium sulfate, the solvent was distilled in vacuo, and the residue was purified by silica gel column chromatography (developing solvent, n-hexane:ethyl acetate=1:1), thereby obtaining 2.38 g of a desired product (yield: 54%).

¹H-NMR data (300 MHz, CDCl₃/TMS δ (ppm)): 7.88-7.93 (2H, m), 6.72-6.77 (2H, m), 4.06 (1H, brs)

### Example 6

### Production of N-(2-bromobenzyl)-N-{4-[5-(trifluoromethyl)-1,2,4-oxadiazol-3-yl]phenyl}thioformamide (Compound of Present Invention: C-0162)

### (1) Production of N-(2-bromobenzyl)-N-{4-[5-(trifluoromethyl)-1,2,4-oxadiazol-3-yl]phenyl}formamide (Compound of Present Invention: A-0162)

0.35 g (1.36 mmol) of N-{4-[5-(trifluoromethyl)-1,2,4-oxadiazol-3-yl]phenyl}formamide was dissolved in 20 ml of DMF, 0.082 g (purity: about 60%, 2.05 mmol) of sodium hydride was added under ice cooling, and the mixture was stirred for 0.5 hours. 0.68 g (2.72 mmol) of 1-bromo-2-(bromomethyl)benzene was added under ice cooling, the temperature was raised to room temperature, and the mixture was stirred for 2 hours. The reaction mixture was cooled to 0° C., ice water was added, the mixture was stirred for 0.5 hours, ethyl acetate was added, the organic layer was washed with brine, drying with anhydrous sodium sulfate, and the solvent was distilled in vacuo. The residue was purified by silica gel column chromatography (developing solvent, n-hexane:ethyl acetate=10:1) to obtain 0.58 g of a desired product (yield: 100%).

¹H-NMR data (300 MHz, CDCl₃/TMS δ (ppm)): 8.61-8.82 (1H, m), 8.06-8.13 (2H, m), 7.09-7.60 (6H, m), 5.01-5.18 (2H, m)

### (2) Production of N-(2-bromobenzyl)-N-{4-[5-(trifluoromethyl)-1,2,4-oxadiazol-3-yl]phenyl}thioformamide (Compound of Present Invention: C-0162)

0.34 g (0.80 mmol) of N-(2-bromobenzyl)-N-{4-[5-(trifluoromethyl)-1,2,4-oxadiazol-3-yl]phenyl}formamide was dissolved in 20 ml of tetrahydrofuran, 0.21 g (0.52 mmol) of a Lawesson's reagent was added, and the mixture was stirred at room temperature for 24 hours. The solvent was distilled in vacuo, and the residue was purified by silica gel column chromatography (developing solvent, n-hexane ethyl acetate=10:1), thereby obtaining 0.33 g of a desired product (yield: 94%).

¹H-NMR data (300 MHz, CDCl₃/TMS δ (ppm)) 9.85-10.02 (1H, m), 8.11-8.16 (2H, m), 7.55-7.58 (1H, dd), 7.34-7.39 (2H, m), 7.11-7.28 (3H, m), 5.17-5.69 (2H, m)

Melting point (° C.): 122 to 123

### Example 7

### Production of ethyl {4-[5-(trifluoromethyl)-1,2,4-oxadiazol-3-yl]phenyl}glycine (Compound of Present Invention: F-0031)

2.30 g (10.04 mmol) of 4-[5-(trifluoromethyl)-1,2,4-oxadiazol-3-yl]aniline was dissolved in 30 ml of N,N-dimethylacetamide, 1.40 g (10.13 mmol) of potassium carbonate, 1.79 g (10.72 mmol) of ethyl bromoacetate, and 0.50 g (3.01 mmol) of potassium iodide were added, and the mixture was stirred at 130° C. for 2 hours. Cooling was performed at room temperature, water was added, and extraction with ethyl acetate was performed. Washing with brine was performed, drying with anhydrous magnesium sulfate was performed, the solvent was distilled in vacuo, and the residue was purified by silica gel column chromatography (developing solvent, n-hexane:ethyl acetate=5:1), thereby obtaining 0.65 g of a desired product (yield: 21%).

¹H-NMR data (300 MHz, CDCl₃/TMS δ (ppm)) 7.92-7.96 (2H, m), 6.65-6.69 (2H, m), 4.74 (1H, brs), 4.25-4.32 (2H,q), 3.97 (2H, s) 1.30-1.34 (3H, s)

### Example 8

### Production of N-[2-(trifluoromethyl)benzyl]-N-{4-[5-(trifluoromethyl)-1,2,4-oxadiazol-3-yl]phenyl}amine (Compound of Present Invention: F-0213)

### (1) Production of t-butyl N-[2-(trifluoromethyl)benzyl]-N-{4-[5-(trifluoromethyl)-1,2,4-oxadiazol-3-yl]phenyl}carbamate

0.40 g (1.21 mmol) of t-butyl {4-[5-(trifluoromethyl)-1,2,4-oxadiazol-3-yl]phenyl}carbamate was dissolved in 5 ml of N,N-dimethylformamide, 0.06 g (purity: about 60%, 1.50 mmol) of sodium hydride was added under ice cooling, and the mixture was stirred for 0.5 hours. 0.32 g (1.34 mmol) of 2-(trifluoromethyl)benzyl bromide was added, the temperature was raised to room temperature, and the mixture was stirred for 15 hours. Thereafter, water was added, and extraction with ethyl acetate was performed. Drying with anhydrous magnesium sulfate was performed, the solvent was distilled in vacuo, and the residue was purified by silica gel column chromatography (developing solvent, n-hexane ethyl acetate=9:1), thereby obtaining 0.54 g of a desired product (yield: 91%).

¹H-NMR data (300 MHz, CDCl₃/TMS δ (ppm)) 8.01-8.06 (2H, m), 7.65-7.72 (1H, m), 7.29-7.58 (5H, m), 5.10-5.14 (2H, m), 1.36-1.41 (9H, m)

### (2) Production of N-[2-(trifluoromethyl)benzyl]-N-{4-[5-(trifluoromethyl)-1,2,4-oxadiazol-3-yl]phenyl}amine (Compound of Present Invention: F-0213)

0.54 g (1.11 mmol) of t-butyl N-[2-(trifluoromethyl)benzyl]-N-{4-[5-(trifluoromethyl)-1,2,4-oxadiazol-3-yl]phenyl}carbamate was dissolved in 20 ml of dichloromethane, 4.8 ml of trifluoroacetic acid was added under ice cooling, and the mixture was stirred for 2 hours. 60 mL of a saturated sodium bicarbonate aqueous solution was added, extraction with dichloromethane was performed, washing with brine was performed, drying with anhydrous magnesium sulfate was performed, and the solvent was distilled in vacuo. The residue was purified by silica gel column chromatography (developing solvent, n-hexane:ethyl acetate=7:3) to obtain 0.23 g of a desired product (yield: 54%).

¹H-NMR data (300 MHz, CDCl₃/TMS δ (ppm)): 7.88-7.93 (2H, m), 7.71 (1H, d), 7.49-7.61 (2H, m), 7.40 (1H, t), 6.64-6.91 (2H, m), 4.62-4.64 (3H, m)

The physical property values (the melting point, the refractive index, and the ¹H-NMR spectrum data) of the compound [I] of the present invention synthesized according to each Example and the values in each Example are shown in Tables 203 to 218, the physical property values (the melting point, the refractive index, and the ¹H-NMR spectrum data) of the compound [II] of the present invention are also shown in Tables 219 to 222, and the physical property values (the melting point, the refractive index, and the ¹H-NMR spectrum data) of the compound [III] of the present invention are also shown in Tables 223 to 225. The compound numbers and symbols in the tables are as defined above.

TABLE 203

| | |
|---|---|
| Compound | Physical property value |

| Number | Melting point (° C.) | Refractive index (n_{D}²⁰) |
|---|---|---|
| A-0001 | 130-132 | |
| A-0002 | 71-72 | |
| A-0003 | 56-57 | |
| A-0004 | | 1.5196 |
| A-0006 | | 1.5153 |
| A-0007 | 64-66 | |
| A-0011 | | 1.5111 |
| A-0012 | | 1.5085 |
| A-0014 | 52-54 | |
| A-0018 | 67-69 | |
| A-0026 | 62-65 | |
| A-0027 | | 1.4979 |
| A-0029 | | 1.4541 |
| A-0032 | 31-34 | |
| A-0034 | 95-98 | |
| A-0035 | 108-110 | |
| A-0038 | 122-125 | |
| A-0039 | | 1.5041 |
| A-0041 | 93-95 | |
| A-0042 | 154-157 | |
| A-0045 | 167-169 | |
| A-0057 | 91-94 | |
| A-0064 | 68-71 | |
| A-0066 | | 1.5177 |
| A-0067 | | 1.5098 |
| A-0072 | 81-84 | |
| A-0074 | 40-43 | |
| A-0075 | 33-34 | |
| A-0080 | | 1.5009 |
| A-0081 | | 1.4995 |
| A-0082 | | 1.5692 |
| A-0095 | | 1.5267 |
| A-0096 | 64-65 | |
| A-0097 | | 1.5558 |
| A-0098 | 35-38 | |
| A-0099 | | 1.5504 |
| A-0103 | 87-90 | |
| A-0104 | 145-147 | |
| A-0105 | 168-171 | |
| A-0107 | | 1.5443 |
| A-0144 | | 1.5564 |

TABLE 204

| | |
|---|---|
| Compound | Physical property value |

| Number | Melting point (° C.) | Refractive index (n_{D}²⁰) |
|---|---|---|
| A-0151 | | 1.5786 |
| A-0156 | 67-68 | |
| A-0157 | 55-57 | |
| A-0158 | 67-70 | |
| A-0159 | 75-78 | |
| A-0160 | 53-56 | |
| A-0161 | 104-105 | |
| A-0162 | 73-76 | |
| A-0163 | 75-77 | |
| A-0164 | 122-125 | |
| A-0165 | 99-102 | |
| A-0166 | 113-116 | |
| A-0167 | 120-123 | |
| A-0168 | 102-103 | |
| A-0169 | 77-79 | |
| A-0170 | 102-103 | |
| A-0204 | | 1.5611 |
| A-0205 | 53-54 | |
| A-0206 | 71-74 | |
| A-0216 | 65-68 | |
| A-0217 | 78-80 | |
| A-0218 | 83-85 | |
| A-0219 | | 1.4995 |
| A-0220 | 57-59 | |
| A-0221 | 83-85 | |
| A-0240 | 93-95 | |
| A-0241 | 87-90 | |
| A-0242 | 85-88 | |
| A-0255 | 94-96 | |
| A-0256 | 85-88 | |
| A-0257 | 112-113 | |
| A-0259 | 58-60 | |
| A-0260 | 91-94 | |
| A-0279 | | 1.5602 |
| A-0280 | 84-87 | |
| A-0281 | 120-122 | |
| A-0282 | | 1.5548 |
| A-0283 | | 1.5479 |
| A-0284 | 104-107 | |
| A-0315 | 108-110 | |
| A-0316 | 87-89 | |

TABLE 205

| | Physical property value | |
|---|---|---|
| Compound Number | Melting point (° C.) | Refractive index (n_{D}²⁰) |
| A-0317 | 125-127 | |
| A-0334 | 63-66 | |
| A-0335 | 120-123 | |
| A-0351 | 54-57 | |
| A-0352 | 48-50 | |
| A-0353 | | 1.4727 |
| A-0354 | | 1.5200 |
| A-0355 | 57-60 | |
| A-0356 | 73-76 | |
| A-0481 | 98-100 | |
| A-0482 | | 1.5414 |
| A-0483 | 108-110 | |
| A-0484 | | 1.5580 |
| A-0485 | 114-116 | |
| A-0486 | | 1.5362 |
| A-0487 | 126-127 | |
| A-0488 | 70-71 | |
| A-0490 | 57-60 | |
| A-0491 | 154-157 | |
| A-0492 | 98-100 | |
| A-0493 | 191-193 | |
| A-0494 | 124-126 | |
| A-0497 | 155-157 | |
| A-0498 | 124-127 | |
| A-0504 | 119-122 | |
| A-0505 | | 1.5410 |
| A-0506 | | 1.5468 |
| A-0507 | | 1.5426 |
| A-0508 | 97-100 | |
| A-0509 | 89-91 | |
| A-0510 | 69-72 | |
| A-0512 | 48-51 | |
| A-0515 | | 1.5498 |
| A-0516 | 82-84 | |
| A-0517 | 72-75 | |
| A-0518 | 78-80 | |
| A-0519 | 103-104 | |
| A-0520 | 116-117 | |
| A-0521 | 71-73 | |
| A-0522 | 68-70 | |
| A-0523 | 100-103 | |

TABLE 206

| | Physical property value | |
|---|---|---|
| Compound Number | Melting point (° C.) | Refractive index(n_{D}²⁰) |
| A-0524 | 59-61 | |
| A-0525 | 127-130 | |
| A-0527 | 72-74 | |
| A-0528 | 76-79 | |
| A-0537 | 122-124 | |
| A-0538 | 126-129 | |
| A-0539 | 100-103 | |
| A-0546 | 169-172 | |
| A-0549 | | 1.4961 |
| A-0551 | | 1.5120 |
| A-0552 | | 1.5122 |
| A-0553 | 55-57 | |
| A-0554 | 119-121 | |
| A-0557 | 128-130 | |
| A-0560 | 110-113 | |
| A-0561 | 83-86 | |
| A-0562 | 97-99 | |
| A-0563 | 141-142 | |
| A-0564 | 130-133 | |
| A-0566 | 112-115 | |
| A-0567 | 124-126 | |
| A-0569 | 114-116 | |
| A-0572 | 97-99 | |
| A-0573 | 97-100 | |
| A-0574 | 77-80 | |
| A-0575 | 140-143 | |
| A-0576 | 114-117 | |
| A-0577 | 142-145 | |
| A-0578 | 42-45 | |
| A-0580 | 81-84 | |
| A-0582 | 103-106 | |
| A-0583 | 97-100 | |
| A-0584 | 80-83 | |
| A-0585 | 85-87 | |
| A-0586 | 105-108 | |
| A-0588 | 144-147 | |
| A-0590 | | 1.5489 |
| A-0591 | 129-132 | |
| A-0592 | 106-108 | |
| A-0593 | 104-107 | |
| A-0594 | 90-93 | |

TABLE 207

| | Physical property value | |
|---|---|---|
| Compound Number | Melting point (° C.) | Refractive index (n_{D}²⁰) |
| A-0595 | 98-100 | |
| A-0596 | | 1.5295 |
| A-0597 | 61-64 | |
| A-0598 | | 1.5150 |
| A-0599 | 141-144 | |
| A-0600 | 147-149 | |
| A-0601 | 149-151 | |
| A-0602 | 96-98 | |
| A-0603 | 141-143 | |
| A-0604 | 156-159 | |
| A-0605 | 164-167 | |
| A-0608 | 165-168 | |
| A-0609 | 181-184 | |
| A-0613 | 136-139 | |
| A-0619 | 173-176 | |
| A-0622 | 107-109 | |
| A-0623 | 137-140 | |
| A-0624 | 187-189 | |
| A-0626 | 114-117 | |
| A-0651 | | 1.5093 |
| A-0652 | 60-63 | |
| A-0654 | 35-36 | |
| A-0657 | | 1.4937 |
| A-0658 | | 1.5160 |
| A-0660 | 52-55 | |
| A-0667 | 77-79 | |
| A-0706 | 131-133 | |
| A-0717 | 63-64 | |
| A-0718 | 86-89 | |
| A-0719 | | 1.5067 |
| A-0720 | | 1.5060 |
| A-0729 | 42-45 | |
| A-0734 | | 1.5085 |
| A-0736 | | 1.5070 |
| A-0739 | 87-90 | |
| A-0742 | 61-62 | |
| A-0743 | | 1.5177 |
| A-0744 | 35-37 | |
| A-0753 | 111-114 | |
| A-0754 | 121-122 | |
| A-0757 | 124-127 | |

TABLE 208

| | Physical property value | |
|---|---|---|
| Compound Number | Melting point (° C.) | Refractive index (n_{D}²⁰) |
| A-0758 | 142-144 | |
| A-0767 | 91-94 | |
| A-0774 | 50-53 | |
| A-0797 | 62-65 | |
| A-0798 | 77-80 | |
| A-0799 | 57-60 | |
| A-0800 | 65-68 | |
| A-0801 | | 1.5252 |
| A-0810 | 116-119 | |
| A-0812 | 82-84 | |
| A-0814 | | 1.4983 |
| A-0857 | 166-169 | |
| A-0859 | 159-162 | |
| A-0860 | 72-74 | |
| A-0861 | 74-76 | |
| A-0862 | 121-124 | |
| A-0863 | | 1.5121 |
| A-0864 | 55-57 | |
| A-0865 | 64-67 | |
| A-0866 | 82-84 | |
| A-0867 | 70-72 | |
| A-0868 | 62-65 | |
| A-0869 | 92-93 | |
| A-0870 | 70-73 | |
| A-0872 | | 1.5129 |
| A-0873 | 147-150 | |
| A-0874 | 143-145 | |
| A-0875 | 127-130 | |
| A-0876 | | 1.4967 |
| A-0877 | 83-86 | |
| A-0878 | 90-93 | |

TABLE 209

| Compound | | |
|---|---|---|
| Number | Physical property value ¹H-NMR data [TMS δ(ppm)] | |
| A-0023 | 300 MHz | 8.52(1H, s), 8.16-8.21(2H, m), 7.37-7.41(2H, |
| | CDCl₃ | m), 3.76-3.79(2H, m), 0.95-1.09(1H, m), 0.18- |
| | | 0.59(4H, m) |
| A-0065 | 300 MHz | 8.70(1H, s), 8.14-8.20(2H, m), 7.46-7.51(2H, |
| | CDCl₃ | m), 5.07-5.24(2H, m), 3.64-3.71(2H, m), 1.22- |
| | | 1.27(3H, m) |
| A-0550 | 300 MHz | 8.52(1H, s), 8.16-8.19(2H, m), 7.50-7.52(2H, |
| | CDCl₃ | m), 5.69(1H, s), 4.19-4.27(4H, m), 1.19- |
| | | 1.27(6H, m) |
| A-0871 | 300 MHz | 8.64-8.72(1H, m), 8.07-8.11(2H, m), 7.52- |
| | DMSO-d₆ | 7.61(2H, m), 4.11-4.15(2H, t), 3.85-3.91(2H, |
| | | m), 1.32-1.42(9H, m) |

TABLE 210

| | Physical property value | |
|---|---|---|
| Compound Number | Melting point (° C.) | Refractive index (n_{D}²⁰) |
| B-0001 | 130-133 | |
| B-0002 | 131-134 | |
| B-0003 | 154-156 | |
| B-0004 | 115-117 | |
| B-0007 | 152-155 | |
| B-0008 | 102-105 | |
| B-0009 | 96-99 | |
| B-0010 | 71-73 | |
| B-0011 | 94-96 | |
| B-0012 | 77-80 | |
| B-0013 | 82-85 | |
| B-0014 | 96-99 | |
| B-0016 | 103-106 | |
| B-0017 | 76-78 | |
| B-0018 | 97-100 | |
| B-0019 | 74-77 | |
| B-0020 | 110-112 | |
| B-0021 | 90-91 | |
| B-0025 | 82-84 | |
| B-0029 | 72-73 | |
| B-0033 | 120-123 | |
| B-0041 | 105-107 | |
| B-0042 | 54-55 | |
| B-0044 | 59-61 | |
| B-0046 | 93-96 | |
| B-0047 | 95-98 | |
| B-0049 | | 1.5708 |
| B-0050 | 76-79 | |
| B-0051 | 98-100 | |
| B-0055 | 104-105 | |
| B-0059 | 82-83 | |
| B-0063 | 123-126 | |
| B-0067 | 77-80 | |
| B-0072 | 55-57 | |
| B-0076 | | 1.5672 |
| B-0077 | | 1.5671 |
| B-0088 | 100-103 | |
| B-0104 | 62-65 | |
| B-0127 | 74-77 | |
| B-0133 | 85-86 | |
| B-0135 | 116-119 | |

TABLE 211

| | Physical property value | |
|---|---|---|
| Compound Number | Melting point (° C.) | Refractive index (n_{D}²⁰) |
| B-0143 | 132-134 | |
| B-0150 | 118-120 | |
| B-0189 | 63-66 | |
| B-0214 | | 1.5729 |
| B-0237 | 68-70 | |
| B-0260 | 65-68 | |
| B-0310 | | 1.4834 |
| B-0316 | 75-77 | |
| B-0341 | 73-74 | |
| B-0359 | 89-92 | |
| B-0375 | 81-84 | |
| B-0377 | 80-82 | |
| B-0418 | 106-109 | |
| B-0436 | 116-118 | |
| B-0454 | 107-109 | |
| B-0470 | 114-117 | |
| B-0488 | 87-90 | |
| B-0508 | 105-107 | |
| B-0531 | 133-135 | |
| B-0533 | 84-85 | |
| B-0549 | 68-71 | |
| B-0565 | | 1.5783 |
| B-0569 | 83-86 | |
| B-0619 | | 1.5766 |
| B-0630 | 87-89 | |
| B-0646 | 45-46 | |
| B-0662 | 103-104 | |
| B-0678 | 74-75 | |
| B-0744 | 117-120 | |
| B-0751 | 78-80 | |
| B-0762 | 161-164 | |
| B-0770 | 65-68 | |
| B-0779 | 51-53 | |
| B-0782 | 75-77 | |
| B-0794 | | 1.4898 |
| B-0797 | | 1.5240 |
| B-0810 | | 1.5273 |
| B-0812 | | 1.5280 |
| B-0814 | 122-125 | |
| B-0823 | | 1.4781 |
| B-0835 | 50-52 | |

TABLE 212

| | Physical property value | |
|---|---|---|
| Compound Number | Melting point (° C.) | Refractive index (n_{D}²⁰) |
| B-0837 | 73-75 | |
| B-0838 | 57-60 | |
| B-0850 | 86-88 | |
| B-0851 | 71-74 | |
| B-0852 | 114-116 | |

TABLE 213

| Compound | | |
|---|---|---|
| Number | Physical property value ¹H-NMR data [TMS δ(ppm)] | |
| B-0048 | 300 MHz | 8.68-8.91(1H, m), 8.30-8.32(1H, dd), 8.01- |
| | DMSO-d₆ | 8.08(2H, m), 7.71-7.78(1H, dd), 7.62-7.67(2H, |
| | | m), 7.33-7.40(1H, dd), 5.12-5.19(2H, m) |

TABLE 214

| | Physical property value | |
|---|---|---|
| Compound Number | Melting point (° C.) | Refractive index (n_{D}²⁰) |
| C-0001 | 203-206 | |
| C-0002 | 76-79 | |
| C-0003 | 65-67 | |
| C-0004 | 68-70 | |
| C-0038 | 102-104 | |
| C-0067 | 55-56 | |
| C-0080 | 50-51 | |
| C-0081 | 37-39 | |
| C-0096 | | 1.6172 |
| C-0156 | 85-87 | |
| C-0157 | 91-94 | |
| C-0162 | 122-123 | |
| C-0163 | | 1.6313 |
| C-0164 | 122-123 | |
| C-0166 | 90-93 | |
| C-0167 | 142-143 | |
| C-0216 | 88-90 | |
| C-0218 | | 1.5703 |
| C-0219 | 90-92 | |
| C-0281 | 102-104 | |
| C-0355 | | 1.5981 |
| C-0356 | | 1.5945 |
| C-0490 | | 1.6319 |
| C-0504 | | 1.5250 |
| C-0517 | 83-88 | |
| C-0549 | | 1.5364 |
| C-0552 | 68-69 | |
| C-0554 | 116-118 | |
| C-0557 | 132-134 | |
| C-0563 | 152-155 | |
| C-0564 | 107-109 | |
| C-0751 | 82-85 | |

TABLE 215

| Compound | | |
|---|---|---|
| Number | Physical property value ¹H-NMR data [TMS δ(ppm)] | |
| C-0011 | 300 MHz | 9.69(1H, s), 8.14-8.27(2H, m), 7.37-7.42(2H, |
| | CDCl₃ | m), 4.33-4.38(2H, m), 0.85-1.76(9H, m) |
| C-0525 | 300 MHz | 9.81(1H, s), 8.15-8.20(2H, m), 7.38-7.74(2H, |
| | CDCl₃ | m), 5.26(2H, s), 2.05-2.09(1H, m), 1.17- |
| | | 1.20(2H, m), 1.02-1.06(2H, m) |

TABLE 216

| | Physical property value | |
|---|---|---|
| Compound Number | Melting point (° C.) | Refractive index (n_{D}²⁰) |
| D-0002 | 128-131 | |
| D-0003 | 140-143 | |
| D-0008 | | 1.6193 |
| D-0010 | 75-78 | |
| D-0011 | 92-93 | |
| D-0012 | 97-99 | |
| D-0017 | 92-94 | |
| D-0104 | | 1.6154 |
| D-0135 | 119-121 | |
| D-0508 | 102-104 | |
| D-0531 | 107-110 | |
| D-0662 | | 1.5859 |
| D-0838 | 112-114 | |

TABLE 217

| Compound | | |
|---|---|---|
| Number | Physical property value ¹H-NMR data [TMS δ(ppm)] | |
| D-0569 | 300 MHz | 9.68(1H, s), 8.19-8.22(2H, m), 7.71(1H, |
| | CDCl₃ | s), 7.35-7.38(2H, m), 5.43-5.66(2H, m) |

TABLE 218

| | Physical property value | |
|---|---|---|
| Compound Number | Melting point (° C.) | Refractive index (n_{D}²⁰) |
| E-0001 | | 1.5421 |
| E-0199 | | 1.5455 |
| E-0397 | | 1.5793 |
| E-0496 | 121-122 | |
| E-0595 | 88-90 | |
| E-0793 | 127-129 | |
| E-1156 | | 1.5847 |

TABLE 219

| | Physical property value | |
|---|---|---|
| Compound Number | Melting point (° C.) | Refractive index (n_{D}²⁰) |
| F-0025 | 63-66 | |
| F-0031 | 94-97 | |
| F-0038 | 132-134 | |
| F-0059 | | 1.6242 |
| F-0074 | 86-88 | |
| F-0114 | 107-108 | |
| F-0115 | 59-62 | |
| F-0116 | 80-82 | |
| F-0117 | | 1.5873 |
| F-0118 | 59-60 | |
| F-0119 | 93-95 | |
| F-0126 | 80-82 | |
| F-0127 | | 1.5750 |
| F-0128 | 98-100 | |
| F-0162 | 99-102 | |
| F-0163 | 62-64 | |
| F-0164 | 53-55 | |
| F-0177 | 41-44 | |
| F-0178 | | 1.5547 |
| F-0200 | 153-156 | |
| F-0213 | 85-88 | |
| F-0241 | 89-91 | |
| F-0274 | 124-126 | |
| F-0292 | 106-107 | |
| F-0309 | 70-72 | |
| F-0313 | 79-82 | |
| F-0491 | 107-108 | |
| F-0650 | | 1.5512 |

TABLE 220

| Compound | | |
|---|---|---|
| Number | Physical property value ¹H-NMR data [TMS δ(ppm)] | |
| F-0497 | 300 MHz | 7.89-7.95(2H, d), 6.48-6.76(2H, m), 4.23- |
| | CDCl₃ | 4.30(1H, m), 3.75-3.94(2H, m), 0.72-0.79(4H, |
| | | m) |

TABLE 221

| | Physical property value | |
|---|---|---|
| Compound Number | Melting point (° C.) | Refractive index (n_{D}²⁰) |
| G-0004 | 127-130 | |
| G-0007 | 178-180 | |
| G-0012 | 122-125 | |
| G-0017 | 96-99 | |
| G-0025 | 96-98 | |
| G-0042 | 127-129 | |
| G-0072 | 137-139 | |
| G-0135 | 107-109 | |
| G-0237 | 43-45 | |
| G-0310 | 104-106 | |
| G-0339 | 108-110 | |
| G-0533 | 77-80 | |
| G-0710 | 72-73 | |
| G-0838 | 60-62 | |

TABLE 222

| Compound | | |
|---|---|---|
| Number | Physical property value ¹H-NMR data [TMS δ(ppm)] | |
| G-0850 | 300 MHz | 7.91-7.94(2H, m), 7.67-7.74(1H, t), 7.34- |
| | CDCl₃ | 7.36(1H, d), 7.21-7.23(1H, d), 6.73- |
| | | 6.76(2H, m), 5.38-5.44(1H, m), 4.62(2H, |
| | | s), 4.50-4.52(2H, d), 3.51(3H, s) |

TABLE 223

| | Physical property value | |
|---|---|---|
| Compound Number | Melting point (° C.) | Refractive index (n_{D}²⁰) |
| H-0032 | 125-128 | |
| H-0038 | 110-113 | |
| H-0045 | | 1.5950 |
| H-0067 | 90-93 | |
| H-0096 | 50-53 | |
| H-0156 | 150-153 | |
| H-0157 | 121-124 | |
| H-0158 | 153-155 | |
| H-0159 | 177-180 | |
| H-0160 | 147-148 | |
| H-0161 | 134-137 | |
| H-0168 | 195-198 | |
| H-0169 | 129-130 | |
| H-0170 | 132-135 | |
| H-0204 | 183-186 | |
| H-0205 | 104-107 | |
| H-0206 | 119-122 | |
| H-0219 | 130-133 | |
| H-0220 | 132-135 | |
| H-0255 | 140-143 | |
| H-0257 | 152-155 | |
| H-0283 | 107-110 | |
| H-0316 | 145-148 | |
| H-0351 | 166-169 | |
| H-0355 | 134-137 | |
| H-0495 | 164-167 | |
| H-0496 | 167-170 | |
| H-0527 | 130-133 | |
| H-0530 | 120-122 | |
| H-0533 | 130-133 | |
| H-0537 | 165-168 | |
| H-0539 | 186-189 | |
| H-0602 | 165-168 | |
| H-0682 | 197-200 | |

TABLE 224

| | Physical property value | |
|---|---|---|
| Compound Number | Melting point (° C.) | Refractive index (n_{D}²⁰) |
| I-0012 | 168-170 | |
| I-0013 | 151-154 | |
| I-0017 | 129-131 | |
| I-0042 | 190-193 | |
| I-0072 | 171-174 | |
| I-0237 | 136-139 | |
| I-0678 | 139-142 | |
| I-0810 | 148-150 | |
| I-0838 | 86-89 | |
| I-0855 | 230-233 | |

TABLE 225

| Compound | | |
|---|---|---|
| Number | Physical property value ¹H-NMR data [TMS δ(ppm)] | |
| I-0025 | 300 MHz | 9.62-9.65(1H, m), 8.58-8.75(1H, m), 7.58- |
| | DMSO-d₆ | 7.67(3H, m), 7.38-7.41(2H, m), 7.02-7.11(2H, |
| | | m), 5.77-5.82(2H, m), 5.03-5.08(2H, m), |
| | | 2.24(s, 3H) |
| I-0812 | 300 MHz | 9.67-9.68(1H, m), 8.36-8.52(1H, m), 7.69- |
| | DMSO-d₆ | 7.74(2H, m), 7.36-7.41(2H, m), 5.86(2H, |
| | | s), 3.36-3.91(5H, m), 3.30-3.36(1H, m), |
| | | 2.27-2.36(1H, m), 1.73-1.87(1H, m), 1.44- |
| | | 1.55(1H, m) |

Next, Formulation Examples of the agricultural and horticultural plant disease control agent of the present invention using the formamide derivative of the present invention produced as described above, or the agriculturally acceptable salt thereof will be specifically described. It is to be noted that the compound, the type and mixing ratios of compounds and additives are not limited thereto, and can be changed in a wide range. In the following description, “part(s)” mean part(s) by mass.

[Formulation Example 1] Emulsifiable concentrate

| | | |
|---|---|---|
| Each compound described in Tables 1 to 225 | 10 | parts |
| Cyclohexanone | 30 | parts |
| Polyoxyethylene alkyl aryl ether | 11 | parts |
| Calcium alkylbenzenesulfonate | 4 | parts |
| Methylnaphthalene | 45 | parts |

The above components were uniformly dissolved to obtain an emulsifiable concentrate.

[Formulation Example 2] Wettable powder

| | | |
|---|---|---|
| Each compound described in Tables 1 to 225 | 10 | parts |
| Naphthalenesulfonic acid formalin condensate sodium salt | 0.5 | parts |
| Polyoxyethylene alkyl aryl ether | 0.5 | parts |
| Diatomaceous earth | 24 | parts |
| Clay | 65 | parts |

The above components were uniformly mixed and pulverized to obtain a wettable powder.

[Formulation Example 3] Powder

| | | |
|---|---|---|
| Each compound described in Tables 1 to 225 | 2 | parts |
| Diatomaceous earth | 5 | parts |
| Clay | 93 | parts |

The above components were uniformly mixed and pulverized to obtain a powder.

[Formulation Example 4] Granules

| | | |
|---|---|---|
| Each compound described in Tables 1 to 225 | 5 | parts |
| Sodium salt of lauryl alcohol sulfuric acid ester | 2 | parts |
| Sodium ligninsulfonate | 5 | parts |
| Carboxymethylcellulose | 2 | parts |
| Clay | 86 | parts |

The above components were uniformly mixed and pulverized. An amount corresponding to 20 parts of water was added to the mixture. The mixture was kneaded and processed into a granular shape of 14 to 32 mesh using an extrusion granulator, followed by drying to obtain granules.

[Formulation Example 5] Flowable concentrate

| | | |
|---|---|---|
| Each compound described in Tables 1 to 225 | 20 | parts |
| Polyoxyethylene styrenated phenyl ether sulfate | 4 | parts |
| Ethylene glycol | 7 | parts |
| Silicone AF-118N (manufactured by Asahi Kasei | 0.02 | parts |
| Corporation) | | |
| Water | 68.98 | parts |

The above components were mixed for 30 minutes with a high-speed stirrer, and pulverized with a wet pulverizer to obtain a flowable concentrate.

[Formulation Example 6] Wettable granules

| | | |
|---|---|---|
| Each compound described in Tables 1 to 225 | 10 | parts |
| Sodium ligninsulfonate | 5 | parts |
| Polyoxyethylene alkyl aryl ether | 1 | part |
| Sodium polycarboxylate | 3 | parts |
| White carbon | 5 | parts |
| Pre-gelatinized starch | 1 | part |
| Calcium carbonate | 65 | parts |
| Water | 10 | parts |

The above components were mixed, kneaded, and pressed to obtain a granule. The obtained granule was dried in a fluidized bed drier to obtain wettable granules.

Next, the effect exhibited by the disease control agent of the present invention will be described with reference to Test Examples.

### [Test Example 1] Soybean Rust (Phakopsora pachyrhizi) Control Effect Test

Soybean seeds were sown in a pot and sprayed on the grown seedlings so that the amount of the active ingredient was 100 g a.i./ha. Thereafter, a soybean rust fungus was spray-inoculated, the pot was transferred to a wet room and was kept under dark conditions, and invasion of pathogens was promoted. Thereafter, the pot was managed in a greenhouse, after 12 to 14 days, a degree of disease severity for each first leaf was examined according to the criteria in Table 226 to determine a degree of disease severity by a calculation formula of Equation 1, and a control value was determined by a calculation formula of Equation 2.

TABLE 226
Disease severity index

| | |
|---|---|
| 0: | The infection was not observed. |
| 1: | The infection area was less than 25% of the area of the leaf. |
| 2: | The infection area was 25% or more and less than 50% of the |
| | area of the leaf. |
| 3: | The infection area was 50% or more and less than 75% of the |
| | area of the leaf. |
| 4: | The infection area was 75% or more of the area of the leaf. |

$$\begin{matrix}Degree of \\ disease severity\end{matrix} = \frac{n 0 \times 0 + n 1 \times 1 + n 2 \times 2 + n 3 \times 3 + n 4 \times 4}{4 N} \times 100 \qquad [ Equation 1 ]$$

[in the formula, N represents the total number of examined leaves, n0 represents the number of leaves having a disease severity index of 0, n1 represents the number of leaves having a disease severity index of 1, n2 represents the number of leaves having a disease severity index of 2, n3 represents the number of leaves having a disease severity index of 3, and n4 represents the number of leaves having a disease severity index of 4.]

$$\begin{matrix}Control \\ value\end{matrix} = ( 1 - \frac{\begin{matrix}Degree of \\ disease \\ severity of \\ treated \\ section\end{matrix}}{\begin{matrix}Degree of \\ disease \\ severity of \\ untreated \\ section\end{matrix}} \times 100 ) \qquad [ Equation 2 ]$$

The comparative compound in the table is the compound 7 of the present invention described in Table 1 of [0060] of WO 2017/110862 A, and has the following structure.

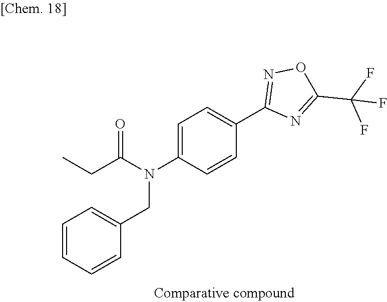 [SMILES: CCC(=O)N(Cc1ccccc1)c1ccc(-c2noc(C(F)(F)F)n2)cc1]

As a result of the test, the control value of the comparative compound was less than 60, the control value of each of A-0002, A-0006, A-0011, A-0014, A-0026, A-0064, A-0098, A-0103, A-0151, A-0216, A-0256, A-0315, A-0483, A-0486, A-0490, A-0494, A-0515, A-0549, A-0553, A-0567, A-0574, A-0575, A-0577, A-0585, A-0593, A-0594, A-0601, A-0608, A-0609, A-0652, A-0657, A-0667, A-0706, A-0717, A-0739, A-0758, A-0774, A-0814, A-0859, A-0862, A-0870, A-0871, A-0874, A-0876, A-0877, A-0921, B-0003, B-0004, B-0009, B-0067, B-0762, B-0782, B-0823, B-0835, B-0837, B-0852, C-0080, C-0163, C-0552, D-0012, E-0199, and G-0025 was 60 or more and less than 80, and the control value of each of A-0001, A-0007, A-0018, A-0027, A-0029, A-0032, A-0034, A-0035, A-0038, A-0039, A-0041, A-0042, A-0045, A-0057, A-0065, A-0066, A-0067, A-0072, A-0074, A-0075, A-0082, A-0095, A-0096, A-0097, A-0099, A-0107, A-0144, A-0156, A-0157, A-0158, A-0159, A-0160, A-0161, A-0168, A-0169, A-0170, A-0204, A-0205, A-0206, A-0219, A-0220, A-0240, A-0241, A-0242, A-0259, A-0260, A-0279, A-0282, A-0283, A-0316, A-0334, A-0351, A-0352, A-0353, A-0354, A-0355, A-0356, A-0482, A-0484, A-0491, A-0492, A-0497, A-0498, A-0505, A-0506, A-0507, A-0508, A-0512, A-0516, A-0517, A-0518, A-0519, A-0520, A-0521, A-0522, A-0523, A-0524, A-0525, A-0527, A-0528, A-0537, A-0539, A-0550, A-0551, A-0552, A-0554, A-0557, A-0560, A-0561, A-0563, A-0566, A-0569, A-0573, A-0592, A-0595, A-0596, A-0597, A-0598, A-0599, A-0600, A-0602, A-0603, A-0604, A-0613, A-0619, A-0622, A-0623, A-0626, A-0651, A-0654, A-0658, A-0660, A-0718, A-0719, A-0720, A-0729, A-0734, A-0736, A-0744, A-0753, A-0754, A-0757, A-0767, A-0797, A-0798, A-0799, A-0800, A-0801, A-0810, A-0812, A-0860, A-0861, A-0864, A-0867, A-0872, A-0873, B-0008, B-0011, B-0012, B-0013, B-0014, B-0016, B-0017, B-0018, B-0019, B-0020, B-0021, B-0025, B-0029, B-0033, B-0042, B-0044, B-0046, B-0047, B-0048, B-0049, B-0050, B-0051, B-0055, B-0059, B-0063, B-0072, B-0076, B-0077, B-0088, B-0104, B-0127, B-0133, B-0135, B-0143, B-0150, B-0189, B-0214, B-0237, B-0260, B-0310, B-0316, B-0341, B-0359, B-0375, B-0377, B-0418, B-0436, B-0454, B-0470, B-0488, B-0508, B-0531, B-0533, B-0549, B-0565, B-0569, B-0630, B-0646, B-0662, B-0678, B-0744, B-0751, B-0770, B-0779, B-0794, B-0797, B-0810, B-0812, B-0814, B-0838, B-0850, B-0851, C-0001, C-0038, C-0067, C-0096, C-0156, C-0157, C-0219, C-0355, C-0517, C-0525, C-0549, C-0554, C-0557, C-0563, C-0564, D-0008, D-0017, D-0104, D-0135, D-0508, D-0531, D-0662, D-0838, E-0397, E-0496, E-0595, E-0793, F-0038, G-0533, and G-0710 was 80 or more.

[Test Example 2] Wheat rust (Puccinia recondita) control effect test Wheat seeds were sown in a pot, and a chemical solution was sprayed on the grown seedlings so that the concentration of the active ingredient was 50 ppm. Thereafter, a wheat rust fungus was spray-inoculated, the pot was transferred to a wet room and was kept under dark conditions, and invasion of pathogens was promoted. Thereafter, the pot was managed in a greenhouse, after 9 days, a degree of disease severity for each first leaf was examined according to the criteria in Table 220 to determine a degree of disease severity by a calculation formula of Equation 1, and a control value was determined by a calculation formula of Equation Formula 2.

TABLE 227
Disease severity index

| | |
|---|---|
| 0: | The infection was not observed. |
| 1: | The infection area was less than 5% of the area of the leaf. |
| 2: | The infection area was 5% or more and less than 33% of the |
| | area of the leaf. |
| 3: | The infection area was 33% or more and less than 67% of the |
| | area of the leaf. |
| 4: | The infection area was 67% or more of the area of the leaf. |

As a result of the test, the control value of the comparative compound was 12.5 or less, the control value of each of A-0002, A-0080, A-0103, A-0104, A-0105, A-0162, A-0165, A-0204, A-0216, A-0221, A-0255, A-0256, A-0257, A-0260, A-0280, A-0283, A-0284, A-0316, A-0317, A-0335, A-0351, A-0352, A-0481, A-0487, A-0504, A-0507, A-0510, A-0523, A-0575, A-0576, A-0577, A-0582, A-0584, A-0623, A-0624, A-0706, B-0007, B-0619, B-0751, C-0167, C-0219, C-0281, C-0355, D-0002, D-0011, F-0025, F-0059, F-0119, F-0164, F-0177, F-0292, F-0313, G-0004, and G-0838 was more than 12.5 and 50 or less, the control value of each of A-0026, A-0027, A-0029, A-0035, A-0039, A-0081, A-0095, A-0098, A-0099, A-0144, A-0151, A-0159, A-0163, A-0166, A-0167, A-0168, A-0219, A-0220, A-0279, A-0281, A-0282, A-0334, A-0354, A-0355, A-0483, A-0490, A-0492, A-0493, A-0505, A-0506, A-0522, A-0524, A-0537, A-0538, A-0550, A-0566, A-0569, A-0574, A-0583, A-0588, A-0591, A-0592, A-0593, A-0594, A-0605, A-0608, A-0609, A-0622, A-0626, A-0667, A-0797, A-0801, A-0859, A-0860, A-0864, B-0002, B-0003, B-0009, B-0011, B-0018, B-0048, B-0049, B-0454, B-0569, B-0782, C-0011, C-0081, C-0552, C-0751, D-0010, G-0017, G-0310, and G-0850 was more than 50 and 75 or less, and the control value of each of A-0001, A-0003, A-0004, A-0006, A-0007, A-0011, A-0012, A-0014, A-0018, A-0023, A-0032, A-0034, A-0038, A-0041, A-0042, A-0045, A-0057, A-0065, A-0066, A-0067, A-0072, A-0074, A-0075, A-0082, A-0096, A-0097, A-0156, A-0157, A-0158, A-0164, A-0169, A-0170, A-0205, A-0206, A-0240, A-0241, A-0242, A-0259, A-0315, A-0353, A-0356, A-0482, A-0484, A-0485, A-0491, A-0494, A-0497, A-0498, A-0508, A-0509, A-0512, A-0515, A-0516, A-0517, A-0518, A-0519, A-0520, A-0521, A-0525, A-0527, A-0528, A-0539, A-0551, A-0552, A-0553, A-0554, A-0557, A-0560, A-0561, A-0562, A-0564, A-0567, A-0572, A-0573, A-0585, A-0586, A-0590, A-0595, A-0598, A-0599, A-0604, A-0613, A-0619, A-0651, A-0652, A-0654, A-0657, A-0658, A-0660, A-0717, A-0718, A-0719, A-0720, A-0729, A-0734, A-0736, A-0739, A-0742, A-0743, A-0744, A-0753, A-0754, A-0767, A-0774, A-0798, A-0799, A-0800, A-0812, A-0814, A-0862, A-0875, A-0876, A-0921, B-0004, B-0012, B-0013, B-0014, B-0016, B-0017, B-0019, B-0020, B-0021, B-0025, B-0033, B-0041, B-0042, B-0044, B-0046, B-0047, B-0050, B-0051, B-0055, B-0059, B-0063, B-0067, B-0072, B-0076, B-0077, B-0088, B-0104, B-0127, B-0133, B-0135, B-0143, B-0150, B-0189, B-0214, B-0237, B-0310, B-0316, B-0341, B-0359, B-0375, B-0377, B-0418, B-0436, B-0470, B-0488, B-0508, B-0531, B-0533, B-0549, B-0565, B-0630, B-0646, B-0662, B-0678, B-0744, B-0762, B-0770, B-0794, B-0797, B-0810, B-0812, B-0814, B-0823, B-0837, B-0838, B-0850, B-0851, B-0852, C-0001, C-0002, C-0003, C-0004, C-0038, C-0067, C-0080, C-0156, C-0157, C-0517, C-0525, C-0549, C-0554, C-0557, C-0564, D-0012, D-0017, D-0104, D-0135, D-0508, D-0531, D-0662, D-0838, E-0397, G-0012, G-0135, G-0533, and G-0710 was more than 75.

### [Test Example 3] Rice Sheath Blight Disease (Rhizoctonia solani) Control Effect Test

Rice seeds were sown in a pot, and a chemical solution was sprayed on the grown seedlings so that the concentration of the active ingredient was 50 ppm. Thereafter, a bran medium in which a rice sheath blight fungi was cultured was inoculated into the strain, the bran medium was transferred to a wet room, and invasion of pathogens was promoted. Thereafter, the pot was managed in a greenhouse, after 5 days, a lesion height was examined according to the criteria in Table 221 to determine a degree of disease severity by a calculation formula of Equation 1, and a control value was determined by a calculation formula of Equation 2.

TABLE 228
Disease severity index

| | |
|---|---|
| 0: | The infection was not observed. |
| 1: | The lesion height was less than 25% of that of the |
| | untreated section. |
| 2: | The lesion height was 25% or more and less than 50% |
| | of that of the untreated section. |
| 3: | The lesion height was 50% or more and less than 75% |
| | of that of the untreated section. |
| 4: | The lesion height was 75% or more of that of the |
| | untreated section. |

As a result of the test, the control value of the comparative compound was 12.5 or less, the control value of each of A-0041, A-0042, A-0072, A-0151, A-0156, A-0204, A-0217, A-0279, A-0315, A-0316, A-0317, A-0334, A-0352, A-0353, A-0484, A-0490, A-0505, A-0508, A-0562, A-0564, A-0567, A-0572, A-0575, A-0591, A-0592, A-0597, A-0605, A-0667, A-0706, A-0718, A-0743, A-0799, A-0864, A-0872, A-0876, A-0921, B-0003, B-0011, B-0044, B-0046, B-0048, B-0049, B-0050, B-0310, B-0454, B-0569, B-0770, B-0814, B-0838, C-0038, C-0525, E-0199, E-0595, E-1156, F-0038, F-0491, H-0169, H-0351, and I-0678 was more than 12.5 and 50 or less, the control value of each of A-0075, A-0081, A-0097, A-0159, A-0168, A-0220, A-0280, A-0284, A-0491, A-0497, A-0507, A-0518, A-0522, A-0537, A-0561, A-0578, A-0582, A-0593, A-0594, A-0598, A-0622, A-0623, A-0624, A-0658, A-0729, A-0742, A-0744, A-0757, A-0798, A-0801, A-0810, A-0860, A-0862, A-0875, B-0020, B-0051, B-0104, B-0127, B-0143, B-0359, B-0549, B-0565, B-0852, C-0156, C-0355, C-0557, D-0011, D-0662, F-0213, G-0042, and 1-0042 was more than 50 and 75 or less, and the control value of each of A-0035, A-0080, A-0096, A-0098, A-0144, A-0157, A-0158, A-0160, A-0169, A-0170, A-0205, A-0206, A-0240, A-0241, A-0256, A-0259, A-0283, A-0335, A-0355, A-0356, A-0492, A-0498, A-0509, A-0517, A-0519, A-0520, A-0523, A-0525, A-0527, A-0528, A-0554, A-0557, A-0566, A-0569, A-0595, A-0599, A-0600, A-0603, A-0608, A-0609, A-0619, A-0758, A-0767, A-0774, A-0859, A-0870, A-0873, A-0874, B-0004, B-0009, B-0012, B-0014, B-0016, B-0017, B-0018, B-0019, B-0021, B-0025, B-0041, B-0047, B-0055, B-0059, B-0067, B-0076, B-0077, B-0088, B-0135, B-0150, B-0237, B-0341, B-0375, B-0418, B-0533, B-0762, B-0850, B-0851, C-0281, C-0554, C-0564, D-0012, D-0017, D-0135, D-0531, E-0496, and E-0793 was more than 75.

### INDUSTRIAL APPLICABILITY

The present invention provides a novel compound having excellent bactericidal activity and a production intermediate thereof, is useful in pesticide fields and agricultural fields, and has industrial applicability.

LENGTHY TABLES
The patent application contains a lengthy table section. A copy of the table is available in electronic form from the USPTO web site (https://seqdata.uspto.gov/?pageRequest=docDetail&DocID=US20240358020A1). An electronic copy of the table will also be available from the USPTO upon request and payment of the fee set forth in 37 CFR 1.19(b)(3).

1. A formamide derivative of formula [I] or an agriculturally acceptable salt thereof, 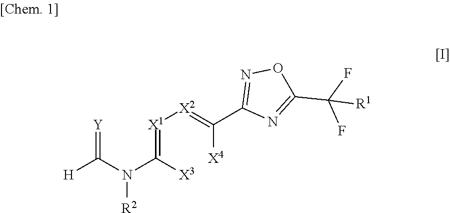
wherein in the formula [I],
X¹, X², X³, and X⁴ each independently represent a carbon atom (the carbon atom is optionally substituted with R³) or a nitrogen atom,
Y represents an oxygen atom or a sulfur atom,
R¹ represents a hydrogen atom or a halogen atom,
R² represents a hydrogen atom, a (C1-C6)alkyl (the (C1-C6)alkyl is optionally mono-substituted or poly-substituted with R⁴), a (C2-C6)alkenyl (the (C2-C6)alkenyl is optionally mono-substituted or poly-substituted with R⁴), a (C2-C6)alkynyl (the (C2-C6)alkynyl is optionally mono-substituted or poly-substituted with R⁴), a (C3-C6)cycloalkyl (the (C3-C6)cycloalkyl is optionally mono-substituted or poly-substituted with R⁴), a (C6-C12)aryl (the (C6-C12)aryl is optionally mono-substituted or poly-substituted with R⁵), a 3- to 6-membered heterocyclyl (the 3- to 6-membered heterocyclyl is optionally mono-substituted or poly-substituted with R⁵), or a 5- to 12-membered heteroaryl (the 5- to 12-membered heteroaryl is optionally mono-substituted or poly-substituted with R⁵),
R³ each independently represent a hydrogen atom, a halogen atom, or a (C1-C6)alkyl,
R⁴ each independently represent a hydrogen atom, a halogen atom, a cyano, a thiocyanato, a thiol, a hydroxy, an amino, a tri((C1-C6)alkyl)silyl, a (C3-C6)cycloalkyl, a (C3-C6)halocycloalkyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6)cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryl (the (C6-C12)aryl is optionally mono-substituted or poly-substituted with R⁵), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyl (the 3- to 6-membered heterocyclyl is optionally mono-substituted or poly-substituted with R⁵), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryl (the 5- to 12-membered heteroaryl is optionally mono-substituted or poly-substituted with R⁵), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a 1,3-benzodioxole (the 1,3-benzodioxole is optionally mono-substituted or poly-substituted with R⁵), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the(C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl,
R⁵ each independently represent a hydrogen atom, a halogen atom, a cyano, a nitro, an amino, a hydroxy, a thiol, a formyl, an oxo, a (C1-C6)alkyl, a (C2-C6)alkenyl, a (C2-C6)alkynyl, a (C3-C6)cycloalkyl, a (C1-C6)haloalkyl, a (C1-C6)alkoxy, a (C1-C6)haloalkoxy, a (C1-C6)alkoxy (C1-C6)alkyl, a (C1-C6)haloalkoxy (C1-C6)alkyl, a cyano (C1-C6)alkyl, a cyano (C3-C6)cycloalkyl, a (C1-C6)alkylthio, a (C1-C6)haloalkylthio, a (C1-C6)alkylsulfinyl, a (C1-C6)haloalkylsulfinyl, a (C1-C6)alkylsulfonyl, a (C1-C6)haloalkylsulfonyl, a (C1-C6)alkylcarbonylthio, a (C1-C6)alkylamino (the amino moiety is optionally substituted with R⁶), a (C1-C6)haloalkylamino (the amino moiety is optionally substituted with R⁶), a formylamino (the amino moiety is optionally substituted with R⁶), a (C1-C6)alkylcarbonylamino (the amino moiety is optionally substituted with R⁶), a carbamoyl, a (C1-C6)alkylaminocarbonyl (the amino moiety is optionally substituted with R⁶), a carboxyl, a (C1-C6)alkylcarbonyl, a (C1-C6)haloalkylcarbonyl, a (C1-C6)alkoxycarbonyl, a hydroxyimino (C1-C6)alkyl, a (C1-C6)alkoxyimino (C1-C6)alkyl, a (C6-C12)aryl (the (C6-C12)aryl is optionally mono-substituted or poly-substituted with R⁷), a (C6-C12)aryl (C1-C6)alkyl (the (C6-C12)aryl moiety is optionally mono-substituted or poly-substituted with R⁷), a (C6-C12)arylcarbonyl (the (C6-C12)arylcarbonyl is optionally mono-substituted or poly-substituted with R⁷), a (C6-C12)aryloxy (the (C6-C12)aryloxy is optionally mono-substituted or poly-substituted with R⁷), a (C6-C12)arylthio (the (C6-C12)arylthio is optionally mono-substituted or poly-substituted with R⁷), a (C6-C12)arylsulfinyl (the (C6-C12)arylsulfinyl is optionally mono-substituted or poly-substituted with R⁷), a (C6-C12)arylsulfonyl (the (C6-C12)arylsulfonyl is optionally mono-substituted or poly-substituted with R⁷), a (C6-C12)arylamino (the (C6-C12)aryl moiety is optionally mono-substituted or poly-substituted with R⁷, and the amino moiety is optionally substituted with R⁶), a 3- to 6-membered heterocyclyl (the 3- to 6-membered heterocyclyl is optionally mono-substituted or poly-substituted with R⁷), a 3- to 6-membered heterocyclyl (C1-C6)alkyl (the 3- to 6-membered heterocyclyl moiety is optionally mono-substituted or poly-substituted with R⁷), a 3- to 6-membered heterocyclylcarbonyl (the 3- to 6-membered heterocyclylcarbonyl is optionally mono-substituted or poly-substituted with R⁷), a 3- to 6-membered heterocyclyloxy (the 3- to 6-membered heterocyclyloxy is optionally mono-substituted or poly-substituted with R⁷), a 3- to 6-membered heterocyclylthio (the 3- to 6-membered heterocyclylthio is optionally mono-substituted or poly-substituted with R⁷), a 3- to 6-membered heterocyclylsulfinyl (the 3- to 6-membered heterocyclylsulfinyl is optionally mono-substituted or poly-substituted with R⁷), 3- to 6-membered heterocyclylsulfonyl (the 3- to 6-membered heterocyclylsulfonyl is optionally mono-substituted or poly-substituted with R⁷), a 3- to 6-membered heterocyclylamino (the 3- to 6-membered heterocyclyl moiety is optionally mono-substituted or poly-substituted with R⁷, and the amino moiety is optionally substituted with R⁶), a 5- to 12-membered heteroaryl (the 5- to 12-membered heteroaryl is optionally mono-substituted or poly-substituted with R⁷), a 5- to 12-membered heteroaryl (C1-C6)alkyl (the 5- to 12-membered heteroaryl moiety is optionally mono-substituted or poly-substituted with R⁷), a 5- to 12-membered heteroarylcarbonyl (the 5- to 12-membered heteroarylcarbonyl is optionally mono-substituted or poly-substituted with R⁷), a 5- to 12-membered heteroaryloxy (the 5- to 12-membered heteroaryloxy is optionally mono-substituted or poly-substituted with R⁷), a 5- to 12-membered heteroarylthio (the 5- to 12-membered heteroarylthio is optionally mono-substituted or poly-substituted with R⁷), a 5- to 12-membered heteroarylsulfinyl (the 5- to 12-membered heteroarylsulfinyl is optionally mono-substituted or poly-substituted with R⁷), a 5- to 12-membered heteroarylsulfonyl (the 5- to 12-membered heteroarylsulfonyl is optionally mono-substituted or poly-substituted with R⁷), or a 5- to 12-membered heteroarylamino (the 5- to 12-membered heteroaryl moiety is optionally mono-substituted or poly-substituted with R⁷, and the amino moiety is optionally substituted with R⁶),
R⁶ each independently represent a hydrogen atom, a (C1-C6)alkyl, a (C1-C6)haloalkyl, a (C3-C6)cycloalkyl, a (C1-C6)alkoxy (C1-C6)alkyl, a (C1-C6)haloalkoxy (C1-C6)alkyl, a cyano (C1-C6)alkyl, a (C1-C6)alkylcarbonyl, a (C1-C6)haloalkylcarbonyl, a (C1-C6)alkoxycarbonyl, or a (C6-C12)arylcarbonyl (the (C6-C12)arylcarbonyl is optionally mono-substituted or poly-substituted with R⁷),
R⁷ each independently represent a hydrogen atom, a halogen atom, a cyano, a (C1-C6)alkyl, or a (C1-C6)alkoxy, and
R⁸ each independently represent a halogen atom, a cyano, a (C1-C6)haloalkyl, a (C3-C6)cycloalkyl, a (C1-C6)alkoxy, a (C1-C6)alkylcarbonyl, a (C1-C6)alkoxycarbonyl, a (C1-C6)haloalkoxycarbonyl, a (C6-C12)aryl (the (C6-C12)aryl is optionally mono-substituted or poly-substituted with R⁷), a 3- to 6-membered heterocyclyl (the 3- to 6-membered heterocyclyl is optionally mono-substituted or poly-substituted with R⁷), or a 5- to 12-membered heteroaryl (the 5- to 12-membered heteroaryl is optionally mono-substituted or poly-substituted with R⁷).

2. The formamide derivative of formula [I] or the agriculturally acceptable salt thereof according to claim 1, wherein in the formula [I], R² represents a (C1-C6)alkyl (the (C1-C6)alkyl is optionally mono-substituted or poly-substituted with R⁴), a (C3-C6)cycloalkyl (the (C3-C6)cycloalkyl is optionally mono-substituted or poly-substituted with R⁴), or a 3- to 6-membered heterocyclyl (the 3- to 6-membered heterocyclyl is optionally mono-substituted or poly-substituted with R⁵), and
R⁴ each independently represent a (C3-C6)cycloalkyl, a (C3-C6)halocycloalkyl, a (C6-C12)aryl (the (C6-C12)aryl is optionally mono-substituted or poly-substituted with R⁵), a 3- to 6-membered heterocyclyl (the 3- to 6-membered heterocyclyl is optionally mono-substituted or poly-substituted with R⁵), a 5- to 12-membered heteroaryl (the 5- to 12-membered heteroaryl is optionally mono-substituted or poly-substituted with R⁵), or a 1,3-benzodioxole (the 1,3-benzodioxole is optionally mono-substituted or poly-substituted with R⁵).

3. The formamide derivative of formula [I] or the agriculturally acceptable salt thereof according to claim 2, wherein in the formula [I], R² represents a (C1-C6)alkyl (the (C1-C6)alkyl is optionally mono-substituted or poly-substituted with R⁴), and
R⁵ each independently represent a hydrogen atom, a halogen atom, a cyano, a nitro, a formyl, an oxo, a (C1-C6)alkyl, a (C1-C6)haloalkyl, a (C1-C6)alkoxy, a (C1-C6)haloalkoxy, a (C1-C6)alkoxy (C1-C6)alkyl, or a (C1-C6)alkoxycarbonyl.

4. The formamide derivative of formula [I] or the agriculturally acceptable salt thereof according to claim 1, wherein in the formula [I], R⁴ each independently represent a halogen atom, a cyano, a thiocyanato, a thiol, a hydroxy, an amino, a tri((C1-C6)alkyl)silyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6)cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the(C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl group.

5. The formamide derivative of formula [I] or the agriculturally acceptable salt thereof according to claim 4, wherein in the formula [I], R² represents a hydrogen atom, a (C1-C6)alkyl (the (C1-C6)alkyl is optionally mono-substituted or poly-substituted with R⁴), a (C2-C6)alkenyl (the (C2-C6)alkenyl is optionally mono-substituted or poly-substituted with R⁴), a (C2-C6)alkynyl (the (C2-C6)alkynyl is optionally mono-substituted or poly-substituted with R⁴), a (C6-C12)aryl (the (C6-C12)aryl is optionally mono-substituted or poly-substituted with R⁵), or a 3- to 6-membered heterocyclyl (the 3- to 6-membered heterocyclyl is optionally mono-substituted or poly-substituted with R⁵),
R⁴ each independently represent a halogen atom, a cyano, a thiol, a hydroxy, a tri((C1-C6)alkyl)silyl, a (C1-C6)alkylamino (the (C1-C6)alkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C1-C6)alkylcarbonyloxy (the (C1-C6)alkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C1-C6)alkoxycarbonyloxy (the (C1-C6)alkoxycarbonyloxy is optionally mono-substituted or poly-substituted with R⁸), a hydrazinocarbonyl (the hydrazino moiety is optionally substituted with R⁶), or a pentafluorosulfanyl,
R⁵ each independently represent a hydrogen atom, a halogen atom, a cyano, a nitro, a formyl, an oxo, a (C1-C6)alkyl, a (C1-C6)haloalkyl, a (C1-C6)alkoxy, a (C1-C6)haloalkoxy, a (C1-C6)alkoxy (C1-C6)alkyl, or a (C1-C6)alkoxycarbonyl, and
R⁶ each independently represent a hydrogen atom, a (C1-C6)alkyl, a (C1-C6)haloalkylcarbonyl, or a (C1-C6)alkoxycarbonyl.

6. The formamide derivative of formula [I] or the agriculturally acceptable salt thereof according to claim 2, wherein in the formula [I], X¹, X², X³, and X⁴ each independently represent a carbon atom (the carbon atom is optionally substituted with R³), and R¹ is a halogen atom.

7. The formamide derivative of formula [I] or the agriculturally acceptable salt thereof according to claim 2, wherein in the formula [I], R³ is a hydrogen atom.

8. The formamide derivative of formula [I] or the agriculturally acceptable salt thereof according to claim 2, wherein in the formula [I], R² represents a (C1-C6)alkyl mono-substituted or poly-substituted with substituent(s) selected from a substituent group R⁴.

9. The formamide derivative of formula [I] or the agriculturally acceptable salt thereof according to claim 4, wherein in the formula [I], X¹, X², X³, and X⁴ each independently represent a carbon atom (the carbon atom is optionally substituted with R³), and R¹ is a halogen atom.

10. The formamide derivative of formula [I] or the agriculturally acceptable salt thereof according to claim 4, wherein in General Formula [I], R³ is a hydrogen atom.

11. The formamide derivative of formula [I] or the agriculturally acceptable salt thereof according to claim 4, wherein in the formula [I], R² represents a hydrogen atom, a (C1-C6)alkyl (the (C1-C6)alkyl is optionally mono-substituted or poly-substituted with R⁴), a (C2-C6)alkenyl (the (C2-C6)alkenyl is optionally mono-substituted or poly-substituted with R⁴), a (C2-C6)alkynyl (the (C2-C6)alkynyl is optionally mono-substituted or poly-substituted with R⁴), a (C3-C6)cycloalkyl (the (C3-C6)cycloalkyl is optionally mono-substituted or poly-substituted with R⁴), a (C6-C12)aryl (the (C6-C12)aryl is optionally mono-substituted or poly-substituted with R⁵), or a 5- to 12-membered heteroaryl (the 5- to 12-membered heteroaryl is optionally mono-substituted or poly-substituted with R⁵).

12. The formamide derivative of formula [I] or the agriculturally acceptable salt thereof according to claim 4, wherein in the formula [I], R² represents a hydrogen atom or a (C1-C6)alkyl mono-substituted or poly-substituted with substituent(s) selected from a substituent group R⁴.

13. An agricultural and horticultural plant disease control agent comprising the formamide derivative of formula [I] or the agriculturally acceptable salt thereof according to claim 1 as an active ingredient.

14. The agricultural and horticultural plant disease control agent according to claim 13, wherein phytopathogenic fungi are basidiomycetes, ascomycetes, deuteromycetes, oomycetes, and/or zygomycetes.

15. An agricultural and horticultural plant disease control agent composition comprising the formamide derivative of formula [I] or the agriculturally acceptable salt thereof according to claim 1, and an agriculturally acceptable carrier.

16. A method for controlling a disease of a useful plant, the method comprising a step of spraying an effective amount of the formamide derivative of formula [I] or the agriculturally acceptable salt thereof according to claim 1 on the useful plant, a part of the useful plant, or a place where the useful plant grows.

17. The method according to claim 16, wherein phytopathogenic fungi are basidiomycetes, ascomycetes, deuteromycetes, oomycetes, and/or zygomycetes.

18. A use of the formamide derivative of formula [I] or the agriculturally acceptable salt thereof according to claim 1 as an agricultural and horticultural plant disease control agent for a useful plant.

19. An aniline derivative of formula [II] or an agriculturally acceptable salt thereof, 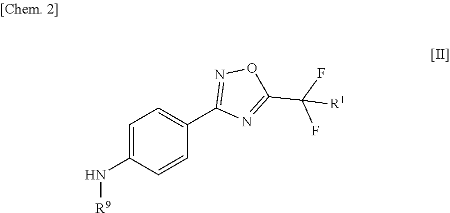
wherein in the formula [II],
R¹ represents a hydrogen atom or a halogen atom,
R⁹ represents a (C3-C6)alkyl, a (C1-C6)alkyl mono-substituted or poly-substituted with substituent(s) selected from a substituent group R¹⁰, a (C2-C6)alkenyl mono-substituted or poly-substituted with substituent(s) selected from a substituent group R¹¹, or a (C2-C6)alkynyl mono-substituted or poly-substituted with substituent(s) selected from a substituent group R¹²,
R¹⁰ and R¹² each independently represent a halogen atom, a cyano, a thiocyanato, a thiol, a hydroxy, an amino, a tri((C1-C6)alkyl)silyl, a (C3-C6)cycloalkyl, a (C3-C6)halocycloalkyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6)cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryl (the (C6-C12)aryl is optionally mono-substituted or poly-substituted with R⁵), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyl (the 3- to 6-membered heterocyclyl is optionally mono-substituted or poly-substituted with R⁵), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryl (the 5- to 12-membered heteroaryl is optionally mono-substituted or poly-substituted with R⁵), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a 1,3-benzodioxole (the 1,3-benzodioxole is optionally mono-substituted or poly-substituted with R⁵), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the(C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl,
R¹¹ each independently represent a halogen atom, a thiocyanato, a thiol, a hydroxy, an amino, a tri((C1-C6)alkyl)silyl, a (C3-C6)cycloalkyl, a (C3-C6)halocycloalkyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6)cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryl (the (C6-C12)aryl is optionally mono-substituted or poly-substituted with R⁵), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyl (the 3- to 6-membered heterocyclyl is optionally mono-substituted or poly-substituted with R⁵), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryl (the 5- to 12-membered heteroaryl is optionally mono-substituted or poly-substituted with R⁵), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a 1,3-benzodioxole (the 1,3-benzodioxole is optionally mono-substituted or poly-substituted with R⁵), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the(C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl,
R⁵ each independently represent a hydrogen atom, a halogen atom, a cyano, a nitro, an amino, a hydroxy, a thiol, a formyl, an oxo, a (C1-C6)alkyl, a (C2-C6)alkenyl, a (C2-C6)alkynyl, a (C3-C6)cycloalkyl, a (C1-C6)haloalkyl, a (C1-C6)alkoxy, a (C1-C6)haloalkoxy, a (C1-C6)alkoxy (C1-C6)alkyl, a (C1-C6)haloalkoxy (C1-C6)alkyl, a cyano (C1-C6)alkyl, a cyano (C3-C6)cycloalkyl, a (C1-C6)alkylthio, a (C1-C6)haloalkylthio, a (C1-C6)alkylsulfinyl, a (C1-C6)haloalkylsulfinyl, a (C1-C6)alkylsulfonyl, a (C1-C6)haloalkylsulfonyl, a (C1-C6)alkylcarbonylthio, a (C1-C6)alkylamino (the amino moiety is optionally substituted with R⁶), a (C1-C6)haloalkylamino (the amino moiety is optionally substituted with R⁶), a formylamino (the amino moiety is optionally substituted with R⁶), a (C1-C6)alkylcarbonylamino (the amino moiety is optionally substituted with R⁶), a carbamoyl, a (C1-C6)alkylaminocarbonyl (the amino moiety is optionally substituted with R⁶), a carboxyl, a (C1-C6)alkylcarbonyl, a (C1-C6)haloalkylcarbonyl, a (C1-C6)alkoxycarbonyl, a hydroxyimino (C1-C6)alkyl, a (C1-C6)alkoxyimino (C1-C6)alkyl, a (C6-C12)aryl (the (C6-C12)aryl is optionally mono-substituted or poly-substituted with R⁷), a (C6-C12)aryl (C1-C6)alkyl (the (C6-C12)aryl moiety is optionally mono-substituted or poly-substituted with R⁷), a (C6-C12)arylcarbonyl (the (C6-C12)arylcarbonyl is optionally mono-substituted or poly-substituted with R⁷), a (C6-C12)aryloxy (the (C6-C12)aryloxy is optionally mono-substituted or poly-substituted with R⁷), a (C6-C12)arylthio (the (C6-C12)arylthio is optionally mono-substituted or poly-substituted with R⁷), a (C6-C12)arylsulfinyl (the (C6-C12)arylsulfinyl is optionally mono-substituted or poly-substituted with R⁷), a (C6-C12)arylsulfonyl (the (C6-C12)arylsulfonyl is optionally mono-substituted or poly-substituted with R⁷), a (C6-C12)arylamino (the (C6-C12)aryl moiety is optionally mono-substituted or poly-substituted with R⁷, and the amino moiety is optionally substituted with R⁶), a 3- to 6-membered heterocyclyl (the 3- to 6-membered heterocyclyl is optionally mono-substituted or poly-substituted with R⁷), a 3- to 6-membered heterocyclyl (C1-C6)alkyl (the 3- to 6-membered heterocyclyl moiety is optionally mono-substituted or poly-substituted with R⁷), a 3- to 6-membered heterocyclylcarbonyl (the 3- to 6-membered heterocyclylcarbonyl is optionally mono-substituted or poly-substituted with R⁷), a 3- to 6-membered heterocyclyloxy (the 3- to 6-membered heterocyclyloxy is optionally mono-substituted or poly-substituted with R⁷), a 3- to 6-membered heterocyclylthio (the 3- to 6-membered heterocyclylthio is optionally mono-substituted or poly-substituted with R⁷), a 3- to 6-membered heterocyclylsulfinyl (the 3- to 6-membered heterocyclylsulfinyl is optionally mono-substituted or poly-substituted with R⁷), 3- to 6-membered heterocyclylsulfonyl (the 3- to 6-membered heterocyclylsulfonyl is optionally mono-substituted or poly-substituted with R⁷), a 3- to 6-membered heterocyclylamino (the 3- to 6-membered heterocyclyl moiety is optionally mono-substituted or poly-substituted with R⁷, and the amino moiety is optionally substituted with R⁶), a 5- to 12-membered heteroaryl (the 5- to 12-membered heteroaryl is optionally mono-substituted or poly-substituted with R⁷), a 5- to 12-membered heteroaryl (C1-C6)alkyl (the 5- to 12-membered heteroaryl moiety is optionally mono-substituted or poly-substituted with R⁷), a 5- to 12-membered heteroarylcarbonyl (the 5- to 12-membered heteroarylcarbonyl is optionally mono-substituted or poly-substituted with R⁷), a 5- to 12-membered heteroaryloxy (the 5- to 12-membered heteroaryloxy is optionally mono-substituted or poly-substituted with R⁷), a 5- to 12-membered heteroarylthio (the 5- to 12-membered heteroarylthio is optionally mono-substituted or poly-substituted with R⁷), a 5- to 12-membered heteroarylsulfinyl (the 5- to 12-membered heteroarylsulfinyl is optionally mono-substituted or poly-substituted with R⁷), a 5- to 12-membered heteroarylsulfonyl (the 5- to 12-membered heteroarylsulfonyl is optionally mono-substituted or poly-substituted with R⁷), or a 5- to 12-membered heteroarylamino (the 5- to 12-membered heteroaryl moiety is optionally mono-substituted or poly-substituted with R⁷, and the amino moiety is optionally substituted with R⁶),
R⁶ each independently represent a hydrogen atom, a (C1-C6)alkyl, a (C1-C6)haloalkyl, a (C3-C6)cycloalkyl, a (C1-C6)alkoxy (C1-C6)alkyl, a (C1-C6)haloalkoxy (C1-C6)alkyl, a cyano (C1-C6)alkyl, a (C1-C6)alkylcarbonyl, a (C1-C6)haloalkylcarbonyl, a (C1-C6)alkoxycarbonyl, or a (C6-C12)arylcarbonyl (the (C6-C12)arylcarbonyl is optionally mono-substituted or poly-substituted with R⁷),
R⁷ each independently represent a hydrogen atom, a halogen atom, a cyano, a (C1-C6)alkyl, or a (C1-C6)alkoxy, and
R⁸ each independently represent a halogen atom, a cyano, a (C1-C6)haloalkyl, a (C3-C6)cycloalkyl, a (C1-C6)alkoxy, a (C1-C6)alkylcarbonyl, a (C1-C6)alkoxycarbonyl, a (C1-C6)haloalkoxycarbonyl, a (C6-C12)aryl (the (C6-C12)aryl is optionally mono-substituted or poly-substituted with R⁷), a 3- to 6-membered heterocyclyl (the 3- to 6-membered heterocyclyl is optionally mono-substituted or poly-substituted with R⁷), or a 5- to 12-membered heteroaryl (the 5- to 12-membered heteroaryl is optionally mono-substituted or poly-substituted with R⁷).

20-23. (canceled)

24. An amidoxime derivative of formula [III] or an agriculturally acceptable salt thereof, 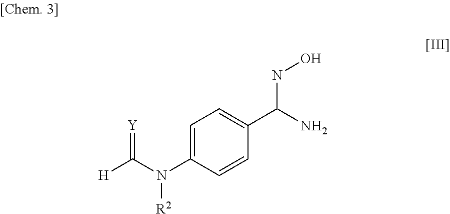
wherein in the formula [III],
Y represents an oxygen atom or a sulfur atom,
R² represents a hydrogen atom, a (C1-C6)alkyl (the (C1-C6)alkyl is optionally mono-substituted or poly-substituted with R⁴), a (C2-C6)alkenyl (the (C2-C6)alkenyl is optionally mono-substituted or poly-substituted with R⁴), a (C2-C6)alkynyl (the (C2-C6)alkynyl is optionally mono-substituted or poly-substituted with R⁴), a (C3-C6)cycloalkyl (the (C3-C6)cycloalkyl is optionally mono-substituted or poly-substituted with R⁴), a (C6-C12)aryl (the (C6-C12)aryl is optionally mono-substituted or poly-substituted with R⁵), a 3- to 6-membered heterocyclyl (the 3- to 6-membered heterocyclyl is optionally mono-substituted or poly-substituted with R⁵), or a 5- to 12-membered heteroaryl (the 5- to 12-membered heteroaryl is optionally mono-substituted or poly-substituted with R⁵),
R⁴ each independently represent a hydrogen atom, a halogen atom, a cyano, a thiocyanato, a thiol, a hydroxy, an amino, a tri((C1-C6)alkyl)silyl, a (C3-C6)cycloalkyl, a (C3-C6)halocycloalkyl, a (C1-C6)alkylamino (the (C1-C6)alkyl moiety is optionally mono-substituted or poly-substituted with R⁸, and the amino moiety is optionally substituted with R⁶), a (C2-C6)alkenylamino (the amino moiety is optionally substituted with R⁶), a (C2-C6)alkynylamino (the amino moiety is optionally substituted with R⁶), a (C3-C6)cycloalkylamino (the (C3-C6)cycloalkyl moiety is optionally mono-substituted or poly-substituted with R⁸, and the amino moiety is optionally substituted with R⁶), a formyl, a (C1-C6)alkylcarbonyl (the (C1-C6)alkylcarbonyl is optionally mono-substituted or poly-substituted with R⁸), a (C2-C6)alkenylcarbonyl, a (C2-C6)alkynylcarbonyl, a (C3-C6)cycloalkylcarbonyl (the (C3-C6)cycloalkylcarbonyl is optionally mono-substituted or poly-substituted with R⁸), a (C6-C12)arylcarbonyl (the (C6-C12)arylcarbonyl is optionally mono-substituted or poly-substituted with R⁵), a 3- to 6-membered heterocyclylcarbonyl (the 3- to 6-membered heterocyclylcarbonyl is optionally mono-substituted or poly-substituted with R⁵), a 5- to 12-membered heteroarylcarbonyl (the 5- to 12-membered heteroarylcarbonyl is optionally mono-substituted or poly-substituted with R⁵), a carboxyl, a (C1-C6)alkoxycarbonyl (the (C1-C6)alkoxycarbonyl is optionally mono-substituted or poly-substituted with R⁸), a (C2-C6)alkenyloxycarbonyl, a (C2-C6)alkynyloxycarbonyl, a (C3-C6)cycloalkyloxycarbonyl (the (C3-C6)cycloalkyloxycarbonyl is optionally mono-substituted or poly-substituted with R⁸), a (C6-C12)aryloxycarbonyl (the (C6-C12)aryloxycarbonyl is optionally mono-substituted or poly-substituted with R⁵), a 3- to 6-membered heterocyclyloxycarbonyl (the 3- to 6-membered heterocyclyloxycarbonyl is optionally mono-substituted or poly-substituted with R⁵), a 5- to 12-membered heteroaryloxycarbonyl (the 5- to 12-membered heteroaryloxycarbonyl is optionally mono-substituted or poly-substituted with R⁵), a carbamoyl, a (C1-C6)alkylaminocarbonyl (the (C1-C6)alkyl moiety is optionally mono-substituted or poly-substituted with R⁸, and the amino moiety is optionally substituted with R⁶), a (C2-C6)alkenylaminocarbonyl (the amino moiety is optionally substituted with R⁶), a (C2-C6)alkynylaminocarbonyl (the amino moiety is optionally substituted with R⁶), a (C3-C6)cycloalkylaminocarbonyl (the (C3-C6)cycloalkyl moiety is optionally mono-substituted or poly-substituted with R⁸, and the amino moiety is optionally substituted with R⁶), a (C6-C12)arylaminocarbonyl (the (C6-C12)aryl moiety is optionally mono-substituted or poly-substituted with R⁵, and the amino moiety is optionally substituted with R⁶), a 3- to 6-membered heterocyclylaminocarbonyl (the 3- to 6-membered heterocyclyl moiety is optionally mono-substituted or poly-substituted with R⁵, and the amino moiety is optionally substituted with R⁶), a 5- to 12-membered heteroarylaminocarbonyl (the 5- to 12-membered heteroaryl moiety is optionally mono-substituted or poly-substituted with R⁵, and the amino moiety is optionally substituted with R⁶), a formylamino (the amino moiety is optionally substituted with R⁶), a (C1-C6)alkoxycarbonylamino (the amino moiety is optionally substituted with R⁶), a (C1-C6)alkylcarbonylamino (the (C1-C6)alkyl moiety is optionally mono-substituted or poly-substituted with R⁸, and the amino moiety is optionally substituted with R⁶), a (C2-C6)alkenylcarbonylamino (the amino moiety is optionally substituted with R⁶), a (C2-C6)alkynylcarbonylamino (the amino moiety is optionally substituted with R⁶), a (C3-C6)cycloalkylcarbonylamino (the (C3-C6)cycloalkyl moiety is optionally mono-substituted or poly-substituted with R⁸, and the amino moiety is optionally substituted with R⁶), a (C6-C12)arylcarbonylamino (the (C6-C12)aryl moiety is optionally mono-substituted or poly-substituted with R⁵, and the amino moiety is optionally substituted with R⁶), a 3- to 6-membered heterocyclylcarbonylamino (the 3- to 6-membered heterocyclyl moiety is optionally mono-substituted or poly-substituted with R⁵, and the amino moiety is optionally substituted with R⁶), a 5- to 12-membered heteroarylcarbonylamino (the 5- to 12-membered heteroaryl moiety is optionally mono-substituted or poly-substituted with R⁵, and the amino moiety is optionally substituted with R⁶), a formyloxy, a (C1-C6)alkylcarbonyloxy (the (C1-C6)alkylcarbonyloxy is optionally mono-substituted or poly-substituted with R⁸), a (C2-C6)alkenylcarbonyloxy, a (C2-C6)alkynylcarbonyloxy, a (C3-C6)cycloalkylcarbonyloxy (the (C3-C6)cycloalkylcarbonyloxy is optionally mono-substituted or poly-substituted with R⁸), a (C6-C12)arylcarbonyloxy (the (C6-C12)arylcarbonyloxy is optionally mono-substituted or poly-substituted with R⁵), a 3- to 6-membered heterocyclylcarbonyloxy (the 3- to 6-membered heterocyclylcarbonyloxy is optionally mono-substituted or poly-substituted with R⁵), a 5- to 12-membered heteroarylcarbonyloxy (the 5- to 12-membered heteroarylcarbonyloxy is optionally mono-substituted or poly-substituted with R⁵), a (C1-C6)alkoxy (the (C1-C6)alkoxy is optionally mono-substituted or poly-substituted with R⁸), a (C1-C6)haloalkoxy, a (C2-C6)alkenyloxy, a (C2-C6)alkynyloxy, a (C3-C6)cycloalkyloxy (the (C3-C6)cycloalkyloxy is optionally mono-substituted or poly-substituted with R⁸), a (C1-C6)alkylsulfonylamino (the (C1-C6)alkyl moiety is optionally mono-substituted or poly-substituted with R⁸, and the amino moiety is optionally substituted with R⁶), a (C2-C6)alkenylsulfonylamino (the amino moiety is optionally substituted with R⁶), a (C2-C6)alkynylsulfonylamino (the amino moiety is optionally substituted with R⁶), a (C3-C6)cycloalkylsulfonylamino (the (C3-C6)cycloalkyl moiety is optionally mono-substituted or poly-substituted with R⁸, and the amino moiety is optionally substituted with R⁶), a (C6-C12)arylsulfonylamino (the (C6-C12)aryl moiety is optionally mono-substituted or poly-substituted with R⁵, and the amino moiety is optionally substituted with R⁶), a 3- to 6-membered heterocyclylsulfonylamino (the 3- to 6-membered heterocyclyl moiety is optionally mono-substituted or poly-substituted with R⁵, and the amino moiety is optionally substituted with R⁶), a 5- to 12-membered heteroarylsulfonylamino (the 5- to 12-membered heteroaryl moiety is optionally mono-substituted or poly-substituted with R⁵, and the amino moiety is optionally substituted with R⁶), a (C1-C6)alkylaminosulfonyl (the (C1-C6)alkyl moiety is optionally mono-substituted or poly-substituted with R⁸, and the amino moiety is optionally substituted with R⁶), a (C2-C6)alkenylaminosulfonyl (the amino moiety is optionally substituted with R⁶), a (C2-C6)alkynylaminosulfonyl (the amino moiety is optionally substituted with R⁶), a (C3-C6)cycloalkylaminosulfonyl (the (C3-C6)cycloalkyl moiety is optionally mono-substituted or poly-substituted with R⁸, and the amino moiety is optionally substituted with R⁶), a (C6-C12)arylaminosulfonyl (the (C6-C12)aryl moiety is optionally mono-substituted or poly-substituted with R⁵, and the amino moiety is optionally substituted with R⁶), a 3- to 6-membered heterocyclylaminosulfonyl (the 3- to 6-membered heterocyclyl moiety is optionally mono-substituted or poly-substituted with R⁵, and the amino moiety is optionally substituted with R⁶), a 5- to 12-membered heteroarylaminosulfonyl (the 5- to 12-membered heteroaryl moiety is optionally mono-substituted or poly-substituted with R⁵, and the amino moiety is optionally substituted with R⁶), a (C1-C6)alkylcarbonylthio, a (C1-C6)alkylthio (the (C1-C6)alkylthio is optionally mono-substituted or poly-substituted with R⁸), a (C1-C6)alkylsulfinyl (the (C1-C6)alkylsulfinyl is optionally mono-substituted or poly-substituted with R⁸), a (C1-C6)alkylsulfonyl (the (C1-C6)alkylsulfonyl is optionally mono-substituted or poly-substituted with R⁸), a (C1-C6)haloalkylthio, a (C1-C6)haloalkylsulfinyl, a (C1-C6)haloalkylsulfonyl, a (C2-C6)alkenylthio, a (C2-C6)alkenylsulfinyl, a (C2-C6)alkenylsulfonyl, a (C2-C6)alkynylthio, a (C2-C6)alkynylsulfinyl, a (C2-C6)alkynylsulfonyl, a (C3-C6)cycloalkylthio (the (C3-C6)cycloalkylthio is optionally mono-substituted or poly-substituted with R⁸), a (C3-C6)cycloalkylsulfinyl (the (C3-C6)cycloalkylsulfinyl is optionally mono-substituted or poly-substituted with R⁸), a (C3-C6)cycloalkylsulfonyl (the (C3-C6)cycloalkylsulfonyl is optionally mono-substituted or poly-substituted with R⁸), a (C6-C12)aryl (the (C6-C12)aryl is optionally mono-substituted or poly-substituted with R⁵), a (C6-C12)aryloxy (the (C6-C12)aryloxy is optionally mono-substituted or poly-substituted with R⁵), a (C6-C12)arylthio (the (C6-C12)arylthio is optionally mono-substituted or poly-substituted with R⁵), a (C6-C12)arylsulfinyl (the (C6-C12)arylsulfinyl is optionally mono-substituted or poly-substituted with R⁵), a (C6-C12)arylsulfonyl (the (C6-C12)arylsulfonyl is optionally mono-substituted or poly-substituted with R⁵), a (C6-C12)arylamino (the (C6-C12)aryl moiety is optionally mono-substituted or poly-substituted with R⁵, and the amino moiety is optionally substituted with R⁶), a 3- to 6-membered heterocyclyl (the 3- to 6-membered heterocyclyl is optionally mono-substituted or poly-substituted with R⁵), a 3- to 6-membered heterocyclyloxy (the 3- to 6-membered heterocyclyloxy is optionally mono-substituted or poly-substituted with R⁵), a 3- to 6-membered heterocyclylthio (the 3- to 6-membered heterocyclylthio is optionally mono-substituted or poly-substituted with R⁵), a 3- to 6-membered heterocyclylsulfinyl (the 3- to 6-membered heterocyclylsulfinyl is optionally mono-substituted or poly-substituted with R⁵), a 3- to 6-membered heterocyclylsulfonyl (the 3- to 6-membered heterocyclylsulfonyl is optionally mono-substituted or poly-substituted with R⁵), a 3- to 6-membered heterocyclylamino (the 3- to 6-membered heterocyclyl moiety is optionally mono-substituted or poly-substituted with R⁵, and the amino moiety is optionally substituted with R⁶), a 5- to 12-membered heteroaryl (the 5- to 12-membered heteroaryl is optionally mono-substituted or poly-substituted with R⁵), a 5- to 12-membered heteroaryloxy (the 5- to 12-membered heteroaryloxy is optionally mono-substituted or poly-substituted with R⁵), a 5- to 12-membered heteroarylthio (the 5- to 12-membered heteroarylthio is optionally mono-substituted or poly-substituted with R⁵), a 5- to 12-membered heteroarylsulfinyl (the 5- to 12-membered heteroarylsulfinyl is optionally mono-substituted or poly-substituted with R⁵), a 5- to 12-membered heteroarylsulfonyl (the 5- to 12-membered heteroarylsulfonyl is optionally mono-substituted or poly-substituted with R⁵), a 5- to 12-membered heteroarylamino (the 5- to 12-membered heteroaryl moiety is optionally mono-substituted or poly-substituted with R⁵, and the amino moiety is optionally substituted with R⁶), a 1,3-benzodioxole (the 1,3-benzodioxole is optionally mono-substituted or poly-substituted with R⁵), a hydroxyimino (C1-C6)alkyl, a (C1-C6)alkoxyimino (C1-C6)alkyl (the (C1-C6)alkoxy moiety is optionally mono-substituted or poly-substituted with R⁸), a (C2-C6)alkenyloxyimino (C1-C6)alkyl, a (C2-C6)alkynyloxyimino (C1-C6)alkyl, a (C3-C6)cycloalkyloxyimino (C1-C6)alkyl (the (C3-C6)cycloalkyl moiety is optionally mono-substituted or poly-substituted with R⁸), a (C6-C12)aryloxyimino (C1-C6)alkyl (the (C6-C12)aryl moiety is optionally mono-substituted or poly-substituted with R⁵), a 3- to 6-membered heterocyclyloxyimino (C1-C6)alkyl (the 3- to 6-membered heterocyclyl moiety is optionally mono-substituted or poly-substituted with R⁵), a 5- to 12-membered heteroaryloxyimino (C1-C6)alkyl (the 5- to 12-membered heteroaryl moiety is optionally mono-substituted or poly-substituted with R⁵), a (C1-C6)alkoxycarbonyloxy (the (C1-C6)alkoxycarbonyloxy is optionally mono-substituted or poly-substituted with R⁸), a (C2-C6)alkenyloxycarbonyloxy, a (C2-C6)alkenyloxycarbonyloxy, a (C3-C6)cycloalkyloxycarbonyloxy (the (C3-C6)cycloalkyloxycarbonyloxy is optionally mono-substituted or poly-substituted with R⁸), a (C6-C12)aryloxycarbonyloxy (the(C6-C12)aryloxycarbonyloxy is optionally mono-substituted or poly-substituted with R⁵), a 3- to 6-membered heterocyclyloxycarbonyloxy (the 3- to 6-membered heterocyclyloxycarbonyloxy is optionally mono-substituted or poly-substituted with R⁵), a 5- to 12-membered heteroaryloxycarbonyloxy (the 5- to 12-membered heteroaryloxycarbonyloxy is optionally mono-substituted or poly-substituted with R⁵), a (C1-C6)alkylaminocarbonylamino (the (C1-C6)alkyl moiety is optionally mono-substituted or poly-substituted with R⁸, and the amino moiety is optionally substituted with R⁶), a (C2-C6)alkenylaminocarbonylamino (the amino moiety is optionally substituted with R⁶), a (C2-C6)alkynylaminocarbonylamino (the amino moiety is optionally substituted with R⁶), a (C3-C6)cycloalkylaminocarbonylamino (the (C3-C6)cycloalkyl moiety is optionally mono-substituted or poly-substituted with R⁸, and the amino moiety is optionally substituted with R⁶), a (C6-C12)arylaminocarbonylamino (the (C6-C12)aryl moiety is optionally mono-substituted or poly-substituted with R⁵, and the amino moiety is optionally substituted with R⁶), a 3- to 6-membered heterocyclylaminocarbonylamino (the 3- to 6-membered heterocyclyl moiety is optionally mono-substituted or poly-substituted with R⁵, and the amino moiety is optionally substituted with R⁶), a 5- to 12-membered heteroarylaminocarbonylamino (the 5- to 12-membered heteroaryl moiety is optionally mono-substituted or poly-substituted with R⁵, and the amino moiety is optionally substituted with R⁶), a hydrazinocarbonyl (the hydrazino moiety is optionally substituted with R⁶), or a pentafluorosulfanyl,
R⁵ each independently represent a hydrogen atom, a halogen atom, a cyano, a nitro, an amino, a hydroxy, a thiol, a formyl, an oxo, a (C1-C6)alkyl, a (C2-C6)alkenyl, a (C2-C6)alkynyl, a (C3-C6)cycloalkyl, a (C1-C6)haloalkyl, a (C1-C6)alkoxy, a (C1-C6)haloalkoxy, a (C1-C6)alkoxy (C1-C6)alkyl, a (C1-C6)haloalkoxy (C1-C6)alkyl, a cyano (C1-C6)alkyl, a cyano (C3-C6)cycloalkyl, a (C1-C6)alkylthio, a (C1-C6)haloalkylthio, a (C1-C6)alkylsulfinyl, a (C1-C6)haloalkylsulfinyl, a (C1-C6)alkylsulfonyl, a (C1-C6)haloalkylsulfonyl, a (C1-C6)alkylcarbonylthio, a (C1-C6)alkylamino (the amino moiety is optionally substituted with R⁶), a (C1-C6)haloalkylamino (the amino moiety is optionally substituted with R⁶), a formylamino (the amino moiety is optionally substituted with R⁶), a (C1-C6)alkylcarbonylamino (the amino moiety is optionally substituted with R⁶), a carbamoyl, a (C1-C6)alkylaminocarbonyl (the amino moiety is optionally substituted with R⁶), a carboxyl, a (C1-C6)alkylcarbonyl, a (C1-C6)haloalkylcarbonyl, a (C1-C6)alkoxycarbonyl, a hydroxyimino (C1-C6)alkyl, a (C1-C6)alkoxyimino (C1-C6)alkyl, a (C6-C12)aryl (the (C6-C12)aryl is optionally mono-substituted or poly-substituted with R⁷), a (C6-C12)aryl (C1-C6)alkyl (the (C6-C12)aryl moiety is optionally mono-substituted or poly-substituted with R⁷), a (C6-C12)arylcarbonyl (the (C6-C12)arylcarbonyl is optionally mono-substituted or poly-substituted with R⁷), a (C6-C12)aryloxy (the (C6-C12)aryloxy is optionally mono-substituted or poly-substituted with R⁷), a (C6-C12)arylthio (the (C6-C12)arylthio is optionally mono-substituted or poly-substituted with R⁷), a (C6-C12)arylsulfinyl (the (C6-C12)arylsulfinyl is optionally mono-substituted or poly-substituted with R⁷), a (C6-C12)arylsulfonyl (the (C6-C12)arylsulfonyl is optionally mono-substituted or poly-substituted with R⁷), a (C6-C12)arylamino (the (C6-C12)aryl moiety is optionally mono-substituted or poly-substituted with R⁷, and the amino moiety is optionally substituted with R⁶), a 3- to 6-membered heterocyclyl (the 3- to 6-membered heterocyclyl is optionally mono-substituted or poly-substituted with R⁷), a 3- to 6-membered heterocyclyl (C1-C6)alkyl (the 3- to 6-membered heterocyclyl moiety is optionally mono-substituted or poly-substituted with R⁷), a 3- to 6-membered heterocyclylcarbonyl (the 3- to 6-membered heterocyclylcarbonyl is optionally mono-substituted or poly-substituted with R⁷), a 3- to 6-membered heterocyclyloxy (the 3- to 6-membered heterocyclyloxy is optionally mono-substituted or poly-substituted with R⁷), a 3- to 6-membered heterocyclylthio (the 3- to 6-membered heterocyclylthio is optionally mono-substituted or poly-substituted with R⁷), a 3- to 6-membered heterocyclylsulfinyl (the 3- to 6-membered heterocyclylsulfinyl is optionally mono-substituted or poly-substituted with R⁷), 3- to 6-membered heterocyclylsulfonyl (the 3- to 6-membered heterocyclylsulfonyl is optionally mono-substituted or poly-substituted with R⁷), a 3- to 6-membered heterocyclylamino (the 3- to 6-membered heterocyclyl moiety is optionally mono-substituted or poly-substituted with R⁷, and the amino moiety is optionally substituted with R⁶), a 5- to 12-membered heteroaryl (the 5- to 12-membered heteroaryl is optionally mono-substituted or poly-substituted with R⁷), a 5- to 12-membered heteroaryl (C1-C6)alkyl (the 5- to 12-membered heteroaryl moiety is optionally mono-substituted or poly-substituted with R⁷), a 5- to 12-membered heteroarylcarbonyl (the 5- to 12-membered heteroarylcarbonyl is optionally mono-substituted or poly-substituted with R⁷), a 5- to 12-membered heteroaryloxy (the 5- to 12-membered heteroaryloxy is optionally mono-substituted or poly-substituted with R⁷), a 5- to 12-membered heteroarylthio (the 5- to 12-membered heteroarylthio is optionally mono-substituted or poly-substituted with R⁷), a 5- to 12-membered heteroarylsulfinyl (the 5- to 12-membered heteroarylsulfinyl is optionally mono-substituted or poly-substituted with R⁷), a 5- to 12-membered heteroarylsulfonyl (the 5- to 12-membered heteroarylsulfonyl is optionally mono-substituted or poly-substituted with R⁷), or a 5- to 12-membered heteroarylamino (the 5- to 12-membered heteroaryl moiety is optionally mono-substituted or poly-substituted with R⁷, and the amino moiety is optionally substituted with R⁶),
R⁶ each independently represent a hydrogen atom, a (C1-C6)alkyl, a (C1-C6)haloalkyl, a (C3-C6)cycloalkyl, a (C1-C6)alkoxy (C1-C6)alkyl, a (C1-C6)haloalkoxy (C1-C6)alkyl, a cyano (C1-C6)alkyl, a (C1-C6)alkylcarbonyl, a (C1-C6)haloalkylcarbonyl, a (C1-C6)alkoxycarbonyl, or a (C6-C12)arylcarbonyl (the (C6-C12)arylcarbonyl is optionally mono-substituted or poly-substituted with R⁷),
R⁷ each independently represent a hydrogen atom, a halogen atom, a cyano, a (C1-C6)alkyl, or a (C1-C6)alkoxy, and
R⁸ each independently represent a halogen atom, a cyano, a (C1-C6)haloalkyl, a (C3-C6)cycloalkyl, a (C1-C6)alkoxy, a (C1-C6)alkylcarbonyl, a (C1-C6)alkoxycarbonyl, a (C1-C6)haloalkoxycarbonyl, a (C6-C12)aryl (the (C6-C12)aryl is optionally mono-substituted or poly-substituted with R⁷), a 3- to 6-membered heterocyclyl (the 3- to 6-membered heterocyclyl is optionally mono-substituted or poly-substituted with R⁷), or a 5- to 12-membered heteroaryl (the 5- to 12-membered heteroaryl is optionally mono-substituted or poly-substituted with R⁷).

25-28. (canceled)
